(19) United States
(12) Patent Application Publication
(10) Pub. No.: US 2013/0309209 A1
(43) Pub. Date: Nov. 21, 2013

(54) FORMATION OF HEMATOPOIETIC PROGENITOR CELLS FROM MESENCHYMAL STEM CELLS

(71) Applicants: Juan Carlos Izpisua-Belmonte, La Jolla, CA (US); Emmanuel Nivet, San Diego, CA (US); Ignacio Sancho-Martinez, San Diego, CA (US); Leo Kurian, San Diego, CA (US); Julian Andres Pulecio Rojas, Barcelona (ES)

(72) Inventors: Juan Carlos Izpisua-Belmonte, La Jolla, CA (US); Emmanuel Nivet, San Diego, CA (US); Ignacio Sancho-Martinez, San Diego, CA (US); Leo Kurian, San Diego, CA (US); Julian Andres Pulecio Rojas, Barcelona (ES)

(73) Assignees: Center for Regenerative Medicine of Barcelona, Barcelona (ES); The Salk Institute for Biological Studies, La Jolla, CA (US)

(21) Appl. No.: 13/880,677

(22) PCT Filed: Oct. 24, 2011
(86) PCT No.: PCT/US11/57542

§ 371 Date: Jul. 19, 2013

Related U.S. Application Data
(60) Provisional application No. 61/406,062, filed on Oct. 22, 2010.
(60) Provisional application No. 61/438,326, filed on Feb. 1, 2011.

(51) Int. Cl. C12N 5/0789

(52) U.S. Cl. CPC C12N 5/0647
USPC 424/93.7; 435/377; 435/372

(57) ABSTRACT

There are provided, inter alia, methods for forming (e.g. transgeneration of) hematopoietic stem cells from mesenchymal stem cells.

### CROSS-REFERENCES TO RELATED APPLICATIONS

This application claims the benefit of U.S. Provisional Appl. No. 61/406,062, filed Oct. 22, 2010, and U.S. Provisional Appl. No. 61/438,326, filed Feb. 1, 2011, all of which are hereby incorporated in their entirety and for all purposes.

### BACKGROUND OF THE INVENTION

Pluripotent cells, such as progenitor cells and stem cells, are increasingly desired for regenerative therapies for disorders such as diabetes, neutropenia, and Alzheimer's Disease, to name but a few. Forming pluripotent cells using the standard technology applied to induced pluripotent stem (iPS) cells raises serious safety concerns regarding the safe use of genetically modified cells in a clinical setting. The possibility of reprogramming cells towards an iPS state in the absence of integrative approaches would thus represent an advance in the safe application of iPS. Yet current techniques are often time-consuming, risky, and result in low efficiency of reprogramming. The methods provided herein cure these and other defects in the art.

Mesenchymal Stem Cells (MSCs) present several characteristics making them an attractive cell population for cell-therapy. For example, MSCs are “immune privileged.”Thus, MSCs are less likely to cause Graft Versus Host Disease or require immunosuppressants for cell therapy regimens. MSCs are readily available from a variety of adult tissues (e.g., olfactory, bone, adipose, bone marrow) allowing for autologous transplantation without the need for highly invasive techniques. Indeed, MSC cultures can be established by methods known in the art, and the high proliferation rate allows for rapid expansion of initial cultures. Moreover, MSCs have been shown to be an adequate cell source for differentiation into a variety of different cell types including e.g., osteocytes, chondrocytes, smooth muscle, cardiomyocytes and adipocytes.

Direct lineage conversion could represent a complementary approach to iPS technology. Both iPS technology and lineage conversion build on the knowledge of the signaling pathways involved in lineage commitment. Direct lineage conversion (i.e., transdifferentiation, transgeneration and/or transdetermination) does not involve reversion towards a pluripotent state, and thus reduces the time required for obtaining the desired cell types. In addition, the absence of pluripotent stem cells during transplantation reduces cancer risk associated with such self-renewing cells. Thus, there is a need in the art for forming hematopoietic through direct lineage conversion a complementary approach to iPS technology. Provided herein, inter alia, a methods and materials for forming hematopoietic progenitor cells (HPCs) from mesenchymal stem cells (MSCs) thereby solving these and other needs in the art.

### BRIEF SUMMARY OF THE INVENTION

Presented herein are, inter alia, methods, compositions and kits of forming hematopoietic progenitor cells (HPCs) from mesenchymal stem cells (MSCs). In one aspect, a method of forming a HPC is provided. The method includes contacting a mesenchymal stem cell (MSC) with a SOX2 signaling agonist and allowing the MSC to form a HPC.

In another aspect, a method of forming a hematopoietic progenitor cell (HPC) is provided. The methods includes transducing a mesenchymal stem cell (MSC) with a SOX2 protein or a SOX2 nucleic acid and allowing the MSC to form a HPC.

In another aspect, a kit for forming a HPC is provided. The kit includes a MSC, a SOX2 signaling agonist and instructions to culture the MSC under conditions suitable for forming a HPC.

In another aspect, a kit for forming a HPC is provided. The kit includes a MSC, a SOX2 protein or a SOX2 nucleic acid and instructions to culture the MSC under conditions suitable for forming a HPC.

In another aspect, a kit for forming a HPC is provided. The kit includes a SOX2 protein or a SOX2 nucleic acid, a SOX2 signaling agonist, and instructions to administer the components of to a cell under conditions suitable for forming a HPC.

In one aspect, a mesenchymal stem cell including a SOX2 signaling agonist is provided.

In another aspect, a mesenchymal stem cell including a SOX2 protein or a SOX2 nucleic acid is provided.

### BRIEF DESCRIPTION OF THE DRAWINGS

FIG. 1. Sox2 directly converts human MSCs into HPCs bypassing iPS generation. (FIG. 1A) Representative bright-field pictures showing the different morphologies between HPCs and iPS colonies. (FIG. 1B) Standard iPS procedures allow for the efficient generation of CD34+ HPCs. (FIG. 1C) For each group of the histogram the entries depicted from left to right are KM (Klf4, cMyc); SK (Sox2, Klf4); SM (Sox2, cMyc); M (cMyc); K (Klf4); and S (Sox2). Legend: CD34 (filled); CD45 (diagonal stripe lower left to upper right); CD34/CD45 (diagonal stripe upper left to lower right). iPS non-permissive conditions in the absence of Oct4 do not impair HPC generation. (FIG. 1D) Schematic representation of the transdetermination procedure. Feature legend: 101=human MSCs; 102=Sox2 retroviral transduction; 103=resting day; 104=media switch; 105=Day −1; 106=Day 0, 1; 107=Day 2; 108=Day 3-8. (FIG. 1E) MSCs derived from different tissues present different transdetermination potential. For each group of the histogram the entries depicted from left to right are OE-MSCs, AT-MSCs, UC-MSCs, and BM-MSCs. Legend: CD34 (filled); CD45 (diagonal stripe lower left to upper right); CD34/CD45 (diagonal stripe upper left to lower right). (FIG. 1F) Representative plots showing expression of the early progenitor marker CD34 and the pan-hematopoietic marker CD45. (FIG. 1G) RNA levels show strong upregulation of hematopoietic markers and demonstrate the non-pluripotent nature of the transdetermined HPCs. Legend: hematopoiesis-related markers (filled); hematopoiesis-unrelated markers (unfilled). (FIG. 1H) mRNA expression levels show the non-pluripotent nature of transdetermined HPCs.

FIG. 2. Transdetermined HPCs can be rapidly obtained and expanded in vitro. (FIG. 2A) OE-MSCs rapidly transdetermine towards a hematopoietic fate. Kinetics of expression of hemtapoietic markers is shown. (FIG. 2B and FIG. 2C) Representative contour plots of the transdetermination process. Feature legend: 201=day 2 isotype; 202=day 4 isoptype; 203=day 6 isotype; 204=day 8 isotype; 205=day 2; 206=day 4; 207=day6; 208=day 8. (FIG. 2D) OE-MSC transdetermination generates different populations of hemtapoietic progenitors representing the transition from early CD43+ to late CD43-HPCs. For each group of the histogram the entries are depicted day 2, 4, 6, 8 and 10 in the order left to right. Legend: CD34 (filled); CD45 (diagonal stripe lower left to upper right); CD34/CD45 (diagonal stripe upper left to lower right). (FIG. 2E) In vitro generated HPCS show multilineagepotential and are able to generate all major blood lineages in colony forming assays. (FIG. 2F) May Grunwald/Giemsa stained images of CFU/BFU-derived cells. (FIG. 2G) Cell proliferation studies showing homogenous reduction of CFSE fluorescence intensity on both transdetermined MSCs and transdetermined CD34+ OE-MSCs demonstrates the proliferation capacity of transdetermined HPCs. Feature legend: 209=day 0; 210=day 2; 211=day 4; 212=day 1; 213=day 3; 214=day5. (FIG. 2H) Representative plots showing the relative percentages of CD34+ and CD34+/CD45+ cells with (right) or without (left) depletion of CD34+ cells. Feature legend: 215=day 10 post transduction; 216=isotype; 217=day 6 post depletion.

FIG. 3. Transdetermination of MSCs leads to global gene expression changes. (FIG. 3A) Heat-map showing significant hierarchical clustering of hematopoietic signature genes between OE-MSCs and transdetermined CD34+ OEMSCs by Pearson correlation. (FIG. 3B) Heat-map showing no significant clustering of pluripotency signature genes between OE-MSCs and transdetermined CD34+ OEMSCs by Pearson correlation. (FIG. 3C) Heat-map showing significant hierarchical clustering of homeostatic processes signature genes between OE-MSCs and transdetermined CD34+ OEMSCs by Pearson correlation. (FIG. 3D) Heat-map showing significant hierarchical clustering of signature genes related to T-cell activation between OE-MSCs and transdetermined CD34+ OEMSCs by Pearson correlation. (FIG. 3E) Upregulation of specific HPC markers during transdetermination faithfully recapitulates the progression through the different developmental stages and demonstrates the hematopoietic nature of the transdetermined cells. See also Tables 1-5. Data are represented as mean+/−STD. *p<0.05, **p<0.01, ***p<0.001.

FIG. 4. TGFβ signaling contributes to the generation of CD45+ cell populations. (FIG. 4A) Chronic inhibition of TGFβ signaling alone results in the generation of HPCs in the absence of integrative approaches. Percentages shown represent 8 days of transdetermination in OE-MSCs. (FIG. 4B) Chronic inhibition of TGFβ signaling is sufficient to upregulate endogenous Sox2 and Oct4. (FIG. 4C) Sox2 transduction in combination with TGFβRI inhibitor does not impair the generation of CD34+ cells and contributes to higher transdetermination over the first 4 days. Feature legend: 401=day 2; 402=day 4; 403=day 6; 404=day 8. (FIG. 4D) Sox2 transduction in combination with TGFβRI inhibitor shows significant reduction of newly generated CD45+ cells. Feature legend: 405=day 2; 406=day 4; 407=day 6; 408=day 8. Data are represented as mean+/−STD. *p<0.05, **p<0.01, ***p<0.001.

FIG. 5. Transdetermined HPCs can be obtained by safe non-integrative approaches. (FIG. 5A) Daily administration of 5 μg recombinant Sox2-TAT during five days allows for the generation of HPCs from OE-MSCs in the absence of integrative approaches. CD34 (filled); CD45 (diagonal stripe lower left to upper right); CD34/CD45 (diagonal stripe upper left to lower right). (FIG. 5B) Chronic inhibition of TGFβ signaling results in the generation of HPCs in the absence of integrative approaches. Percentages shown represent 4 days of transdetermination. CD34 (filled); CD45 (diagonal stripe lower left to upper right); CD34/CD45 (diagonal stripe upper left to lower right). (FIG. 5C) Chronic inhibition of TGFβ signaling provokes significant upregulation of HPC-related markers at the mRNA level as well as mRNA upregulation of hematopoietic-related transcription factors. Feature legend: 501=mRNA fold change (normalized control; 502=day 2; 503=day 4; 404=day 6; 505=day 7. (FIG. 5D) as compared to the observed basal levels. Feature legend: 501=mRNA fold change (normalized control; 502=day 2; 503=day 4; 404=day 6; 505=day 7. (FIG. 5E) Chronic inhibition of TGFβ signaling in combination with Sox2 transduction significantly abolishes generation of CD34+CD45+ and CD45+ cell populations. Percentages shown represent 4 days of transdetermination. CD34 (filled); CD45 (diagonal stripe lower left to upper right); CD34/CD45 (diagonal stripe upper left to lower right). Feature legend: 506=% of positive cells; 507=SOX2 and DMSO; 508=SOX2 and SB431542. (FIG. 5F) Inhibition of TGFβ signaling blocks the progression towards more mature HPCs stages whereas ERK inhibition results in the efficient generation of CD34+ HPCs. (FIG. 5G) Upregulation of specific HPC markers during transdetermination faithfully recapitulates developmental stages. Of note is the strong repression observed upon TGFβ inhibition. Data are represented as mean+/−STD. *p<0.05, **p<0.01, ***p<0.001.

FIG. 6. Representative model showing the relative contribution of different transcription factors to the transdetermination process. Exogenous Sox2 or chronic inhibition of TGFβ signaling upregulate endogenous Sox2 and components of the TGFβ signaling pathway. Later on, inhibition of TGFβ signaling precludes the generation of CD45+ cells. Feature legend: 601=plasma membrane; 602=nuclear envelope; 603=SMADs; 604=Sox2; 605=exogenous Sox2; 606=SB 431542; 607=exogenous Sox2; 608=CD34; 609=CD45.

FIG. 7. Sox2 contributes to the generation of CD34+ in AT-MSCs and human Fibroblasts. (FIG. 7A) Transduction of Sox2 into human fibroblast results in the efficient generation of CD34+ cells whereas showing limited potential for the generation of CD45+ cells. CD34 (filled); CD45 (diagonal stripe lower left to upper right); CD34/CD45 (diagonal stripe upper left to lower right). (FIG. 7B) Kinetics of appearance of the different HPC populations during transdetermination of AT-MSCs. For each group of the histogram the entries are depicted day 2, 4, 6, 8 and 10 in the order left to right. CD34 (filled); CD45 (diagonal stripe lower left to upper right); CD34/CD45 (diagonal stripe upper left to lower right). (FIG. 7C) Transdetermined CD34+ cells do not show endothelial potential. On the left panel, OE- and AT-MSCs transduced with Sox2 were analyzed for surface expression of the endothelial marker CD31. Both lines show marginal expression of CD31. On the right panel, sorted CD34+ subjected to endothelial differentiation conditions failed to increase CD31 expression. (FIG. 7D) Representative pictures of hematopoietic colonies derived from transdetermined AT-MSCs. (FIG. 7E) Effect of the TGF beta and ERK inhibitors on the expression levels of CD34 and CD45. (FIG. 7F) Cell proliferation analysis showing homogenous reduction of the CFSE fluorescence intensity of both AT-MSCs and AT-MSCs-derived-CD34+. On the right panel, reduction of the mean fluorescence intensity over time is shown. Data are represented as mean+/−STD.

FIG. 8. MSCs spontaneously differentiate into the erythroid lineage in vitro. (FIG. 8A) Flow cytometry surface expression of the erythroid lineage marker CD235a in adipose tissue derived MSCs by overexpression of Sox2 (S), Sox2 plus KLF4 (SK) and Sox2 plus c-Myc (SM). A small percentage of CD235a positive cells spontaneously differentiate in long-term cultures of up to one month. (FIG. 8B) Expression of adult Hemoglobin assessed by western blot. (FIG. 8C) Brightfield photography of AT-MSC before (right panel) and 30 days after (left panel) Sox2 transduction.

FIG. 9. Optimization of the transdetermination procedure #1 by using additional transduction of miRNA(s) (#2) allows for the generation of HPCs able to repopulate the hematopoietic system after transplantation in mice. (FIG. 9A) Cartoon depicting the in vivo strategy used to assess the functionality of transdetermined cells after transplantation into irradiated NSG mice (2.5Gy). Feature legend: 901=mesenchymal stem cells; 902=transdifferentiation; 903=sorted cells; 904=irradiated mouse; 905=cell transplantation; 906=intrafemoral/intravenous injection; 907=short term reconstitution; 908=long term reconstitution; 909=4 weeks; 910=6 weeks; 911=8 weeks; 912=10 weeks; 913=12 weeks; 914=blood collection; 915=peripheral blood/bone marrow/spleen collection. (FIG. 9B) transdetermination procedure #2 involved an additional transduction step to overexpress HSC-related miRNA(s) 9 days after Sox2 transduction (see table4 for a complete list of the targeted miRNAs) and the use of the so called hematopoietic transdetermination media, before transplantation into irradiated NSG mice. Feature legend: 916=somatic cells; 917=Sox2/has-miR125b transduction; 918=hematopoietic transdifferentiation media; 919=intrafemural transplantation into sublethally irradiated NCG mice. (FIG. 9C) Transdetermination procedure #2 using the miRNA125b allow for the generation of non-adherent cells expressing the CD34 marker. (FIG. 9D) Ten weeks after transplantation of sorted CD34+ cells generated with the transdetermination procedure#2 (miR125b), injected cells show the capacity to repopulate the hematopoietic system. Human CD45+ cells were found in the three structure analyzed, i.e. peripheral blood, bone marrow and spleen. Percentages of human CD45+ cells are indicated for each structure.

### DETAILED DESCRIPTION OF THE INVENTION

### I. Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

A “hematopoietic progenitor cell (HPC)” or “hematopoietic stem cell (HSC)” is a self renewing pluripotent cell capable of ultimately differentiating into cell types of the hematopoietic system, including B cells T cells, NK cells, lymphoid dendritic cells, myeloid dendritic cells, granulocytes, macrophages, megakaryocytes, and erythroid cells. As with other cells of the hematopoietic system, HSCs are typically defined by the presence of a characteristic set of cell markers. In humans, HSCs are typically characterized as CD34+, though CD34 surface expression is not an absolute determinative factor. HPCs display a range of pluripotency and surface marker expression changes with increasing differentiation. Additional HPC markers are described herein. Descriptions of marker phenotypes for various hematopoietic and myeloid progenitor cells are also described in, for example, Metcalf (2007) Stem Cells 25:2390-95; U.S. Pat. Nos. 6,465,247 and 6,761,883; Akashi (2000) Nature 404: 193-97; and Manz (2002) Proc. Natl. Acad. Sd. USA 9911872-77.

HPCs give rise to committed lymphoid or myeloid progenitor (MP) cells. As used herein committed myeloid progenitor cells refer to cell populations capable of differentiating into any of the terminally differentiated cells of the myeloid lineage. Encompassed within the myeloid progenitor cells are the common myeloid progenitor cells (CMP), a cell population characterized by limited or non-self-renewal capacity but which is capable of cell division to form granulocyte/macrophage progenitor cells (GMP) and megakaryocyte/erythroid progenitor cells (MEP).

A mesenchymal stem cell (MSC) is a pluripotent cell that can differentiate into a number of different cell types. MSCs are commonly harvested from bone marrow, but can be found in and isolated from other tissues such as adipose, liver, olfactory, and fetal tissues. MSCs are heterogenous and express a number of cell surface markers. MSCs typically do not express CD34 or CD45, but can express CD105, CD73, CD44, CD90 (Thy-1), CD71, and CD106. See, e.g., Campagnoli et al. (2001) Blood 98:2396-2402.

The term “feeder-free,” refers to the absence of feeder cells. The term “feeder cell” is known in the art, and includes all cells used to support the propagation of stem cells, e.g., during the process of reprogramming Feeder cells can be irradiated prior to being co-cultured with the stem cells in order to avoid the feeder cells outgrowing the stem cells. Feeder cells provide a layer physical support for attachment, and produce growth factors and extracellular matrix proteins that support cells. Examples of feeder cells include fibroblasts (e.g., embryonic fibroblasts), splenocytes, macrophages and thymocytes.

The term “reprogramming” refers to the process of dedifferentiating a non-pluripotent cell into a cell exhibiting pluripotent stem cell characteristics.

The terms “transdetermination,” “transgeneration,” and “transdifferentiation” refer to generation of a cell of a certain lineage (e.g., a hematopoietic progenitor cell) from a different type of cell (e.g., a mesenchymal stem cell) without the intermediate reprogramming step.

A “stem cell” is a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

“Self renewal” refers to the ability of a cell to divide and generate at least one daughter cell with the self-renewing characteristics of the parent cell. The second daughter cell may commit to a particular differentiation pathway. For example, a self-renewing hematopoietic stem cell can divide and form one daughter stem cell and another daughter cell committed to differentiation in the myeloid or lymphoid pathway. A committed progenitor cell has typically lost the self-renewal capacity, and upon cell division produces two daughter cells that display a more differentiated (i.e., restricted) phenotype. Non-self renewing cells refers to cells that undergo cell division to produce daughter cells, neither of which have the differentiation potential of the parent cell type, but instead generates differentiated daughter cells.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells.

“Pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

The terms “induced pluripotent stem cell,” “iPS” and the like refer to a pluripotent stem cell artificially derived from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells.

An adult stem cell is an undifferentiated cell found in an individual after embryonic development. Adult stem cells multiply by cell division to replenish dying cells and regenerate damaged tissue. An adult stem cell has the ability to divide and create another cell like itself or to create a more differentiated cell. Even though adult stem cells are associated with the expression of pluripotency markers such as Rex1, Nanog, Oct4 or Sox2, they do not have the ability of pluripotent stem cells to differentiate into the cell types of all three germ layers. Adult stem cells have a limited ability to self renew and generate progeny of distinct cell types. Adult stem cells can include hematopoietic stem cell, a cord blood stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and has been immortalized for long-lived in vitro cell culture.

An “adipose-derived stem cell” as used herein is a stem cell derived from adipose tissue. The term includes stem cells derived from progenitor cells, mesenchymal stem cells, pre-adipocyte cells (e.g. white pre-adipocytes) and hematopoietic cells residing in adipose tissue.

A “somatic cell” is a cell forming the body of an organism. Somatic cells include cells making up organs, skin, blood, bones and connective tissue in an organism, but not germ cells.

“Allogeneic” refers to deriving from, originating in, or being members of the same species, where the members are genetically related or genetically unrelated but genetically similar. An “allogeneic transplant” refers to transfer of cells or organs from a donor to a recipient, where the recipient is the same species as the donor.

“Autologous” refers to deriving from or originating in the same subject or patient. An “autologous transplant” refers to collection and retransplant of a subject's own cells or organs.

“Graft-versus-host response” or “GVH” or “GVHD” refers to a cellular response that occurs when lymphocytes of a different MHC class are introduced into a host, resulting in the reaction of the lymphocytes against the host.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

A variety of methods of specific DNA and RNA measurements that use nucleic acid hybridization techniques are known to those of skill in the art (see, Sambrook, Id.). Some methods involve electrophoretic separation (e.g., Southern blot for detecting DNA, and Northern blot for detecting RNA), but measurement of DNA and RNA can also be carried out in the absence of electrophoretic separation (e.g., quantitative PCR, dot blot, or array).

The sensitivity of the hybridization assays may be enhanced through use of a nucleic acid amplification system that multiplies the target nucleic acid being detected. Amplification can also be used for direct detection techniques. Examples of such systems include the polymerase chain reaction (PCR) system and the ligase chain reaction (LCR) system. Other methods include the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman® and molecular beacon probes can be used to monitor amplification reaction products in real time.

A “short hairpin RNA” or “small hairpin RNA” is a ribonucleotide sequence forming a hairpin turn which can be used to silence gene expression. After processing by cellular factors the short hairpin RNA interacts with a complementary RNA thereby interfering with the expression of the complementary RNA.

The words “protein”, “peptide”, and “polypeptide” are used interchangeably to denote an amino acid polymer or a set of two or more interacting or bound amino acid polymers.

A “dominant negative protein” is a modified form of a wild-type protein that adversely affects the function of the wild-type protein within the same cell. As a modified version of a wild-type protein the dominant negative protein may carry a mutation, a deletion, an insertion, a post-translational modification or combinations thereof. Any additional modifications of a nucleotide or polypeptide sequence known in the art are included. The dominant-negative protein may interact with the same cellular elements as the wild-type protein thereby blocking some or all aspects of its function.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “episomal” refers to the extra-chromosomal state of a plasmid in a cell. Episomal plasmids are nucleic acid molecules that are not part of the chromosomal DNA and replicate independently thereof.

A “vector” is a nucleic acid that is capable of transporting another nucleic acid into a cell. A vector is capable of directing expression of a protein or proteins encoded by one or more genes carried by the vector when it is present in the appropriate environment.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

A “cell culture” is an in vitro population of cells residing outside of an organism. The cell culture can be established from primary cells isolated from a cell bank or animal, or secondary cells that are derived from one of these sources and immortalized for long-term in vitro cultures.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

Expression of a transfected gene can occur transiently or stably in a host cell. During “transient expression” the transfected nucleic acid is not integrated into the host cell genome, and is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision.

The term “Yamanaka factors” refers to Oct3/4, Sox2, Klf4, and c-Myc, which factors are highly expressed in embryonic stem (ES) cells. Yamanaka factors can induce pluripotency in somatic cells from a variety of species, e.g., mouse and human somatic cells. See e.g., Yamanaka, 2009, Cell 137: 13-17.

A “KLF4 protein” as referred to herein includes any of the naturally-occurring forms of the KLF4 transcription factor, or variants thereof that maintain KLF4 transcription factor activity (e.g. within at least 50%, 80%, 90%, 95%, 96%, 97%, 98%, 99% or 100% activity compared to KLF4). In some aspects, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KLF4 polypeptide (e.g. SEQ ID NO:1). In other aspects, the KLF4 protein is the protein as identified by the NCBI reference gi:194248077 (SEQ ID NO:1) or a variant having substantial identity to SEQ ID NO:1.

An “OCT4 protein” as referred to herein includes any of the naturally-occurring forms of the Octomer 4 transcription factor, or variants thereof that maintain Oct4 transcription factor activity (e.g. within at least 50%, 80%, 90%, 95%, 96%, 97%, 98%, 99% or 100% activity compared to Oct4). In some aspects, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Oct4 polypeptide (e.g. SEQ ID NO:2, SEQ ID NO:3 or SEQ ID N04). In other aspects, the Oct4 protein is the protein as identified by the NCBI reference gi:42560248 corresponding to isoform 1 (SEQ ID NO:2), gi:116235491 and gi:291167755 corresponding to isoform 2 (SEQ ID NO:3 and SEQ ID NO:4), or a variant having substantial identity to SEQ ID NOs:2-4.

A “SOX2 protein” as referred to herein includes any of the naturally-occurring forms of the SOX2 transcription factor, or variants thereof that maintain SOX2 transcription factor activity (e.g. at least 50%, 80%, 90%, 95%, 96%, 97%, 98%, 99% or 100% activity compared to Sox2). In some aspects, variants have at least 90%, 95%, 96%, 97%, 98%, 99% or 100% amino acid sequence identity across the whole sequence or a portion of the sequence (e.g. a 50, 100, 150 or 200 continuous amino acid portion, e.g., the DNA-binding region) compared to a naturally occurring Sox2 polypeptide (e.g. SEQ ID NO:5). In some aspects, the SOX2 protein is the protein as identified by the NCBI reference gi:28195386 (SEQ ID NO:5) or a variant having substantial identity to SEQ ID NO:5.

A “cMYC protein” as referred to herein includes any of the naturally-occurring forms of the cMyc transcription factor, or variants thereof that maintain cMyc transcription factor activity (e.g. within at least 50%, 80%, 90%, 95%, 96%, 97%, 98%, 99% or 100% activity compared to cMyc). In some aspects, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cMyc polypeptide (e.g. SEQ ID NO:6). In other aspects, the cMyc protein is the protein as identified by the NCBI reference gi:71774083 (SEQ ID NO:6), or a variant having substantial identity to SEQ ID NO:6.

The terms “agonist,” “activator,” “upregulator,” etc. refer to a substance capable of detectably increasing the expression or activity of a given gene or activity. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or more higher than the expression or activity in the absence of the agonist.

A “SOX2 agonist” or “SOX2 signaling agonist” is a substance that increases the expression or activity of SOX2 in a cell. SOX2 expression can be increased, e.g., by addition or activation of a positive regulatory factor upstream of SOX2 expression. SOX2 activity can be increased, e.g., by addition or activation of a positive regulatory factor upstream of SOX2 activity. In some aspects, the SOX2 agonist is an inhibitor of an agent that represses SOX2 expression or activity.

The terms “inhibitor,” “repressor” or “antagonist” or “downregulator” interchangeably refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life or engraftment potential) or therapeutic measures (e.g., comparison of side effects). Controls can be designed for in vitro applications, e.g., testing the activity of various SOX2 signaling agonists. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “therapy”, “treatment,” and “amelioration” refer to any reduction in the severity of symptoms, e.g., of neutropenia or hematopoietic cell deficiency. As used herein, the terms “treat” and “prevent” are not intended to be absolute terms. Treatment can refer to any delay in onset, amelioration of symptoms, improvement in patient survival, reduction in immunodeficiency, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

“Subject,” “patient,” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision.

In the context of the present invention, i.e., methods for forming HPCs, a subject in need of treatment can refer to an individual that is deficient in one or more hematopoietic cell population. The deficiency can be due to a genetic defect, radiation or chemotherapy, or pathogenic infection.

A “transplant,” as used herein, refers to cells, e.g., hematopoietic cells, introduced into a subject. The source of the transplanted material can be cultured cells, cells from another individual, or cells from the same individual (e.g., after the cells are cultured in vitro).

### II. Methods for Transgeneration of Hematopoietic Progenitor Cells

The methods provided herein can be used to form a HPC from an MSC (e.g. through transgeneration of a MSC). A person of skill in the art will immediately recognize that the methods provided herein are typically performed with a plurality of MSCs thereby forming a plurality of HPCs. Therefore, any method provided herein for forming (e.g. transdifferentiating, transgenerating) a HPC from a MSC can be directly applied to a plurality of MSCs forming a plurality of HPCs. The methods can be accomplished in a matter of days, resulting in high efficiency generation of HPCs. The methods can be applied to a wide range of applications, e.g., to generate a population of HPCs for transplantation into a subject having a hematopoietic deficiency, e.g., neutropenia. The HPCs formed by the methods provided herein may be autogolous to the subject, i.e., the MSCs are obtained from the subject and then reintroduced after transdetermination into HPCs. The HPCs may also be allogeneic, i.e., the MSCs are obtained from a different individual or group of individuals.

In one aspect, a method of forming a HPC is provided. The method includes contacting a mesenchymal stem cell (MSC) with a SOX2 signaling agonist and allowing the MSC to form a HPC. The allowing may include culturing the MSC for sufficient time and under conditions suitable for the MSC to undergo division, thereby forming a HPC. Thus, in some embodiments, the allowing includes culturing the MSC. In some embodiments, the culturing is conducted in the absence of feeder cells (i.e. under a feeder-free conditions). Therefore, the HPC is formed by contacting the MSC with a SOX2 signaling agonist and culturing the MSC in the absence of feeder cells and under conditions suitable for the MSC to undergo division, thereby forming a HPC. In some embodiments, the MSCs are contacted with a SOX2 signaling agonist for a short term, for example for 1, 2, 3, 4, 5, 6, 12, 18 or 24 hours or for, 1, 2, 3, 4, 5, 6, 7, 8, 9, 10 or more days, or for 2-3 weeks. In some embodiments, the MSCs are contacted with the SOX2 signaling agonist for 1 month.

In the methods, compositions and kits provided herein the SOX2 signaling agonist may be a TGF beta signaling antagonist or an ERK signaling antagonist. Thus, in some embodiments, the SOX2 signaling agonist is a TGF beta signaling antagonist or an ERK signaling antagonist. In some embodiments, the SOX2 signaling agonist is a TGF beta signaling antagonist. A TGF beta signaling antagonist as provided herein may be a small molecule, a peptide antagonist (e.g., a dominant negative form of TGF beta or TGF beta receptor), or nucleic acid antagonist (e.g., siRNA, shRNA or antisense sequence). Examples of a TGF beta signaling antagonist are without limitation SB431542 (e.g., Selleck Chemicals), LY364947 (e.g., Sigma-Aldrich), or Stemolecule™ (e.g., Stemgent). In some embodiments, the TGF beta signaling antagonist is SB431542. In other embodiments, the SOX2 signaling agonist is an ERK signaling antagonist. An ERK signaling antagonist as provided herein may be a small molecule, a peptide antagonist, or nucleic acid antagonist (e.g., siRNA, shRNA or antisense sequence). Examples of an ERK signaling antagonist are without limitation U0126 (e.g., Sigma-Aldrich), ERK inhibitor PKI-ERK-005 (e.g., B-Bridge Int'l) or PD0324901 (e.g., Cayman Chemical). In some embodiments, the ERK signaling antagonist is U0126.

One of skill will further understand that more than one SOX2 signaling agonist can be applied in any combination, sequentially or simultaneously. Thus, in some embodiments, the method of forming a HPC includes contacting the MSC with at least one SOX2 signaling agonist and allowing the MSC to form a HPC. In some embodiments, the method includes contacting the MSC with a TGF beta signaling antagonist and an ERK signaling antagonist.

The methods provided herein may be carried out in the absence of exogenous Yamanaka factors other than SOX2. In other words, the MSC, or plurality of MSCs provided herein, may lack exogenous expression of any of the Yamanaka factors except for SOX2. Thus, in some embodiments, the MSC lacks an exogenous nucleic acid encoding a KLF4 protein, a OCT4 protein or a cMYC protein. In other embodiments, the MSC lacks an exogenous nucleic acid encoding a KLF4 protein, a OCT4 protein and a cMYC protein. In some embodiments, the MSC lacks an exogenous KLF4 protein, an exogenous OCT4 protein and an exogenous cMYC protein. In some embodiments, the methods provided herein are carried out in the absence of any detectable exogenous nucleic acids.

A person of ordinary skill in the art will immediately recognize that the methods provided herein are typically performed with a plurality of MSCs. The methods provided herein may be carried out with a plurality (population) of MSCs to form a plurality (population) of HPCs. Thus, in some embodiments, the method includes contacting a plurality of MSCs with a plurality of SOX2 signaling agonists and allowing the plurality of MSCs to form a population of cells comprising a plurality of HPCs.

As the MSCs form (transdifferentiate, transgenerate into) HPCs using the methods provided herein, the HPCs can be separated from the non-HPCs (i.e., non-transdetermined cells) in the population. Thus. in some embodiments, the method further includes separating the plurality of HPCs from the remainder of the population of cells, thereby forming a plurality of separated HPCs. The separation of the plurality of HPCs from non-HPCs (i.e. the remainder of the cell population) can be performed using cell separation techniques known in the art (differential size fractionation, FACS-based cell sorting, or affinity based methods such as magnetic or chromatographic separation). In some embodiments, the separating is carried out 4 or more days after said contacting. In some embodiments, the separating is carried out 7 days after said contacting. In other embodiments, the separating is carried out 8 or more days after the contacting.

The methods provided herein may include transfecting the HPC or plurality of HPCs with a siRNA or plurality of siRNAs, thereby forming a siRNA HPC or a plurality of siRNA HPCs. The HPC or plurality of HPCs may be transfected with a siRNA or plurality of siRNAs before or after the HPC or plurality of HPCs have been separated from the remainder of the cell population being formed during the transgeneration. Thus, in some embodiments, the plurality of HPCs is transfected with a plurality of siRNAs, thereby forming a plurality of siRNA HPCs. In other embodiments, the plurality of separated HPCs is transduced with a plurality of siRNAs, thereby forming a plurality of separated siRNA HPCs. The methods provided herein further include transfecting a MSC or plurality of MSCs with a siRNA or plurality of siRNAs. In some embodiments, the MSC or plurality of MSCs is transfected with a siRNA or plurality of siRNAs, thereby forming a siRNA MSC or plurality of siRNA MSCs. The MSC or plurality of MSCs may be transfected with a siRNA or plurality of siRNAs before or after the MSC or plurality of MSCs have been contacted with a SOX2 signaling agonist or a plurality of SOX2 signaling agonists. In some embodiments, the MSC or plurality of MSCs is transfected with a siRNA or plurality of siRNAs at the same time as the contacting with the SOX2 signaling agonist or the plurality of SOX2 signaling agonists occurs.

The methods provided herein may further include transducing a MSC or a plurality of MSCs with a SOX2 protein or a SOX2 nucleic acid or a plurality of SOX2 proteins or a plurality of SOX2 nucleic acids. A SOX2 protein is a protein including SOX2 or any functional equivalent thereof. A SOX2 nucleic acid is a nucleic acid encoding a SOX2 protein or any functional equivalent thereof. In some embodiments, the MSC or plurality of MSCs is contacted with a SOX2 protein, thereby transducing the MSC or plurality of MSCs with the SOX2 protein. In other embodiments, the MSC or plurality of MSCs is contacted with a SOX2 nucleic acid, thereby transducing the MSC or plurality of MSCs with the SOX2 nucleic acid. In some embodiments, the method further includes contacting a MSC with a SOX2 signaling agonist and transducing the MSC with a SOX2 protein or a SOX2 nucleic acid. In other embodiments, the method further includes contacting a plurality of MSCs with a plurality of SOX2 signaling agonists and transducing the plurality of MSCs with a plurality of SOX2 proteins or a plurality of SOX2 nucleic acids. One of skill will further understand that the contacting with a SOX2 signaling agonist and the transducing with a SOX2 protein or a SOX2 nucleic acid may occur sequentially or simultaneously.

The invention provides methods for preparing an HPC, or a plurality of HPCs, that includes introducing a nucleic acid vector (i.e., an exogenous nucleic acid vector) encoding a SOX2 protein (a SOX2 nucleic acid) into an MSC, or plurality of MSCs, and allowing the MSC(s) to form HPC(s). The allowing may include culturing the MSC to undergo cell division. The allowing may further include culturing the MSC under conditions suitable for transdetermination, thereby preparing an HPC. Further methods are provided that include introducing a SOX2 protein (i.e. exogenous SOX2 protein) into an MSC, or plurality of MSC and allowing the MSC(s) to form HPC(s). The allowing may include culturing the MSC to undergo cell division. The allowing may further include culturing the MSC under conditions suitable for transdetermination, thereby preparing an HPC.

### III. Mesenchymal Stem Cells

Further provided herein are cell compositions, including MSCs, HPCs, and cells undergoing transdetermination. The invention provides an isolated mesenchymal stem cell (MSC) comprising a SOX signaling agonist (e.g., a SOX2 signaling agonist), wherein the MSC gives rise to or forms an hematopoietic progenitor cell (HPC).

Thus, in one aspect, a mesenchymal cell including a SOX2 signaling agonist is provided. The SOX signaling agonist may be bound to the MSC, e.g., to a receptor on the MSC. In some embodiments, the SOX2 signaling agonist is a TGF beta signaling antagonist or a ERK signaling antagonist. In other embodiments, the SOX2 signaling agonist is a TGF beta signaling antagonist and a ERK signaling antagonist.

In another aspect, a mesenchymal stem cell including a SOX2 protein or a SOX2 nucleic acid is provided. In some embodiments, the mesenchymal stem cell includes a SOX2 protein and a SOX2 nucleic acid. In some embodiments, the mesenchymal stem cell further includes a SOX2 signaling agonist. In some embodiments, the SOX2 signaling agonist is a TGF beta signaling antagonist or a ERK signaling antagonist. In other embodiments, the SOX2 signaling agonist is a TGF beta signaling antagonist and a ERK signaling antagonist. Thus, in some embodiments, the mesenchymal stem cell lacks an exogenous nucleic acid encoding a KLF4 protein, a OCT4 protein or a cMYC protein. In other embodiments, the mesenchymal stem cell lacks an exogenous nucleic acid encoding a KLF4 protein, a OCT4 protein and a cMYC protein. In some embodiments, the mesenchymal stem cell lacks an exogenous KLF4 protein, an exogenous OCT4 protein and an exogenous cMYC protein.

As described above the MSC provided herein may lack the expression of any other Yamanaka factors except for SOX2. Thus, in some embodiments, the mesenchymal stem cell lacks an exogenous nucleic acid encoding a KLF4 protein, a OCT4 protein or a cMYC protein. In other embodiments, the mesenchymal stem cell lacks an exogenous nucleic acid encoding a KLF4 protein, a OCT4 protein and a cMYC protein. In some embodiments, the mesenchymal stem cell lacks an exogenous KLF4 protein, an exogenous OCT4 protein and an exogenous cMYC protein.

The invention further provides an HPC formed according to the methods provided herein including contacting an isolated MSC with a SOX signaling agonist (e.g., a SOX2 signaling agonist) and allowing the MSC to form an HPC, i.e., transdetermine or transdifferentiate into an HPC. The invention further provides an HPC formed according to the methods provided herein including contacting an isolated MSC with a SOX2 protein (e.g. a recombinant SOX2 protein) or a SOX2 nucleic acid (e.g., a SOX2 encoding expression vector) and allowing the MSC to form an HPC, i.e., transdetermine or transdifferentiate into an HPC. In some aspects, the MSC undergoes cell division during transdetermination.

### IV. Methods of Obtaining MSCs

Mesenchymal stem cells for transdetermination into HPCs can be obtained from any mammal, e.g., a rodent, rabbit, goat, bovine, sheep, horse, non-human primate or human. For therapeutic applications of the HPCs, the MSCs can be obtained from the intended recipient of the HPC transplant. That is, the MSCs and HPCs will be autologous to the recipient of the HPCs. In some aspects, the MSCs can instead be obtained from a different individual or group of individuals, e.g., a close relative. In that case, the MSCs and HPCs will be allogeneic to the recipient of the HPCs.

MSCs can be obtained from a number of tissues, e.g., adipose tissue, olfactory epithelia, bone marrow, liver, amniotic fluid, etc. A number of commercially available products are available for isolation from primary tissues, e.g., RosetteSep® Human MSC Enrichment Cocktail and EasySep® MSC Enrichment Kit. Isolation can be based on MSC cell surface markers, but also account for morphology and size of MSCs. Methods for isolating MSCs are further described herein, and, e.g., in You et al. (2009) Intl J. Gynecol. Obstetrics 103:149-52 and Alhadlaq & Mao (2004) Stem Cells and Development 13:436-448. In some embodiments, the plurality of MSCs are obtained from olfactory tissue or adipose tissue.

### V. Methods of Culturing MSCs for Transdetermination

Suitable culture conditions are described herein, and can include standard tissue culture conditions. For example, the MSCs can be cultured in a buffered media that includes amino acids, nutrients, growth factors, etc, as will be understood in the art. In some aspects, the culture includes feeder cells (e.g., fibroblasts), while in others, the culture is devoid of feeder cells. Cell culture conditions are described in more detail, e.g., in Picot, Human Cell Culture Protocols (Methods in Molecular Medicine) 2010 ed. and Davis, Basic Cell Culture 2002 ed.

In some aspects, the MSCs are cultured and allowed to divide. As explained above, MSCs can give rise to additional pluripotent daughter cells, or to more differentiated cells. According to the methods described herein, the MSCs can divide and produce transdetermined HPCs. Cell division can be determined according to methods known in the art, e.g., detecting incorporation of labeled nucleic acids or amino acids.

### VI. Recombinant Methods

In some aspects, the invention involves recombinant methods, e.g., for construction of vectors encoding SOX2 protein or an antisense construct as described herein. Standard recombinant methods are used for cloning, DNA and RNA isolation, amplification and purification. Generally, enzymatic reactions involving DNA ligase, DNA polymerase, restriction endonucleases and the like are performed according to the manufacturer's specifications. Basic texts disclosing the general methods of use in this invention include Sambrook and Russell eds. (2001) Molecular Cloning: A Laboratory Manual, 3rd edition; the series Ausubel et al. eds. (2007 with updated through 2010) Current Protocols in Molecular Biology, among others known in the art.

In some aspects, a nucleotide sequence that specifically interferes with expression of, e.g. a TGF beta, TGF beta R, MEK, or ERK gene, at the transcriptional or translational level can be used. This approach may utilize, for example, siRNA and/or antisense oligonucleotides to block transcription or translation of a specific mRNA, either by inducing degradation of the mRNA with a siRNA or by masking the mRNA with an antisense nucleic acid.

The siRNA is typically about 5 to about 100 nucleotides in length, more typically about 10 to about 50 nucleotides in length, most typically about 15 to about 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

Antisense nucleic acids are DNA or RNA molecules that are complementary to at least a portion of a specific mRNA molecule (see, e.g., Weintraub, Scientific American, 262:40 (1990)). Typically, synthetic antisense oligonucleotides are generally between 15 and 25 bases in length. Antisense nucleic acids may comprise naturally occurring nucleotides or modified nucleotides such as, e.g., phosphorothioate, methylphosphonate, and -anomeric sugar-phosphate, backbone-modified nucleotides.

Designing and preparing small inhibitory RNAs against target genes is well known in the art and a person of skill will be able to perform such designing and preparing of small inhibitory RNAs without undue experimentation. Further, there are various commercial resources available, which design and prepare appropriate siRNA against any target gene of interest (e.g. Dharmacon, Origen, Invitrogen).

In some aspects, amplification of known sequences may be desirable, e.g., for cloning into appropriate expression vectors. Such methods of amplification are well known to those of skill in the art. Detailed protocols for PCR are provided, e.g., in Innis et al. (1990) PCR Protocols, A Guide to Methods and Applications, Academic Press, Inc. N.Y.). The known nucleic acid sequences for the genes listed herein is sufficient to enable one of skill to routinely select primers to amplify any portion of the gene.

### VII. Methods of Treatment

The transdetermined HPCs of the invention can be used for transplantation into a subject in need thereof. In some aspects, the subject is deficient in at least one type of hematopoietic cell, e.g., white or red blood cell. In some aspects the subject suffers from leukopenia (deficient white blood cells). The hematopoietic deficiency can be genetic (e.g., anemia or congenital neutropenia) or due to an external cause (e.g., radiation or chemotherapy, arsenic poisoning, particular blood cancers, pathogenic infection). Thus, in some embodiments, a method is provided for treating neutropenia in a patient in need thereof. The method includes administering (e.g. introducing or transplanting) an effective amount of one or more transdetermined HPCs (i.e. HPCs formed using the methods and kits provided herein) to the patient (e.g. recipient).

In some aspects, the method of treatment includes contacting an MSC with a SOX2 signaling agonist; allowing the MSC to form a HPC, and administering the HPC to a subject in need thereof. A person of skill in the art will immediately recognize that the methods provided herein are typically performed with a plurality of MSCs thereby forming a plurality of HPCs. The allowing may include culturing the MSC (plurality of MSCs) for sufficient time and under conditions suitable for the MSC (plurality of MSCs) to undergo division, thereby forming a HPC (plurality of HPCs). Thus, in some embodiments, the allowing includes culturing the MSC. In some embodiments, the SOX2 signaling agonist is a TGF beta signaling antagonist or a ERK signaling antagonist. One of skill will further understand that more than one SOX2 agonist can be applied in any combination, sequentially or simultaneously. Thus, in some embodiments, the method of forming a HPC includes contacting the MSC with at least one SOX2 signaling agonist and allowing the MSC to form a HPC. In some embodiments, the method includes contacting the MSC with a TGF beta signaling antagonist. In other embodiments, the method includes contacting the MSC with an ERK signaling antagonist. In some embodiments, the method includes contacting the MSC with a TGF beta signaling antagonist and an ERK signaling antagonist. The methods provided herein may further include transducing a MSC or a plurality of MSCs with a SOX2 protein or a SOX2 nucleic acid or a plurality of SOX2 proteins or a plurality of SOX2 nucleic acids. A SOX2 protein is a protein including SOX2 or any functional equivalent thereof. A SOX2 nucleic acid is a nucleic acid encoding a SOX2 protein or any functional equivalent thereof. In some embodiments, the method further includes contacting a MSC with a SOX2 signaling agonist and transducing the MSC with a SOX2 protein or a SOX2 nucleic acid. In some embodiments, the method further includes contacting a MSC with at least one SOX2 signaling agonist and transducing the MSC with a SOX2 protein or a SOX2 nucleic acid. In other embodiments, the method further includes contacting a plurality of MSCs with a plurality of SOX2 signaling agonists and transducing the plurality of MSCs with a plurality of SOX2 proteins or a plurality of SOX2 nucleic acids. In other embodiments, the method further includes contacting a plurality of MSCs with a plurality of at least one SOX2 signaling agonist and transducing the plurality of MSCs with a plurality of SOX2 proteins or a plurality of SOX2 nucleic acids. One of skill will further understand that the contacting with a SOX2 signaling agonist and the transducing with a SOX2 protein or a SOX2 nucleic acid may occur sequentially or simultaneously.

As mentioned above, the method can further comprise, prior to the contacting step, a step of obtaining the MSC from a donor subject. The HPCs formed by the methods provided herein may be introduced into a recipient. In some embodiments, the plurality of separated HPCs is introduced into a mammal. In some embodiments, the mammal is selected from a mouse, rat, rabbit, non-human primate, and human. In some cases, the donor subject is the recipient of the transdifferentiated HPCs, that is, the transplant is autologous. Thus, in some embodiments, the plurality of separated HPCs are autologous to the mammal. In some cases, the donor subject is a different individual, and the HPC for administration will be allogeneic to the recipient subject. In some embodiments, the plurality of separated HPCs are allogeneic to the mammal.

In some embodiments, the HPCs for transplantation are separated from other cell types in the culture prior to treatment. In some aspects, the HPCs are further differentiated and separated, e.g., into erythroid, granulocyte, macrophage, megakaryocyte progenitors. In some aspects, the HPCs are separated from the culture and then further differentiated e.g., into erythroid, granulocyte, macrophage, megakaryocyte progenitors. In these methods, the more distinct hematopoietic cell lineages can be applied to hematodeficiency disorders characterized by deficiencies of specific hematopoietic cell types.

Typically, the HPCs will be administered to the subject by injection, e.g., intravenously. The administration can be either in a bolus or in an infusion. The HPC compositions of the invention can comprise a pharmaceutically acceptable carrier. Pharmaceutically acceptable carriers are determined in part by the particular method used to administer the composition, but are typically isotonic, buffered saline solutions. Accordingly, there are a wide variety of suitable formulations of pharmaceutical compositions of the present invention (see, e.g., Remington's Pharmaceutical Sciences, 17th ed., 1989). The HPC compositions of the invention can be administered in a single dose, multiple doses, or on a regular basis (e.g., daily) for a period of time (e.g., 2, 3, 4, 5, 6, days, weeks, months, or as long as the condition persists).

The dose administered to the subject, in the context of the present invention should be sufficient to effect a beneficial response in the subject over time, e.g., a reduction of hematodeficient symptoms, reduction in immunodeficiency, increase in circulating hematopoietic cell numbers, or a combination thereof. The optimal dose level for any patient will depend on a variety of factors including the efficacy of the specific modulator employed, the age, body weight, physical activity, and diet of the patient, on a possible combination with other drugs, and on the severity of the hematopoietic deficiency. The size of the dose also will be determined by the existence, nature, and extent of any adverse side-effects that accompany the administration of the HSCs in a particular subject.

In some aspects, the method of treatment includes obtaining MSCs from the subject prior to treatment. Isolation of MSCs can be accomplished as described herein. In some embodiments, MSCs are harvested more than once, or routinely, and freshly transdetermined into HPCs prior to administration (reintroduction) into the subject.

Aqueous solutions of the transdetermined HPCs or subpopulations thereof can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized compositions. In some aspects, the transdetermined HPC population can be preserved at −20 C or −70 C in a standard preservation solution comprising, e.g., DMSO.

### VIII. Kits

In some aspects, the invention provides kits for transdetermination of MSCs into HPCs. The kit can optionally include written instructions or electronic instructions (e.g., on a CD-ROM or DVD). The kits of the invention may include a case or container for holding the reagents in the kit, which can be included separately or in combination.

In another aspect, a kit for forming a HPC is provided. The kit includes a MSC, a SOX2 signaling agonist and instructions to culture the MSC under conditions suitable for forming a HPC. In some embodiments, the SOX2 signaling agonist is a TGF beta signaling antagonist or a ERK signaling antagonist. In other embodiments, the kit further includes a SOX2 protein or a SOX2 nucleic acid.

In another aspect, a kit for forming a HPC is provided. The kit includes a MSC, a SOX2 protein or a SOX2 nucleic acid and instructions to culture the MSC under conditions suitable for forming a HPC. In some embodiments, the kit further includes a SOX2 signaling agonist. In another embodiment, the SOX2 signaling agonist is a TGF beta signaling antagonist or a ERK signaling antagonist. In the case where the kit includes a SOX2 nucleic acid (e.g. a vector encoding a SOX2 protein), appropriate transfection reagents can also be included. Similarly, where the kit includes a SOX2 protein, the kit can include protein transduction reagents.

In another aspect, a kit for forming a HPC is provided. The kit includes a SOX2 protein or a SOX2 nucleic acid, a SOX2 signaling agonist, and instructions to administer the components of to a cell under conditions suitable for forming a HPC.

In some aspects, the kit includes reagents for separating MSCs from a tissue or cell sample from a subject, such as those described herein (e.g., magnetic beads or other affinity based separation materials, stock buffers, etc.). Thus the kit can include antibodies or other reagents capable of specifically binding to at least one MSC-specific marker. The kit can optionally include a device for collecting the subject sample. The kit can also include tubes or other containers for holding the sample during processing.

In some aspects, the kit further includes reagents for identifying cell populations, e.g., before and after isolation of MSCs from the subject sample, MSCs during the transdetermination process into HPCs, HPCs, and hematopoietic cells of different lineages. Such reagents can include labeled reagents such as antibodies that specifically bind particular cell surface markers (e.g., CD34, CD45, etc.), as well as appropriate buffers and/or light-protected containers.

In some aspects, the kit includes culturing reagents for transdetermination, e.g., culture media, appropriate additives, tissue culture plates or bottles, etc.

### IX. Examples

### Introduction

Despite the therapeutic promise of iPS-derived HSCs, current reprogramming and iPS cells generation technologies raise serious concerns regarding their safety (Seifinejad et al. (2010) Stem Cell Rev. 6:297-306). While several attempts to derive hematopoietic stem cells from iPS cells have been reported, the lack of robust and highly efficient differentiation protocols also strongly hampers therapeutic development (Lengerke & Daley (2010) Blood Rev. 24:27). Indeed, transplantation of cell populations contaminated with undifferentiated cells represent a considerable risk for patients. The present results show that SOX2 transduction is sufficient to induce human MSCs to develop into multipotent HPCs, which are able to give rise to all blood lineages. Overall, Applicants' results demonstrate a role of SOX2 in the transition from CD34−CD45− MSCs towards the CD34+CD45+, and CD45+ hematopoietic stages. While SOX2 is not normally expressed in hematopoietic stem cells, Applicants' results indicate that it can substitute for other members of the family which may be important for human hematopoietic development.

Applicants demonstrate that SOX2 can rapidly and efficiently induce progression of human MSCs into HPCs (CD34+/CD34+CD45+/CD45+), minoring normal hematopoiesis in several aspects. Importantly, these newly generated HPCs exhibit in vitro proliferative capacity. Applicants' results also demonstrate that similar lineage conversion can be achieved by either using recombinant SOX2 protein or TGFβR1 inhibitor.

The present invention represents a novel protocol for generation of Hematopoietic Stem Cells (HSCs) in vitro. Moreover, the technology applied in this endeavor involves generation of up to 70% hematopoietic progenitors in a rapid time frame, e.g., not exceeding 8 days of in vitro differentiation in feeder-free conditions. The findings reported here constitute a step forward to the safe manipulation of hematopoietic stem cells and transition to the clinic.

### Example 1

### Materials and Methods

Reagents and Antibodies:

The following antibodies were used for flow cytometry and western blotting experiments respectively: mouse anti-human CD34-APC (130-046-703, Miltenyi), mouse anti-human CD45-FITC (130-080-202, Miltenyi), mouse anti-human CD133/2 (293C3)-PE (130-090-853, Miltenyi), mouse anti-human CD43-FITC (560978, BD biosciences), mouse APC isotype control (555751, BD biosciences), mouse FITC isotype control (555748, BD biosciences), The TGFβRI SB431542 (S4317, Sigma-Aldrich) and MEK/ERK U0126 (U120, Sigma-Aldrich) inhibitors were diluted in DMSO accordingly to the manufacturers' instruction and used at a final concentration of 25 and 10 μM respectively during the duration of the experiments with media changes every second day unless otherwise stated. Equal concentration of the solvent alone was used as a negative control.

Human Olfactory Epithelial MSC and Adipose Tissue MSC Cell Culture:

Human nasal mucosa were obtained by biopsy during routine nasal surgery with the patient under general anesthesia. Briefly, the patients were chosen among people undergoing surgery for septoplasty or turbinectomy. During the surgery, the ENT surgeon excised a 2 mm² biopsy on either the dorsomedial and dorsoposterior areas of the superior turbinate or the dorsomedial and dorsoposterior areas of the septum. Samples were obtained under a protocol that was approved by the local ethics committee. Human OE-MSCs were cultivated in DMEM/HAM′S F12 (Invitrogen, Carlsbad, Calif.) supplemented with fetal bovine serum (FBS, 10%).

Human mesenchymal stem cells derived from adipose tissue (hMSC-AT) were obtained from PromoCell (Heidelberg, Germany). Human MSC-AT were cultivated in α-minimum essential medium (Invitrogen, Carlsbad, Calif.) supplemented with 10% FBS or 1 ng/mL fibroblast growth factor 2 (FGF2), respectively.

Both cell types were maintained in an incubator (37° C., 5% CO2) with media changes every 2 to 3 days and passaged by using TrypLE (invitrogen) when they reached 90-95% confluency.

Production of and Infection with Retroviral Constructs:

pMXs-hSOX2, pMXs-hKLF4-cDNA retroviral vectors and packaging constructs were transfected into 293FT cells using Lipofectamine 2000 (Invitrogen), according to the manufacturer's directions. Eight hours after transfection, the DNA-lipofectamine-complex was removed and the medium was replaced the next day. Virus-containing supernatants were collected after 48 hr and filtered through a 0.45-μm filter.

Human OE-MSC and MSC-AT cells (passage 7) were infected (day 0) with pMX-based retroviruses by spinfection of the cells at 1850 rpm for 1 hour in the presence of polybrene (4 μg/ml). Cells were maintained in the retroviruses-containing media for 12 hours and then the media was removed and replaced by the cell-specific culture medium. Twenty four hours after the first infection (day 1), a second round of infection was performed. The day after (day 2), cells were maintained in their respective media for 24 hours before being switched to a Wicell media containing DMEM/F12 (Invitrogen) supplemented with 20% Knockout Serum Replacement (Invitrogen), 1 mM L-glutamine, 0.1 mM non-essential aminoacids, 55 mM β-mercaptoethanol and 10 ng/ml bFGF.

CFSE Cell Proliferation Assays:

CFSE stainings (CellTrace CFSE Cell Proliferation Kit, C34554, Molecular Probes) were conducted according to the manufacturer's instructions with a reduced final concentration of 2.5 μM.

Hematopoietic Colony Forming Assays:

Hematopoietic clonogenic assays were performed in 35-mm low adherent plastic dishes (Stem Cell Technologies, Vancouver, BC, Canada) using 1.1 ml/dish of methylcellulose semisolid medium (MethoCult H4434 classic, Stem Cell Technologies) according to the manufacturer's instructions. Briefly, enriched CD34+ OE-MSC-derived cells were sorted and immediately plated at various densities: 1.5×10³/ml, 3×10³/ml and 6×10³/ml. All assays were performed in duplicate. Colony-forming units (CFU) and Burst-forming units (BFU) were scored after 7 to 14 days of incubation according to their colony morphology as erythroid (CFU-E and BFU-E), granulocyte, erythroid, macrophage, megakaryocyte (CFU-GEMM), granulocyte-macrophage (CFU-GM), and macrophage (CFU-M).

Flow Cytometry Surface Staining:

Human MSCs undergoing hematopoietic transdetermination were harvested at the indicated time points. Cells were washed once with PBS and further incubated with the corresponding antibodies in the presence of FACS blocking buffer (1×PBS/10% FCS) for 1 hour on ice in the absence of light. After incubation, cells were washed three times with 1 ml FACS blocking buffer and resuspended in a total volume of 200 μl prior to analysis. A minimum of 10,000 living cells were analyzed.

Protein Transduction:

2.5 μg of recombinant human Sox2-TAT (110-03T, Peprotech) was added to the culture media every 12 hours prior to analysis of transdetermination.

Magnetic Cell Sorting (MACS):

Transdifferentiated MSCs were firstly depleted for specific hematopoietic lineages by using Lineage (Lin) specific depletion kit (130-092-211, Miltenyi) according to the manufacturer's instructions with slight modifications. CD34+ and/or CD34+CD45+ cells present in the Lin-fraction were further purified by incubation with CD34-coupled magnetic beads (130-046-703, Miltenyi). Briefly, up to 10⁹ cells were incubated with rotation at 4° C. with 100 μl of the corresponding magnetic beads in the presence of 100 μl of Fc-blocking solution in a total volume of 1 ml FACS blocking buffer. After 1 hour, cells were sorted by two consecutive rounds of column separation in order to increase purity by applying MACS separation magnets. Shortly, cells were passed through the first MS separation column allowing binding of labeled cells. Non-labeled cells were washed thoroughly with 3 ml FACS blocking buffer prior to elution of the labeled fraction. Eluted labeled cells were then subjected to a second purification step as described above.

Microarray Analysis:

fRMA tools were used to preprocess the data in order to allow for further comparisons. Cel files were generated using Affymetrix software and imported into Chipinspector. The data were analyzed by Genomatix Chipinspector as described by the manufacturer's guidelines (Genomatix GmbH, Munich, Germany, available at genomatix.de). Bone Marrow CD34+ was downloaded from the NCBI dataset browser (GDS2397). dChip software was used for hierarchical clustering of datasets (available at biosunl.harvard.edu/complab/dchip). A gene ontology study was performed using EASE. For each gene ontology category, a Fisher's exact p-value was calculated and adjusted using Bonferroni method. A 5% p-value was applied as a cut-off.

RNA Isolation and Real Time PCR (RT-PCR) Analysis:

Total RNA was isolated using Trizol Reagent (Invitrogen) according to the manufacturer's recommendations. 1 μg of DNAse1 (invitrogen) treated total RNA was used for cDNA synthesis using the SuperScript II Reverse Transcriptase kit for RT-PCR (Invitrogen). Real-time PCR was performed using the SYBR-Green PCR Master mix (Applied Biosystems). The levels of expression of respective genes were normalized to corresponding GAPDH values and are shown as fold change relative to the value of the control sample. All the samples were done in triplicate. The list of the primers used for real time-PCR experiments are listed in Table 1. The sequences correspond to SEQ ID NOs:7-68, and are listed in order, i.e., SEQ ID NO:7 (OCT4 endogenous 5′ oligo), SEQ ID NO:8 (OCT4 endogenous 3′oligo), SEQ ID NO:9 (NANOG 5′ oligo), etc.

TABLE 1

| Gene | 5′ oligo | 3′ Oligo |
|---|---|---|
| OCT4 | GGGTTTTTGGGATTAAGTTCTTCA | GCCCCCACCCTTTGTGTT |
| (endogenous) | | |
| NANOG | ACAACTGGCCGAAGAATAGCA | GGTTCCCAGTCGGGTTCAC |
| SOX2 | CAAAAATGGCCATGCAGGTT | AGTTGGGATCGAACAAAAGCTATT |
| (endogenous) | | |
| SOX2 | GGCACCCCTGGCATGGCTCTTGGC | TTATCGTCGACCACTGTGCTGCTG |
| (exogenous) | TC | |
| CD9 | GGATATTCCCACAAGGATGAGGT | GATGGCTTTCAGCGTTTCCC |
| CD11b | ACTTGCAGTGAGAACACGTATG | AGAGCCATCAATCAAGAAGGC |
| CD14 | ACGCCAGAACCTTGTGAGC | GCATGGATCTCCACCTCTACTG |
| CD19 | GGCCCGAGGAACCTCTAGT | ACTCCCGAGACCAGGTCAG |
| CD29 | TTATTGGCCTTGCATTACTGCT | CCACAGTTGTTACGGCACTCT |
| CD34 | CCTAAGTGACATCAAGGCAGAA | GCAAGGAGCAGGGAGCATA |
| CD38 | AGACTGCCAAAGTGTATGGGA | GCAAGGTACGGTCTGAGTTCC |
| CD41 | AGCTGCGAGGGAACTCCTT | GGTGTGCTCCACTTTGGGT |
| CD43 | GCCAACAACCTACCAGGAAGT | GTTCCACCTGTCACGGTGTG |
| CD44 | GCACAGACAGAATCCCTGCTA | GCCATTTGTGTTGTTGTGTGAA |
| CD45 | ACAGCCAGCACCTTTCCTAC | GTGCAGGTAAGGCAGCAGA |
| CD90 | TCGCTCTCCTGCTAACAGTCT | CTCGTACTGGATGGGTGAACT |
| CD150 | AAGCTACGGAACAGGTGGG | GTGATTTTGCCATTGTGACGAC |
| CD166 | CCCGATGGCTCCCCAGTAT | ACGTTGTCCTCAGTTACTAGCA |
| B220 | ACAGCCAGCACCTTTCCTAC | GTGCAGGTAAGGCAGCAGA |
| EMR1 | GGAAGGGCACATAAGACCCAC | GGGCACAAGGTACTGTCTCTA |
| HOXB4 | GTGAGCACGGTAAACCCCAAT | CGAGCGGATCTTGGTGTTG |
| GATA1 | AGAAGCGCCTGATTGTCAGTA | AGAGACTTGGGTTGTCCAGAA |
| GATA2 | GGCCCACTCTCTGTGTACC | CATCTTCATGCTCTCCGTCAG |
| RUNX1 | AGAACCTCGAAGACATCGGC | GGCTGAGGGTTAAAGGCAGTG |
| CXCR4 | CACCGCATCTGGAGAACCA | GCCCATTTCCTCGGTGTAGTT |
| LMO2 | GGACCCTTCAGAGGAACCAGT | GGCCCAGTTTGTAGTAGAGGC |
| SCL | CAAAGTTGTGCGGCGTATCTT | TCATTCTTGCTGAGCTTCTTGTC |
| TEL | AAACTTCATCCGATGGGAGGA | CGCAGGGCTCTGGACATTTT |
| FOXA1 | CCAAGGCCGCCTTACTCCTACA | CGCAGATGAAGACGCTTGGAGA |
| AC133 | CATCCACAGATGCTCCTAAGGC | AAGAGAATGCCAATGGGTCCA |
| GAPDH | GGACTCATGACCACAGTCCATGCC | TCAGGGATGACCTTGCCCACAG |

TABLE 2
Microarray analysis of OE-MSCs versus transdetermined CD34 + OE-MSCs
Table 2: Significantly differentially regulated genes in CD34 derived OE-MSCs compared to OE-MSCs at 5% FDR.
TABLE 1A

| Affymetrix ID | Entrenz Gene ID | Ensemble Gene ID | Unigene ID | Gene Name | Gene Symbol | Fold Change |
|---|---|---|---|---|---|---|
| 1255_G_AT | 2978 | ENSG00000048545 | Hs.92858 | guanylate cyclase activator 1A (retina) | GUCA1A | 3.463084549 |
| 1552390_A_AT | 203111 | ENSG00000177459 | Hs.171455 | chromosome 8 open reading frame 47 | C8orf47 | 3.837002879 |
| 1553102_A_AT | 26112 | ENSG00000198624 | Hs.655336 | coiled-coil domain containing 69 | CCDC69 | 4.117230955 |
| 1553388_AT | 221301 | — | Hs.350750 | family with sequence similarity 26, member D | FAM26D | 45.43887954 |
| 1553394_A_AT | 7021 | ENSG00000008196 | Hs.33102 | transcription factor AP-2 beta (activating enhancer binding protein 2 beta) | TFAP2B | 4.911091791 |
| 1553605_A_AT | 154664 | ENSG00000179869 | Hs.226568 | ATP-binding cassette, sub-family A (ABC1), member 13 | ABCA13 | 2.919856846 |
| 1553994_AT | 4907 | — | Hs.153952 | 5′-nucleotidase, ecto (CD73) | NT5E | 3.144455389 |
| 1553995_A_AT | 4907 | ENSG00000135318 | Hs.153952 | 5′-nucleotidase, ecto (CD73) | NT5E | 2.937925352 |
| 1554195_A_AT | 389336 | — | Hs.660038 | chromosome 5 open reading frame 46 | C5orf46 | 6.746311985 |
| 1554500_A_AT | 6000 | ENSG00000182901 | Hs.655739 | regulator of G-protein signaling 7 | RGS7 | 0.148510845 |
| 1554640_AT | 114299 | ENSG00000243444 | — | paralemmin 2 | PALM2 | 0.373986637 |
| 1554712_A_AT | 219970 | ENSG00000156689 | Hs.254271 | glycine-N-acyltransferase-like 2 | GLYATL2 | 5.332851019 |
| 1554812_AT | 49861 | ENSG00000171217 | Hs.567491 | claudin 20 | CLDN20 | 42.19964976 |
| 1554830_A_AT | 55240 | ENSG00000115107 | Hs.647822 | STEAP family member 3 | STEAP3 | 0.318685918 |
| 1555059_AT | — | — | — | — | — | 2.855072924 |
| 1555926_A_AT | — | — | Hs.534689 | — | — | 6.610798695 |
| 1555928_AT | — | — | Hs.534689 | — | — | 6.060183785 |
| 1555929_S_AT | — | — | Hs.545794 | — | — | 13.44252304 |
| 1556329_A_AT | — | — | Hs.654741 | — | — | 4.219811294 |
| 1557348_AT | — | — | Hs.623520 | — | — | 3.454233234 |
| 1557636_A_AT | 136288 | ENSG00000164746 | Hs.258357 | chromosome 7 open reading frame 57 | C7orf57 | 106.0112674 |
| 1558076_AT | 84250 | ENSG00000133302 | Hs.657315 | ankyrin repeat domain 32 | ANKRD32 | 3.43155434 |
| 1558501_AT | 26052 | — | Hs.654775 | dynamin 3 | DNM3 | 33.98516265 |
| 1558502_S_AT | 26052 | ENSG00000197959 | Hs.654775 | dynamin 3 | DNM3 | 3.146580422 |
| 1558517_S_AT | 84230 | ENSG00000171488 | Hs.412836 | leucine rich repeat containing 8 family, member C | LRRC8C | 3.379938908 |
| 1558662_S_AT | 55024 | ENSG00000153064 | Hs.480400 | B-cell scaffold protein with ankyrin repeats 1 | BANK1 | 8.140580387 |
| 1558815_AT | 8470 | — | Hs.655143 | sorbin and SH3 domain containing 2 | SORBS2 | 26.21966121 |
| 1558871_AT | — | — | Hs.662627 | — | — | 4.633397608 |
| 1560019_AT | 84777 | — | Hs.659053 | hypothetical LOC84777 | MGC11082 | 4.306338546 |
| 1560359_AT | 53918 | — | Hs.644352 | Pelota homolog (Drosophila) | PELO | 7.199861177 |
| 1560474_AT | — | — | Hs.680634 | — | — | 9.053059836 |
| 1560652_AT | — | — | Hs.407141 | — | — | 5.363611197 |
| 1560679_AT | 151438 | — | Hs.516245 | hypothetical protein LOC151438 | LOC151438 | 50.40046293 |
| 1562301_AT | 116328 | ENSG00000165084 | Hs.491941 | chromosome 8 open reading frame 34 | C8orf34 | 2.671505468 |
| 1562367_AT | 400360 | ENSG00000175746 | Hs.376109 | chromosome 15 open reading frame 54 | C15orf54 | 12.39706728 |
| 1563498_S_AT | 283130 | — | Hs.661604 | solute carrier family 25, member 45 | SLC25A45 | 3.031505113 |
| 1568618_A_AT | 2589 | ENSG00000141429 | Hs.514806 | UDP-N-acetyl-alpha-D-galactosamine:polypeptide N-acetylgalactosaminyltransferase 1 | GALNT1 | 4.306187928 |
| | | | | (GalNAc-T1) | | |
| 1569290_S_AT | 2892 | ENSG00000125675 | Hs.377070 | glutamate receptor, ionotrophic, AMPA 3 | GRIA3 | 3.818225138 |
| 1569512_AT | — | — | Hs.684405 | — | — | 2.164262975 |
| 200789_AT | 1891 | — | Hs.196176 | enoyl CoA hydratase 1, peroxisomal | ECH1 | 2.621615462 |
| 200878_AT | 2034 | ENSG00000116016 | Hs.468410 | endothelial PAS domain protein 1 | EPAS1 | 0.199924789 |
| 200904_AT | 3133 | — | Hs.650174 | major histocompatibility complex, class I, E | HLA-E | 2.618098669 |
| 200923_AT | 3959 | ENSG00000108679 | Hs.514535 | lectin, galactoside-binding, soluble, 3 binding protein | LGALS3BP | 2.851263072 |
| 200953_S_AT | 894 | ENSG00000118971 | Hs.376071 | cyclin D2 | CCND2 | 2.797546426 |
| 201186_AT | 4043 | ENSG00000163956 | Hs.533136 | low density lipoprotein receptor-related protein associated protein 1 | LRPAP1 | 2.051181901 |
| 201195_S_AT | 8140 | ENSG00000103257 | Hs.513797 | solute carrier family 7 (cationic amino acid transporter, y+ system), member 5 | SLC7A5 | 0.151772306 |
| 201243_S_AT | 481 | ENSG00000143153 | Hs.291196 | ATPase, Na+/K+ transporting, beta 1 polypeptide | ATP1B1 | 0.19733733 |
| 201340_S_AT | 8507 | ENSG00000171617 | Hs.104925 | ectodermal-neural cortex 1 (with BTB-like domain) | ENC1 | 6.281697689 |
| 201362_AT | 10625 | ENSG00000116679 | Hs.497183 | influenza virus NS1A binding protein | IVNS1ABP | 3.73439235 |
| 201422_AT | 10437 | — | Hs.14623 | interferon, gamma-inducible protein 30 | IFI30 | 2.263065326 |
| 201539_S_AT | 2273 | ENSG00000022267 | Hs.435369 | four and a half LIM domains 1 | FHL1 | 0.065193709 |
| 201540_AT | 2273 | ENSG00000022267 | Hs.435369 | four and a half LIM domains 1 | FHL1 | 0.141598537 |
| 201565_S_AT | 3398 | ENSG00000115738 | Hs.180919 | inhibitor of DNA binding 2, dominant negative helix-loop-helix protein | ID2 | 0.110505297 |
| 201566_X_AT | 3398 | — | Hs.180919 | inhibitor of DNA binding 2, dominant negative helix-loop-helix protein | ID2 | 0.074426597 |
| 201580_S_AT | 56255 | ENSG00000125827 | Hs.169358 | thioredoxin-related transmembrane protein 4 | TMX4 | 6.414693465 |
| 201581_AT | 56255 | ENSG00000125827 | Hs.169358 | thioredoxin-related transmembrane protein 4 | TMX4 | 5.196041255 |
| 201596_X_AT | 3875 | ENSG00000111057 | Hs.406013 | keratin 18 | KRT18 | 0.151990128 |
| 201641_AT | 684 | ENSG00000130303 | Hs.118110 | bone marrow stromal cell antigen 2 | BST2 | 5.559665483 |
| 201650_AT | 3880 | ENSG00000171345 | Hs.654568 | keratin 19 | KRT19 | 0.076576632 |
| 201719_S_AT | 2037 | ENSG00000079819 | Hs.486470 | erythrocyte membrane protein band 4.1-like 2 | EPB41L2 | 2.808406055 |
| 201722_S_AT | 2589 | ENSG00000141429 | Hs.514806 | UDP-N-acetyl-alpha-D-galactosamine:polypeptide N-acetylgalactosaminyltransferase 1 | GALNT1 | 3.525816563 |
| | | | | (GalNAc-T1) | | |
| 201723_S_AT | 2589 | ENSG00000141429 | Hs.514806 | UDP-N-acetyl-alpha-D-galactosamine:polypeptide N-acetylgalactosaminyltransferase 1 | GALNT1 | 4.196758321 |
| | | | | (GalNAc-T1) | | |
| 201724_S_AT | 2589 | ENSG00000141429 | Hs.514806 | UDP-N-acetyl-alpha-D-galactosamine:polypeptide N-acetylgalactosaminyltransferase 1 | GALNT1 | 4.085679583 |
| | | | | (GalNAc-T1) | | |
| 201743_AT | 929 | ENSG00000170458 | Hs.163867 | CD14 molecule | CD14 | 18.59127035 |
| 201860_S_AT | 5327 | ENSG00000104368 | Hs.491582 | plasminogen activator, tissue | PLAT | 5.426520108 |
| 201957_AT | 4660 | ENSG00000077157 | Hs.444403 | protein phosphatase 1, regulatory (inhibitor) subunit | PPP1R12B | 0.207087963 |
| | | | | 12B | | |
| 201998_AT | 6480 | ENSG00000073849 | Hs.207459 | ST6 beta-galactosamide alpha-2,6-sialyltranferase 1 | ST6GAL1 | 3.302453489 |
| 202013_S_AT | 2132 | ENSG00000151348 | Hs.368404 | exostosis 2 | EXT2 | 2.57204633 |
| 202016_AT | 4232 | ENSG00000106484 | Hs.270978 | mesoderm specific transcript homolog (mouse) | MEST | 0.079445146 |
| 202071_AT | 6385 | ENSG00000124145 | Hs.632267 | syndecan 4 | SDC4 | 0.261571051 |
| 202086_AT | 4599 | ENSG00000157601 | Hs.517307 | myxovirus (influenza virus) resistance 1, interferon- | MX1 | 56.37489589 |
| | | | | inducible protein p78 (mouse) | | |
| 202150_S_AT | 4739 | ENSG00000111859 | Hs.37982 | neural precursor cell expressed, developmentally | NEDD9 | 3.710012129 |
| | | | | down-regulated 9 | | |
| 202267_AT | 3918 | ENSG00000058085 | Hs.591484 | laminin, gamma 2 | LAMC2 | 4.865048346 |
| 202289_S_AT | 10579 | ENSG00000138162 | Hs.501252 | transforming, acidic coiled-coil containing protein 2 | TACC2 | 0.321326887 |
| 202409_AT | 3481 /// 723961 | ENSG00000167244 | Hs.272259 | insulin-like growth factor 2 (somatomedin A) /// INS- | IGF2 /// INS- | 50.03248254 |
| | | | | IGF2 readthrough transcript | IGF2 | |
| 202410_X_AT | 3481 /// 723961 | ENSG00000129965 /// | Hs.272259 | insulin-like growth factor 2 (somatomedin A) /// INS- | IGF2 /// INS- | 23.44784394 |
| | | ENSG00000167244 | | IGF2 readthrough transcript | IGF2 | |
| 202411_AT | 3429 | ENSG00000165949 | Hs.532634 | interferon, alpha-inducible protein 27 | IFI27 | 12.4443568 |
| 202430_S_AT | 5359 | ENSG00000188313 | Hs.130759 | phospholipid scramblase 1 | PLSCR1 | 7.274225444 |
| 202446_S_AT | 5359 | ENSG00000188313 | Hs.130759 | phospholipid scramblase 1 | PLSCR1 | 6.544460808 |
| 202454_S_AT | 2065 | ENSG00000065361 | Hs.118681 | v-erb-b2 erythroblastic leukemia viral oncogene | ERBB3 | 0.165936578 |
| | | | | homolog 3 (avian) | | |
| 202481_AT | 9249 | ENSG00000162496 | Hs.289347 | dehydrogenase/reductase (SDR family) member 3 | DHRS3 | 8.519856856 |
| 202565_S_AT | 6840 | ENSG00000197321 | Hs.499209 | supervillin | SVIL | 6.177105736 |
| 202566_S_AT | 6840 | ENSG00000197321 | Hs.499209 | supervillin | SVIL | 4.303698742 |
| 202594_AT | 23484 | ENSG00000104660 | Hs.146585 | leptin receptor overlapping transcript-like 1 | LEPROTL1 | 3.204655142 |
| 202595_S_AT | 23484 | ENSG00000104660 | Hs.146585 | leptin receptor overlapping transcript-like 1 | LEPROTL1 | 3.255925611 |
| 202668_AT | 1948 | ENSG00000125266 | Hs.149239 | ephrin-B2 | EFNB2 | 14.24456854 |
| 202687_S_AT | 8743 | ENSG00000121858 | Hs.478275 | tumor necrosis factor (ligand) superfamily, member | TNFSF10 | 9.535611965 |
| | | | | 10 | | |
| 202688_AT | 8743 | ENSG00000121858 | Hs.478275 | tumor necrosis factor (ligand) superfamily, member | TNFSF10 | 5.532932158 |
| | | | | 10 | | |
| 202709_AT | 2331 | ENSG00000122176 | Hs.519168 | fibromodulin | FMOD | 2.752216312 |
| 202728_S_AT | 4052 | ENSG00000049323 | Hs.619315 | latent transforming growth factor beta binding protein 1 | LTBP1 | 3.550016977 |
| 202729_S_AT | 4052 | ENSG00000049323 | Hs.619315 | latent transforming growth factor beta binding protein 1 | LTBP1 | 2.254116381 |
| 202838_AT | 2517 | ENSG00000179163 | Hs.370858 | fucosidase, alpha-L-1, tissue | FUCA1 | 3.246428708 |
| 202869_AT | 4938 | — | Hs.524760 | 2′,5′-oligoadenylate synthetase 1, 40/46 kDa | OAS1 | 12.92037094 |
| 202948_AT | 3554 | ENSG00000115594 | Hs.701982 | interleukin 1 receptor, type I | IL1R1 | 3.402463481 |
| 202956_AT | 10565 | ENSG00000066777 | Hs.656902 | ADP-ribosylation factor guanine nucleotide-exchange | ARFGEF1 | 3.641702112 |
| | | | | factor 1(brefeldin A-inhibited) | | |
| 202962_AT | 23303 | ENSG00000197892 | Hs.444767 | kinesin family member 13B | KIF13B | 4.571846135 |
| 202986_AT | 9915 | ENSG00000172379 | Hs.459070 | aryl-hydrocarbon receptor nuclear translocator 2 | ARNT2 | 0.159256444 |
| 203083_AT | 7058 | ENSG00000186340 | Hs.371147 | thrombospondin 2 | THBS2 | 0.264834407 |
| 203108_AT | 9052 | ENSG00000013588 | Hs.631733 | G protein-coupled receptor, family C, group 5, | GPRC5A | 0.097804861 |
| | | | | member A | | |
| 203140_AT | 604 | ENSG00000113916 | Hs.478588 | B-cell CLL/lymphoma 6 | BCL6 | 3.333889213 |
| 203188_AT | 11041 | ENSG00000174684 | Hs.8526 | UDP-GlcNAc:betaGal beta-1,3-N- | B3GNT1 | 2.218621612 |
| | | | | acetylglucosaminyltransferase 1 | | |
| 203206_AT | 9679 | ENSG00000189319 | Hs.129195 | family with sequence similarity 53, member B | FAM53B | 2.326199169 |
| 203221_AT | 7088 | ENSG00000196781 | Hs.197320 | transducin-like enhancer of split 1 (E(sp1) homolog, | TLE1 | 6.849572806 |
| | | | | Drosophila) | | |
| 203222_S_AT | 7088 | ENSG00000196781 | Hs.197320 | transducin-like enhancer of split 1 (E(sp1) homolog, | TLE1 | 6.147511221 |
| | | | | Drosophila) | | |
| 203234_AT | 7378 | ENSG00000183696 | Hs.488240 | uridine phosphorylase 1 | UPP1 | 4.95505796 |
| 203333_AT | 22920 | ENSG00000075945 | Hs.433442 | kinesin-associated protein 3 | KIFAP3 | 3.474482911 |
| 203349_S_AT | 2119 | ENSG00000244405 | Hs.43697 | ets variant 5 | ETV5 | 2.415849302 |
| 203382_S_AT | 348 | ENSG00000130203 | Hs.654439 | apolipoprotein E | APOE | 2.852019851 |
| 203395_S_AT | 3280 | ENSG00000114315 | Hs.250666 | hairy and enhancer of split 1, (Drosophila) | HES1 | 3.812688994 |
| 203414_AT | 23531 | ENSG00000108960 | Hs.463483 | monocyte to macrophage differentiation-associated | MMD | 2.857230687 |
| 203439_S_AT | 8614 | ENSG00000113739 | Hs.233160 | stanniocalcin 2 | STC2 | 0.222844409 |
| 203476_AT | 7162 | ENSG00000146242 | Hs.82128 | trophoblast glycoprotein | TPBG | 3.055099063 |
| 203477_AT | 1306 | ENSG00000204291 | Hs.409034 | collagen, type XV, alpha 1 | COL15A1 | 29.49590407 |
| 203560_AT | 8836 | ENSG00000137563 | Hs.78619 | gamma-glutamyl hydrolase (conjugase, | GGH | 3.692737503 |
| | | | | folylpolygammaglutamyl hydrolase) | | |
| 203638_S_AT | 2263 | ENSG00000066468 | Hs.533683 | fibroblast growth factor receptor 2 | FGFR2 | 0.099360829 |
| 203680_AT | 5577 | ENSG00000005249 | Hs.433068 | protein kinase, cAMP-dependent, regulatory, type II, | PRKAR2B | 19.19216135 |
| | | | | beta | | |
| 203699_S_AT | 1734 | — | Hs.202354 | deiodinase, iodothyronine, type II | DIO2 | 4.901890228 |
| 203700_S_AT | 1734 | — | Hs.202354 | deiodinase, iodothyronine, type II | DIO2 | 16.8008537 |
| 203710_AT | 3708 | ENSG00000150995 | Hs.567295 | inositol 1,4,5-triphosphate receptor, type 1 | ITPR1 | 3.448251161 |
| 203738_AT | 55322 | ENSG00000082213 | Hs.519246 | chromosome 5 open reading frame 22 | C5orf22 | 3.346259493 |
| 203779_S_AT | 10205 | ENSG00000149573 | Hs.116651 | myelin protein zero-like 2 | MPZL2 | 6.077366063 |
| 203780_AT | 10205 | ENSG00000149573 | Hs.116651 | myelin protein zero-like 2 | MPZL2 | 88.86423632 |
| 203786_S_AT | 7164 | ENSG00000111907 | Hs.591347 | tumor protein D52-like 1 | TPD52L1 | 0.216738103 |
| 203799_AT | 9936 | ENSG00000241399 | Hs.130014 | CD302 molecule | CD302 | 5.283159249 |
| 203817_AT | 2983 | ENSG00000061918 | Hs.77890 | guanylate cyclase 1, soluble, beta 3 | GUCY1B3 | 20.74163365 |
| 203824_AT | 7103 | ENSG00000127324 | Hs.170563 | tetraspanin 8 | TSPAN8 | 3.265874688 |
| 203868_S_AT | 7412 | ENSG00000162692 | Hs.109225 | vascular cell adhesion molecule 1 | VCAM1 | 0.097652882 |
| 203880_AT | 10063 | ENSG00000138495 | Hs.534383 | COX17 cytochrome c oxidase assembly homolog (S. cerevisiae) | COX17 | 2.625226801 |
| 203886_S_AT | 2199 | ENSG00000163520 | Hs.198862 | fibulin 2 | FBLN2 | 0.20643269 |
| 203896_S_AT | 5332 | — | Hs.472101 | phospholipase C, beta 4 | PLCB4 | 0.157312707 |
| 203903_S_AT | 9843 | ENSG00000089472 | Hs.31720 | hephaestin | HEPH | 3.985949343 |
| 203921_AT | 9435 | ENSG00000175040 | Hs.8786 | carbohydrate (N-acetylglucosamine-6-O) | CHST2 | 6.038229953 |
| | | | | sulfotransferase 2 | | |
| 203963_AT | 771 | ENSG00000074410 | Hs.210995 | carbonic anhydrase XII | CA12 | 0.209890205 |
| 203998_S_AT | 6857 | ENSG00000067715 | Hs.310545 | synaptotagmin I | SYT1 | 6.02996106 |
| 204035_AT | 7857 | ENSG00000171951 | Hs.516726 | secretogranin II | SCG2 | 68.97585945 |
| 204105_S_AT | 4897 | ENSG00000091129 | Hs.21422 | neuronal cell adhesion molecule | NRCAM | 28.72018554 |
| 204112_S_AT | 3176 | ENSG00000150540 | Hs.42151 | histamine N-methyltransferase | HNMT | 0.195293231 |
| 204115_AT | 2791 | ENSG00000127920 | Hs.83381 | guanine nucleotide binding protein (G protein), | GNG11 | 2.807997618 |
| | | | | gamma 11 | | |
| 204140_AT | 8460 | ENSG00000169902 | Hs.724538 | tyrosylprotein sulfotransferase 1 | TPST1 | 2.933178408 |
| 204288_S_AT | 8470 | ENSG00000154556 | Hs.619806 | sorbin and SH3 domain containing 2 | SORBS2 | 44.59312821 |
| 204298_S_AT | 4015 | ENSG00000113083 | Hs.102267 | lysyl oxidase | LOX | 4.394380499 |
| 204320_AT | 1301 | — | Hs.523446 | collagen, type XI, alpha 1 | COL11A1 | 4.145962572 |
| 204404_AT | 6558 | ENSG00000064651 | Hs.162585 | solute carrier family 12 (sodium/potassium/chloride | SLC12A2 | 10.40883056 |
| | | | | transporters), member 2 | | |
| 204415_AT | 2537 | ENSG00000126709 | Hs.523847 | interferon, alpha-inducible protein 6 | IFI6 | 12.05942463 |
| 204439_AT | 10964 | ENSG00000137959 | Hs.724492 | interferon-induced protein 44-like | IFI44L | 4.762194633 |
| 204508_S_AT | 771 | ENSG00000074410 | Hs.210995 | carbonic anhydrase XII | CA12 | 0.280927342 |
| 204526_S_AT | 11138 | ENSG00000204634 | Hs.442657 | TBC1 domain family, member 8 (with GRAM | TBC1D8 | 3.765790947 |
| | | | | domain) | | |
| 204529_S_AT | 9760 | ENSG00000198846 | Hs.491805 | thymocyte selection-associated high mobility group | TOX | 0.088913609 |
| | | | | box | | |
| 204575_S_AT | 4327 | ENSG00000123342 | Hs.591033 | matrix metallopeptidase 19 | MMP19 | 4.518958001 |
| 204584_AT | 3897 | ENSG00000198910 | Hs.522818 | L1 cell adhesion molecule | L1CAM | 0.262542115 |
| 204688_AT | 8910 | ENSG00000127990 | Hs.371199 | sarcoglycan, epsilon | SGCE | 2.911280731 |
| 204689_AT | 3087 | — | Hs.118651 | hematopoietically expressed homeobox | HHEX | 31.02449464 |
| 204796_AT | 2009 | ENSG00000066629 | Hs.12451 | echinoderm microtubule associated protein like 1 | EML1 | 3.972766648 |
| 204867_AT | 2644 | ENSG00000137880 | Hs.631717 | GTP cyclohydrolase I feedback regulator | GCHFR | 6.343023897 |
| 204875_S_AT | 2762 | ENSG00000112699 | Hs.144496 | GDP-mannose 4,6-dehydratase | GMDS | 5.069100925 |
| 204922_AT | 79703 | ENSG00000173715 | Hs.292088 | chromosome 11 open reading frame 80 | C11orf80 | 2.895898551 |
| 204932_AT | 4982 | ENSG00000164761 | Hs.81791 | tumor necrosis factor receptor superfamily, member | TNFRSF11B | 0.041821591 |
| | | | | 11b | | |
| 204933_S_AT | 4982 | ENSG00000164761 | Hs.81791 | tumor necrosis factor receptor superfamily, member | TNFRSF11B | 0.03801027 |
| | | | | 11b | | |
| 204972_AT | 4939 | — | Hs.414332 | 2′-5′-oligoadenylate synthetase 2, 69/71 kDa | OAS2 | 24.77110983 |
| 204994_AT | 4600 | ENSG00000183486 | Hs.926 | myxovirus (influenza virus) resistance 2 (mouse) | MX2 | 8.313178914 |
| 205111_S_AT | 51196 | ENSG00000138193 | Hs.655033 | phospholipase C, epsilon 1 | PLCE1 | 4.180455948 |
| 205174_S_AT | 25797 | ENSG00000115828 | Hs.79033 | glutaminyl-peptide cyclotransferase | QPCT | 3.05397697 |
| 205200_AT | 7123 | ENSG00000163815 | Hs.476092 | C-type lectin domain family 3, member B | CLEC3B | 0.081105693 |
| 205201_AT | 2737 | ENSG00000106571 | Hs.21509 | GLI family zinc finger 3 | GLI3 | 4.138575473 |
| 205248_AT | 9980 | ENSG00000142197 | Hs.204575 | dopey family member 2 | DOPEY2 | 0.244970836 |
| 205258_AT | 3625 | ENSG00000163083 | Hs.1735 | inhibin, beta B | INHBB | 0.049141267 |
| 205280_AT | 2743 | ENSG00000109738 | Hs.32973 | glycine receptor, beta | GLRB | 2.762107666 |
| 205285_S_AT | 2533 | ENSG00000082074 | Hs.370503 | FYN binding protein | FYB | 2.939191733 |
| 205299_S_AT | 10385 | ENSG00000124508 | Hs.373938 | butyrophilin, subfamily 2, member A2 | BTN2A2 | 2.87730496 |
| 205376_AT | 8821 | ENSG00000109452 | Hs.658245 | inositol polyphosphate-4-phosphatase, type II, | INPP4B | 0.156079561 |
| | | | | 105 kDa | | |
| 205403_AT | 7850 | ENSG00000115590 | Hs.25333 | interleukin 1 receptor, type II | IL1R2 | 33.39336752 |
| 205475_AT | 11341 | ENSG00000164106 | Hs.7122 | stimulator of chondrogenesis 1 | SCRG1 | 4.469689582 |
| 205483_S_AT | 9636 | ENSG00000187608 | Hs.458485 | ISG15 ubiquitin-like modifier | ISG15 | 9.398387466 |
| 205552_S_AT | 4938 | ENSG00000089127 | Hs.524760 | 2′,5′-oligoadenylate synthetase 1, 40/46 kDa | OAS1 | 12.4825101 |
| 205573_S_AT | 51375 | ENSG00000162627 | Hs.197015 | sorting nexin 7 | SNX7 | 3.740166342 |
| 205590_AT | 10125 | ENSG00000172575 | Hs.591127 | RAS guanyl releasing protein 1 (calcium and DAG- | RASGRP1 | 0.102216569 |
| | | | | regulated) | | |
| 205602_X_AT | 5676 | ENSG00000221878 | Hs.709203 | pregnancy specific beta-1-glycoprotein 7 | PSG7 | 0.130635018 |
| | | | | (gene/pseudogene) | | |
| 205606_AT | 4040 | ENSG00000070018 | Hs.584775 | low density lipoprotein receptor-related protein 6 | LRP6 | 3.165495117 |
| 205741_S_AT | 1837 | ENSG00000134769 | Hs.643454 | dystrobrevin, alpha | DTNA | 3.00218919 |
| 205771_S_AT | 9465 | ENSG00000118507 | Hs.486483 | A kinase (PRKA) anchor protein 7 | AKAP7 | 3.402934544 |
| 205805_S_AT | 4919 | ENSG00000185483 | Hs.654491 | receptor tyrosine kinase-like orphan receptor 1 | ROR1 | 0.270768381 |
| 205828_AT | 4314 | ENSG00000149968 | Hs.375129 | matrix metallopeptidase 3 (stromelysin 1, | MMP3 | 0.121050602 |
| | | | | progelatinase) | | |
| 205832_AT | 51200 | — | Hs.93764 | carboxypeptidase A4 | CPA4 | 0.086107627 |
| 205890_S_AT | 10537 /// 2550 | | Hs.167017 | gamma-aminobutyric acid (GABA) B receptor, 1 /// | GABBR1 /// | 18.08261971 |
| | | | | ubiquitin D | UBD | |
| 205904_AT | 4276 | ENSG00000183214 | Hs.130838 | MHC class I polypeptide-related sequence A | MICA | 5.891148199 |
| 205905_S_AT | 4276 /// 4277 | ENSG00000183214 /// | Hs.719929 | MHC class I polypeptide-related sequence A /// MHC class I polypeptide-related sequence B | MICA /// MICB | 4.891679691 |
| | | ENSG00000204516 /// | | | | |
| | | ENSG00000204520 /// | | | | |
| | | ENSG00000206449 /// | | | | |
| | | ENSG00000224378 /// | | | | |
| | | ENSG00000227772 /// | | | | |
| | | ENSG00000231179 /// | | | | |
| | | ENSG00000231225 /// | | | | |
| | | ENSG00000231372 /// | | | | |
| | | ENSG00000233051 /// | | | | |
| | | ENSG00000234218 /// | | | | |
| | | ENSG00000235233 /// | | | | |
| | | ENSG00000238289 | | | | |
| 206001_AT | 4852 | ENSG00000122585 | Hs.1832 | neuropeptide Y | NPY | 8.150303583 |
| 206025_S_AT | 7130 | ENSG00000123610 | Hs.437322 | tumor necrosis factor, alpha-induced protein 6 | TNFAIP6 | 15.72437817 |
| 206026_S_AT | 7130 | ENSG00000123610 | Hs.437322 | tumor necrosis factor, alpha-induced protein 6 | TNFAIP6 | 23.039848 |
| 206062_AT | 2978 | ENSG00000048545 | Hs.92858 | guanylate cyclase activator 1A (retina) | GUCA1A | 4.484313198 |
| 206082_AT | 10866 | ENSG00000206337 /// | — | HLA complex P5 | HCP5 | 7.169342768 |
| | | ENSG00000227429 /// | | | | |
| | | ENSG00000230389 /// | | | | |
| | | ENSG00000237105 | | | | |
| 206084_AT | 5801 | ENSG00000153233 | Hs.506076 | protein tyrosine phosphatase, receptor type, R | PTPRR | 0.269094855 |
| 206157_AT | 5806 | ENSG00000163661 | Hs.591286 | pentraxin 3, long | PTX3 | 0.093262421 |
| 206204_AT | 2888 | ENSG00000115290 | Hs.411881 | growth factor receptor-bound protein 14 | GRB14 | 28.39476896 |
| 206290_S_AT | 6000 | ENSG00000182901 | Hs.655739 | regulator of G-protein signaling 7 | RGS7 | 0.06583198 |
| 206302_S_AT | 11163 /// 440672 | ENSG00000173598 | Hs.506325 | nudix (nucleoside diphosphate linked moiety X)-type | NUDT4 /// | 4.180594003 |
| | | | | motif 4 /// nudix (nucleoside diphosphate linked | NUDT4P1 | |
| | | | | moiety X)-type motif 4 pseudogene 1 | | |
| 206303_S_AT | 11163 | ENSG00000173598 | Hs.506325 | nudix (nucleoside diphosphate linked moiety X)-type | NUDT4 | 4.737334058 |
| | | | | motif 4 | | |
| 206336_AT | 6372 | — | Hs.164021 | chemokine (C—X—C motif) ligand 6 (granulocyte | CXCL6 | 22.73329459 |
| | | | | chemotactic protein 2) | | |
| 206421_S_AT | 8710 | ENSG00000166396 | Hs.138202 | serpin peptidase inhibitor, clade B (ovalbumin), | SERPINB7 | 6.543117392 |
| | | | | member 7 | | |
| 206518_S_AT | 8787 | ENSG00000108370 | Hs.664380 | regulator of G-protein signaling 9 | RGS9 | 9.495194262 |
| 206522_AT | 8972 | ENSG00000179087 | Hs.122785 | maltase-glucoamylase (alpha-glucosidase) | MGAM | 0.13476016 |
| 206526_AT | 26150 | ENSG00000128408 | Hs.475110 | RIB43A domain with coiled-coils 2 | RIBC2 | 2.823873292 |
| 206584_AT | 23643 | ENSG00000154589 | Hs.660766 | lymphocyte antigen 96 | LY96 | 7.776127283 |
| 206653_AT | 10622 | ENSG00000113356 | Hs.282387 | polymerase (RNA) III (DNA directed) polypeptide G | POLR3G | 0.225153695 |
| | | | | (32 kD) | | |
| 206707_X_AT | 9750 | ENSG00000111913 | Hs.559459 | family with sequence similarity 65, member B | FAM65B | 7.068833096 |
| 206730_AT | 2892 | ENSG00000125675 | Hs.377070 | glutamate receptor, ionotrophic, AMPA 3 | GRIA3 | 6.579596381 |
| 206740_X_AT | 6847 | ENSG00000198765 | Hs.112743 | synaptonemal complex protein 1 | SYCP1 | 0.39861654 |
| 206756_AT | 56548 | ENSG00000147119 | Hs.129955 | carbohydrate (N-acetylglucosamine 6-O) | CHST7 | 3.850881623 |
| | | | | sulfotransferase 7 | | |
| 206757_AT | 8654 | — | Hs.587281 | phosphodiesterase 5A, cGMP-specific | PDE5A | 6.998056707 |
| 206765_AT | 3759 | ENSG00000123700 | Hs.1547 | potassium inwardly-rectifying channel, subfamily J, | KCNJ2 | 25.31335494 |
| | | | | member 2 | | |
| 206825_AT | 5021 | ENSG00000180914 | Hs.2820 | oxytocin receptor | OXTR | 0.355959964 |
| 206857_S_AT | 2281 | ENSG00000119782 | Hs.709461 | FK506 binding protein 1B, 12.6 kDa | FKBP1B | 2.581981705 |
| 206938_AT | 6716 | ENSG00000049319 | Hs.458345 | steroid-5-alpha-reductase, alpha polypeptide 2 (3-oxo- | SRD5A2 | 2.730514297 |
| | | | | 5 alpha-steroid delta 4-dehydrogenase alpha 2) | | |
| 207030_S_AT | 1466 | ENSG00000175183 | Hs.530904 | cysteine and glycine-rich protein 2 | CSRP2 | 5.930896094 |
| 207069_S_AT | 4091 | ENSG00000137834 | Hs.153863 | SMAD family member 6 | SMAD6 | 0.251037916 |
| 207076_S_AT | 445 | ENSG00000130707 | Hs.160786 | argininosuccinate synthase 1 | ASS1 | 0.223368856 |
| 207145_AT | 2660 | ENSG00000138379 | Hs.41565 | myostatin | MSTN | 0.250445722 |
| 207238_S_AT | 5788 | ENSG00000081237 | Hs.654514 | protein tyrosine phosphatase, receptor type, C | PTPRC | 18.21723381 |
| 207334_S_AT | 7048 | ENSG00000163513 | Hs.82028 | transforming growth factor, beta receptor II | TGFBR2 | 3.350901472 |
| | | | | (70/80 kDa) | | |
| 207370_AT | 3381 | ENSG00000029559 | Hs.518726 | integrin-binding sialoprotein | IBSP | 33.97189816 |
| 207717_S_AT | 5318 | ENSG00000057294 | Hs.164384 | plakophilin 2 | PKP2 | 0.272503601 |
| 207761_S_AT | 25840 | ENSG00000185432 | Hs.723867 | methyltransferase like 7A | METTL7A | 32.60921553 |
| 207808_S_AT | 5627 | ENSG00000184500 | Hs.64016 | protein S (alpha) | PROS1 | 8.214553802 |
| 207826_S_AT | 3399 | ENSG00000117318 | Hs.76884 | inhibitor of DNA binding 3, dominant negative helix- | ID3 | 0.160178956 |
| | | | | loop-helix protein | | |
| 207876_S_AT | 2318 | ENSG00000128591 | Hs.58414 | filamin C, gamma | FLNC | 0.26950312 |
| 208091_S_AT | 81552 | ENSG00000154978 | Hs.488307 | vesicular, overexpressed in cancer, prosurvival protein 1 | VOPP1 | 2.362349583 |
| 208134_X_AT | 5670 | ENSG00000242221 | Hs.709200 | pregnancy specific beta-1-glycoprotein 2 | PSG2 | 0.398244543 |
| 208257_X_AT | 5669 | ENSG00000231924 | Hs.709192 | pregnancy specific beta-1-glycoprotein 1 | PSG1 | 0.076092167 |
| 208436_S_AT | 3665 | ENSG00000185507 | Hs.166120 | interferon regulatory factor 7 | IRF7 | 7.153585188 |
| 208447_S_AT | 5631 | ENSG00000147224 | Hs.56 | phosphoribosyl pyrophosphate synthetase 1 | PRPS1 | 0.187293773 |
| 208926_AT | 4758 | ENSG00000184494 /// | Hs.520037 | sialidase 1 (lysosomal sialidase) | NEU1 | 3.127653485 |
| | | ENSG00000204386 /// | | | | |
| | | ENSG00000223957 /// | | | | |
| | | ENSG00000227129 /// | | | | |
| | | ENSG00000227315 /// | | | | |
| | | ENSG00000228691 /// | | | | |
| | | ENSG00000234343 /// | | | | |
| | | ENSG00000234846 | | | | |
| 208937_S_AT | 3397 | ENSG00000125968 | Hs.504609 | inhibitor of DNA binding 1, dominant negative helix- | ID1 | 0.074466994 |
| | | | | loop-helix protein | | |
| 208944_AT | 7048 | ENSG00000163513 | Hs.82028 | transforming growth factor, beta receptor II | TGFBR2 | 8.374643442 |
| | | | | (70/80 kDa) | | |
| 209030_S_AT | 23705 | ENSG00000182985 | Hs.370510 | cell adhesion molecule 1 | CADM1 | 11.9943899 |
| 209031_AT | 23705 | ENSG00000182985 | Hs.370510 | cell adhesion molecule 1 | CADM1 | 11.39553478 |
| 209032_S_AT | 23705 | ENSG00000182985 | Hs.370510 | cell adhesion molecule 1 | CADM1 | 12.34977109 |
| 209035_AT | 4192 | ENSG00000110492 | Hs.82045 | midkine (neurite growth-promoting factor 2) | MDK | 3.24444128 |
| 209160_AT | 8644 | ENSG00000196139 | Hs.78183 | aldo-keto reductase family 1, member C3 (3-alpha | AKR1C3 | 17.48283508 |
| | | | | hydroxysteroid dehydrogenase, type II) | | |
| 209211_AT | 688 | ENSG00000102554 | Hs.508234 | Kruppel-like factor 5 (intestinal) | KLF5 | 4.182440502 |
| 209212_S_AT | 688 | ENSG00000102554 | Hs.508234 | Kruppel-like factor 5 (intestinal) | KLF5 | 6.401068416 |
| 209230_S_AT | 26471 | ENSG00000176046 | Hs.513463 | nuclear protein, transcriptional regulator, 1 | NUPR1 | 0.121224345 |
| 209387_S_AT | 4071 | ENSG00000169908 | Hs.723828 | transmembrane 4 L six family member 1 | TM4SF1 | 0.316674927 |
| 209420_S_AT | 6609 | ENSG00000166311 | Hs.498173 | sphingomyelin phosphodiesterase 1, acid lysosomal | SMPD1 | 4.984732415 |
| 209462_AT | 333 | ENSG00000105290 | Hs.74565 | amyloid beta (A4) precursor-like protein 1 | APLP1 | 3.122499509 |
| 209465_X_AT | 5764 | ENSG00000105894 | Hs.371249 | pleiotrophin | PTN | 7.282947698 |
| 209466_X_AT | 5764 | ENSG00000105894 | Hs.371249 | pleiotrophin | PTN | 7.3129428 |
| 209493_AT | 23037 | ENSG00000133401 | Hs.481819 | PDZ domain containing 2 | PDZD2 | 22.53153979 |
| 209539_AT | 9459 | ENSG00000129675 | Hs.522795 | Rac/Cdc42 guanine nucleotide exchange factor (GEF) 6 | ARHGEF6 | 3.084953469 |
| 209595_AT | 2963 | ENSG00000188342 | Hs.654582 | general transcription factor IIF, polypeptide 2, 30 kDa | GTF2F2 | 5.614223112 |
| 209596_AT | 25878 | ENSG00000101825 | Hs.369422 | matrix-remodelling associated 5 | MXRA5 | 3.481072616 |
| 209598_AT | 10687 | — | Hs.591838 | paraneoplastic antigen MA2 | PNMA2 | 0.159001619 |
| 209619_AT | 972 | ENSG00000019582 | Hs.436568 | CD74 molecule, major histocompatibility complex, | CD74 | 4.54731358 |
| | | | | class II invariant chain | | |
| 209631_S_AT | 2861 | ENSG00000170775 | Hs.723816 | G protein-coupled receptor 37 (endothelin receptor | GPR37 | 16.27483304 |
| | | | | type B-like) | | |
| 209708_AT | 26002 | ENSG00000079931 | Hs.6909 | monooxygenase, DBH-like 1 | MOXD1 | 2.655684258 |
| 209719_X_AT | 6317 | ENSG00000057149 | Hs.227948 | serpin peptidase inhibitor, clade B (ovalbumin), | SERPINB3 | 108.5618238 |
| | | | | member 3 | | |
| 209720_S_AT | 6317 | ENSG00000057149 | Hs.227948 | serpin peptidase inhibitor, clade B (ovalbumin), | SERPINB3 | 285.5423381 |
| | | | | member 3 | | |
| 209758_S_AT | 8076 | ENSG00000197614 | Hs.512842 | microfibrillar associated protein 5 | MFAP5 | 56.44742425 |
| 209763_AT | 91851 | ENSG00000101938 | Hs.496587 | chordin-like 1 | CHRDL1 | 0.180046056 |
| 209765_AT | 8728 | ENSG00000135074 | Hs.483944 | ADAM metallopeptidase domain 19 (meltrin beta) | ADAM19 | 4.747463808 |
| 209771_X_AT | 100133941 | ENSG00000185275 | Hs.644105 | CD24 molecule | CD24 | 0.22272565 |
| 209792_S_AT | 5655 | ENSG00000129451 | Hs.275464 | kallikrein-related peptidase 10 | KLK10 | 12.20068261 |
| 209829_AT | 9750 | ENSG00000111913 | Hs.559459 | family with sequence similarity 65, member B | FAM65B | 22.59284714 |
| 209839_AT | 26052 | ENSG00000197959 | Hs.654775 | dynamin 3 | DNM3 | 26.33714156 |
| 209840_S_AT | 54674 | ENSG00000173114 | Hs.3781 | leucine rich repeat neuronal 3 | LRRN3 | 5.871787003 |
| 209841_S_AT | 54674 | ENSG00000173114 | Hs.3781 | leucine rich repeat neuronal 3 | LRRN3 | 5.31041496 |
| 209875_S_AT | 6696 | ENSG00000118785 | Hs.313 | secreted phosphoprotein 1 | SPP1 | 142.8904442 |
| 209894_AT | 3953 | ENSG00000116678 | Hs.723178 | leptin receptor | LEPR | 0.102322833 |
| 209909_S_AT | 7042 | ENSG00000092969 | Hs.133379 | transforming growth factor, beta 2 | TGFB2 | 0.212614802 |
| 210015_S_AT | 4133 | ENSG00000078018 | Hs.368281 | microtubule-associated protein 2 | MAP2 | 4.864593206 |
| 210042_S_AT | 1522 | ENSG00000101160 | Hs.252549 | cathepsin Z | CTSZ | 3.136941278 |
| 210126_AT | 5678 | ENSG00000183668 | Hs.502092 | pregnancy specific beta-1-glycoprotein 9 | PSG9 | 0.290278453 |
| 210130_S_AT | 7108 | ENSG00000149809 | Hs.31130 | transmembrane 7 superfamily member 2 | TM7SF2 | 4.025993031 |
| 210195_S_AT | 5669 | ENSG00000231924 | Hs.709192 | pregnancy specific beta-1-glycoprotein 1 | PSG1 | 0.096442758 |
| 210196_S_AT | 5669 | ENSG00000231924 | Hs.709192 | pregnancy specific beta-1-glycoprotein 1 | PSG1 | 0.117845527 |
| 210299_S_AT | 2273 | ENSG00000022267 | Hs.435369 | four and a half LIM domains 1 | FHL1 | 0.062776843 |
| 210347_S_AT | 53335 | ENSG00000119866 | Hs.370549 | B-cell CLL/lymphoma 11A (zinc finger protein) | BCL11A | 5.703973748 |
| 210413_X_AT | 6317 /// 6318 | ENSG00000206073 | Hs.123035 | serpin peptidase inhibitor, clade B (ovalbumin), | SERPINB3 /// | 88.60759874 |
| | | | | member 3 /// serpin peptidase inhibitor, clade B | SERPINB4 | |
| | | | | (ovalbumin), member 4 | | |
| 210473_S_AT | 166647 | ENSG00000152990 | Hs.99195 | G protein-coupled receptor 125 | GPR125 | 3.196951076 |
| 210511_S_AT | 3624 | ENSG00000122641 | Hs.583348 | inhibin, beta A | INHBA | 3.451352739 |
| 210517_S_AT | 9590 | ENSG00000131016 | Hs.371240 | A kinase (PRKA) anchor protein 12 | AKAP12 | 2.475435495 |
| 210619_S_AT | 3373 | ENSG00000114378 | Hs.75619 | hyaluronoglucosaminidase 1 | HYAL1 | 2.887661592 |
| 210757_X_AT | 1601 | ENSG00000153071 | Hs.481980 | disabled homolog 2, mitogen-responsive | DAB2 | 0.324500742 |
| | | | | phosphoprotein (Drosophila) | | |
| 210980_S_AT | 427 | ENSG00000104763 | Hs.527412 | N-acylsphingosine amidohydrolase (acid ceramidase) 1 | ASAH1 | 4.165603031 |
| 211126_S_AT | 1466 | ENSG00000175183 | Hs.530904 | cysteine and glycine-rich protein 2 | CSRP2 | 6.287088722 |
| 211355_X_AT | 3953 | ENSG00000116678 | Hs.723178 | leptin receptor | LEPR | 0.154769706 |
| 211356_X_AT | 3953 | ENSG00000116678 | Hs.723178 | leptin receptor | LEPR | 0.153115331 |
| 211372_S_AT | 7850 | ENSG00000115590 | Hs.25333 | interleukin 1 receptor, type II | IL1R2 | 16.76268411 |
| 211679_X_AT | 9568 | ENSG00000136928 | Hs.198612 | gamma-aminobutyric acid (GABA) B receptor, 2 | GABBR2 | 2.493617142 |
| 211732_X_AT | 3176 | ENSG00000150540 | Hs.42151 | histamine N-methyltransferase | HNMT | 0.365633305 |
| 211737_X_AT | 5764 | ENSG00000105894 | Hs.371249 | pleiotrophin | PTN | 7.761048277 |
| 211795_S_AT | 2533 | ENSG00000082074 | Hs.370503 | FYN binding protein | FYB | 30.44105868 |
| 211799_X_AT | 3107 | — | Hs.654404 | major histocompatibility complex, class I, C | HLA-C | 3.397125918 |
| 211828_S_AT | 23043 | ENSG00000154310 | Hs.34024 | TRAF2 and NCK interacting kinase | TNIK | 6.882437612 |
| 211906_S_AT | 6318 | ENSG00000206073 | Hs.123035 | serpin peptidase inhibitor, clade B (ovalbumin), | SERPINB4 | 115.7698134 |
| | | | | member 4 | | |
| 212070_AT | 9289 | ENSG00000205336 | Hs.513633 | G protein-coupled receptor 56 | GPR56 | 0.160896012 |
| 212124_AT | 57178 | ENSG00000108175 | Hs.193118 | zinc finger, MIZ-type containing 1 | ZMIZ1 | 0.237569432 |
| 212181_S_AT | 11163 /// 440672 | ENSG00000173598 | Hs.506325 | nudix (nucleoside diphosphate linked moiety X)-type | NUDT4 /// | 3.171017177 |
| | | | | motif 4 /// nudix (nucleoside diphosphate linked | NUDT4P1 | |
| | | | | moiety X)-type motif 4 pseudogene 1 | | |
| 212183_AT | 11163 | ENSG00000173598 | Hs.506325 | nudix (nucleoside diphosphate linked moiety X)-type | NUDT4 | 4.455353115 |
| | | | | motif 4 | | |
| 212190_AT | 5270 | ENSG00000135919 | Hs.38449 | serpin peptidase inhibitor, clade E (nexin, | SERPINE2 | 7.483146043 |
| | | | | plasminogen activator inhibitor type 1), member 2 | | |
| 212242_AT | 7277 | ENSG00000127824 | Hs.75318 | tubulin, alpha 4a | TUBA4A | 4.597319715 |
| 212327_AT | 22998 | ENSG00000064042 | Hs.335163 | LIM and calponin homology domains 1 | LIMCH1 | 3.536298154 |
| 212334_AT | 2799 | ENSG00000135677 | Hs.334534 | glucosamine (N-acetyl)-6-sulfatase | GNS | 2.457244326 |
| 212335_AT | 2799 | ENSG00000135677 | Hs.334534 | glucosamine (N-acetyl)-6-sulfatase | GNS | 2.776148188 |
| 212338_AT | 4642 | ENSG00000176658 | Hs.602063 | myosin ID | MYO1D | 2.606396225 |
| 212444_AT | — | — | Hs.632997 | — | — | 0.062639967 |
| 212558_AT | 10252 | ENSG00000164056 | Hs.436944 | sprouty homolog 1, antagonist of FGF signaling | SPRY1 | 5.403605284 |
| | | | | (Drosophila) | | |
| 212587_S_AT | 5788 | ENSG00000081237 | Hs.654514 | protein tyrosine phosphatase, receptor type, C | PTPRC | 50.99523078 |
| 212588_AT | 5788 | ENSG00000081237 | Hs.654514 | protein tyrosine phosphatase, receptor type, C | PTPRC | 86.4552217 |
| 212624_S_AT | 1123 | ENSG00000128656 | Hs.380138 | chimerin (chimaerin) 1 | CHN1 | 7.893190144 |
| 212650_AT | 23301 | ENSG00000115504 | Hs.271667 | EH domain binding protein 1 | EHBP1 | 4.557335754 |
| 212653_S_AT | 23301 | ENSG00000115504 | Hs.271667 | EH domain binding protein 1 | EHBP1 | 3.798328664 |
| 212658_AT | 10184 | ENSG00000145685 | Hs.79299 | lipoma HMGIC fusion partner-like 2 | LHFPL2 | 2.477070062 |
| 212719_AT | 23239 | ENSG00000081913 | Hs.465337 | PH domain and leucine rich repeat protein | PHLPP1 | 2.369422345 |
| | | | | phosphatase 1 | | |
| 212813_AT | 83700 | ENSG00000166086 | Hs.150718 | junctional adhesion molecule 3 | JAM3 | 2.663065656 |
| 212830_AT | 1955 | ENSG00000106780 | Hs.494977 | multiple EGF-like-domains 9 | MEGF9 | 3.145943527 |
| 212850_S_AT | 4038 | ENSG00000134569 | Hs.4930 | low density lipoprotein receptor-related protein 4 | LRP4 | 5.519731741 |
| 212886_AT | 26112 | ENSG00000198624 | Hs.655336 | coiled-coil domain containing 69 | CCDC69 | 2.35755419 |
| 212909_AT | 116372 | ENSG00000150551 | Hs.432395 | LY6/PLAUR domain containing 1 | LYPD1 | 4.694002197 |
| 212950_AT | 221395 | ENSG00000069122 | Hs.362806 | G protein-coupled receptor 116 | GPR116 | 12.70835634 |
| 212951_AT | 221395 | ENSG00000069122 | Hs.362806 | G protein-coupled receptor 116 | GPR116 | 5.012614641 |
| 212978_AT | 23507 | ENSG00000197147 | Hs.482017 | leucine rich repeat containing 8 family, member B | LRRC8B | 5.11732797 |
| 213017_AT | 171586 | ENSG00000158201 | Hs.397978 | abhydrolase domain containing 3 | ABHD3 | 5.551182616 |
| 213085_S_AT | 23286 | ENSG00000113645 | Hs.484047 | WW and C2 domain containing 1 | WWC1 | 0.204604916 |
| 213107_AT | 23043 | ENSG00000154310 | Hs.34024 | TRAF2 and NCK interacting kinase | TNIK | 5.536293692 |
| 213145_AT | 144699 | — | Hs.367956 | F-box and leucine-rich repeat protein 14 | FBXL14 | 2.496460871 |
| 213221_S_AT | 23235 | — | Hs.269128 | salt-inducible kinase 2 | SIK2 | 5.920007292 |
| 213222_AT | 23236 | ENSG00000182621 | Hs.431173 | phospholipase C, beta 1 (phosphoinositide-specific) | PLCB1 | 4.225933822 |
| 213413_AT | 11037 | ENSG00000243244 | Hs.44385 | stonin 1 | STON1 | 2.710684316 |
| 213438_AT | 23114 | ENSG00000163531 | Hs.13349 | neurofascin homolog (chicken) | NFASC | 0.142941859 |
| 213506_AT | 2150 | ENSG00000164251 | Hs.154299 | coagulation factor II (thrombin) receptor-like 1 | F2RL1 | 28.93155244 |
| 213659_AT | 7626 | ENSG00000186376 | Hs.533540 | zinc finger protein 75D | ZNF75D | 4.008340417 |
| 213702_X_AT | 427 | ENSG00000104763 | Hs.527412 | N-acylsphingosine amidohydrolase (acid ceramidase) 1 | ASAH1 | 4.144545581 |
| 213721_AT | 6657 | ENSG00000181449 | Hs.518438 | SRY (sex determining region Y)-box 2 | SOX2 | 36.56644535 |
| 213764_S_AT | 8076 | ENSG00000197614 | Hs.512842 | microfibrillar associated protein 5 | MFAP5 | 71.1228272 |
| 213765_AT | 8076 | ENSG00000197614 | Hs.512842 | microfibrillar associated protein 5 | MFAP5 | 47.70652849 |
| 213800_AT | 3075 | ENSG00000000971 | Hs.363396 | complement factor H | CFH | 4.254884284 |
| 213802_AT | — | — | Hs.445857 | — | — | 4.947331588 |
| 213902_AT | 427 | ENSG00000104763 | Hs.527412 | N-acylsphingosine amidohydrolase (acid ceramidase) 1 | ASAH1 | 2.641870195 |
| 214164_X_AT | 771 | ENSG00000074410 | Hs.210995 | carbonic anhydrase XII | CA12 | 0.182752804 |
| 214446_AT | 22936 | ENSG00000118985 | Hs.192221 | elongation factor, RNA polymerase II, 2 | ELL2 | 0.17659959 |
| 214453_S_AT | 10561 | ENSG00000137965 | Hs.82316 | interferon-induced protein 44 | IFI44 | 8.23883363 |
| 214505_S_AT | 2273 | ENSG00000022267 | Hs.435369 | four and a half LIM domains 1 | FHL1 | 0.069066282 |
| 214660_AT | 53918 | — | Hs.644352 | Pelota homolog (Drosophila) | PELO | 17.6572153 |
| 214719_AT | 283537 | ENSG00000139508 | Hs.117167 | solute carrier family 46, member 3 | SLC46A3 | 3.75243609 |
| 214985_AT | 2131 | ENSG00000182197 | Hs.492618 | exostosis 1 | EXT1 | 0.308235253 |
| 215388_S_AT | 3075 /// 3078 | ENSG00000000971 /// | Hs.363396 | complement factor H /// complement factor H-related 1 | CFH /// CFHR1 | 7.338239151 |
| | | ENSG00000244414 | | | | |
| 215446_S_AT | 4015 | ENSG00000113083 | Hs.102267 | lysyl oxidase | LOX | 2.855908308 |
| 215506_S_AT | 9077 | ENSG00000162595 | Hs.194695 | DIRAS family, GTP-binding RAS-like 3 | DIRAS3 | 46.12919232 |
| 215867_X_AT | 771 | ENSG00000074410 | Hs.210995 | carbonic anhydrase XII | CA12 | 0.183444237 |
| 215933_S_AT | 3087 | ENSG00000152804 | Hs.118651 | hematopoietically expressed homeobox | HHEX | 33.50773616 |
| 215955_X_AT | 23092 | ENSG00000145819 | Hs.654668 | Rho GTPase activating protein 26 | ARHGAP26 | 2.722587871 |
| 216074_X_AT | 23286 | ENSG00000113645 | Hs.484047 | WW and C2 domain containing 1 | WWC1 | 0.281911287 |
| 216230_X_AT | 6609 | ENSG00000166311 | Hs.498173 | sphingomyelin phosphodiesterase 1, acid lysosomal | SMPD1 | 6.00857847 |
| 216933_X_AT | 324 | ENSG00000134982 | Hs.158932 | adenomatous polyposis coli | APC | 2.884860854 |
| 217350_AT | 160313 | ENSG00000216306 | Hs.527883 | keratin 19 pseudogene 2 | KRT19P2 | 0.193891902 |
| 217707_X_AT | 6595 | ENSG00000080503 | Hs.298990 | SWI/SNF related, matrix associated, actin dependent | SMARCA2 | 3.617053082 |
| | | | | regulator of chromatin, subfamily a, member 2 | | |
| 217974_AT | 51768 | ENSG00000064115 | Hs.438641 | transmembrane 7 superfamily member 3 | TM7SF3 | 4.034207184 |
| 217979_AT | 27075 | — | Hs.364544 | tetraspanin 13 | TSPAN13 | 11.71868576 |
| 218313_S_AT | 51809 | ENSG00000109586 | Hs.548088 | UDP-N-acetyl-alpha-D-galactosamine:polypeptide N- | GALNT7 | 2.599288727 |
| | | | | acetylgalactosaminyltransferase 7 (GalNAc-T7) | | |
| 218400_AT | 4940 | ENSG00000111331 | Hs.528634 | 2′-5′-oligoadenylate synthetase 3, 100 kDa | OAS3 | 5.849582587 |
| 218546_AT | 79762 | ENSG00000162817 | Hs.519839 | chromosome 1 open reading frame 115 | C1orf115 | 3.329144684 |
| 218589_AT | 10161 | ENSG00000139679 | Hs.123464 | lysophosphatidic acid receptor 6 | LPAR6 | 3.431585811 |
| 218723_S_AT | 28984 | ENSG00000102760 | Hs.507866 | chromosome 13 open reading frame 15 | C13orf15 | 41.77246447 |
| 218802_AT | 55013 | ENSG00000005059 | Hs.234149 | coiled-coil domain containing 109B | CCDC109B | 2.087934184 |
| 218824_AT | 55228 | ENSG00000182013 | Hs.8395 | PNMA-like 1 | PNMAL1 | 5.317122999 |
| 218839_AT | 23462 | ENSG00000164683 | Hs.234434 | hairy/enhancer-of-split related with YRPW motif 1 | HEY1 | 7.872792953 |
| 218843_AT | 64838 | ENSG00000115226 | Hs.724632 | fibronectin type III domain containing 4 | FNDC4 | 3.630241489 |
| 218865_AT | 64757 | ENSG00000186205 | Hs.497816 | MOCO sulphurase C-terminal domain containing 1 | MOSC1 | 8.649979279 |
| 218885_S_AT | 79695 | ENSG00000119514 | Hs.47099 | UDP-N-acetyl-alpha-D-galactosamine:polypeptide N- | GALNT12 | 23.82401494 |
| | | | | acetylgalactosaminyltransferase 12 (GalNAc-T12) | | |
| 218927_S_AT | 55501 | ENSG00000136213 | Hs.213088 | carbohydrate (chondroitin 4) sulfotransferase 12 | CHST12 | 2.229247768 |
| 218953_S_AT | 78991 | ENSG00000145882 | Hs.644397 | prenylcysteine oxidase 1 like | PCYOX1L | 2.689764411 |
| 218980_AT | 80206 | ENSG00000134775 | Hs.719944 | formin homology 2 domain containing 3 | FHOD3 | 6.704048452 |
| 218995_S_AT | 1906 | ENSG00000078401 | Hs.511899 | endothelin 1 | EDN1 | 7.96324246 |
| 218999_AT | 55281 | — | Hs.724675 | transmembrane protein 140 | TMEM140 | 4.250227357 |
| 219033_AT | 79668 | ENSG00000151883 | Hs.369581 | poly (ADP-ribose) polymerase family, member 8 | PARP8 | 6.245904266 |
| 219087_AT | 54829 | ENSG00000106819 | Hs.435655 | asporin | ASPN | 0.164843921 |
| 219147_S_AT | 54981 | ENSG00000106733 | Hs.494186 | chromosome 9 open reading frame 95 | C9orf95 | 3.522050531 |
| 219169_S_AT | 51106 | ENSG00000029639 | Hs.279908 | transcription factor B1, mitochondrial | TFB1M | 2.462742128 |
| 219181_AT | 9388 | ENSG00000101670 | Hs.465102 | lipase, endothelial | LIPG | 24.24554048 |
| 219209_AT | 64135 | ENSG00000115267 | Hs.163173 | interferon induced with helicase C domain 1 | IFIH1 | 5.861140516 |
| 219211_AT | 11274 | ENSG00000184979 | Hs.38260 | ubiquitin specific peptidase 18 | USP18 | 3.098152941 |
| 219213_AT | 58494 | ENSG00000154721 | Hs.517227 | junctional adhesion molecule 2 | JAM2 | 4.312603648 |
| 219295_S_AT | 26577 | ENSG00000163710 | Hs.8944 | procollagen C-endopeptidase enhancer 2 | PCOLCE2 | 6.314366586 |
| 219429_AT | 79152 | ENSG00000103089 | Hs.461329 | fatty acid 2-hydroxylase | FA2H | 98.27951566 |
| 219497_S_AT | 53335 | ENSG00000119866 | Hs.370549 | B-cell CLL/lymphoma 11A (zinc finger protein) | BCL11A | 4.445657505 |
| 219523_S_AT | 55714 | — | Hs.130438 | odz, odd Oz/ten-m homolog 3 (Drosophila) | ODZ3 | 3.268033115 |
| 219667_S_AT | 55024 | ENSG00000153064 | Hs.480400 | B-cell scaffold protein with ankyrin repeats 1 | BANK1 | 5.239438639 |
| 219682_S_AT | 6926 | ENSG00000135111 | Hs.129895 | T-box 3 | TBX3 | 3.177706801 |
| 219686_AT | 55351 | ENSG00000152953 | Hs.133062 | serine/threonine kinase 32B | STK32B | 3.933156218 |
| 219773_AT | 50507 | ENSG00000086991 | Hs.371036 | NADPH oxidase 4 | NOX4 | 33.95918834 |
| 219797_AT | 11320 | ENSG00000071073 | Hs.177576 | mannosyl (alpha-1,3-)-glycoprotein beta-1,4-N- | MGAT4A | 14.84453126 |
| | | | | acetylglucosaminyltransferase, isozyme A | | |
| 219829_AT | 26548 | ENSG00000147166 | Hs.109999 | integrin beta 1 binding protein (melusin) 2 | ITGB1BP2 | 0.266895088 |
| 219975_X_AT | 55301 | ENSG00000152463 | Hs.24309 | oleoyl-ACP hydrolase | OLAH | 12.73883995 |
| 219999_AT | 4122 | ENSG00000196547 | Hs.116459 | mannosidase, alpha, class 2A, member 2 | MAN2A2 | 2.502525545 |
| 220108_AT | 9630 | ENSG00000156049 | Hs.657795 | guanine nucleotide binding protein (G protein), alpha | GNA14 | 5.278341916 |
| | | | | 14 | | |
| 220115_S_AT | 1008 | ENSG00000040731 | Hs.92489 | cadherin 10, type 2 (T2-cadherin) | CDH10 | 14.79413943 |
| 220301_AT | 79839 | ENSG00000150636 | Hs.280781 | coiled-coil domain containing 102B | CCDC102B | 16.41744064 |
| 220488_S_AT | 54828 | ENSG00000141376 | Hs.655028 | breast carcinoma amplified sequence 3 | BCAS3 | 5.502528156 |
| 220639_AT | 79853 | ENSG00000168955 | Hs.156652 | transmembrane 4 L six family member 20 | TM4SF20 | 0.155520954 |
| 221104_S_AT | 55335 | ENSG00000165028 | Hs.720820 | nipsnap homolog 3B (C. elegans) | NIPSNAP3B | 11.51399051 |
| 221172_AT | 80099 | — | Hs.287647 | chromosome 7 open reading frame 69 | C7orf69 | 0.275630106 |
| 221530_S_AT | 79365 | ENSG00000123095 | Hs.177841 | basic helix-loop-helix family, member e41 | BHLHE41 | 0.323825085 |
| 221599_AT | 28971 | ENSG00000087884 | Hs.503357 | chromosome 11 open reading frame 67 | C11orf67 | 4.583283525 |
| 221600_S_AT | 28971 | ENSG00000087884 | Hs.503357 | chromosome 11 open reading frame 67 | C11orf67 | 4.765621306 |
| 221701_S_AT | 64220 | ENSG00000137868 | Hs.24553 | stimulated by retinoic acid gene 6 homolog (mouse) | STRA6 | 5.183142038 |
| 221756_AT | 113791 | ENSG00000100100 | Hs.26670 | phosphoinositide-3-kinase interacting protein 1 | PIK3IP1 | 2.09988674 |
| 221766_S_AT | 55603 | ENSG00000112773 | Hs.10784 | family with sequence similarity 46, member A | FAM46A | 2.821278753 |
| 221810_AT | 376267 | ENSG00000139998 | Hs.512492 | RAB15, member RAS onocogene family | RAB15 | 5.132419407 |
| 221911_AT | 2115 | ENSG00000006468 | Hs.22634 | ets variant 1 | ETV1 | 21.77779008 |
| 221958_S_AT | 79971 | ENSG00000116729 | Hs.647659 | wntless homolog (Drosophila) | WLS | 2.623332748 |
| 222020_S_AT | 50863 | ENSG00000182667 | Hs.504352 | neurotrimin | NTM | 5.69863005 |
| 222108_AT | 347902 | ENSG00000139211 | Hs.121520 | adhesion molecule with Ig-like domain 2 | AMIGO2 | 3.666860538 |
| 222118_AT | 55839 | ENSG00000166451 | Hs.55028 | centromere protein N | CENPN | 5.767648507 |
| 222125_S_AT | 54681 | ENSG00000178467 | Hs.654944 | prolyl 4-hydroxylase, transmembrane (endoplasmic | P4HTM | 2.542283751 |
| | | | | reticulum) | | |
| 222173_S_AT | 55357 | ENSG00000095383 | Hs.371016 | TBC1 domain family, member 2 | TBC1D2 | 0.161818114 |
| 222477_S_AT | 51768 | ENSG00000064115 | Hs.438641 | transmembrane 7 superfamily member 3 | TM7SF3 | 3.326179346 |
| 222696_AT | 8313 | ENSG00000168646 | Hs.156527 | axin 2 | AXIN2 | 10.28105682 |
| 222773_S_AT | 79695 | ENSG00000119514 | Hs.47099 | UDP-N-acetyl-alpha-D-galactosamine:polypeptide N- | GALNT12 | 6.75832594 |
| | | | | acetylgalactosaminyltransferase 12 (GalNAc-T12) | | |
| 222802_AT | 1906 | ENSG00000078401 | Hs.511899 | endothelin 1 | EDN1 | 10.54171127 |
| 222819_AT | 56474 | ENSG00000047230 | Hs.227049 | CTP synthase II | CTPS2 | 3.772911205 |
| 222833_AT | 54947 | ENSG00000087253 | Hs.460857 | lysophosphatidylcholine acyltransferase 2 | LPCAT2 | 9.055890827 |
| 222871_AT | 55220 | ENSG00000162873 | Hs.10414 | kelch domain containing 8A | KLHDC8A | 3.419511958 |
| 222891_S_AT | 53335 | ENSG00000119866 | Hs.370549 | B-cell CLL/lymphoma 11A (zinc finger protein) | BCL11A | 6.124237738 |
| 223062_S_AT | 29968 | ENSG00000135069 | Hs.494261 | phosphoserine aminotransferase 1 | PSAT1 | 0.250785091 |
| 223063_AT | 84886 | ENSG00000119280 | Hs.520494 | chromosome 1 open reading frame 198 | C1orf198 | 0.337617214 |
| 223177_AT | 221294 | ENSG00000178425 | Hs.724533 | 5′-nucleotidase domain containing 1 | NT5DC1 | 2.312627983 |
| 223178_S_AT | 221294 | ENSG00000178425 | Hs.724533 | 5′-nucleotidase domain containing 1 | NT5DC1 | 2.686104106 |
| 223217_S_AT | 64332 | ENSG00000144802 | Hs.319171 | nuclear factor of kappa light polypeptide gene | NFKBIZ | 3.610474481 |
| | | | | enhancer in B-cells inhibitor, zeta | | |
| 223430_AT | 23235 | ENSG00000170145 | Hs.269128 | salt-inducible kinase 2 | SIK2 | 4.603680364 |
| 223454_AT | 58191 | ENSG00000161921 | Hs.724659 | chemokine (C—X—C motif) ligand 16 | CXCL16 | 3.376972845 |
| 223530_AT | 11022 | ENSG00000182134 | Hs.144439 | tudor and KH domain containing | TDRKH | 3.323152299 |
| 223677_AT | 83734 | ENSG00000152348 | Hs.713698 | ATG10 autophagy related 10 homolog (S. cerevisiae) | ATG10 | 2.183235749 |
| 223734_AT | 84709 | ENSG00000137463 | Hs.710036 | chromosome 4 open reading frame 49 | C4orf49 | 0.160104793 |
| 223748_AT | 83959 | ENSG00000088836 | Hs.105607 | solute carrier family 4, sodium borate transporter, | SLC4A11 | 2.629883754 |
| | | | | member 11 | | |
| 223764_X_AT | 55335 | ENSG00000165028 | Hs.720820 | nipsnap homolog 3B (C. elegans) | NIPSNAP3B | 13.56114573 |
| 223796_AT | 79937 | ENSG00000106714 /// | Hs.658328 | contactin associated protein-like 3 | CNTNAP3 | 8.094005191 |
| | | ENSG00000154529 /// | | | | |
| | | ENSG00000185020 /// | | | | |
| | | ENSG00000227921 /// | | | | |
| | | ENSG00000236029 | | | | |
| 223877_AT | 114905 | ENSG00000163145 | Hs.153714 | C1q and tumor necrosis factor related protein 7 | C1QTNF7 | 7.56274169 |
| 224022_X_AT | 51384 | ENSG00000002745 | Hs.272375 | wingless-type MMTV integration site family, member | WNT16 | 33.85791441 |
| | | | | 16 | | |
| 224216_AT | — | — | — | — | — | 3.247621826 |
| 224220_X_AT | 7223 | ENSG00000133107 | Hs.262960 | transient receptor potential cation channel, subfamily | TRPC4 | 0.241145089 |
| | | | | C, member 4 | | |
| 224516_S_AT | 51523 | ENSG00000171604 | Hs.189119 | CXXC finger 5 | CXXC5 | 0.199855422 |
| 224707_AT | 84418 | ENSG00000120306 | Hs.529798 | chromosome 5 open reading frame 32 | C5orf32 | 3.529028351 |
| 224722_AT | 57534 | ENSG00000101752 | Hs.140903 | mindbomb homolog 1 (Drosophila) | MIB1 | 3.644878041 |
| 224901_AT | 79966 | ENSG00000145284 | Hs.379191 | stearoyl-CoA desaturase 5 | SCD5 | 3.348145775 |
| 224917_AT | 406991 | — | — | microRNA 21 | MIR21 | 3.01761591 |
| 224964_S_AT | 54331 | ENSG00000186469 | Hs.187772 | guanine nucleotide binding protein (G protein), | GNG2 | 34.83537453 |
| | | | | gamma 2 | | |
| 224998_AT | 146223 | ENSG00000183723 | Hs.643961 | CKLF-like MARVEL transmembrane domain | CMTM4 | 2.264289538 |
| | | | | containing 4 | | |
| 225009_AT | 146223 | ENSG00000183723 | Hs.643961 | CKLF-like MARVEL transmembrane domain | CMTM4 | 2.217420426 |
| | | | | containing 4 | | |
| 225016_AT | 147495 | ENSG00000154856 | Hs.293274 | adenomatosis polyposis coli down-regulated 1 | APCDD1 | 11.81746265 |
| 225207_AT | 5166 | ENSG00000004799 | Hs.8364 | pyruvate dehydrogenase kinase, isozyme 4 | PDK4 | 106.9142723 |
| 225314_AT | 132299 | ENSG00000145247 | Hs.95835 | OCIA domain containing 2 | OCIAD2 | 3.189330332 |
| 225532_AT | 91768 | ENSG00000134508 | Hs.11108 | Cdk5 and Abl enzyme substrate 1 | CABLES1 | 4.218154757 |
| 225540_AT | 4133 | ENSG00000078018 | Hs.368281 | microtubule-associated protein 2 | MAP2 | 11.94488559 |
| 225544_AT | 6926 | ENSG00000135111 | Hs.129895 | T-box 3 | TBX3 | 2.804732711 |
| 225627_S_AT | 57685 | ENSG00000158966 | Hs.443891 | cache domain containing 1 | CACHD1 | 5.876293653 |
| 225666_AT | 84899 | ENSG00000125247 | Hs.724501 | transmembrane and tetratricopeptide repeat containing 4 | TMTC4 | 7.159486557 |
| 225674_AT | 55973 | ENSG00000075790 | Hs.303787 | B-cell receptor-associated protein 29 | BCAP29 | 2.491630147 |
| 225677_AT | 55973 | ENSG00000075790 | Hs.303787 | B-cell receptor-associated protein 29 | BCAP29 | 2.83110875 |
| 225728_AT | 8470 | ENSG00000154556 | Hs.655143 | sorbin and SH3 domain containing 2 | SORBS2 | 43.52252516 |
| 225825_AT | 25943 | ENSG00000088854 | Hs.516853 | chromosome 20 open reading frame 194 | C20orf194 | 6.032675774 |
| 225835_AT | 6558 | ENSG00000064651 | Hs.162585 | solute carrier family 12 (sodium/potassium/chloride | SLC12A2 | 7.925294717 |
| | | | | transporters), member 2 | | |
| 225972_AT | 169200 | ENSG00000180694 | Hs.567759 | transmembrane protein 64 | TMEM64 | 3.64583505 |
| 226039_AT | 11320 | ENSG00000071073 | Hs.177576 | mannosyl (alpha-1,3-)-glycoprotein beta-1,4-N- | MGAT4A | 20.00421914 |
| | | | | acetylglucosaminyltransferase, isozyme A | | |
| 226153_S_AT | 246175 | ENSG00000138767 | Hs.592519 | CCR4-NOT transcription complex, subunit 6-like | CNOT6L | 2.186155082 |
| 226198_AT | 146691 | ENSG00000175662 | Hs.462379 | target of myb1-like 2 (chicken) | TOM1L2 | 3.305042731 |
| 226213_AT | 2065 | — | Hs.118681 | v-erb-b2 erythroblastic leukemia viral oncogene | ERBB3 | 0.156646981 |
| | | | | homolog 3 (avian) | | |
| 226225_AT | 4163 | ENSG00000171444 | Hs.593171 | mutated in colorectal cancers | MCC | 6.802475507 |
| 226490_AT | 57224 | ENSG00000135540 | Hs.652741 | NHS-like 1 | NHSL1 | 4.247580645 |
| 226498_AT | 2321 | — | Hs.594454 | fms-related tyrosine kinase 1 (vascular endothelial | FLT1 | 0.187063946 |
| | | | | growth factor/vascular permeability factor receptor) | | |
| 226552_AT | 389792 | ENSG00000188483 | Hs.529857 | immediate early response 5-like | IER5L | 0.268107729 |
| 226576_AT | 23092 | ENSG00000145819 | Hs.654668 | Rho GTPase activating protein 26 | ARHGAP26 | 5.341987736 |
| 226586_AT | 203286 | ENSG00000165138 | Hs.406890 | ankyrin repeat and sterile alpha motif domain | ANKS6 | 2.394078307 |
| | | | | containing 6 | | |
| 226607_AT | 25943 | ENSG00000088854 | Hs.516853 | chromosome 20 open reading frame 194 | C20orf194 | 6.052998781 |
| 226629_AT | 124935 | ENSG00000167703 | Hs.160550 | solute carrier family 43, member 2 | SLC43A2 | 2.264244604 |
| 226636_AT | 5337 | ENSG00000075651 | Hs.382865 | phospholipase D1, phosphatidylcholine-specific | PLD1 | 3.302222897 |
| 226647_AT | 84866 | ENSG00000149582 | Hs.564188 | transmembrane protein 25 | TMEM25 | 2.521810098 |
| 226701_AT | 2702 | ENSG00000143140 | Hs.447968 | gap junction protein, alpha 5, 40 kDa | GJA5 | 0.040736972 |
| 226731_AT | 53918 | — | Hs.644352 | Pelota homolog (Drosophila) | PELO | 22.8661376 |
| 226763_AT | 91404 | ENSG00000187231 | Hs.30977 | SEC14 and spectrin domains 1 | SESTD1 | 4.032878479 |
| 226789_AT | 133418 | ENSG00000170571 | Hs.561411 | embigin homolog (mouse) | EMB | 2.377151237 |
| 226824_AT | 119587 | ENSG00000121898 | Hs.656887 | carboxypeptidase X (M14 family), member 2 | CPXM2 | 4.378545172 |
| 226936_AT | 387103 | ENSG00000203760 | Hs.486401 | centromere protein W | CENPW | 3.29880902 |
| 226955_AT | 134265 | ENSG00000157510 | Hs.483793 | actin filament associated protein 1-like 1 | AFAP1L1 | 0.315595043 |
| 226961_AT | 222171 | ENSG00000176532 | Hs.91109 | proline rich 15 | PRR15 | 3.463813369 |
| 226982_AT | 22936 | ENSG00000118985 | Hs.192221 | elongation factor, RNA polymerase II, 2 | ELL2 | 0.222052814 |
| 227041_AT | 91404 | ENSG00000187231 | Hs.30977 | SEC14 and spectrin domains 1 | SESTD1 | 3.681816053 |
| 227058_AT | 84935 | ENSG00000102802 | Hs.147880 | chromosome 13 open reading frame 33 | C13orf33 | 13.1241816 |
| 227059_AT | 10082 | — | Hs.444329 | glypican 6 | GPC6 | 5.631168539 |
| 227088_AT | 8654 | ENSG00000138735 | Hs.647971 | phosphodiesterase 5A, cGMP-specific | PDE5A | 18.5169205 |
| 227095_AT | 54741 | — | Hs.23581 | leptin receptor overlapping transcript | LEPROT | 2.335016257 |
| 227115_AT | — | — | Hs.503862 | — | — | 0.246804977 |
| 227176_AT | 114134 | ENSG00000151229 | Hs.558595 | solute carrier family 2 (facilitated glucose | SLC2A13 | 3.683457429 |
| | | | | transporter), member 13 | | |
| 227202_AT | 1272 | — | Hs.143434 | Contactin 1 | CNTN1 | 4.652311642 |
| 227222_AT | 26267 | ENSG00000147912 | Hs.709527 | F-box protein 10 | FBXO10 | 2.815583378 |
| 227231_AT | 57482 | ENSG00000109265 | Hs.596667 | KIAA1211 | KIAA1211 | 9.457229086 |
| 227266_S_AT | 2533 | ENSG00000082074 | Hs.370503 | FYN binding protein | FYB | 31.74657387 |
| 227314_AT | 3673 | ENSG00000164171 | Hs.482077 | integrin, alpha 2 (CD49B, alpha 2 subunit of VLA-2 | ITGA2 | 3.334717503 |
| | | | | receptor) | | |
| 227376_AT | 2737 | ENSG00000106571 | Hs.21509 | GLI family zinc finger 3 | GLI3 | 4.241232245 |
| 227529_S_AT | 9590 | ENSG00000131016 | Hs.371240 | A kinase (PRKA) anchor protein 12 | AKAP12 | 2.590360879 |
| 227566_AT | 50863 | ENSG00000182667 | Hs.504352 | neurotrimin | NTM | 6.869137985 |
| 227618_AT | — | — | Hs.496303 | — | — | 0.186608853 |
| 227623_AT | — | — | Hs.592625 | — | — | 3.295171815 |
| 227717_AT | 389337 | ENSG00000183111 | Hs.256206 | Rho guanine nucleotide exchange factor (GEF) 37 | ARHGEF37 | 15.86551603 |
| 227747_AT | 196264 | ENSG00000160588 | Hs.15396 | myelin protein zero-like 3 | MPZL3 | 2.76427219 |
| 227764_AT | 130574 | — | Hs.21929 | LY6/PLAUR domain containing 6 | LYPD6 | 5.757888134 |
| 227826_S_AT | — | — | Hs.481342 | — | — | 2.859453591 |
| 227827_AT | — | — | Hs.481342 | — | — | 3.443950532 |
| 227889_AT | 54947 | ENSG00000087253 | Hs.460857 | lysophosphatidylcholine acyltransferase 2 | LPCAT2 | 12.62361509 |
| 227899_AT | 5212 | ENSG00000205221 | Hs.137415 | vitrin | VIT | 0.142350287 |
| 227929_AT | — | — | Hs.586760 | — | — | 6.065299085 |
| 227976_AT | 644538 | — | Hs.42239 | hypothetical protein LOC644538 | LOC644538 | 2.401870917 |
| 228058_AT | 124220 | ENSG00000162078 | Hs.105887 | zymogen granule protein 16 homolog B (rat) | ZG16B | 0.288294748 |
| 228088_AT | 91404 | ENSG00000187231 | Hs.30977 | SEC14 and spectrin domains 1 | SESTD1 | 4.651780713 |
| 228101_AT | 320 | ENSG00000107282 | Hs.171939 | amyloid beta (A4) precursor protein-binding, family | APBA1 | 9.235513621 |
| | | | | A, member 1 | | |
| 228121_AT | 7042 | ENSG00000092969 | Hs.133379 | transforming growth factor, beta 2 | TGFB2 | 0.288580251 |
| 228186_S_AT | 84870 | ENSG00000146374 | Hs.135254 | R-spondin 3 homolog (Xenopus laevis) | RSPO3 | 4.087287795 |
| 228235_AT | 84848 | ENSG00000223749 | Hs.416379 | hypothetical protein MGC16121 | MGC16121 | 0.315251771 |
| 228284_AT | 7088 | ENSG00000196781 | Hs.197320 | transducin-like enhancer of split 1 (E(sp1) homolog, | TLE1 | 4.211704967 |
| | | | | Drosophila) | | |
| 228302_X_AT | 55450 | ENSG00000162545 | Hs.197922 | calcium/calmodulin-dependent protein kinase II | CAMK2N1 | 3.335759815 |
| | | | | inhibitor 1 | | |
| 228314_AT | 84230 | ENSG00000171488 | Hs.412836 | leucine rich repeat containing 8 family, member C | LRRC8C | 3.731738785 |
| 228396_AT | 5592 | ENSG00000185532 | Hs.654556 | protein kinase, cGMP-dependent, type I | PRKG1 | 0.264487614 |
| 228531_AT | 54809 | ENSG00000205413 | Hs.65641 | sterile alpha motif domain containing 9 | SAMD9 | 3.437781275 |
| 228559_AT | 55839 | ENSG00000166451 | Hs.55028 | centromere protein N | CENPN | 6.859758915 |
| 228573_AT | 118429 | — | Hs.162963 | anthrax toxin receptor 2 | ANTXR2 | 3.982823092 |
| 228574_AT | 160335 | — | Hs.577775 | Transmembrane and tetratricopeptide repeat | TMTC2 | 11.49653696 |
| | | | | containing 2 | | |
| 228607_AT | 4939 | ENSG00000111335 | Hs.414332 | 2′-5′-oligoadenylate synthetase 2, 69/71 kDa | OAS2 | 9.688667229 |
| 228635_AT | 57575 | ENSG00000138650 | Hs.192859 | protocadherin 10 | PCDH10 | 6.536169242 |
| 228653_AT | 389432 | ENSG00000203727 | Hs.567973 | sterile alpha motif domain containing 5 | SAMD5 | 5.216866805 |
| 228692_AT | — | — | Hs.715647 | — | — | 8.204690412 |
| 228707_AT | 137075 | — | Hs.183617 | claudin 23 | CLDN23 | 6.096420374 |
| 228740_AT | — | — | Hs.26766 | — | — | 9.63384635 |
| 228750_AT | — | — | Hs.657621 | — | — | 2.318935445 |
| 228758_AT | 604 | — | Hs.478588 | B-cell CLL/lymphoma 6 | BCL6 | 6.865472147 |
| 228777_AT | 143879 | ENSG00000182359 | Hs.101949 | kelch repeat and BTB (POZ) domain containing 3 | KBTBD3 | 2.628467075 |
| 228890_AT | 84913 | ENSG00000168874 | Hs.135569 | atonal homolog 8 (Drosophila) | ATOH8 | 0.2191201 |
| 228949_AT | 79971 | ENSG00000116729 | Hs.647659 | wntless homolog (Drosophila) | WLS | 3.813590834 |
| 228962_AT | 5144 | ENSG00000113448 | Hs.117545 | phosphodiesterase 4D, cAMP-specific | PDE4D | 3.839890396 |
| | | | | (phosphodiesterase E3 dunce homolog, Drosophila) | | |
| 228964_AT | 639 | ENSG00000057657 | Hs.436023 | PR domain containing 1, with ZNF domain | PRDM1 | 7.651259811 |
| 229015_AT | 286367 | — | Hs.720355 | FP944 | LOC286367 | 5.199707348 |
| 229134_AT | 81839 | ENSG00000173218 | Hs.515130 | vang-like 1 (van gogh, Drosophila) | VANGL1 | 3.217615578 |
| 229187_AT | 283788 | — | Hs.702270 | FSHD region gene 1 pseudogene | LOC283788 | 2.925513326 |
| 229256_AT | 283209 | ENSG00000165434 | Hs.26612 | phosphoglucomutase 2-like 1 | PGM2L1 | 2.934813677 |
| 229302_AT | 130733 | ENSG00000152154 | Hs.40808 | transmembrane protein 178 | TMEM178 | 16.74738316 |
| 229339_AT | 93649 | ENSG00000141052 | Hs.567641 | myocardin | MYOCD | 0.133593551 |
| 229376_AT | 5629 | ENSG00000117707 | Hs.585369 | prospero homeobox 1 | PROX1 | 4.674409741 |
| 229393_AT | 84456 | ENSG00000198945 | Hs.658051 | l(3)mbt-like 3 (Drosophila) | L3MBTL3 | 4.20727306 |
| 229429_X_AT | 728855 | ENSG00000232151 /// | Hs.456578 | hypothetical LOC728855 | LOC728855 | 2.485292629 |
| | | ENSG00000235398 /// | | | | |
| | | ENSG00000236943 | | | | |
| 229435_AT | 169792 | ENSG00000107249 | Hs.162125 | GUS family zinc finger 3 | GLIS3 | 4.275044818 |
| 229553_AT | 283209 | ENSG00000165434 | Hs.26612 | phosphoglucomutase 2-like 1 | PGM2L1 | 2.659060799 |
| 229576_S_AT | 6926 | ENSG00000135111 | Hs.129895 | T-box 3 | TBX3 | 3.112173003 |
| 229618_AT | 64089 | ENSG00000104497 | Hs.492121 | sorting nexin 16 | SNX16 | 4.352445188 |
| 229623_AT | 441027 | — | Hs.724062 | Transmembrane protein 150C | TMEM150C | 6.374956057 |
| 229656_S_AT | 400954 | ENSG00000214595 | Hs.656692 | echinoderm microtubule associated protein like 6 | EML6 | 3.448175515 |
| 229900_AT | 135228 | ENSG00000156535 | Hs.399891 | CD109 molecule | CD109 | 2.473948212 |
| 230036_AT | 219285 | ENSG00000177409 | Hs.489118 | sterile alpha motif domain containing 9-like | SAMD9L | 2.436920319 |
| 230144_AT | 2892 | ENSG00000125675 | Hs.377070 | glutamate receptor, ionotrophic, AMPA 3 | GRIA3 | 8.886386761 |
| 230254_AT | 254228 | ENSG00000178033 | Hs.660142 | family with sequence similarity 26, member E | FAM26E | 6.34713061 |
| 230258_AT | 169792 | ENSG00000107249 | Hs.162125 | GUS family zinc finger 3 | GLIS3 | 4.216952126 |
| 230319_AT | — | — | Hs.90250 | — | — | 6.254086561 |
| 230518_AT | 10205 | ENSG00000149573 | Hs.116651 | myelin protein zero-like 2 | MPZL2 | 17.46717317 |
| 230538_AT | 399694 | ENSG00000185634 | Hs.642615 | SHC (Src homology 2 domain containing) family, | SHC4 | 36.65962457 |
| | | | | member 4 | | |
| 230560_AT | 29091 | ENSG00000168952 | Hs.508958 | syntaxin binding protein 6 (amisyn) | STXBP6 | 0.267628825 |
| 230574_AT | 100130938 | — | Hs.710069 | hypothetical LOC100130938 | LOC100130938 | 5.243414009 |
| 230654_AT | — | — | Hs.106532 | — | — | 2.353651716 |
| 230831_AT | 84978 | ENSG00000171877 | Hs.578544 | FERM domain containing 5 | FRMD5 | 13.17637898 |
| 231313_AT | 400761 | ENSG00000231999 | — | hypothetical gene supported by AK130864 | FLJ27354 | 2.525030213 |
| 231380_AT | 116328 | ENSG00000165084 | Hs.491941 | chromosome 8 open reading frame 34 | C8orf34 | 2.857690432 |
| 231513_AT | — | — | Hs.597550 | — | — | 11.52614571 |
| 231798_AT | 9241 | ENSG00000183691 | Hs.248201 | noggin | NOG | 0.033558863 |
| 231867_AT | 57451 | ENSG00000145934 | Hs.631957 | odz, odd Oz/ten-m homolog 2 (Drosophila) | ODZ2 | 0.109457 |
| 231872_AT | 85444 | ENSG00000133739 | Hs.193115 | leucine rich repeat and coiled-coil domain containing 1 | LRRCC1 | 2.725032126 |
| 232034_AT | 203274 | — | Hs.599821 | Hypothetical protein LOC203274 | LOC203274 | 7.172703169 |
| 232060_AT | 4919 | ENSG00000185483 | Hs.654491 | receptor tyrosine kinase-like orphan receptor 1 | ROR1 | 0.131131718 |
| 232082_X_AT | 6707 | ENSG00000163209 | Hs.139322 | small proline-rich protein 3 | SPRR3 | 7.012171039 |
| 232165_AT | 83481 | — | Hs.200412 | epiplakin 1 | EPPK1 | 0.227964918 |
| 232267_AT | 283383 | ENSG00000111452 | Hs.719239 | G protein-coupled receptor 133 | GPR133 | 35.68203799 |
| 232327_AT | 80731 | ENSG00000144229 | Hs.68533 | thrombospondin, type I, domain containing 7B | THSD7B | 0.147397524 |
| 232368_AT | 100128327 | ENSG00000173626 | Hs.134795 | BET3 like (S. cerevisiae) | BET3L | 59.51977114 |
| 232434_AT | 729582 | — | Hs.572788 | disrupted in renal carcinoma 3 | DIRC3 | 5.462497498 |
| 232752_AT | 100287616 | — | Hs.599785 | Hypothetical protein LOC100287616 | LOC100287616 | 0.311712051 |
| 233401_AT | — | — | Hs.636858 | — | — | 3.056771938 |
| 233485_AT | — | — | Hs.670741 | — | — | 4.622746912 |
| 233720_AT | 8470 | — | Hs.655143 | Sorbin and SH3 domain containing 2 | SORBS2 | 3.225977988 |
| 234051_AT | — | — | Hs.531728 | — | — | 3.762562537 |
| 234305_S_AT | 56169 | ENSG00000147697 | Hs.133244 | gasdermin C | GSDMC | 6.998951274 |
| 234488_S_AT | 64395 /// 64396 | ENSG00000087338 /// | Hs.293971 | germ cell-less homolog 1 (Drosophila) /// germ cell-less homolog 1 (Drosophila)-like | GMCL1 /// | 2.27628059 |
| | | ENSG00000226607 | | | GMCL1L | |
| 234811_AT | 55839 | ENSG00000166451 | Hs.55028 | centromere protein N | CENPN | 6.470698745 |
| 235046_AT | — | — | Hs.176376 | — | — | 0.13364792 |
| 235099_AT | 152189 | ENSG00000170293 | Hs.154986 | CKLF-like MARVEL transmembrane domain | CMTM8 | 2.704607301 |
| | | | | containing 8 | | |
| 235238_AT | 399694 | ENSG00000185634 | Hs.642615 | SHC (Src homology 2 domain containing) family, | SHC4 | 56.28716689 |
| | | | | member 4 | | |
| 235367_AT | 84665 | ENSG00000138347 | Hs.55205 | myopalladin | MYPN | 0.06036166 |
| 235392_AT | — | — | Hs.720470 | — | — | 0.355716621 |
| 235489_AT | 57381 | ENSG00000126785 | Hs.656339 | ras homolog gene family, member J | RHOJ | 3.828803162 |
| 235591_AT | 6751 | ENSG00000139874 | Hs.248160 | somatostatin receptor 1 | SSTR1 | 0.064925058 |
| 235668_AT | 639 | — | Hs.436023 | PR domain containing 1, with ZNF domain | PRDM1 | 6.366901854 |
| 235746_S_AT | — | ENSG00000153246 | — | — | — | 3.975765932 |
| 235776_X_AT | — | ENSG00000225511 | — | — | — | 6.115248156 |
| 235874_AT | 167681 | ENSG00000146250 | Hs.98381 | protease, serine, 35 | PRSS35 | 12.73857258 |
| 235889_AT | — | — | Hs.618649 | — | — | 3.228022552 |
| 235944_AT | 83872 | ENSG00000143341 | Hs.58877 | hemicentin 1 | HMCN1 | 32.18721617 |
| 235945_AT | 6716 | ENSG00000049319 | Hs.458345 | steroid-5-alpha-reductase, alpha polypeptide 2 (3-oxo- | SRD5A2 | 2.361463775 |
| | | | | 5 alpha-steroid delta 4-dehydrogenase alpha 2) | | |
| 236028_AT | 3381 | ENSG00000029559 | Hs.518726 | integrin-binding sialoprotein | IBSP | 528.6564811 |
| 236029_AT | 120114 | ENSG00000165323 | Hs.98523 | FAT tumor suppressor homolog 3 (Drosophila) | FAT3 | 3.521884419 |
| 236436_AT | 283130 | ENSG00000162241 | Hs.661604 | solute carrier family 25, member 45 | SLC25A45 | 2.88404571 |
| 236439_AT | — | — | Hs.660734 | — | — | 5.687805083 |
| 236576_AT | — | — | Hs.21375 | — | — | 7.335285078 |
| 236599_AT | — | — | — | — | — | 5.759608501 |
| 236843_AT | 50507 | ENSG00000086991 | Hs.371036 | NADPH oxidase 4 | NOX4 | 12.76176442 |
| 237056_AT | 387755 | ENSG00000188487 | Hs.591997 | inscuteable homolog (Drosophila) | INSC | 9.66004489 |
| 237206_AT | 93649 | ENSG00000141052 | Hs.567641 | myocardin | MYOCD | 0.133279902 |
| 237227_AT | 152110 | ENSG00000163491 | Hs.506115 | NIMA (never in mitosis gene a)-related kinase 10 | NEK10 | 4.033839624 |
| 237329_AT | — | ENSG00000235904 | — | — | — | 2.775793761 |
| 237372_AT | — | — | Hs.682656 | — | — | 0.279041597 |
| 237597_AT | — | — | Hs.118228 | — | — | 15.47683159 |
| 238050_AT | 118429 | — | Hs.162963 | anthrax toxin receptor 2 | ANTXR2 | 3.771513138 |
| 238513_AT | 79056 | — | Hs.471695 | Proline rich Gla (G-carboxyglutamic acid) 4 | PRRG4 | 3.715619247 |
| | | | | (transmembrane) | | |
| 238681_AT | 284161 | — | Hs.631744 | glycerophosphodiester phosphodiesterase domain | GDPD1 | 3.601354703 |
| | | | | containing 1 | | |
| 238727_AT | — | — | Hs.723465 | — | — | 12.68673531 |
| 238751_AT | — | — | Hs.481342 | — | — | 6.02855103 |
| 238901_AT | — | ENSG00000224259 | Hs.445045 | — | — | 0.073833501 |
| 238905_AT | 57381 | ENSG00000126785 | Hs.656339 | ras homolog gene family, member J | RHOJ | 3.295108746 |
| 239201_AT | 65061 | ENSG00000138395 | Hs.348711 | cyclin-dependent kinase 15 | CDK15 | 0.175333414 |
| 239283_AT | 50999 | ENSG00000117500 | Hs.482873 | transmembrane emp24 protein transport domain | TMED5 | 2.543070197 |
| | | | | containing 5 | | |
| 239288_AT | 23043 | — | Hs.34024 | TRAF2 and NCK interacting kinase | TNIK | 3.011052607 |
| 239349_AT | 114905 | ENSG00000163145 | Hs.153714 | C1q and tumor necrosis factor related protein 7 | C1QTNF7 | 21.31725699 |
| 239370_AT | — | ENSG00000224259 | — | — | — | 0.207097193 |
| 239472_AT | — | — | Hs.596724 | — | — | 2.465909428 |
| 239579_AT | 253152 | ENSG00000172031 | Hs.201555 | epoxide hydrolase 4 | EPHX4 | 30.35081154 |
| 239598_S_AT | 54947 | ENSG00000087253 | Hs.460857 | lysophosphatidylcholine acyltransferase 2 | LPCAT2 | 4.645553765 |
| 239787_AT | 386618 | ENSG00000180332 | Hs.23406 | potassium channel tetramerisation domain containing 4 | KCTD4 | 3.339118585 |
| 239896_AT | 100289098 | ENSG00000237310 | Hs.721866 | Hypothetical protein LOC100289098 | LOC100289098 | 2.316516049 |
| 239903_AT | — | — | Hs.71947 | — | — | 3.352819985 |
| 239942_AT | — | — | Hs.547771 | — | — | 5.725071237 |
| 240088_AT | 8654 | ENSG00000138735 | Hs.587281 | phosphodiesterase 5A, cGMP-specific | PDE5A | 5.571664518 |
| 240120_AT | — | — | Hs.658732 | — | — | 4.606247292 |
| 240167_AT | 152742 | — | Hs.135435 | hypothetical protein LOC152742 | LOC152742 | 0.04916805 |
| 240512_X_AT | 386618 | ENSG00000180332 | Hs.23406 | potassium channel tetramerisation domain containing 4 | KCTD4 | 3.49741429 |
| 240616_AT | — | — | Hs.721829 | — | — | 3.90341532 |
| 241255_AT | — | — | — | — | — | 9.175010398 |
| 241353_S_AT | — | ENSG00000232533 | — | — | — | 4.161821336 |
| 241470_X_AT | — | — | Hs.247150 | — | — | 4.968316159 |
| 241652_X_AT | — | — | Hs.624171 | — | — | 5.350218666 |
| 242794_AT | 55534 | ENSG00000196782 | Hs.586165 | mastermind-like 3 (Drosophila) | MAML3 | 3.496322615 |
| 243100_AT | 93034 | ENSG00000185013 | Hs.120319 | 5′-nucleotidase, cytosolic IB | NT5C1B | 3.784009425 |
| 243179_AT | 100130360 | — | Hs.408455 | Hypothetical LOC100130360 | LOC100130360 | 3.729042418 |
| 243220_AT | — | — | Hs.411848 | — | — | 3.585183546 |
| 243337_AT | 166752 | ENSG00000183090 | Hs.252714 | FRAS1 related extracellular matrix 3 | FREM3 | 3.605800848 |
| 243386_AT | 54897 | ENSG00000130940 | Hs.439894 | castor zinc finger 1 | CASZ1 | 3.621754217 |
| 243481_AT | 57381 | — | Hs.656339 | ras homolog gene family, member J | RHOJ | 4.172069717 |
| 243580_AT | 9630 | ENSG00000156049 | Hs.657795 | guanine nucleotide binding protein (G protein), alpha | GNA14 | 6.648908274 |
| | | | | 14 | | |
| 243582_AT | 153769 | — | Hs.443728 | SH3 domain containing ring finger 2 | SH3RF2 | 0.165321065 |
| 243610_AT | 138255 | ENSG00000204711 | Hs.444459 | chromosome 9 open reading frame 135 | C9orf135 | 14.80838027 |
| 243688_AT | 90768 | — | Hs.175465 | hypothetical LOC90768 | MGC45800 | 2.307000893 |
| 244008_AT | 79668 | — | Hs.369581 | poly (ADP-ribose) polymerase family, member 8 | PARP8 | 7.061954966 |
| 244050_AT | 401494 | ENSG00000188921 | Hs.716678 | protein tyrosine phosphatase-like A domain | PTPLAD2 | 3.225389272 |
| | | | | containing 2 | | |
| 244096_AT | — | — | — | — | — | 7.24900177 |
| 244261_AT | 163702 | ENSG00000185436 | Hs.221375 | interleukin 28 receptor, alpha (interferon, lambda | IL28RA | 3.185666972 |
| | | | | receptor) | | |
| 244680_AT | 2743 | ENSG00000109738 | Hs.32973 | glycine receptor, beta | GLRB | 3.086008413 |
| 244802_AT | — | — | Hs.624328 | — | — | 3.262661269 |
| 266_S_AT | 100133941 | ENSG00000185275 | Hs.644105 | CD24 molecule | CD24 | 0.185055404 |
| 34697_AT | 4040 | ENSG00000070018 | Hs.584775 | low density lipoprotein receptor-related protein 6 | LRP6 | 3.294783474 |
| 37892_AT | 1301 | ENSG00000060718 | Hs.523446 | collagen, type XI, alpha 1 | COL11A1 | 4.066670273 |
| 44783_S_AT | 23462 | ENSG00000164683 | Hs.234434 | hairy/enhancer-of-split related with YRPW motif 1 | HEY1 | 11.74162773 |
| 49077_AT | 51400 | ENSG00000214517 | Hs.503251 | protein phosphatase methylesterase 1 | PPME1 | 0.308229327 |
| 59697_AT | 376267 | ENSG00000139998 | Hs.512492 | RAB15, member RAS oncogene family | RAB15 | 5.35923754 |

TABLE 1B

| | | | | | | Pop | Fold | | | |
|---|---|---|---|---|---|---|---|---|---|---|
| Term | Count | % | Genes | List Total | Pop Hits | Total | Enrichment | PValue | Benjamini | FDR |
| hsa04514: Cell adhesion | 14 | 2.886597938 | 228707_AT, 219213_AT, 212587_S_AT, | 160 | 132 | 5085 | 3.370738636 | 2.14E−04 | 0.026192531 | 0.246862738 |
| molecules (CAMs) | | | 203868_S_AT, 200904_AT, 212813_AT, | | | | | | | |
| | | | 211799_X_AT, 233401_AT, 204105_S_AT, | | | | | | | |
| | | | 209030_S_AT, 227202_AT, 213438_AT, | | | | | | | |
| | | | 209032_S_AT, 212588_AT, 209031_AT, | | | | | | | |
| | | | 1554812_AT, 207238_S_AT, 202071_AT, | | | | | | | |
| | | | 204584_AT | | | | | | | |
| hsa04350: TGF-beta signaling | 11 | 2.268041237 | 208944_AT, 207069_S_AT, 201565_S_AT, | 160 | 87 | 5085 | 4.018318966 | 3.39E−04 | 0.02077461 | 0.390237299 |
| pathway | | | 201566_X_AT, 202728_S_AT, 203083_AT, | | | | | | | |
| | | | 228121_AT, 231798_AT, 209909_S_AT, | | | | | | | |
| | | | 205258_AT, 202729_S_AT, 207826_S_AT, | | | | | | | |
| | | | 208937_S_AT, 207334_S_AT, 210511_S_AT | | | | | | | |
| hsa04512: ECM-receptor interaction | 7 | 1.443298969 | 202267_AT, 203083_AT, 209875_S_AT, | 160 | 84 | 5085 | 2.6484375 | 0.046725259 | 0.861638836 | 42.45307537 |
| | | | 227314_AT, 236028_AT, 204320_AT, | | | | | | | |
| | | | 37892_AT, 207370_AT, 202071_AT | | | | | | | |
| hsa04730: Long-term | 6 | 1.237113402 | 203710_AT, 203896_S_AT, 213222_AT, | 160 | 69 | 5085 | 2.763586957 | 0.063613512 | 0.869650525 | 53.18554615 |
| depression | | | 206730_AT, 228396_AT, 1569290_S_AT, | | | | | | | |
| | | | 230144_AT, 203817_AT | | | | | | | |
| hsa04060: Cytokine-cytokine | 13 | 2.680412371 | 205403_AT, 208944_AT, 211355_X_AT, | 160 | 262 | 5085 | 1.576932252 | 0.117039041 | 0.954359623 | 76.2445752 |
| receptor interaction | | | 211356_X_AT, 209894_AT, 202688_AT, | | | | | | | |
| | | | 202948_AT, 206336_AT, 211372_S_AT, | | | | | | | |
| | | | 244261_AT, 204932_AT, 226498_AT, | | | | | | | |
| | | | 223454_AT, 227095_AT, 228121_AT, | | | | | | | |
| | | | 202687_S_AT, 205258_AT, 209909_S_AT, | | | | | | | |
| | | | 204933_S_AT, 210511_S_AT, 207334_S_AT | | | | | | | |
| hsa04144: Endocytosis | 10 | 2.06185567 | 208944_AT, 226213_AT, 200904_AT, | 160 | 184 | 5085 | 1.727241848 | 0.121017544 | 0.930456329 | 77.45164419 |
| | | | 1558502_S_AT, 202454_S_AT, | | | | | | | |
| | | | 211799_X_AT, 209839_AT, 226498_AT, | | | | | | | |
| | | | 239472_AT, 203638_S_AT, 210757_X_AT, | | | | | | | |
| | | | 1558501_AT, 207334_S_AT, 226636_AT | | | | | | | |
| hsa04960: Aldosterone- | 4 | 0.824742268 | 202410_X_AT, 235392_AT, 202409_AT, | 160 | 41 | 5085 | 3.100609756 | 0.135422711 | 0.92405051 | 81.3685771 |
| regulated sodium reabsorption | | | 1560652_AT, 201243_S_AT | | | | | | | |
| hsa04540: Gap junction | 6 | 1.237113402 | 212242_AT, 203710_AT, 203896_S_AT, | 160 | 89 | 5085 | 2.14255618 | 0.144933926 | 0.911693033 | 83.60277234 |
| | | | 213222_AT, 228396_AT, 203817_AT | | | | | | | |
| hsa04142: Lysosome | 7 | 1.443298969 | 210619_S_AT, 209420_S_AT, 202838_AT, | 160 | 117 | 5085 | 1.901442308 | 0.1587137 | 0.90755305 | 86.40768815 |
| | | | 212334_AT, 208926_AT, 210980_S_AT, | | | | | | | |
| | | | 213902_AT, 212335_AT, 213702_X_AT, | | | | | | | |
| | | | 210042_S_AT, 216230_X_AT | | | | | | | |
| hsa00760: Nicotinate and | 3 | 0.618556701 | 1553995_A_AT, 243100_AT, 1553994_AT, | 160 | 24 | 5085 | 3.97265625 | 0.171820761 | 0.903452328 | 88.66175515 |
| nicotinamide metabolism | | | 219147_S_AT | | | | | | | |
| hsa04510: Focal adhesion | 10 | 2.06185567 | 209875_S_AT, 202267_AT, 236028_AT, | 160 | 201 | 5085 | 1.581156716 | 0.176422717 | 0.8878606 | 89.36833562 |
| | | | 37892_AT, 207876_S_AT, 207370_AT, | | | | | | | |
| | | | 203083_AT, 226498_AT, 227314_AT, | | | | | | | |
| | | | 235238_AT, 200953_S_AT, 204320_AT, | | | | | | | |
| | | | 230538_AT | | | | | | | |
| hsa04950: Maturity onset | 3 | 0.618556701 | 202410_X_AT, 204689_AT, 202409_AT, | 160 | 25 | 5085 | 3.81375 | 0.183136601 | 0.876345236 | 90.32722649 |
| diabetes of the young | | | 215933_S_AT, 203395_S_AT | | | | | | | |
| hsa04310: Wnt signaling | 8 | 1.649484536 | 222696_AT, 200953_S_AT, 229134_AT, | 160 | 151 | 5085 | 1.683774834 | 0.192776554 | 0.870322799 | 91.5663371 |
| pathway | | | 203896_S_AT, 213222_AT, 216933_X_AT, | | | | | | | |
| | | | 224022_X_AT, 34697_AT, 205606_AT | | | | | | | |
| hsa04070: Phosphatidylinositol | 5 | 1.030927835 | 205111_S_AT, 203710_AT, 203896_S_AT, | 160 | 74 | 5085 | 2.147381757 | 0.200124667 | 0.861625937 | 92.41149875 |
| signaling system | | | 213222_AT, 205376_AT | | | | | | | |
| hsa00562: Inositol phosphate | 4 | 0.824742268 | 205111_S_AT, 203896_S_AT, 213222_AT, | 160 | 54 | 5085 | 2.354166667 | 0.237910972 | 0.894176888 | 95.66021546 |
| metabolism | | | 205376_AT | | | | | | | |
| hsa00512: O-Glycan | 3 | 0.618556701 | 201722_S_AT, 201724_S_AT, 222773_S_AT, | 160 | 30 | 5085 | 3.178125 | 0.240966043 | 0.88196132 | 95.85691713 |
| biosynthesis | | | 201723_S_AT, 218885_S_AT, 218313_S_AT, | | | | | | | |
| | | | 1568618_A_AT | | | | | | | |
| hsa05217: Basal cell | 4 | 0.824742268 | 227376_AT, 222696_AT, 205201_AT, | 160 | 55 | 5085 | 2.311363636 | 0.246330162 | 0.872900234 | 96.18269645 |
| carcinoma | | | 216933_X_AT, 224022_X_AT | | | | | | | |
| hsa05130: Pathogenic | 4 | 0.824742268 | 212242_AT, 201596_X_AT, 201743_AT, | 160 | 57 | 5085 | 2.230263158 | 0.263303235 | 0.878166412 | 97.06556067 |
| Escherichia coli infection | | | 206584_AT | | | | | | | |
| hsa04270: Vascular smooth | 6 | 1.237113402 | 203710_AT, 203896_S_AT, 213222_AT, | 160 | 112 | 5085 | 1.702566964 | 0.272280001 | 0.87435781 | 97.45292727 |
| muscle contraction | | | 228396_AT, 203817_AT, 201957_AT | | | | | | | |
| hsa05200: Pathways in cancer | 13 | 2.680412371 | 202267_AT, 227376_AT, 201650_AT, | 160 | 328 | 5085 | 1.259622713 | 0.328151379 | 0.915065748 | 98.98742445 |
| | | | 208944_AT, 205201_AT, 216933_X_AT, | | | | | | | |
| | | | 224022_X_AT, 222696_AT, 227314_AT, | | | | | | | |
| | | | 228121_AT, 203638_S_AT, 209909_S_AT, | | | | | | | |
| | | | 202986_AT, 207334_S_AT, 200878_AT, | | | | | | | |
| | | | 226636_AT | | | | | | | |
| hsa00230: Purine metabolism | 7 | 1.443298969 | 1553995_A_AT, 243100_AT, 206757_AT, | 160 | 153 | 5085 | 1.454044118 | 0.344903862 | 0.917714182 | 99.24352651 |
| | | | 1553994_AT, 228962_AT, 227088_AT, | | | | | | | |
| | | | 203817_AT, 240088_AT, 208447_S_AT, | | | | | | | |
| | | | 206653_AT | | | | | | | |
| hsa00240: Pyrimidine | 5 | 1.030927835 | 1553995_A_AT, 243100_AT, 1553994_AT, | 160 | 95 | 5085 | 1.672697368 | 0.345897317 | 0.908606896 | 99.25666823 |
| metabolism | | | 203234_AT, 222819_AT, 206653_AT | | | | | | | |
| hsa00600: Sphingolipid | 3 | 0.618556701 | 209420_S_AT, 208926_AT, 210980_S_AT, | 160 | 39 | 5085 | 2.444711538 | 0.345902541 | 0.898591956 | 99.25673677 |
| metabolism | | | 213902_AT, 213702_X_AT, 216230_X_AT | | | | | | | |
| hsa04920: Adipocytokine | 4 | 0.824742268 | 235392_AT, 227095_AT, 211356_X_AT, | 160 | 67 | 5085 | 1.89738806 | 0.349516218 | 0.891593553 | 99.30279638 |
| signaling pathway | | | 211355_X_AT, 209894_AT, 206001_AT, | | | | | | | |
| | | | 1560652_AT | | | | | | | |
| hsa00533: Keratan sulfate | 2 | 0.412371134 | 203188_AT, 203921_AT | 160 | 14 | 5085 | 4.540178571 | 0.359385171 | 0.890169981 | 99.4156264 |
| biosynthesis | | | | | | | | | | |
| hsa04940: Type I diabetes | 3 | 0.618556701 | 202410_X_AT, 202409_AT, 200904_AT, | 160 | 42 | 5085 | 2.270089286 | 0.380039373 | 0.897732764 | 99.59974452 |
| mellitus | | | 211799_X_AT | | | | | | | |
| hsa05212: Pancreatic cancer | 4 | 0.824742268 | 228121_AT, 208944_AT, 209909_S_AT, | 160 | 72 | 5085 | 1.765625 | 0.392491188 | 0.898621657 | 99.68334473 |
| | | | 209539_AT, 207334_S_AT, 226636_AT | | | | | | | |
| hsa00511: Other glycan | 2 | 0.412371134 | 202838_AT, 208926_AT | 160 | 16 | 5085 | 3.97265625 | 0.398931858 | 0.895058357 | 99.72001478 |
| degradation | | | | | | | | | | |
| hsa00510: N-Glycan | 3 | 0.618556701 | 201998_AT, 219999_AT, 219797_AT, | 160 | 46 | 5085 | 2.072690217 | 0.42434661 | 0.90570649 | 99.82998993 |
| biosynthesis | | | 226039_AT | | | | | | | |
| hsa05412: Arrhythmogenic | 4 | 0.824742268 | 201650_AT, 227314_AT, 227623_AT, | 160 | 76 | 5085 | 1.672697368 | 0.426366205 | 0.899455832 | 99.83675144 |
| right ventricular | | | 207717_S_AT | | | | | | | |
| cardiomyopathy (ARVC) | | | | | | | | | | |
| hsa04930: Type II diabetes | 3 | 0.618556701 | 202410_X_AT, 235392_AT, 202409_AT, | 160 | 47 | 5085 | 2.028590426 | 0.435172084 | 0.898219493 | 99.86345777 |
| mellitus | | | 1560652_AT | | | | | | | |
| hsa04010: MAPK signaling | 10 | 2.06185567 | 208944_AT, 205403_AT, 205590_AT, | 160 | 267 | 5085 | 1.190308989 | 0.457955243 | 0.906806376 | 99.91512355 |
| pathway | | | 201743_AT, 202948_AT, 207876_S_AT, | | | | | | | |
| | | | 211372_S_AT, 227623_AT, 228121_AT, | | | | | | | |
| | | | 203638_S_AT, 209909_S_AT, 207334_S_AT, | | | | | | | |
| | | | 206084_AT | | | | | | | |
| hsa04020: Calcium signaling | 7 | 1.443298969 | 220108_AT, 205111_S_AT, 203710_AT, | 160 | 176 | 5085 | 1.264026989 | 0.474448805 | 0.910835947 | 99.94059552 |
| pathway | | | 243580_AT, 203896_S_AT, 213222_AT | | | | | | | |
| | | | 226213_AT, 202454_S_AT, 206825_AT | | | | | | | |
| hsa04612: Antigen processing | 4 | 0.824742268 | 201422_AT, 200904_AT, 209619_AT, | 160 | 83 | 5085 | 1.531626506 | 0.483946329 | 0.910426139 | 99.95187601 |
| and presentation | | | 211799_X_AT | | | | | | | |
| hsa00531: Glycosaminoglycan | 2 | 0.412371134 | 210619_S_AT, 212334_AT, 212335_AT | 160 | 21 | 5085 | 3.026785714 | 0.487502261 | 0.906355977 | 99.95556904 |
| degradation | | | | | | | | | | |
| hsa05210: Colorectal cancer | 4 | 0.824742268 | 222696_AT, 228121_AT, 208944_AT, | 160 | 84 | 5085 | 1.513392857 | 0.491955268 | 0.902949984 | 99.95982824 |
| | | | 209909_S_AT, 216933_X_AT, 207334_S_AT | | | | | | | |
| hsa00532: Chondroitin sulfate | 2 | 0.412371134 | 206756_AT, 218927_S_AT | 160 | 22 | 5085 | 2.889204545 | 0.503593719 | 0.904359064 | 99.96926031 |
| biosynthesis | | | | | | | | | | |
| hsa04670: Leukocyte | 5 | 1.030927835 | 228707_AT, 219213_AT, 203868_S_AT, | 160 | 118 | 5085 | 1.346663136 | 0.507625318 | 0.900936806 | 99.97202284 |
| transendothelial migration | | | 1554812_AT, 212813_AT | | | | | | | |
| hsa04640: Hematopoietic cell | 4 | 0.824742268 | 227314_AT, 205403_AT, 202948_AT, | 160 | 86 | 5085 | 1.478197674 | 0.507789544 | 0.894998011 | 99.9721304 |
| lineage | | | 201743_AT, 211372_S_AT | | | | | | | |
| hsa00910: Nitrogen | 2 | 0.412371134 | 203963_AT, 214164_X_AT, 244802_AT, | 160 | 23 | 5085 | 2.763586957 | 0.519183013 | 0.896691183 | 99.97873422 |
| metabolism | | | 204508_S_AT, 215867_X_AT | | | | | | | |
| hsa04062: Chemokine | 7 | 1.443298969 | 223454_AT, 235238_AT, 204115_AT, | 160 | 187 | 5085 | 1.18967246 | 0.534251468 | 0.900834497 | 99.98527688 |
| signaling pathway | | | 203896_S_AT, 213222_AT, 224964_S_AT, | | | | | | | |
| | | | 206336_AT, 230538_AT | | | | | | | |
| hsa00534: Heparan sulfate | 2 | 0.412371134 | 214985_AT, 202013_S_AT | 160 | 26 | 5085 | 2.444711538 | 0.563091283 | 0.913244627 | 99.99296225 |
| biosynthesis | | | | | | | | | | |
| hsa04912: GnRH signaling | 4 | 0.824742268 | 203710_AT, 203896_S_AT, 213222_AT, | 160 | 98 | 5085 | 1.297193878 | 0.597009471 | 0.927258799 | 99.997232 |
| pathway | | | 226636_AT | | | | | | | |
| hsa04650: Natural killer cell | 5 | 1.030927835 | 202687_S_AT, 235238_AT, 202688_AT, | 160 | 133 | 5085 | 1.194783835 | 0.603202403 | 0.926091473 | 99.99768527 |
| mediated cytotoxicity | | | 200904_AT, 205905_S_AT, 211799_X_AT, | | | | | | | |
| | | | 230538_AT, 205904_AT | | | | | | | |
| hsa04916: Melanogenesis | 4 | 0.824742268 | 203896_S_AT, 213222_AT, 224022_X_AT, | 160 | 99 | 5085 | 1.284090909 | 0.603958763 | 0.922097509 | 99.99773571 |
| | | | 222802_AT, 218995_S_AT | | | | | | | |
| hsa04530: Tight junction | 5 | 1.030927835 | 228707_AT, 219213_AT, 201719_S_AT, | 160 | 134 | 5085 | 1.185867537 | 0.609171409 | 0.920542105 | 99.99805694 |
| | | | 1554812_AT, 212813_AT | | | | | | | |
| hsa04910: Insulin signaling | 5 | 1.030927835 | 202410_X_AT, 235238_AT, 235392_AT, | 160 | 135 | 5085 | 1.177083333 | 0.61508596 | 0.919449902 | 99.99837065 |
| pathway | | | 202409_AT, 203680_AT, 1560652_AT, | | | | | | | |
| | | | 230538_AT | | | | | | | |
| hsa04620: Toll-like receptor | 4 | 0.824742268 | 209875_S_AT, 201743_AT, 208436_S_AT, | 160 | 101 | 5085 | 1.258663366 | 0.617620917 | 0.916546768 | 99.99849035 |
| signaling pathway | | | 206584_AT | | | | | | | |
| hsa00250: Alanine, aspartate | 2 | 0.412371134 | 244802_AT, 207076_S_AT | 160 | 31 | 5085 | 2.050403226 | 0.627591281 | 0.917885934 | 99.9988873 |
| and glutamate metabolism | | | | | | | | | | |
| hsa04720: Long-term | 3 | 0.618556701 | 203710_AT, 203896_S_AT, 213222_AT | 160 | 68 | 5085 | 1.402113971 | 0.633522326 | 0.9170459 | 99.99907559 |
| potentiation | | | | | | | | | | |
| hsa04610: Complement and | 3 | 0.618556701 | 213800_AT, 215388_S_AT, 201860_S_AT, | 160 | 69 | 5085 | 1.381793478 | 0.6414852 | 0.917426486 | 99.9992827 |
| coagulation cascades | | | 207808_S_AT | | | | | | | |
| hsa05211: Renal cell | 3 | 0.618556701 | 228121_AT, 209909_S_AT, 202986_AT, | 160 | 70 | 5085 | 1.362053571 | 0.649312273 | 0.917811589 | 99.9994441 |
| carcinoma | | | 200878_AT | | | | | | | |
| hsa04622: RIG-I-like receptor | 3 | 0.618556701 | 219209_AT, 208436_S_AT, 205483_S_AT | 160 | 71 | 5085 | 1.342869718 | 0.657004196 | 0.918200401 | 99.9995697 |
| signaling pathway | | | | | | | | | | |
| hsa03022: Basal transcription | 2 | 0.412371134 | 209595_AT, 213413_AT | 160 | 35 | 5085 | 1.816071429 | 0.672302658 | 0.92284177 | 99.99974593 |
| factors | | | | | | | | | | |
| hsa05330: Allograft rejection | 2 | 0.412371134 | 200904_AT, 211799_X_AT | 160 | 36 | 5085 | 1.765625 | 0.682620257 | 0.92478791 | 99.9998244 |
| hsa05220: Chronic myeloid | 3 | 0.618556701 | 235238_AT, 228121_AT, 208944_AT, | 160 | 75 | 5085 | 1.27125 | 0.686436474 | 0.923313161 | 99.9998473 |
| leukemia | | | 209909_S_AT, 230538_AT, 207334_S_AT | | | | | | | |
| hsa04080: Neuroactive ligand- | 8 | 1.649484536 | 218589_AT, 211355_X_AT, 211356_X_AT, | 160 | 256 | 5085 | 0.993164063 | 0.697397349 | 0.925754438 | 99.99989875 |
| receptor interaction | | | 209894_AT, 205280_AT, 211679_X_AT, | | | | | | | |
| | | | 235591_AT, 206825_AT, 213506_AT, | | | | | | | |
| | | | 227095_AT, 244680_AT, 206730_AT, | | | | | | | |
| | | | 1569290_S_AT, 230144_AT | | | | | | | |
| hsa05332: Graft-versus-host | 2 | 0.412371134 | 200904_AT, 211799_X_AT | 160 | 39 | 5085 | 1.629807692 | 0.71167568 | 0.929973339 | 99.99994206 |
| disease | | | | | | | | | | |
| hsa05410: Hypertrophic | 3 | 0.618556701 | 228121_AT, 227314_AT, 209909_S_AT, | 160 | 85 | 5085 | 1.121691176 | 0.751047544 | 0.94619521 | 99.99998937 |
| cardiomyopathy (HCM) | | | 227623_AT | | | | | | | |
| hsa04210: Apoptosis | 3 | 0.618556701 | 202687_S_AT, 202948_AT, 202688_AT, | 160 | 87 | 5085 | 1.095905172 | 0.762515357 | 0.948755558 | 99.99999383 |
| | | | 203680_AT | | | | | | | |
| hsa00140: Steroid hormone | 2 | 0.412371134 | 206938_AT, 209160_AT, 235945_AT | 160 | 46 | 5085 | 1.381793478 | 0.769597032 | 0.949408902 | 99.99999565 |
| biosynthesis | | | | | | | | | | |
| hsa05215: Prostate cancer | 3 | 0.618556701 | 202410_X_AT, 206938_AT, 202409_AT, | 160 | 89 | 5085 | 1.07127809 | 0.773525708 | 0.948709395 | 99.99999643 |
| | | | 203638_S_AT, 235945_AT | | | | | | | |
| hsa04330: Notch signaling | 2 | 0.412371134 | 242794_AT, 203395_S_AT | 160 | 47 | 5085 | 1.352393617 | 0.776867139 | 0.94778352 | 99.999997 |
| pathway | | | | | | | | | | |
| hsa05414: Dilated | 3 | 0.618556701 | 228121_AT, 227314_AT, 209909_S_AT, | 160 | 92 | 5085 | 1.036345109 | 0.789210787 | 0.951027053 | 99.99999844 |
| cardiomyopathy | | | 227623_AT | | | | | | | |
| hsa05320: Autoimmune | 2 | 0.412371134 | 200904_AT, 211799_X_AT | 160 | 51 | 5085 | 1.246323529 | 0.803737517 | 0.955233707 | 99.99999932 |
| thyroid disease | | | | | | | | | | |
| hsa04666: Fc gamma R- | 3 | 0.618556701 | 212587_S_AT, 212588_AT, 1558502_S_AT, | 160 | 95 | 5085 | 1.003618421 | 0.803937464 | 0.95316618 | 99.99999933 |
| mediated phagocytosis | | | 207238_S_AT, 1558501_AT, 209839_AT, | | | | | | | |
| | | | 226636_AT | | | | | | | |
| hsa05213: Endometrial cancer | 2 | 0.412371134 | 222696_AT, 216933_X_AT | 160 | 52 | 5085 | 1.222355769 | 0.80993651 | 0.953716685 | 99.99999953 |
| hsa04810: Regulation of actin | 6 | 1.237113402 | 202410_X_AT, 227314_AT, 202409_AT, | 160 | 215 | 5085 | 0.886918605 | 0.810548566 | 0.9518611 | 99.99999955 |
| cytoskeleton | | | 203638_S_AT, 201743_AT, 216933_X_AT, | | | | | | | |
| | | | 209539_AT | | | | | | | |
| hsa00330: Arginine and | 2 | 0.412371134 | 244802_AT, 207076_S_AT, | 160 | 53 | 5085 | 1.199292453 | 0.8159409 | 0.952241169 | 99.99999967 |
| proline metabolism | | | | | | | | | | |
| hsa04623: Cytosolic DNA- | 2 | 0.412371134 | 208436_S_AT, 206653_AT | 160 | 55 | 5085 | 1.155681818 | 0.827389967 | 0.955483914 | 99.99999985 |
| sensing pathway | | | | | | | | | | |
| hsa04340: Hedgehog signaling | 2 | 0.412371134 | 227376_AT, 205201_AT, 224022_X_AT | 160 | 56 | 5085 | 1.135044643 | 0.832846229 | 0.956026724 | 99.99999989 |
| pathway | | | | | | | | | | |
| hsa04630: Jak-STAT signaling | 4 | 0.824742268 | 200953_S_AT, 227095_AT, 211356_X_AT, | 160 | 155 | 5085 | 0.82016129 | 0.869942363 | 0.970191159 | 99.99999999 |
| pathway | | | 211355_X_AT, 209894_AT, 212558_AT, | | | | | | | |
| | | | 244261_AT | | | | | | | |
| hsa04115: p53 signaling | 2 | 0.412371134 | 200953_S_AT, 1554830_A_AT | 160 | 68 | 5085 | 0.934742647 | 0.886368769 | 0.975132052 | 100 |
| pathway | | | | | | | | | | |
| hsa05120: Epithelial cell | 2 | 0.412371134 | 219213_AT, 212813_AT | 160 | 68 | 5085 | 0.934742647 | 0.886368769 | 0.975132052 | 100 |
| signaling in Helicobacter | | | | | | | | | | |
| pylori infection | | | | | | | | | | |
| hsa05010: Alzheimer's disease | 4 | 0.824742268 | 203710_AT, 203896_S_AT, 213222_AT, | 160 | 163 | 5085 | 0.779907975 | 0.891046209 | 0.975637019 | 100 |
| | | | 203382_S_AT | | | | | | | |
| hsa05416: Viral myocarditis | 2 | 0.412371134 | 200904_AT, 211799_X_AT | 160 | 71 | 5085 | 0.895246479 | 0.8968357 | 0.976609725 | 100 |
| hsa04722: Neurotrophin | 3 | 0.618556701 | 235238_AT, 235392_AT, 1560652_AT, | 160 | 124 | 5085 | 0.76890121 | 0.905410557 | 0.978669234 | 100 |
| signaling pathway | | | 230538_AT | | | | | | | |
| hsa04260: Cardiac muscle | 2 | 0.412371134 | 227623_AT, 201243_S_AT | 160 | 78 | 5085 | 0.814903846 | 0.91768023 | 0.982071699 | 100 |
| contraction | | | | | | | | | | |
| hsa05222: Small cell lung | 2 | 0.412371134 | 202267_AT, 227314_AT | 160 | 84 | 5085 | 0.756696429 | 0.932177849 | 0.986126679 | 100 |
| cancer | | | | | | | | | | |
| hsa04012: ErbB signaling | 2 | 0.412371134 | 235238_AT, 226213_AT, 202454_S_AT, | 160 | 87 | 5085 | 0.730603448 | 0.93844451 | 0.987422085 | 100 |
| pathway | | | 230538_AT | | | | | | | |
| hsa04660: T cell receptor | 2 | 0.412371134 | 205590_AT, 212587_S_AT, 212588_AT, | 160 | 108 | 5085 | 0.588541667 | 0.96882458 | 0.995371832 | 100 |
| signaling pathway | | | 207238_S_AT | | | | | | | |
| hsa04114: Oocyte meiosis | 2 | 0.412371134 | 202410_X_AT, 203710_AT, 202409_AT | 160 | 110 | 5085 | 0.577840909 | 0.970784875 | 0.995522312 | 100 |
| hsa05016: Huntington's | 3 | 0.618556701 | 203710_AT, 203896_S_AT, 213222_AT | 160 | 180 | 5085 | 0.5296875 | 0.979202246 | 0.997139034 | 100 |
| disease | | | | | | | | | | |
| hsa04110: Cell cycle | 2 | 0.412371134 | 200953_S_AT, 228121_AT, 209909_S_AT | 160 | 125 | 5085 | 0.5085 | 0.982064833 | 0.997539191 | 100 |
| hsa04360: Axon guidance | 2 | 0.412371134 | 204584_AT, 202668_AT | 160 | 129 | 5085 | 0.492732558 | 0.984256977 | 0.997819487 | 100 |
| hsa04740: Olfactory | 2 | 0.412371134 | 1255_G_AT, 206062_AT, 228396_AT | 160 | 379 | 5085 | 0.167711082 | 0.999996343 | 0.999999988 | 100 |
| transduction | | | | | | | | | | |

TABLE 1C

| | | | | | | | Fold | | | |
|---|---|---|---|---|---|---|---|---|---|---|
| Term | Count | % | Genes | List Total | Pop Hits | Pop Total | Enrichment | PValue | Benjamini | FDR |
| GO: 0007155~cell adhesion | 42 | 8.659793814 | 266_S_AT, 222108_AT, 228707_AT, | 348 | 700 | 13528 | 2.332413793 | 6.48E−07 | 0.001581259 | 0.001140149 |
| | | | 201650_AT, 203868_S_AT, | | | | | | | |
| | | | 204875_S_AT, 236029_AT, | | | | | | | |
| | | | 204688_AT, 207370_AT, | | | | | | | |
| | | | 209032_S_AT, 203477_AT, | | | | | | | |
| | | | 209031_AT, 243337_AT, 203780_AT, | | | | | | | |
| | | | 227747_AT, 222020_S_AT, | | | | | | | |
| | | | 227566_AT, 209771_X_AT, | | | | | | | |
| | | | 212587_S_AT, 216933_X_AT, | | | | | | | |
| | | | 233401_AT, 203779_S_AT, | | | | | | | |
| | | | 226824_AT, 209030_S_AT, | | | | | | | |
| | | | 228635_AT, 213438_AT, 212070_AT, | | | | | | | |
| | | | 206025_S_AT, 223796_AT, | | | | | | | |
| | | | 228101_AT, 207717_S_AT, | | | | | | | |
| | | | 203476_AT, 202267_AT, | | | | | | | |
| | | | 209875_S_AT, 236028_AT, | | | | | | | |
| | | | 209462_AT, 200923_AT, | | | | | | | |
| | | | 203395_S_AT, 230518_AT, | | | | | | | |
| | | | 203083_AT, 227314_AT, 204320_AT, | | | | | | | |
| | | | 202150_S_AT, 212588_AT, | | | | | | | |
| | | | 1554812_AT, 209493_AT, | | | | | | | |
| | | | 220115_S_AT, 219213_AT, | | | | | | | |
| | | | 37892_AT, 206026_S_AT, | | | | | | | |
| | | | 204105_S_AT, 227202_AT, | | | | | | | |
| | | | 207238_S_AT, 204584_AT | | | | | | | |
| GO: 0022610~biological adhesion | 42 | 8.659793814 | 266_S_AT, 222108_AT, 228707_AT, | 348 | 701 | 13528 | 2.329086527 | 6.57E−07 | 8.02E−04 | 0.001156649 |
| | | | 201650_AT, 203868_S_AT, | | | | | | | |
| | | | 204875_S_AT, 236029_AT, | | | | | | | |
| | | | 204688_AT, 207370_AT, | | | | | | | |
| | | | 209032_S_AT, 203477_AT, | | | | | | | |
| | | | 209031_AT, 243337_AT, 203780_AT, | | | | | | | |
| | | | 227747_AT, 222020_S_AT, | | | | | | | |
| | | | 227566_AT, 209771_X_AT, | | | | | | | |
| | | | 212587_S_AT, 216933_X_AT, | | | | | | | |
| | | | 233401_AT, 203779_S_AT, | | | | | | | |
| | | | 226824_AT, 209030_S_AT, | | | | | | | |
| | | | 228635_AT, 213438_AT, 212070_AT, | | | | | | | |
| | | | 206025_S_AT, 223796_AT, | | | | | | | |
| | | | 228101_AT, 207717_S_AT, | | | | | | | |
| | | | 203476_AT, 202267_AT, | | | | | | | |
| | | | 209875_S_AT, 236028_AT, | | | | | | | |
| | | | 209462_AT, 200923_AT, | | | | | | | |
| | | | 203395_S_AT, 230518_AT, | | | | | | | |
| | | | 203083_AT, 227314_AT, 204320_AT, | | | | | | | |
| | | | 202150_S_AT, 212588_AT, | | | | | | | |
| | | | 1554812_AT, 209493_AT, | | | | | | | |
| | | | 220115_S_AT, 219213_AT, | | | | | | | |
| | | | 37892_AT, 206026_S_AT, | | | | | | | |
| | | | 204105_S_AT, 227202_AT, | | | | | | | |
| | | | 207238_S_AT, 204584_AT | | | | | | | |
| GO: 0001944~vasculature | 22 | 4.536082474 | 224722_AT, 229339_AT, 208944_AT, | 348 | 251 | 13528 | 3.407244585 | 2.04E−06 | 0.001660567 | 0.003592097 |
| development | | | 211355_X_AT, 215446_S_AT, | | | | | | | |
| | | | 204575_S_AT, 202668_AT, | | | | | | | |
| | | | 226701_AT, 218995_S_AT, | | | | | | | |
| | | | 204298_S_AT, 209909_S_AT, | | | | | | | |
| | | | 203477_AT, 229376_AT, | | | | | | | |
| | | | 44783_S_AT, 209211_AT, | | | | | | | |
| | | | 225544_AT, 201860_S_AT, | | | | | | | |
| | | | 207334_S_AT, 203382_S_AT, | | | | | | | |
| | | | 200878_AT, 229576_S_AT, | | | | | | | |
| | | | 211356_X_AT, 209894_AT, | | | | | | | |
| | | | 237206_AT, 218839_AT, 226498_AT, | | | | | | | |
| | | | 204035_AT, 228121_AT, 227095_AT, | | | | | | | |
| | | | 212124_AT, 208937_S_AT, | | | | | | | |
| | | | 209212_S_AT, 219682_S_AT, | | | | | | | |
| | | | 222802_AT | | | | | | | |
| GO: 0007507~heart development | 20 | 4.12371134 | 227376_AT, 224722_AT, 229339_AT, | 348 | 215 | 13528 | 3.616145416 | 2.82E−06 | 0.001721935 | 0.004966582 |
| | | | 208944_AT, 205201_AT, 226213_AT, | | | | | | | |
| | | | 201566_X_AT, 226701_AT, | | | | | | | |
| | | | 218995_S_AT, 204320_AT, | | | | | | | |
| | | | 209909_S_AT, 207826_S_AT, | | | | | | | |
| | | | 229376_AT, 225544_AT, | | | | | | | |
| | | | 207334_S_AT, 205111_S_AT, | | | | | | | |
| | | | 229576_S_AT, 204689_AT, | | | | | | | |
| | | | 37892_AT, 201565_S_AT, | | | | | | | |
| | | | 237206_AT, 202454_S_AT, | | | | | | | |
| | | | 206825_AT, 228121_AT, 212124_AT, | | | | | | | |
| | | | 215933_S_AT, 208937_S_AT, | | | | | | | |
| | | | 209765_AT, 222802_AT, | | | | | | | |
| | | | 219682_S_AT, 207717_S_AT | | | | | | | |
| GO: 0001568~blood vessel | 21 | 4.329896907 | 224722_AT, 229339_AT, 208944_AT, | 348 | 245 | 13528 | 3.332019704 | 5.21E−06 | 0.002543068 | 0.009172301 |
| development | | | 211355_X_AT, 215446_S_AT, | | | | | | | |
| | | | 204575_S_AT, 226701_AT, | | | | | | | |
| | | | 218995_S_AT, 204298_S_AT, | | | | | | | |
| | | | 209909_S_AT, 203477_AT, | | | | | | | |
| | | | 229376_AT, 44783_S_AT, | | | | | | | |
| | | | 209211_AT, 225544_AT, | | | | | | | |
| | | | 201860_S_AT, 207334_S_AT, | | | | | | | |
| | | | 203382_S_AT, 200878_AT, | | | | | | | |
| | | | 229576_S_AT, 211356_X_AT, | | | | | | | |
| | | | 209894_AT, 237206_AT, 218839_AT, | | | | | | | |
| | | | 226498_AT, 204035_AT, 228121_AT, | | | | | | | |
| | | | 227095_AT, 212124_AT, | | | | | | | |
| | | | 208937_S_AT, 209212_S_AT, | | | | | | | |
| | | | 219682_S_AT, 222802_AT | | | | | | | |
| GO: 0009611~response to | 32 | 6.597938144 | 209875_S_AT, 266_S_AT, | 348 | 530 | 13528 | 2.347083062 | 1.66E−05 | 0.006715982 | 0.029125773 |
| wounding | | | 208944_AT, 215446_S_AT, | | | | | | | |
| | | | 215388_S_AT, 226213_AT, | | | | | | | |
| | | | 203921_AT, 213506_AT, | | | | | | | |
| | | | 204298_S_AT, 204140_AT, | | | | | | | |
| | | | 227314_AT, 231798_AT, 202409_AT, | | | | | | | |
| | | | 209909_S_AT, 207826_S_AT, | | | | | | | |
| | | | 235944_AT, 209230_S_AT, | | | | | | | |
| | | | 202446_S_AT, 208436_S_AT, | | | | | | | |
| | | | 201860_S_AT, 207334_S_AT, | | | | | | | |
| | | | 209771_X_AT, 213800_AT, | | | | | | | |
| | | | 201743_AT, 223217_S_AT, | | | | | | | |
| | | | 206336_AT, 206026_S_AT, | | | | | | | |
| | | | 202454_S_AT, 206157_AT, | | | | | | | |
| | | | 202430_S_AT, 207808_S_AT, | | | | | | | |
| | | | 206584_AT, 204035_AT, 207145_AT, | | | | | | | |
| | | | 202410_X_AT, 228121_AT, | | | | | | | |
| | | | 219773_AT, 209035_AT, | | | | | | | |
| | | | 232082_X_AT, 206025_S_AT, | | | | | | | |
| | | | 203799_AT | | | | | | | |
| GO: 0042127~regulation of cell | 40 | 8.24742268 | 266_S_AT, 227376_AT, 235392_AT, | 348 | 787 | 13528 | 1.975784662 | 5.94E−05 | 0.020501235 | 0.10441406 |
| proliferation | | | 208944_AT, 205201_AT, | | | | | | | |
| | | | 204526_S_AT, 228758_AT, | | | | | | | |
| | | | 203868_S_AT, 244261_AT, | | | | | | | |
| | | | 211737_X_AT, 231798_AT, | | | | | | | |
| | | | 202986_AT, 209230_S_AT, | | | | | | | |
| | | | 225544_AT, 209211_AT, | | | | | | | |
| | | | 203382_S_AT, 207334_S_AT, | | | | | | | |
| | | | 229576_S_AT, 209771_X_AT, | | | | | | | |
| | | | 212587_S_AT, 216933_X_AT, | | | | | | | |
| | | | 201565_S_AT, 226498_AT, | | | | | | | |
| | | | 204035_AT, 219773_AT, 201422_AT, | | | | | | | |
| | | | 212124_AT, 236439_AT, 222802_AT, | | | | | | | |
| | | | 229339_AT, 217707_X_AT, | | | | | | | |
| | | | 226213_AT, 201566_X_AT, | | | | | | | |
| | | | 235591_AT, 203395_S_AT, | | | | | | | |
| | | | 213721_AT, 218995_S_AT, | | | | | | | |
| | | | 210619_S_AT, 222696_AT, | | | | | | | |
| | | | 227314_AT, 202409_AT, | | | | | | | |
| | | | 209909_S_AT, 203638_S_AT, | | | | | | | |
| | | | 212588_AT, 229376_AT, 203333_AT, | | | | | | | |
| | | | 204689_AT, 203140_AT, | | | | | | | |
| | | | 202454_S_AT, 237206_AT, | | | | | | | |
| | | | 200953_S_AT, 202410_X_AT, | | | | | | | |
| | | | 228121_AT, 215933_S_AT, | | | | | | | |
| | | | 207238_S_AT, 209212_S_AT, | | | | | | | |
| | | | 209465_X_AT, 209466_X_AT, | | | | | | | |
| | | | 219682_S_AT | | | | | | | |
| GO: 0008284~positive regulation of | 26 | 5.360824742 | 266_S_AT, 235392_AT, 229339_AT, | 348 | 414 | 13528 | 2.441334888 | 6.68E−05 | 0.020171817 | 0.117385638 |
| cell proliferation | | | 208944_AT, 228758_AT, | | | | | | | |
| | | | 204526_S_AT, 203868_S_AT, | | | | | | | |
| | | | 201566_X_AT, 203395_S_AT, | | | | | | | |
| | | | 213721_AT, 218995_S_AT, | | | | | | | |
| | | | 227314_AT, 211737_X_AT, | | | | | | | |
| | | | 231798_AT, 202409_AT, | | | | | | | |
| | | | 209909_S_AT, 203638_S_AT, | | | | | | | |
| | | | 202986_AT, 212588_AT, 229376_AT, | | | | | | | |
| | | | 225544_AT, 209211_AT, | | | | | | | |
| | | | 207334_S_AT, 209771_X_AT, | | | | | | | |
| | | | 229576_S_AT, 203140_AT, | | | | | | | |
| | | | 212587_S_AT, 201565_S_AT, | | | | | | | |
| | | | 237206_AT, 204035_AT, 226498_AT, | | | | | | | |
| | | | 200953_S_AT, 202410_X_AT, | | | | | | | |
| | | | 228121_AT, 212124_AT, 236439_AT, | | | | | | | |
| | | | 207238_S_AT, 209212_S_AT, | | | | | | | |
| | | | 209465_X_AT, 209466_X_AT, | | | | | | | |
| | | | 222802_AT, 219682_S_AT | | | | | | | |
| GO: 0048514~blood vessel | 17 | 3.505154639 | 229339_AT, 208944_AT, | 348 | 211 | 13528 | 3.131993245 | 1.10E−04 | 0.029276554 | 0.19248544 |
| morphogenesis | | | 211355_X_AT, 204575_S_AT, | | | | | | | |
| | | | 218995_S_AT, 209909_S_AT, | | | | | | | |
| | | | 203477_AT, 229376_AT, | | | | | | | |
| | | | 44783_S_AT, 209211_AT, | | | | | | | |
| | | | 201860_S_AT, 207334_S_AT, | | | | | | | |
| | | | 203382_S_AT, 200878_AT, | | | | | | | |
| | | | 211356_X_AT, 209894_AT, | | | | | | | |
| | | | 237206_AT, 218839_AT, 226498_AT, | | | | | | | |
| | | | 204035_AT, 228121_AT, 227095_AT, | | | | | | | |
| | | | 212124_AT, 208937_S_AT, | | | | | | | |
| | | | 209212_S_AT, 222802_AT | | | | | | | |
| GO: 0006952~defense response | 33 | 6.804123711 | 266_S_AT, 209875_S_AT, | 348 | 615 | 13528 | 2.085898514 | 1.16E−04 | 0.027818432 | 0.203058278 |
| | | | 206082_AT, 202948_AT, | | | | | | | |
| | | | 215388_S_AT, 211799_X_AT, | | | | | | | |
| | | | 203921_AT, 200923_AT, 202086_AT, | | | | | | | |
| | | | 205602_X_AT, 204140_AT, | | | | | | | |
| | | | 202409_AT, 205258_AT, 212588_AT, | | | | | | | |
| | | | 209230_S_AT, 208436_S_AT, | | | | | | | |
| | | | 226636_AT, 209771_X_AT, | | | | | | | |
| | | | 213800_AT, 201743_AT, 204994_AT, | | | | | | | |
| | | | 212587_S_AT, 209619_AT, | | | | | | | |
| | | | 223217_S_AT, 206336_AT, | | | | | | | |
| | | | 206026_S_AT, 205905_S_AT, | | | | | | | |
| | | | 210126_AT, 205904_AT, 206157_AT, | | | | | | | |
| | | | 206584_AT, 204035_AT, | | | | | | | |
| | | | 202410_X_AT, 223454_AT, | | | | | | | |
| | | | 219773_AT, 219209_AT, | | | | | | | |
| | | | 206025_S_AT, 207238_S_AT, | | | | | | | |
| | | | 203799_AT, 210511_S_AT | | | | | | | |
| GO: 0045785~positive regulation of | 9 | 1.855670103 | 209875_S_AT, 266_S_AT, | 348 | 60 | 13528 | 5.831034483 | 1.38E−04 | 0.030214092 | 0.242849294 |
| cell adhesion | | | 228121_AT, 203333_AT, 227314_AT, | | | | | | | |
| | | | 209771_X_AT, 203886_S_AT, | | | | | | | |
| | | | 230319_AT, 209909_S_AT, | | | | | | | |
| | | | 216933_X_AT, 204584_AT | | | | | | | |
| GO: 0007267~cell-cell signaling | 32 | 6.597938144 | 266_S_AT, 202688_AT, 205280_AT, | 348 | 600 | 13528 | 2.073256705 | 1.68E−04 | 0.033521206 | 0.294348068 |
| | | | 206001_AT, 224022_X_AT, | | | | | | | |
| | | | 211679_X_AT, 235591_AT, | | | | | | | |
| | | | 213721_AT, 226701_AT, | | | | | | | |
| | | | 218995_S_AT, 202668_AT, | | | | | | | |
| | | | 205741_S_AT, 206857_S_AT, | | | | | | | |
| | | | 202687_S_AT, 203998_S_AT, | | | | | | | |
| | | | 202409_AT, 203439_S_AT, | | | | | | | |
| | | | 209909_S_AT, 225544_AT, | | | | | | | |
| | | | 241652_X_AT, 201860_S_AT, | | | | | | | |
| | | | 203382_S_AT, 209771_X_AT, | | | | | | | |
| | | | 229576_S_AT, 206938_AT, | | | | | | | |
| | | | 204867_AT, 235945_AT, 212558_AT, | | | | | | | |
| | | | 206336_AT, 206026_S_AT, | | | | | | | |
| | | | 205483_S_AT, 206825_AT, | | | | | | | |
| | | | 202410_X_AT, 228121_AT, | | | | | | | |
| | | | 244680_AT, 212070_AT, | | | | | | | |
| | | | 206025_S_AT, 201641_AT, | | | | | | | |
| | | | 210511_S_AT, 222802_AT, | | | | | | | |
| | | | 219682_S_AT, 228101_AT | | | | | | | |
| GO: 0009725~response to hormone | 23 | 4.742268041 | 209875_S_AT, 266_S_AT, | 348 | 367 | 13528 | 2.436217858 | 1.99E−04 | 0.036679323 | 0.349390924 |
| stimulus | | | 213221_S_AT, 235392_AT, | | | | | | | |
| | | | 208944_AT, 211355_X_AT, | | | | | | | |
| | | | 215446_S_AT, 226213_AT, | | | | | | | |
| | | | 224964_S_AT, 206518_S_AT, | | | | | | | |
| | | | 204298_S_AT, 204932_AT, | | | | | | | |
| | | | 227314_AT, 202409_AT, | | | | | | | |
| | | | 209909_S_AT, 203680_AT, | | | | | | | |
| | | | 202986_AT, 207334_S_AT, | | | | | | | |
| | | | 226636_AT, 203560_AT, | | | | | | | |
| | | | 209771_X_AT, 206938_AT, | | | | | | | |
| | | | 204115_AT, 211356_X_AT, | | | | | | | |
| | | | 209894_AT, 235945_AT, | | | | | | | |
| | | | 202454_S_AT, 206825_AT, | | | | | | | |
| | | | 207145_AT, 200953_S_AT, | | | | | | | |
| | | | 202410_X_AT, 228121_AT, | | | | | | | |
| | | | 227095_AT, 204933_S_AT, | | | | | | | |
| | | | 223430_AT | | | | | | | |
| GO: 0040017~positive regulation of | 11 | 2.268041237 | 235392_AT, 216933_X_AT, | 348 | 98 | 13528 | 4.363359137 | 2.05E−04 | 0.035102969 | 0.35978522 |
| locomotion | | | 213506_AT, 218995_S_AT, | | | | | | | |
| | | | 226498_AT, 204035_AT, 227314_AT, | | | | | | | |
| | | | 228121_AT, 202410_X_AT, | | | | | | | |
| | | | 223454_AT, 202409_AT, | | | | | | | |
| | | | 209909_S_AT, 222802_AT, | | | | | | | |
| | | | 226636_AT | | | | | | | |
| GO: 0051272~positive regulation of | 11 | 2.268041237 | 235392_AT, 203140_AT, 228758_AT, | 348 | 98 | 13528 | 4.363359137 | 2.05E−04 | 0.035102969 | 0.35978522 |
| cell motion | | | 216933_X_AT, 213506_AT, | | | | | | | |
| | | | 218995_S_AT, 226498_AT, | | | | | | | |
| | | | 223454_AT, 227314_AT, 228121_AT, | | | | | | | |
| | | | 202410_X_AT, 202409_AT, | | | | | | | |
| | | | 209909_S_AT, 236439_AT, | | | | | | | |
| | | | 222802_AT, 226636_AT | | | | | | | |
| GO: 0006022~aminoglycan | 9 | 1.855670103 | 210619_S_AT, 203188_AT, | 348 | 65 | 13528 | 5.382493369 | 2.44E−04 | 0.038937542 | 0.42829323 |
| metabolic process | | | 214985_AT, 202838_AT, 212334_AT, | | | | | | | |
| | | | 230319_AT, 212335_AT, 206756_AT, | | | | | | | |
| | | | 202013_S_AT, 218927_S_AT | | | | | | | |
| GO: 0009719~response to | 24 | 4.948453608 | 209875_S_AT, 266_S_AT, | 348 | 405 | 13528 | 2.303618561 | 3.20E−04 | 0.047617091 | 0.560829378 |
| endogenous stimulus | | | 213221_S_AT, 235392_AT, | | | | | | | |
| | | | 208944_AT, 211355_X_AT, | | | | | | | |
| | | | 215446_S_AT, 226213_AT, | | | | | | | |
| | | | 224964_S_AT, 206518_S_AT, | | | | | | | |
| | | | 204932_AT, 204298_S_AT, | | | | | | | |
| | | | 227314_AT, 205828_AT, 202409_AT, | | | | | | | |
| | | | 209909_S_AT, 203680_AT, | | | | | | | |
| | | | 202986_AT, 207334_S_AT, | | | | | | | |
| | | | 226636_AT, 203560_AT, | | | | | | | |
| | | | 209771_X_AT, 206938_AT, | | | | | | | |
| | | | 204115_AT, 211356_X_AT, | | | | | | | |
| | | | 209894_AT, 235945_AT, | | | | | | | |
| | | | 202454_S_AT, 206825_AT, | | | | | | | |
| | | | 207145_AT, 200953_S_AT, | | | | | | | |
| | | | 202410_X_AT, 228121_AT, | | | | | | | |
| | | | 227095_AT, 204933_S_AT, | | | | | | | |
| | | | 223430_AT | | | | | | | |
| GO: 0010033~response to organic | 35 | 7.216494845 | 266_S_AT, 235392_AT, 208944_AT, | 348 | 721 | 13528 | 1.887066176 | 4.48E−04 | 0.062313556 | 0.784937585 |
| substance | | | 215446_S_AT, 213902_AT, | | | | | | | |
| | | | 213702_X_AT, 224964_S_AT, | | | | | | | |
| | | | 206518_S_AT, 204298_S_AT, | | | | | | | |
| | | | 206857_S_AT, 202986_AT, | | | | | | | |
| | | | 203382_S_AT, 207334_S_AT, | | | | | | | |
| | | | 203560_AT, 209771_X_AT, | | | | | | | |
| | | | 206938_AT, 211356_X_AT, | | | | | | | |
| | | | 209894_AT, 201565_S_AT, | | | | | | | |
| | | | 207145_AT, 223454_AT, | | | | | | | |
| | | | 210980_S_AT, 204933_S_AT, | | | | | | | |
| | | | 208937_S_AT, 209875_S_AT, | | | | | | | |
| | | | 209420_S_AT, 213221_S_AT, | | | | | | | |
| | | | 211355_X_AT, 202948_AT, | | | | | | | |
| | | | 226213_AT, 201566_X_AT, | | | | | | | |
| | | | 216230_X_AT, 204932_AT, | | | | | | | |
| | | | 205828_AT, 227314_AT, 202409_AT, | | | | | | | |
| | | | 209909_S_AT, 207826_S_AT, | | | | | | | |
| | | | 203680_AT, 226636_AT, 204115_AT, | | | | | | | |
| | | | 201743_AT, 235945_AT, | | | | | | | |
| | | | 202454_S_AT, 206825_AT, | | | | | | | |
| | | | 206584_AT, 200953_S_AT, | | | | | | | |
| | | | 228121_AT, 227095_AT, | | | | | | | |
| | | | 202410_X_AT, 223430_AT | | | | | | | |
| GO: 0030335~positive regulation of | 10 | 2.06185567 | 235392_AT, 216933_X_AT, | 348 | 89 | 13528 | 4.367816092 | 4.53E−04 | 0.059616753 | 0.793976499 |
| cell migration | | | 213506_AT, 218995_S_AT, | | | | | | | |
| | | | 226498_AT, 227314_AT, 228121_AT, | | | | | | | |
| | | | 202410_X_AT, 223454_AT, | | | | | | | |
| | | | 202409_AT, 209909_S_AT, | | | | | | | |
| | | | 222802_AT, 226636_AT | | | | | | | |
| GO: 0030203~glycosaminoglycan | 8 | 1.649484536 | 210619_S_AT, 214985_AT, | 348 | 55 | 13528 | 5.654336468 | 4.84E−04 | 0.06033576 | 0.848283166 |
| metabolic process | | | 202838_AT, 212334_AT, 230319_AT, | | | | | | | |
| | | | 212335_AT, 206756_AT, | | | | | | | |
| | | | 202013_S_AT, 218927_S_AT | | | | | | | |
| GO: 0009615~response to virus | 11 | 2.268041237 | 214453_S_AT, 212587_S_AT, | 348 | 109 | 13528 | 3.923020141 | 4.86E−04 | 0.05767764 | 0.852381078 |
| | | | 204994_AT, 205905_S_AT, | | | | | | | |
| | | | 202086_AT, 205483_S_AT, | | | | | | | |
| | | | 202430_S_AT, 205904_AT, | | | | | | | |
| | | | 219209_AT, 201362_AT, 212588_AT, | | | | | | | |
| | | | 202446_S_AT, 207238_S_AT, | | | | | | | |
| | | | 208436_S_AT, 201641_AT | | | | | | | |
| GO: 0006937~regulation of muscle | 9 | 1.855670103 | 226498_AT, 206857_S_AT, | 348 | 72 | 13528 | 4.859195402 | 4.95E−04 | 0.055972062 | 0.867689997 |
| contraction | | | 229339_AT, 205111_S_AT, | | | | | | | |
| | | | 227314_AT, 237206_AT, 202071_AT, | | | | | | | |
| | | | 222802_AT, 201957_AT, 206825_AT, | | | | | | | |
| | | | 218995_S_AT | | | | | | | |
| GO: 0006897~endocytosis | 16 | 3.298969072 | 235746_S_AT, 266_S_AT, | 348 | 220 | 13528 | 2.827168234 | 5.64E−04 | 0.060701025 | 0.987665119 |
| | | | 209771_X_AT, 208944_AT, | | | | | | | |
| | | | 201743_AT, 213413_AT, | | | | | | | |
| | | | 212850_S_AT, 1558502_S_AT, | | | | | | | |
| | | | 34697_AT, 209462_AT, 209839_AT, | | | | | | | |
| | | | 206157_AT, 223454_AT, 239472_AT, | | | | | | | |
| | | | 210757_X_AT, 209840_S_AT, | | | | | | | |
| | | | 1558501_AT, 203799_AT, | | | | | | | |
| | | | 207334_S_AT, 203382_S_AT, | | | | | | | |
| | | | 205606_AT, 209841_S_AT | | | | | | | |
| GO: 0010324~membrane | 16 | 3.298969072 | 235746_S_AT, 266_S_AT, | 348 | 220 | 13528 | 2.827168234 | 5.64E−04 | 0.060701025 | 0.987665119 |
| invagination | | | 209771_X_AT, 208944_AT, | | | | | | | |
| | | | 201743_AT, 213413_AT, | | | | | | | |
| | | | 212850_S_AT, 1558502_S_AT, | | | | | | | |
| | | | 34697_AT, 209462_AT, 209839_AT, | | | | | | | |
| | | | 206157_AT, 223454_AT, 239472_AT, | | | | | | | |
| | | | 210757_X_AT, 209840_S_AT, | | | | | | | |
| | | | 1558501_AT, 203799_AT, | | | | | | | |
| | | | 207334_S_AT, 203382_S_AT, | | | | | | | |
| | | | 205606_AT, 209841_S_AT | | | | | | | |
| GO: 0045787~positive regulation of | 8 | 1.649484536 | 229576_S_AT, 207069_S_AT, | 348 | 57 | 13528 | 5.455938697 | 6.04E−04 | 0.062102125 | 1.0568044 |
| cell cycle | | | 201565_S_AT, 201566_X_AT, | | | | | | | |
| | | | 218995_S_AT, 228121_AT, | | | | | | | |
| | | | 202410_X_AT, 200953_S_AT, | | | | | | | |
| | | | 202409_AT, 209909_S_AT, | | | | | | | |
| | | | 229376_AT, 225544_AT, 222802_AT, | | | | | | | |
| | | | 219682_S_AT | | | | | | | |
| GO: 0051050~positive regulation of | 16 | 3.298969072 | 227376_AT, 219181_AT, 224722_AT, | 348 | 223 | 13528 | 2.789134581 | 6.49E−04 | 0.06387759 | 1.134895647 |
| transport | | | 235392_AT, 205201_AT, 244802_AT, | | | | | | | |
| | | | 226213_AT, 202454_S_AT, | | | | | | | |
| | | | 206825_AT, 206157_AT, | | | | | | | |
| | | | 218995_S_AT, 227314_AT, | | | | | | | |
| | | | 228121_AT, 202410_X_AT, | | | | | | | |
| | | | 202409_AT, 209030_S_AT, | | | | | | | |
| | | | 205258_AT, 209909_S_AT, | | | | | | | |
| | | | 209032_S_AT, 209031_AT, | | | | | | | |
| | | | 210511_S_AT, 203382_S_AT, | | | | | | | |
| | | | 222802_AT | | | | | | | |
| GO: 0048732~gland development | 12 | 2.474226804 | 227376_AT, 235392_AT, | 348 | 135 | 13528 | 3.455427842 | 7.10E−04 | 0.066998127 | 1.24131591 |
| | | | 229576_S_AT, 204689_AT, | | | | | | | |
| | | | 205201_AT, 226213_AT, | | | | | | | |
| | | | 202454_S_AT, 203395_S_AT, | | | | | | | |
| | | | 206825_AT, 213721_AT, 227314_AT, | | | | | | | |
| | | | 228121_AT, 231798_AT, | | | | | | | |
| | | | 209909_S_AT, 209035_AT, | | | | | | | |
| | | | 215933_S_AT, 225544_AT, | | | | | | | |
| | | | 219682_S_AT | | | | | | | |
| GO: 0016337~cell-cell adhesion | 18 | 3.711340206 | 266_S_AT, 228707_AT, 222108_AT, | 348 | 276 | 13528 | 2.535232384 | 7.89E−04 | 0.071448606 | 1.379019193 |
| | | | 201650_AT, 203868_S_AT, | | | | | | | |
| | | | 204875_S_AT, 236029_AT, | | | | | | | |
| | | | 230518_AT, 204320_AT, | | | | | | | |
| | | | 209032_S_AT, 212588_AT, | | | | | | | |
| | | | 209031_AT, 1554812_AT, | | | | | | | |
| | | | 203780_AT, 220115_S_AT, | | | | | | | |
| | | | 209771_X_AT, 219213_AT, | | | | | | | |
| | | | 212587_S_AT, 37892_AT, | | | | | | | |
| | | | 233401_AT, 203779_S_AT, | | | | | | | |
| | | | 204105_S_AT, 209030_S_AT, | | | | | | | |
| | | | 228635_AT, 207238_S_AT, | | | | | | | |
| | | | 204584_AT, 207717_S_AT | | | | | | | |
| GO: 0032101~regulation of | 13 | 2.680412371 | 266_S_AT, 209875_S_AT, | 348 | 159 | 13528 | 3.178341647 | 8.17E−04 | 0.071258337 | 1.42774826 |
| response to external stimulus | | | 1553995_A_AT, 209771_X_AT, | | | | | | | |
| | | | 203140_AT, 208944_AT, 228758_AT, | | | | | | | |
| | | | 203824_AT, 206001_AT, | | | | | | | |
| | | | 218995_S_AT, 213506_AT, | | | | | | | |
| | | | 204035_AT, 202410_X_AT, | | | | | | | |
| | | | 227314_AT, 202409_AT, | | | | | | | |
| | | | 1553994_AT, 236439_AT, | | | | | | | |
| | | | 203382_S_AT, 207334_S_AT, | | | | | | | |
| | | | 222802_AT | | | | | | | |
| GO: 0060284~regulation of cell | 15 | 3.092783505 | 266_S_AT, 209875_S_AT, | 348 | 205 | 13528 | 2.844407065 | 8.41E−04 | 0.070749359 | 1.469345035 |
| development | | | 229576_S_AT, 209771_X_AT, | | | | | | | |
| | | | 229339_AT, 224722_AT, 237206_AT, | | | | | | | |
| | | | 233401_AT, 203395_S_AT, | | | | | | | |
| | | | 213721_AT, 218995_S_AT, | | | | | | | |
| | | | 200953_S_AT, 202410_X_AT, | | | | | | | |
| | | | 228121_AT, 204105_S_AT, | | | | | | | |
| | | | 231798_AT, 202409_AT, | | | | | | | |
| | | | 209909_S_AT, 229376_AT, | | | | | | | |
| | | | 225544_AT, 203382_S_AT, | | | | | | | |
| | | | 219682_S_AT, 222802_AT | | | | | | | |
| GO: 0070482~response to oxygen | 12 | 2.474226804 | 266_S_AT, 209771_X_AT, | 348 | 141 | 13528 | 3.308388359 | 0.001017954 | 0.082187753 | 1.775936008 |
| levels | | | 203710_AT, 227088_AT, 206825_AT, | | | | | | | |
| | | | 218995_S_AT, 226498_AT, | | | | | | | |
| | | | 227314_AT, 228121_AT, 206757_AT, | | | | | | | |
| | | | 209909_S_AT, 202986_AT, | | | | | | | |
| | | | 201860_S_AT, 200878_AT, | | | | | | | |
| | | | 240088_AT, 222802_AT, | | | | | | | |
| | | | 201243_S_AT | | | | | | | |
| GO: 0051241~negative regulation | 13 | 2.680412371 | 266_S_AT, 209771_X_AT, | 348 | 164 | 13528 | 3.081440987 | 0.001071382 | 0.083558691 | 1.868320166 |
| of multicellular organismal process | | | 203140_AT, 208944_AT, 228758_AT, | | | | | | | |
| | | | 203824_AT, 218995_S_AT, | | | | | | | |
| | | | 204932_AT, 207145_AT, | | | | | | | |
| | | | 202410_X_AT, 206857_S_AT, | | | | | | | |
| | | | 231798_AT, 202409_AT, | | | | | | | |
| | | | 204933_S_AT, 236439_AT, | | | | | | | |
| | | | 201186_AT, 210511_S_AT, | | | | | | | |
| | | | 207334_S_AT, 203382_S_AT, | | | | | | | |
| | | | 222802_AT | | | | | | | |
| GO: 0048638~regulation of | 7 | 1.443298969 | 207145_AT, 209875_S_AT, | 348 | 47 | 13528 | 5.789679628 | 0.001205896 | 0.090673243 | 2.100550475 |
| developmental growth | | | 204105_S_AT, 231798_AT, | | | | | | | |
| | | | 208944_AT, 229376_AT, 233401_AT, | | | | | | | |
| | | | 203382_S_AT, 207334_S_AT | | | | | | | |
| GO: 0042060~wound healing | 14 | 2.886597938 | 208944_AT, 215446_S_AT, | 348 | 191 | 13528 | 2.849371126 | 0.001316885 | 0.095670036 | 2.291776302 |
| | | | 226213_AT, 202454_S_AT, | | | | | | | |
| | | | 202430_S_AT, 213506_AT, | | | | | | | |
| | | | 207808_S_AT, 207145_AT, | | | | | | | |
| | | | 204298_S_AT, 202410_X_AT, | | | | | | | |
| | | | 227314_AT, 228121_AT, 231798_AT, | | | | | | | |
| | | | 202409_AT, 209909_S_AT, | | | | | | | |
| | | | 232082_X_AT, 235944_AT, | | | | | | | |
| | | | 202446_S_AT, 201860_S_AT, | | | | | | | |
| | | | 207334_S_AT | | | | | | | |
| GO: 0035295~tube development | 15 | 3.092783505 | 227376_AT, 229576_S_AT, | 348 | 220 | 13528 | 2.650470219 | 0.00165529 | 0.115376345 | 2.872652651 |
| | | | 224722_AT, 204689_AT, 208944_AT, | | | | | | | |
| | | | 215446_S_AT, 205201_AT, | | | | | | | |
| | | | 213902_AT, 212558_AT, | | | | | | | |
| | | | 213702_X_AT, 203395_S_AT, | | | | | | | |
| | | | 218995_S_AT, 226701_AT, | | | | | | | |
| | | | 204298_S_AT, 226498_AT, | | | | | | | |
| | | | 231798_AT, 210980_S_AT, | | | | | | | |
| | | | 229376_AT, 225544_AT, | | | | | | | |
| | | | 215933_S_AT, 200878_AT, | | | | | | | |
| | | | 207334_S_AT, 219682_S_AT, | | | | | | | |
| | | | 222802_AT | | | | | | | |
| GO: 0009968~negative regulation | 15 | 3.092783505 | 235392_AT, 228758_AT, | 348 | 221 | 13528 | 2.638477142 | 0.001726985 | 0.116748585 | 2.995298479 |
| of signal transduction | | | 206290_S_AT, 226213_AT, | | | | | | | |
| | | | 34697_AT, 206518_S_AT, | | | | | | | |
| | | | 1554500_A_AT, 213721_AT, | | | | | | | |
| | | | 222696_AT, 231798_AT, 212588_AT, | | | | | | | |
| | | | 203222_S_AT, 228284_AT, | | | | | | | |
| | | | 204689_AT, 203140_AT, | | | | | | | |
| | | | 207069_S_AT, 206204_AT, | | | | | | | |
| | | | 212587_S_AT, 216933_X_AT, | | | | | | | |
| | | | 202454_S_AT, 203221_AT, | | | | | | | |
| | | | 215933_S_AT, 236439_AT, | | | | | | | |
| | | | 207238_S_AT, 205606_AT | | | | | | | |
| GO: 0007166~cell surface receptor | 68 | 14.02061856 | 227376_AT, 208944_AT, 34697_AT, | 348 | 1856 | 13528 | 1.424246928 | 0.002085381 | 0.135541444 | 3.606206976 |
| linked signal transduction | | | 211679_X_AT, 206518_S_AT, | | | | | | | |
| | | | 1554500_A_AT, 201860_S_AT, | | | | | | | |
| | | | 207334_S_AT, 211356_X_AT, | | | | | | | |
| | | | 212587_S_AT, 216933_X_AT, | | | | | | | |
| | | | 205904_AT, 220108_AT, 223454_AT, | | | | | | | |
| | | | 209631_S_AT, 203221_AT, | | | | | | | |
| | | | 232060_AT, 244680_AT, 206730_AT, | | | | | | | |
| | | | 1569290_S_AT, 210511_S_AT, | | | | | | | |
| | | | 222802_AT, 224722_AT, | | | | | | | |
| | | | 213221_S_AT, 211355_X_AT, | | | | | | | |
| | | | 232267_AT, 226213_AT, 235591_AT, | | | | | | | |
| | | | 218995_S_AT, 227314_AT, | | | | | | | |
| | | | 243580_AT, 202709_AT, 201743_AT, | | | | | | | |
| | | | 202454_S_AT, 206584_AT, | | | | | | | |
| | | | 202410_X_AT, 228121_AT, | | | | | | | |
| | | | 227095_AT, 227529_S_AT, | | | | | | | |
| | | | 223430_AT, 209466_X_AT, | | | | | | | |
| | | | 266_S_AT, 235392_AT, 212950_AT, | | | | | | | |
| | | | 205201_AT, 206290_S_AT, | | | | | | | |
| | | | 224964_S_AT, 205280_AT, | | | | | | | |
| | | | 224022_X_AT, 209763_AT, | | | | | | | |
| | | | 242794_AT, 231798_AT, | | | | | | | |
| | | | 211737_X_AT, 203108_AT, | | | | | | | |
| | | | 203382_S_AT, 203222_S_AT, | | | | | | | |
| | | | 228284_AT, 218589_AT, | | | | | | | |
| | | | 209771_X_AT, 209894_AT, | | | | | | | |
| | | | 206336_AT, 204404_AT, 226498_AT, | | | | | | | |
| | | | 207145_AT, 212070_AT, | | | | | | | |
| | | | 208937_S_AT, 212444_AT, | | | | | | | |
| | | | 212951_AT, 202948_AT, 206001_AT, | | | | | | | |
| | | | 213506_AT, 222696_AT, | | | | | | | |
| | | | 203439_S_AT, 202409_AT, | | | | | | | |
| | | | 209909_S_AT, 203638_S_AT, | | | | | | | |
| | | | 225835_AT, 202150_S_AT, | | | | | | | |
| | | | 212588_AT, 44783_S_AT, | | | | | | | |
| | | | 228186_S_AT, 228692_AT, | | | | | | | |
| | | | 210473_S_AT, 205805_S_AT, | | | | | | | |
| | | | 205111_S_AT, 204689_AT, | | | | | | | |
| | | | 207069_S_{—} | | | | | | | |
| GO: 0007157~heterophilic cell | 5 | 1.030927835 | 222108_AT, 266_S_AT, | 348 | 22 | 13528 | 8.834900731 | 0.002158469 | 0.136338509 | 3.730344068 |
| adhesion | | | 209771_X_AT, 209030_S_AT, | | | | | | | |
| | | | 203868_S_AT, 209032_S_AT, | | | | | | | |
| | | | 209031_AT, 204584_AT | | | | | | | |
| GO: 0001666~response to hypoxia | 11 | 2.268041237 | 266_S_AT, 209771_X_AT, | 348 | 134 | 13528 | 3.191113399 | 0.002371244 | 0.145029215 | 4.090876793 |
| | | | 203710_AT, 227088_AT, | | | | | | | |
| | | | 218995_S_AT, 226498_AT, | | | | | | | |
| | | | 227314_AT, 228121_AT, 206757_AT, | | | | | | | |
| | | | 209909_S_AT, 202986_AT, | | | | | | | |
| | | | 201860_S_AT, 200878_AT, | | | | | | | |
| | | | 240088_AT, 222802_AT, | | | | | | | |
| | | | 201243_S_AT | | | | | | | |
| GO: 0006023~aminoglycan | 5 | 1.030927835 | 203188_AT, 214985_AT, 206756_AT, | 348 | 23 | 13528 | 8.450774613 | 0.002560667 | 0.151909204 | 4.410767117 |
| biosynthetic process | | | 202013_S_AT, 218927_S_AT | | | | | | | |
| GO: 0006940~regulation of smooth | 6 | 1.237113402 | 226498_AT, 229339_AT, | 348 | 38 | 13528 | 6.137931034 | 0.002700754 | 0.15577608 | 4.64669412 |
| muscle contraction | | | 205111_S_AT, 227314_AT, | | | | | | | |
| | | | 237206_AT, 222802_AT, 206825_AT, | | | | | | | |
| | | | 218995_S_AT | | | | | | | |
| GO: 0030155~regulation of cell | 11 | 2.268041237 | 266_S_AT, 209875_S_AT, | 348 | 137 | 13528 | 3.121235003 | 0.002787935 | 0.156707246 | 4.793241824 |
| adhesion | | | 209771_X_AT, 203333_AT, | | | | | | | |
| | | | 203140_AT, 230319_AT, 228758_AT, | | | | | | | |
| | | | 216933_X_AT, 226213_AT, | | | | | | | |
| | | | 202454_S_AT, 227314_AT, | | | | | | | |
| | | | 228121_AT, 203886_S_AT, | | | | | | | |
| | | | 209909_S_AT, 236439_AT, | | | | | | | |
| | | | 204584_AT | | | | | | | |
| GO: 0043062~extracellular | 12 | 2.474226804 | 236028_AT, 215446_S_AT, | 348 | 163 | 13528 | 2.861857415 | 0.0032252 | 0.175028538 | 5.525069772 |
| structure organization | | | 230319_AT, 37892_AT, | | | | | | | |
| | | | 1558502_S_AT, 209462_AT, | | | | | | | |
| | | | 207370_AT, 209839_AT, 233401_AT, | | | | | | | |
| | | | 204298_S_AT, 204932_AT, | | | | | | | |
| | | | 228121_AT, 204105_S_AT, | | | | | | | |
| | | | 209030_S_AT, 209909_S_AT, | | | | | | | |
| | | | 204320_AT, 204933_S_AT, | | | | | | | |
| | | | 213438_AT, 209032_S_AT, | | | | | | | |
| | | | 209031_AT, 1558501_AT, 227747_AT | | | | | | | |
| GO: 0001501~skeletal system | 18 | 3.711340206 | 227376_AT, 209875_S_AT, | 348 | 319 | 13528 | 2.193492595 | 0.003687157 | 0.193279505 | 6.292457565 |
| development | | | 208944_AT, 205201_AT, 236028_AT, | | | | | | | |
| | | | 207370_AT, 209763_AT, 205200_AT, | | | | | | | |
| | | | 218995_S_AT, 226701_AT, | | | | | | | |
| | | | 204932_AT, 222696_AT, 231798_AT, | | | | | | | |
| | | | 211737_X_AT, 202409_AT, | | | | | | | |
| | | | 204320_AT, 225544_AT, | | | | | | | |
| | | | 202013_S_AT, 207334_S_AT, | | | | | | | |
| | | | 214985_AT, 229576_S_AT, | | | | | | | |
| | | | 37892_AT, 202410_X_AT, | | | | | | | |
| | | | 204933_S_AT, 210511_S_AT, | | | | | | | |
| | | | 209465_X_AT, 209466_X_AT, | | | | | | | |
| | | | 222802_AT, 219682_S_AT | | | | | | | |
| GO: 0050974~detection of | 4 | 0.824742268 | 227314_AT, 204320_AT, 225835_AT, | 348 | 13 | 13528 | 11.96109637 | 0.003952002 | 0.201389232 | 6.729754624 |
| mechanical stimulus involved in | | | 37892_AT, 204404_AT, 213721_AT | | | | | | | |
| sensory perception | | | | | | | | | | |
| GO: 0045321~leukocyte activation | 15 | 3.092783505 | 266_S_AT, 208944_AT, 228758_AT, | 348 | 242 | 13528 | 2.409518381 | 0.003952357 | 0.19731301 | 6.730339634 |
| | | | 203868_S_AT, 218995_S_AT, | | | | | | | |
| | | | 206857_S_AT, 202409_AT, | | | | | | | |
| | | | 212588_AT, 207334_S_AT, | | | | | | | |
| | | | 209771_X_AT, 203140_AT, | | | | | | | |
| | | | 212587_S_AT, 210347_S_AT, | | | | | | | |
| | | | 209619_AT, 216933_X_AT, | | | | | | | |
| | | | 205905_S_AT, 219497_S_AT, | | | | | | | |
| | | | 205904_AT, 202410_X_AT, | | | | | | | |
| | | | 202962_AT, 219667_S_AT, | | | | | | | |
| | | | 1558662_S_AT, 236439_AT, | | | | | | | |
| | | | 207238_S_AT, 201641_AT, | | | | | | | |
| | | | 222891_S_AT, 222802_AT | | | | | | | |
| GO: 0040012~regulation of | 13 | 2.680412371 | 235392_AT, 216933_X_AT, | 348 | 192 | 13528 | 2.632064176 | 0.004004327 | 0.195663642 | 6.815923642 |
| locomotion | | | 212190_AT, 213506_AT, | | | | | | | |
| | | | 218995_S_AT, 226498_AT, | | | | | | | |
| | | | 204035_AT, 223454_AT, 227314_AT, | | | | | | | |
| | | | 228121_AT, 202410_X_AT, | | | | | | | |
| | | | 202409_AT, 209909_S_AT, | | | | | | | |
| | | | 203382_S_AT, 236599_AT, | | | | | | | |
| | | | 222802_AT, 226636_AT | | | | | | | |
| GO: 0048545~response to steroid | 13 | 2.680412371 | 266_S_AT, 209875_S_AT, | 348 | 192 | 13528 | 2.632064176 | 0.004004327 | 0.195663642 | 6.815923642 |
| hormone stimulus | | | 209771_X_AT, 206938_AT, | | | | | | | |
| | | | 208944_AT, 215446_S_AT, | | | | | | | |
| | | | 211355_X_AT, 211356_X_AT, | | | | | | | |
| | | | 209894_AT, 235945_AT, | | | | | | | |
| | | | 206518_S_AT, 206825_AT, | | | | | | | |
| | | | 204932_AT, 204298_S_AT, | | | | | | | |
| | | | 207145_AT, 200953_S_AT, | | | | | | | |
| | | | 227095_AT, 228121_AT, | | | | | | | |
| | | | 209909_S_AT, 204933_S_AT, | | | | | | | |
| | | | 202986_AT, 207334_S_AT | | | | | | | |
| GO: 0048585~negative regulation | 9 | 1.855670103 | 1553995_A_AT, 209875_S_AT, | 348 | 100 | 13528 | 3.49862069 | 0.004150421 | 0.198116317 | 7.056110531 |
| of response to stimulus | | | 235392_AT, 203140_AT, 206204_AT, | | | | | | | |
| | | | 228758_AT, 212587_S_AT, | | | | | | | |
| | | | 228121_AT, 202410_X_AT, | | | | | | | |
| | | | 202409_AT, 1553994_AT, | | | | | | | |
| | | | 209909_S_AT, 212588_AT, | | | | | | | |
| | | | 207238_S_AT, 236439_AT, | | | | | | | |
| | | | 203382_S_AT | | | | | | | |
| GO: 0051270~regulation of cell | 13 | 2.680412371 | 235392_AT, 203140_AT, 228758_AT, | 348 | 193 | 13528 | 2.618426538 | 0.004192671 | 0.196114493 | 7.125463579 |
| motion | | | 216933_X_AT, 212190_AT, | | | | | | | |
| | | | 213506_AT, 218995_S_AT, | | | | | | | |
| | | | 226498_AT, 223454_AT, | | | | | | | |
| | | | 202410_X_AT, 227314_AT, | | | | | | | |
| | | | 228121_AT, 202409_AT, | | | | | | | |
| | | | 209909_S_AT, 236439_AT, | | | | | | | |
| | | | 203382_S_AT, 236599_AT, | | | | | | | |
| | | | 222802_AT, 226636_AT | | | | | | | |
| GO: 0030334~regulation of cell | 12 | 2.474226804 | 235392_AT, 216933_X_AT, | 348 | 169 | 13528 | 2.76025301 | 0.004246154 | 0.194653788 | 7.213185203 |
| migration | | | 212190_AT, 213506_AT, | | | | | | | |
| | | | 218995_S_AT, 226498_AT, | | | | | | | |
| | | | 227314_AT, 228121_AT, | | | | | | | |
| | | | 202410_X_AT, 223454_AT, | | | | | | | |
| | | | 202409_AT, 209909_S_AT, | | | | | | | |
| | | | 203382_S_AT, 236599_AT, | | | | | | | |
| | | | 222802_AT, 226636_AT | | | | | | | |
| GO: 0006029~proteoglycan | 6 | 1.237113402 | 214985_AT, 230319_AT, 204320_AT, | 348 | 43 | 13528 | 5.424218123 | 0.004666683 | 0.207938775 | 7.900209149 |
| metabolic process | | | 37892_AT, 206756_AT, | | | | | | | |
| | | | 202013_S_AT, 218927_S_AT | | | | | | | |
| GO: 0001525~angiogenesis | 11 | 2.268041237 | 208944_AT, 211355_X_AT, | 348 | 148 | 13528 | 2.88925132 | 0.004840647 | 0.211001828 | 8.183012674 |
| | | | 211356_X_AT, 209894_AT, | | | | | | | |
| | | | 204575_S_AT, 218995_S_AT, | | | | | | | |
| | | | 226498_AT, 204035_AT, 227095_AT, | | | | | | | |
| | | | 228121_AT, 209909_S_AT, | | | | | | | |
| | | | 203477_AT, 209211_AT, | | | | | | | |
| | | | 208937_S_AT, 209212_S_AT, | | | | | | | |
| | | | 207334_S_AT, 200878_AT, | | | | | | | |
| | | | 222802_AT | | | | | | | |
| GO: 0010648~negative regulation | 15 | 3.092783505 | 235392_AT, 228758_AT, | 348 | 248 | 13528 | 2.351223582 | 0.004921047 | 0.210387521 | 8.313436185 |
| of cell communication | | | 206290_S_AT, 226213_AT, | | | | | | | |
| | | | 34697_AT, 206518_S_AT, | | | | | | | |
| | | | 1554500_A_AT, 213721_AT, | | | | | | | |
| | | | 222696_AT, 231798_AT, 212588_AT, | | | | | | | |
| | | | 203222_S_AT, 228284_AT, | | | | | | | |
| | | | 204689_AT, 203140_AT, | | | | | | | |
| | | | 207069_S_AT, 206204_AT, | | | | | | | |
| | | | 212587_S_AT, 216933_X_AT, | | | | | | | |
| | | | 202454_S_AT, 203221_AT, | | | | | | | |
| | | | 215933_S_AT, 236439_AT, | | | | | | | |
| | | | 207238_S_AT, 205606_AT | | | | | | | |
| GO: 0043549~regulation of kinase | 19 | 3.917525773 | 266_S_AT, 235392_AT, 208944_AT, | 348 | 357 | 13528 | 2.068901124 | 0.004990244 | 0.209378736 | 8.42554785 |
| activity | | | 218723_S_AT, 218995_S_AT, | | | | | | | |
| | | | 202409_AT, 209909_S_AT, | | | | | | | |
| | | | 203680_AT, 212588_AT, | | | | | | | |
| | | | 215506_S_AT, 202071_AT, | | | | | | | |
| | | | 207334_S_AT, 203382_S_AT, | | | | | | | |
| | | | 209841_S_AT, 205111_S_AT, | | | | | | | |
| | | | 209771_X_AT, 212587_S_AT, | | | | | | | |
| | | | 212558_AT, 209619_AT, | | | | | | | |
| | | | 216933_X_AT, 226498_AT, | | | | | | | |
| | | | 200953_S_AT, 228121_AT, | | | | | | | |
| | | | 202410_X_AT, 209840_S_AT, | | | | | | | |
| | | | 207238_S_AT, 222802_AT | | | | | | | |
| GO: 0022602~ovulation cycle | 7 | 1.443298969 | 207145_AT, 200953_S_AT, | 348 | 62 | 13528 | 4.388950686 | 0.005009902 | 0.206588898 | 8.457373608 |
| process | | | 228121_AT, 227095_AT, | | | | | | | |
| | | | 211356_X_AT, 211355_X_AT, | | | | | | | |
| | | | 205258_AT, 209909_S_AT, | | | | | | | |
| | | | 209894_AT, 210511_S_AT, | | | | | | | |
| | | | 206825_AT | | | | | | | |
| GO: 0046649~lymphocyte | 13 | 2.680412371 | 266_S_AT, 209771_X_AT, | 348 | 199 | 13528 | 2.539479004 | 0.005347956 | 0.215332685 | 9.003042723 |
| activation | | | 203140_AT, 228758_AT, | | | | | | | |
| | | | 212587_S_AT, 203868_S_AT, | | | | | | | |
| | | | 210347_S_AT, 209619_AT, | | | | | | | |
| | | | 216933_X_AT, 205905_S_AT, | | | | | | | |
| | | | 219497_S_AT, 205904_AT, | | | | | | | |
| | | | 202410_X_AT, 206857_S_AT, | | | | | | | |
| | | | 202409_AT, 202962_AT, | | | | | | | |
| | | | 219667_S_AT, 212588_AT, | | | | | | | |
| | | | 1558662_S_AT, 201641_AT, | | | | | | | |
| | | | 207238_S_AT, 236439_AT, | | | | | | | |
| | | | 222891_S_AT | | | | | | | |
| GO: 0048002~antigen processing | 5 | 1.030927835 | 201422_AT, 200904_AT, 209619_AT, | 348 | 28 | 13528 | 6.941707718 | 0.005353929 | 0.21207557 | 9.012657154 |
| and presentation of peptide antigen | | | 205905_S_AT, 211799_X_AT, | | | | | | | |
| | | | 205904_AT | | | | | | | |
| GO: 0007159~leukocyte adhesion | 5 | 1.030927835 | 266_S_AT, 209771_X_AT, | 348 | 28 | 13528 | 6.941707718 | 0.005353929 | 0.21207557 | 9.012657154 |
| | | | 203868_S_AT, 212587_S_AT, | | | | | | | |
| | | | 204875_S_AT, 212588_AT, | | | | | | | |
| | | | 207238_S_AT, 204584_AT | | | | | | | |
| GO: 0043627~response to estrogen | 9 | 1.855670103 | 266_S_AT, 209771_X_AT, | 348 | 105 | 13528 | 3.332019704 | 0.005572449 | 0.216260173 | 9.363712233 |
| stimulus | | | 208944_AT, 211355_X_AT, | | | | | | | |
| | | | 211356_X_AT, 209894_AT, | | | | | | | |
| | | | 206518_S_AT, 206825_AT, | | | | | | | |
| | | | 204932_AT, 207145_AT, | | | | | | | |
| | | | 200953_S_AT, 227095_AT, | | | | | | | |
| | | | 202986_AT, 204933_S_AT, | | | | | | | |
| | | | 207334_S_AT | | | | | | | |
| GO: 0008285~negative regulation | 19 | 3.917525773 | 266_S_AT, 227376_AT, 208944_AT, | 348 | 361 | 13528 | 2.045977011 | 0.005631203 | 0.214892353 | 9.457883273 |
| of cell proliferation | | | 205201_AT, 228758_AT, | | | | | | | |
| | | | 217707_X_AT, 235591_AT, | | | | | | | |
| | | | 244261_AT, 210619_S_AT, | | | | | | | |
| | | | 222696_AT, 231798_AT, | | | | | | | |
| | | | 209909_S_AT, 229376_AT, | | | | | | | |
| | | | 209230_S_AT, 207334_S_AT, | | | | | | | |
| | | | 203382_S_AT, 203333_AT, | | | | | | | |
| | | | 209771_X_AT, 203140_AT, | | | | | | | |
| | | | 216933_X_AT, 204035_AT, | | | | | | | |
| | | | 228121_AT, 219773_AT, 201422_AT, | | | | | | | |
| | | | 236439_AT | | | | | | | |
| GO: 0001701~in utero embryonic | 12 | 2.474226804 | 227376_AT, 224722_AT, | 348 | 176 | 13528 | 2.650470219 | 0.005746135 | 0.215438129 | 9.641828232 |
| development | | | 229576_S_AT, 235668_AT, | | | | | | | |
| | | | 205201_AT, 228964_AT, | | | | | | | |
| | | | 203395_S_AT, 218995_S_AT, | | | | | | | |
| | | | 210757_X_AT, 202986_AT, | | | | | | | |
| | | | 212124_AT, 225544_AT, 200878_AT, | | | | | | | |
| | | | 222802_AT, 219682_S_AT, | | | | | | | |
| | | | 228101_AT, 206084_AT | | | | | | | |
| GO: 0044092~negative regulation | 18 | 3.711340206 | 211355_X_AT, 201566_X_AT, | 348 | 334 | 13528 | 2.094982449 | 0.005802643 | 0.214056166 | 9.732140062 |
| of molecular function | | | 211679_X_AT, 206857_S_AT, | | | | | | | |
| | | | 231798_AT, 202409_AT, | | | | | | | |
| | | | 209909_S_AT, 207826_S_AT, | | | | | | | |
| | | | 212588_AT, 229376_AT, 204415_AT, | | | | | | | |
| | | | 203382_S_AT, 207069_S_AT, | | | | | | | |
| | | | 211356_X_AT, 209894_AT, | | | | | | | |
| | | | 212587_S_AT, 204867_AT, | | | | | | | |
| | | | 216933_X_AT, 212558_AT, | | | | | | | |
| | | | 201565_S_AT, 228121_AT, | | | | | | | |
| | | | 227095_AT, 202410_X_AT, | | | | | | | |
| | | | 208937_S_AT, 207238_S_AT, | | | | | | | |
| | | | 201186_AT | | | | | | | |
| GO: 0030111~regulation of Wnt | 6 | 1.237113402 | 222696_AT, 204689_AT, 203221_AT, | 348 | 46 | 13528 | 5.070464768 | 0.006243616 | 0.225015126 | 10.43397869 |
| receptor signaling pathway | | | 216933_X_AT, 215933_S_AT, | | | | | | | |
| | | | 34697_AT, 203222_S_AT, | | | | | | | |
| | | | 205606_AT, 228284_AT, 213721_AT | | | | | | | |
| GO: 0044057~regulation of system | 17 | 3.505154639 | 205111_S_AT, 229339_AT, | 348 | 309 | 13528 | 2.138674999 | 0.006251576 | 0.22201931 | 10.44660118 |
| process | | | 237206_AT, 201957_AT, 206825_AT, | | | | | | | |
| | | | 218995_S_AT, 207145_AT, | | | | | | | |
| | | | 226498_AT, 202410_X_AT, | | | | | | | |
| | | | 227314_AT, 228121_AT, | | | | | | | |
| | | | 206857_S_AT, 202409_AT, | | | | | | | |
| | | | 205258_AT, 209909_S_AT, | | | | | | | |
| | | | 201860_S_AT, 202071_AT, | | | | | | | |
| | | | 210511_S_AT, 203382_S_AT, | | | | | | | |
| | | | 200878_AT, 222802_AT | | | | | | | |
| GO: 0033674~positive regulation of | 14 | 2.886597938 | 266_S_AT, 235392_AT, | 348 | 231 | 13528 | 2.355973528 | 0.006687701 | 0.232252107 | 11.1355686 |
| kinase activity | | | 209771_X_AT, 205111_S_AT, | | | | | | | |
| | | | 208944_AT, 212587_S_AT, | | | | | | | |
| | | | 209619_AT, 218995_S_AT, | | | | | | | |
| | | | 226498_AT, 200953_S_AT, | | | | | | | |
| | | | 202410_X_AT, 228121_AT, | | | | | | | |
| | | | 202409_AT, 209909_S_AT, | | | | | | | |
| | | | 203680_AT, 209840_S_AT, | | | | | | | |
| | | | 212588_AT, 207238_S_AT, | | | | | | | |
| | | | 202071_AT, 207334_S_AT, | | | | | | | |
| | | | 222802_AT, 209841_S_AT | | | | | | | |
| GO: 0051302~regulation of cell | 6 | 1.237113402 | 202410_X_AT, 228121_AT, | 348 | 47 | 13528 | 4.962582539 | 0.006843344 | 0.233693339 | 11.38023323 |
| division | | | 211737_X_AT, 202409_AT, | | | | | | | |
| | | | 209909_S_AT, 209035_AT, | | | | | | | |
| | | | 216933_X_AT, 225532_AT, | | | | | | | |
| | | | 209465_X_AT, 209466_X_AT | | | | | | | |
| GO: 0030178~negative regulation | 5 | 1.030927835 | 222696_AT, 203221_AT, | 348 | 30 | 13528 | 6.478927203 | 0.006884121 | 0.231704249 | 11.44422697 |
| of Wnt receptor signaling pathway | | | 216933_X_AT, 34697_AT, | | | | | | | |
| | | | 203222_S_AT, 205606_AT, | | | | | | | |
| | | | 228284_AT, 213721_AT | | | | | | | |
| GO: 0051174~regulation of | 23 | 4.742268041 | 266_S_AT, 235392_AT, 208944_AT, | 348 | 485 | 13528 | 1.843488565 | 0.007059413 | 0.23368142 | 11.71883186 |
| phosphorus metabolic process | | | 218723_S_AT, 218995_S_AT, | | | | | | | |
| | | | 227314_AT, 206857_S_AT, | | | | | | | |
| | | | 202409_AT, 209909_S_AT, | | | | | | | |
| | | | 203680_AT, 212588_AT, | | | | | | | |
| | | | 215506_S_AT, 202071_AT, | | | | | | | |
| | | | 207334_S_AT, 203382_S_AT, | | | | | | | |
| | | | 209841_S_AT, 205111_S_AT, | | | | | | | |
| | | | 209771_X_AT, 207069_S_AT, | | | | | | | |
| | | | 212587_S_AT, 216933_X_AT, | | | | | | | |
| | | | 209619_AT, 212558_AT, 226498_AT, | | | | | | | |
| | | | 200953_S_AT, 202410_X_AT, | | | | | | | |
| | | | 228121_AT, 209840_S_AT, | | | | | | | |
| | | | 207238_S_AT, 210511_S_AT, | | | | | | | |
| | | | 222802_AT | | | | | | | |
| GO: 0019220~regulation of | 23 | 4.742268041 | 266_S_AT, 235392_AT, 208944_AT, | 348 | 485 | 13528 | 1.843488565 | 0.007059413 | 0.23368142 | 11.71883186 |
| phosphate metabolic process | | | 218723_S_AT, 218995_S_AT, | | | | | | | |
| | | | 227314_AT, 206857_S_AT, | | | | | | | |
| | | | 202409_AT, 209909_S_AT, | | | | | | | |
| | | | 203680_AT, 212588_AT, | | | | | | | |
| | | | 215506_S_AT, 202071_AT, | | | | | | | |
| | | | 207334_S_AT, 203382_S_AT, | | | | | | | |
| | | | 209841_S_AT, 205111_S_AT, | | | | | | | |
| | | | 209771_X_AT, 207069_S_AT, | | | | | | | |
| | | | 212587_S_AT, 216933_X_AT, | | | | | | | |
| | | | 209619_AT, 212558_AT, 226498_AT, | | | | | | | |
| | | | 200953_S_AT, 202410_X_AT, | | | | | | | |
| | | | 228121_AT, 209840_S_AT, | | | | | | | |
| | | | 207238_S_AT, 210511_S_AT, | | | | | | | |
| | | | 222802_AT | | | | | | | |
| GO: 0007167~enzyme linked | 18 | 3.711340206 | 213221_S_AT, 235392_AT, | 348 | 342 | 13528 | 2.045977011 | 0.00720178 | 0.234656091 | 11.94126451 |
| receptor protein signaling pathway | | | 208944_AT, 226213_AT, 209763_AT, | | | | | | | |
| | | | 231798_AT, 211737_X_AT, | | | | | | | |
| | | | 202409_AT, 209909_S_AT, | | | | | | | |
| | | | 203638_S_AT, 201860_S_AT, | | | | | | | |
| | | | 207334_S_AT, 202709_AT, | | | | | | | |
| | | | 205805_S_AT, 205111_S_AT, | | | | | | | |
| | | | 207069_S_AT, 202454_S_AT, | | | | | | | |
| | | | 226498_AT, 207145_AT, 228121_AT, | | | | | | | |
| | | | 202410_X_AT, 232060_AT, | | | | | | | |
| | | | 223430_AT, 208937_S_AT, | | | | | | | |
| | | | 209466_X_AT, 209465_X_AT | | | | | | | |
| GO: 0050982~detection of | 4 | 0.824742268 | 227314_AT, 204320_AT, 225835_AT, | 348 | 16 | 13528 | 9.718390805 | 0.007309686 | 0.234633276 | 12.1095045 |
| mechanical stimulus | | | 37892_AT, 204404_AT, 213721_AT | | | | | | | |
| GO: 0042698~ovulation cycle | 7 | 1.443298969 | 207145_AT, 200953_S_AT, | 348 | 67 | 13528 | 4.061417053 | 0.00731254 | 0.231697001 | 12.113951 |
| | | | 228121_AT, 227095_AT, | | | | | | | |
| | | | 211356_X_AT, 211355_X_AT, | | | | | | | |
| | | | 205258_AT, 209909_S_AT, | | | | | | | |
| | | | 209894_AT, 210511_S_AT, | | | | | | | |
| | | | 206825_AT | | | | | | | |
| GO: 0007565~female pregnancy | 9 | 1.855670103 | 209875_S_AT, 208944_AT, | 348 | 110 | 13528 | 3.180564263 | 0.00733893 | 0.229481846 | 12.15504714 |
| | | | 211355_X_AT, 211356_X_AT, | | | | | | | |
| | | | 209894_AT, 210195_S_AT, | | | | | | | |
| | | | 210196_S_AT, 210126_AT, | | | | | | | |
| | | | 205602_X_AT, 206825_AT, | | | | | | | |
| | | | 226498_AT, 227095_AT, | | | | | | | |
| | | | 208257_X_AT, 208134_X_AT, | | | | | | | |
| | | | 207334_S_AT | | | | | | | |
| GO: 0001775~cell activation | 16 | 3.298969072 | 266_S_AT, 208944_AT, 228758_AT, | 348 | 287 | 13528 | 2.167167287 | 0.007351582 | 0.226950781 | 12.17474504 |
| | | | 203868_S_AT, 218995_S_AT, | | | | | | | |
| | | | 206857_S_AT, 202409_AT, | | | | | | | |
| | | | 212588_AT, 202446_S_AT, | | | | | | | |
| | | | 207334_S_AT, 209771_X_AT, | | | | | | | |
| | | | 203140_AT, 212587_S_AT, | | | | | | | |
| | | | 210347_S_AT, 216933_X_AT, | | | | | | | |
| | | | 209619_AT, 205905_S_AT, | | | | | | | |
| | | | 219497_S_AT, 205904_AT, | | | | | | | |
| | | | 202430_S_AT, 202410_X_AT, | | | | | | | |
| | | | 202962_AT, 219667_S_AT, | | | | | | | |
| | | | 1558662_S_AT, 236439_AT, | | | | | | | |
| | | | 207238_S_AT, 201641_AT, | | | | | | | |
| | | | 222891_S_AT, 222802_AT | | | | | | | |
| GO: 0051346~negative regulation | 6 | 1.237113402 | 206857_S_AT, 228121_AT, | 348 | 48 | 13528 | 4.859195402 | 0.007482231 | 0.227647481 | 12.37789627 |
| of hydrolase activity | | | 227095_AT, 207069_S_AT, | | | | | | | |
| | | | 211356_X_AT, 211355_X_AT, | | | | | | | |
| | | | 209909_S_AT, 209894_AT, | | | | | | | |
| | | | 204867_AT, 204415_AT | | | | | | | |
| GO: 0051338~regulation of | 19 | 3.917525773 | 266_S_AT, 235392_AT, 208944_AT, | 348 | 372 | 13528 | 1.985477691 | 0.007735123 | 0.231542109 | 12.76986845 |
| transferase activity | | | 218723_S_AT, 218995_S_AT, | | | | | | | |
| | | | 202409_AT, 209909_S_AT, | | | | | | | |
| | | | 203680_AT, 212588_AT, | | | | | | | |
| | | | 215506_S_AT, 202071_AT, | | | | | | | |
| | | | 207334_S_AT, 203382_S_AT, | | | | | | | |
| | | | 209841_S_AT, 205111_S_AT, | | | | | | | |
| | | | 209771_X_AT, 212587_S_AT, | | | | | | | |
| | | | 212558_AT, 209619_AT, | | | | | | | |
| | | | 216933_X_AT, 226498_AT, | | | | | | | |
| | | | 200953_S_AT, 228121_AT, | | | | | | | |
| | | | 202410_X_AT, 209840_S_AT, | | | | | | | |
| | | | 207238_S_AT, 222802_AT | | | | | | | |
| GO: 0005976~polysaccharide | 9 | 1.855670103 | 210619_S_AT, 203188_AT, | 348 | 111 | 13528 | 3.151910531 | 0.007737781 | 0.228833763 | 12.77397936 |
| metabolic process | | | 214985_AT, 202838_AT, 212334_AT, | | | | | | | |
| | | | 230319_AT, 212335_AT, 206756_AT, | | | | | | | |
| | | | 202013_S_AT, 218927_S_AT | | | | | | | |
| GO: 0045859~regulation of protein | 18 | 3.711340206 | 266_S_AT, 208944_AT, | 348 | 345 | 13528 | 2.028185907 | 0.007976398 | 0.232238785 | 13.14229204 |
| kinase activity | | | 218723_S_AT, 218995_S_AT, | | | | | | | |
| | | | 202409_AT, 209909_S_AT, | | | | | | | |
| | | | 203680_AT, 212588_AT, | | | | | | | |
| | | | 215506_S_AT, 202071_AT, | | | | | | | |
| | | | 207334_S_AT, 203382_S_AT, | | | | | | | |
| | | | 209841_S_AT, 205111_S_AT, | | | | | | | |
| | | | 209771_X_AT, 212587_S_AT, | | | | | | | |
| | | | 212558_AT, 209619_AT, | | | | | | | |
| | | | 216933_X_AT, 226498_AT, | | | | | | | |
| | | | 228121_AT, 202410_X_AT, | | | | | | | |
| | | | 200953_S_AT, 209840_S_AT, | | | | | | | |
| | | | 207238_S_AT, 222802_AT | | | | | | | |
| GO: 0048584~positive regulation of | 14 | 2.886597938 | 266_S_AT, 235392_AT, | 348 | 236 | 13528 | 2.306058835 | 0.008038643 | 0.231101182 | 13.23812841 |
| response to stimulus | | | 209771_X_AT, 213800_AT, | | | | | | | |
| | | | 212587_S_AT, 215388_S_AT, | | | | | | | |
| | | | 206001_AT, 205905_S_AT, | | | | | | | |
| | | | 205904_AT, 213506_AT, 204035_AT, | | | | | | | |
| | | | 202410_X_AT, 227314_AT, | | | | | | | |
| | | | 228121_AT, 202409_AT, | | | | | | | |
| | | | 209030_S_AT, 209909_S_AT, | | | | | | | |
| | | | 209032_S_AT, 212588_AT, | | | | | | | |
| | | | 209031_AT, 208436_S_AT, | | | | | | | |
| | | | 207238_S_AT, 206653_AT | | | | | | | |
| GO: 0051347~positive regulation of | 14 | 2.886597938 | 266_S_AT, 235392_AT, | 348 | 240 | 13528 | 2.267624521 | 0.00918492 | 0.256576709 | 14.98526979 |
| transferase activity | | | 209771_X_AT, 205111_S_AT, | | | | | | | |
| | | | 208944_AT, 212587_S_AT, | | | | | | | |
| | | | 209619_AT, 218995_S_AT, | | | | | | | |
| | | | 226498_AT, 200953_S_AT, | | | | | | | |
| | | | 202410_X_AT, 228121_AT, | | | | | | | |
| | | | 202409_AT, 209909_S_AT, | | | | | | | |
| | | | 203680_AT, 209840_S_AT, | | | | | | | |
| | | | 212588_AT, 207238_S_AT, | | | | | | | |
| | | | 202071_AT, 207334_S_AT, | | | | | | | |
| | | | 222802_AT, 209841_S_AT | | | | | | | |
| GO: 0001503~ossification | 9 | 1.855670103 | 209875_S_AT, 214985_AT, | 348 | 115 | 13528 | 3.042278861 | 0.009497977 | 0.261150604 | 15.45662674 |
| | | | 236028_AT, 209763_AT, 207370_AT, | | | | | | | |
| | | | 202410_X_AT, 222696_AT, | | | | | | | |
| | | | 231798_AT, 211737_X_AT, | | | | | | | |
| | | | 202409_AT, 202013_S_AT, | | | | | | | |
| | | | 209466_X_AT, 209465_X_AT | | | | | | | |
| GO: 0016044~membrane | 19 | 3.917525773 | 266_S_AT, 235746_S_AT, | 348 | 381 | 13528 | 1.938576643 | 0.009598457 | 0.260630154 | 15.60739161 |
| organization | | | 208944_AT, 213413_AT, | | | | | | | |
| | | | 212850_S_AT, 209462_AT, | | | | | | | |
| | | | 34697_AT, 209839_AT, 239472_AT, | | | | | | | |
| | | | 239598_S_AT, 202446_S_AT, | | | | | | | |
| | | | 1558501_AT, 207334_S_AT, | | | | | | | |
| | | | 203382_S_AT, 209841_S_AT, | | | | | | | |
| | | | 209771_X_AT, 222833_AT, | | | | | | | |
| | | | 201743_AT, 1558502_S_AT, | | | | | | | |
| | | | 233401_AT, 202430_S_AT, | | | | | | | |
| | | | 206157_AT, 227889_AT, 223454_AT, | | | | | | | |
| | | | 204105_S_AT, 210757_X_AT, | | | | | | | |
| | | | 209840_S_AT, 203799_AT, | | | | | | | |
| | | | 205606_AT | | | | | | | |
| GO: 0006954~inflammatory | 17 | 3.505154639 | 266_S_AT, 209875_S_AT, | 348 | 325 | 13528 | 2.033386384 | 0.009905271 | 0.264873164 | 16.06618364 |
| response | | | 209771_X_AT, 213800_AT, | | | | | | | |
| | | | 201743_AT, 215388_S_AT, | | | | | | | |
| | | | 223217_S_AT, 206336_AT, | | | | | | | |
| | | | 206026_S_AT, 203921_AT, | | | | | | | |
| | | | 206157_AT, 206584_AT, 204035_AT, | | | | | | | |
| | | | 202410_X_AT, 204140_AT, | | | | | | | |
| | | | 202409_AT, 219773_AT, | | | | | | | |
| | | | 206025_S_AT, 209230_S_AT, | | | | | | | |
| | | | 208436_S_AT, 203799_AT | | | | | | | |
| GO: 0045596~negative regulation | 13 | 2.680412371 | 266_S_AT, 209875_S_AT, | 348 | 216 | 13528 | 2.339612601 | 0.010009001 | 0.264396503 | 16.22076271 |
| of cell differentiation | | | 227376_AT, 229576_S_AT, | | | | | | | |
| | | | 209771_X_AT, 224722_AT, | | | | | | | |
| | | | 203140_AT, 205201_AT, 228758_AT, | | | | | | | |
| | | | 216933_X_AT, 209619_AT, | | | | | | | |
| | | | 203395_S_AT, 213721_AT, | | | | | | | |
| | | | 222696_AT, 231798_AT, 225544_AT, | | | | | | | |
| | | | 236439_AT, 210511_S_AT, | | | | | | | |
| | | | 219682_S_AT | | | | | | | |
| GO: 0045987~positive regulation of | 4 | 0.824742268 | 229339_AT, 227314_AT, 237206_AT, | 348 | 18 | 13528 | 8.638569604 | 0.010255631 | 0.267128334 | 16.5872148 |
| smooth muscle contraction | | | 222802_AT, 206825_AT, | | | | | | | |
| | | | 218995_S_AT | | | | | | | |
| GO: 0001889~liver development | 6 | 1.237113402 | 200953_S_AT, 227314_AT, | 348 | 53 | 13528 | 4.400780742 | 0.011306556 | 0.287255862 | 18.13183846 |
| | | | 204689_AT, 209030 S_AT, | | | | | | | |
| | | | 209032_S_AT, 209031_AT, | | | | | | | |
| | | | 229376_AT, 215933_S_AT, | | | | | | | |
| | | | 203395_S_AT | | | | | | | |
| GO: 0043009~chordate embryonic | 17 | 3.505154639 | 227376_AT, 229576_S_AT, | 348 | 331 | 13528 | 1.996527416 | 0.011680168 | 0.292256087 | 18.67443346 |
| development | | | 224722_AT, 235668_AT, 208944_AT, | | | | | | | |
| | | | 205201_AT, 37892_AT, 228964_AT, | | | | | | | |
| | | | 203395_S_AT, 218995_S_AT, | | | | | | | |
| | | | 222696_AT, 231798_AT, | | | | | | | |
| | | | 210757_X_AT, 204320_AT, | | | | | | | |
| | | | 202986_AT, 212124_AT, 229376_AT, | | | | | | | |
| | | | 225544_AT, 207334_S_AT, | | | | | | | |
| | | | 200878_AT, 219682_S_AT, | | | | | | | |
| | | | 222802_AT, 228101_AT, 206084_AT | | | | | | | |
| GO: 0046626~regulation of insulin | 4 | 0.824742268 | 213221_S_AT, 202410_X_AT, | 348 | 19 | 13528 | 8.183908046 | 0.011950704 | 0.294971259 | 19.06521305 |
| receptor signaling pathway | | | 235392_AT, 206204_AT, 202409_AT, | | | | | | | |
| | | | 223430_AT | | | | | | | |
| GO: 0009792~embryonic | 17 | 3.505154639 | 227376_AT, 229576_S_AT, | 348 | 334 | 13528 | 1.978594535 | 0.01263372 | 0.305991877 | 20.04394042 |
| development ending in birth or egg | | | 224722_AT, 235668_AT, 208944_AT, | | | | | | | |
| hatching | | | 205201_AT, 37892_AT, 228964_AT, | | | | | | | |
| | | | 203395_S_AT, 218995_S_AT, | | | | | | | |
| | | | 222696_AT, 231798_AT, | | | | | | | |
| | | | 210757_X_AT, 204320_AT, | | | | | | | |
| | | | 202986_AT, 212124_AT, 229376_AT, | | | | | | | |
| | | | 225544_AT, 207334_S_AT, | | | | | | | |
| | | | 200878_AT, 219682_S_AT, | | | | | | | |
| | | | 222802_AT, 228101_AT, 206084_AT | | | | | | | |
| GO: 0045860~positive regulation of | 13 | 2.680412371 | 266_S_AT, 209771_X_AT, | 348 | 223 | 13528 | 2.266171847 | 0.012649188 | 0.303347875 | 20.06597463 |
| protein kinase activity | | | 205111_S_AT, 208944_AT, | | | | | | | |
| | | | 212587_S_AT, 209619_AT, | | | | | | | |
| | | | 218995_S_AT, 226498_AT, | | | | | | | |
| | | | 200953_S_AT, 202410_X_AT, | | | | | | | |
| | | | 228121_AT, 202409_AT, | | | | | | | |
| | | | 209909_S_AT, 203680_AT, | | | | | | | |
| | | | 209840_S_AT, 212588_AT, | | | | | | | |
| | | | 207238_S_AT, 202071_AT, | | | | | | | |
| | | | 207334_S_AT, 222802_AT, | | | | | | | |
| | | | 209841_S_AT | | | | | | | |
| GO: 0014065~phosphoinositide 3- | 3 | 0.618556701 | 235392_AT, 226213_AT, | 348 | 7 | 13528 | 16.66009852 | 0.012650474 | 0.30047297 | 20.06780662 |
| kinase cascade | | | 202454_S_AT, 222802_AT, | | | | | | | |
| | | | 218995_S_AT | | | | | | | |
| GO: 0042113~B cell activation | 7 | 1.443298969 | 266_S_AT, 209771_X_AT, | 348 | 76 | 13528 | 3.58045977 | 0.013238794 | 0.309148254 | 20.90164513 |
| | | | 203140_AT, 228758_AT, | | | | | | | |
| | | | 212587_S_AT, 203868_S_AT, | | | | | | | |
| | | | 210347_S_AT, 219497_S_AT, | | | | | | | |
| | | | 219667_S_AT, 212588_AT, | | | | | | | |
| | | | 1558662_S_AT, 207238_S_AT, | | | | | | | |
| | | | 236439_AT, 201641_AT, | | | | | | | |
| | | | 222891_S_AT | | | | | | | |
| GO: 0016192~vesicle-mediated | 25 | 5.154639175 | 235746_S_AT, 266_S_AT, | 348 | 576 | 13528 | 1.687220626 | 0.013370654 | 0.308810622 | 21.08740466 |
| transport | | | 208944_AT, 213413_AT, | | | | | | | |
| | | | 212850_S_AT, 205280_AT, | | | | | | | |
| | | | 209462_AT, 34697_AT, 205248_AT, | | | | | | | |
| | | | 209839_AT, 239472_AT, 232368_AT, | | | | | | | |
| | | | 225677_AT, 201596_X_AT, | | | | | | | |
| | | | 202956_AT, 241652_X_AT, | | | | | | | |
| | | | 243220_AT, 1558501_AT, | | | | | | | |
| | | | 207334_S_AT, 203382_S_AT, | | | | | | | |
| | | | 209841_S_AT, 209771_X_AT, | | | | | | | |
| | | | 201743_AT, 1558502_S_AT, | | | | | | | |
| | | | 230560_AT, 206157_AT, 223454_AT, | | | | | | | |
| | | | 210757_X_AT, 209840_S_AT, | | | | | | | |
| | | | 244680_AT, 225674_AT, 201186_AT, | | | | | | | |
| | | | 203799_AT, 205606_AT | | | | | | | |
| GO: 0033261~regu1ation of S phase | 4 | 0.824742268 | 203140_AT, 207069_S_AT, | 348 | 20 | 13528 | 7.774712644 | 0.013796955 | 0.314059147 | 21.68514835 |
| | | | 228758_AT, 212587_S_AT, | | | | | | | |
| | | | 212588_AT, 229376_AT, 236439_AT, | | | | | | | |
| | | | 207238_S_AT | | | | | | | |
| GO: 0060348~bone development | 9 | 1.855670103 | 209875_S_AT, 214985_AT, | 348 | 123 | 13528 | 2.844407065 | 0.013889988 | 0.312953308 | 21.81502742 |
| | | | 236028_AT, 209763_AT, 207370_AT, | | | | | | | |
| | | | 202410_X_AT, 222696_AT, | | | | | | | |
| | | | 231798_AT, 211737_X_AT, | | | | | | | |
| | | | 202409_AT, 202013_S_AT, | | | | | | | |
| | | | 209466_X_AT, 209465_X_AT | | | | | | | |
| GO: 0009100~glycoprotein | 12 | 2.474226804 | 201722_S_AT, 214985_AT, | 348 | 202 | 13528 | 2.309320587 | 0.015321011 | 0.336230567 | 23.78738868 |
| metabolic process | | | 230319_AT, 37892_AT, 201998_AT, | | | | | | | |
| | | | 203824_AT, 206756_AT, | | | | | | | |
| | | | 218927_S_AT, 218313_S_AT, | | | | | | | |
| | | | 219797_AT, 203188_AT, | | | | | | | |
| | | | 201724_S_AT, 201723_S_AT, | | | | | | | |
| | | | 204320_AT, 202013_S_AT, | | | | | | | |
| | | | 226039_AT, 1568618_A_AT | | | | | | | |
| GO: 0006024~glycosaminoglycan | 4 | 0.824742268 | 214985_AT, 206756_AT, | 348 | 21 | 13528 | 7.404488232 | 0.015796103 | 0.34169434 | 24.43175929 |
| biosynthetic process | | | 202013_S_AT, 218927_S_AT | | | | | | | |
| GO: 0045933~positive regulation of | 4 | 0.824742268 | 229339_AT, 227314_AT, 237206_AT, | 348 | 21 | 13528 | 7.404488232 | 0.015796103 | 0.34169434 | 24.43175929 |
| muscle contraction | | | 222802_AT, 206825_AT, | | | | | | | |
| | | | 218995_S_AT | | | | | | | |
| GO: 0030900~forebrain | 10 | 2.06185567 | 227376_AT, 229576_S_AT, | 348 | 152 | 13528 | 2.557471264 | 0.016511813 | 0.351138967 | 25.3927868 |
| development | | | 204689_AT, 205201_AT, 209462_AT, | | | | | | | |
| | | | 203395_S_AT, 213721_AT, | | | | | | | |
| | | | 206825_AT, 202289_S_AT, | | | | | | | |
| | | | 231798_AT, 215933_S_AT, | | | | | | | |
| | | | 225544_AT, 228396_AT, | | | | | | | |
| | | | 219682_S_AT | | | | | | | |
| GO: 0007178~transmembrane | 8 | 1.649484536 | 207145_AT, 228121_AT, 231798_AT, | 348 | 103 | 13528 | 3.019305881 | 0.016693895 | 0.351272928 | 25.63543416 |
| receptor protein serine/threonine | | | 207069_S_AT, 208944_AT, | | | | | | | |
| kinase signaling pathway | | | 209909_S_AT, 208937_S_AT, | | | | | | | |
| | | | 209763_AT, 207334_S_AT, | | | | | | | |
| | | | 202709_AT | | | | | | | |
| GO: 0051781~positive regulation of | 5 | 1.030927835 | 202410_X_AT, 228121_AT, | 348 | 39 | 13528 | 4.983790156 | 0.017263647 | 0.357879199 | 26.38989984 |
| cell division | | | 211737_X_AT, 202409_AT, | | | | | | | |
| | | | 209909_S_AT, 209035_AT, | | | | | | | |
| | | | 216933_X_AT, 209465_X_AT, | | | | | | | |
| | | | 209466_X_AT | | | | | | | |
| GO: 0048511~rhythmic process | 9 | 1.855670103 | 211355_X_AT, 211356_X_AT, | 348 | 128 | 13528 | 2.733297414 | 0.017295403 | 0.355464614 | 26.43173766 |
| | | | 209894_AT, 226213_AT, | | | | | | | |
| | | | 202454_S_AT, 206825_AT, | | | | | | | |
| | | | 207145_AT, 227095_AT, 228121_AT, | | | | | | | |
| | | | 200953_S_AT, 209909_S_AT, | | | | | | | |
| | | | 205258_AT, 212719_AT, | | | | | | | |
| | | | 210511_S_AT | | | | | | | |
| GO: 0042325~regulation of | 21 | 4.329896907 | 266_S_AT, 235392_AT, 208944_AT, | 348 | 466 | 13528 | 1.751812935 | 0.017317027 | 0.35292431 | 26.46021533 |
| phosphorylation | | | 218723_S_AT, 218995_S_AT, | | | | | | | |
| | | | 202409_AT, 209909_S_AT, | | | | | | | |
| | | | 203680_AT, 212588_AT, | | | | | | | |
| | | | 215506_S_AT, 202071_AT, | | | | | | | |
| | | | 207334_S_AT, 203382_S_AT, | | | | | | | |
| | | | 209841_S_AT, 205111_S_AT, | | | | | | | |
| | | | 209771_X_AT, 207069_S_AT, | | | | | | | |
| | | | 212587_S_AT, 209619_AT, | | | | | | | |
| | | | 216933_X_AT, 212558_AT, | | | | | | | |
| | | | 226498_AT, 200953_S_AT, | | | | | | | |
| | | | 228121_AT, 202410_X_AT, | | | | | | | |
| | | | 209840_S_AT, 207238_S_AT, | | | | | | | |
| | | | 210511_S_AT, 222802_AT | | | | | | | |
| GO: 0051100~negative regulation | 6 | 1.237113402 | 231798_AT, 207826_S_AT, | 348 | 59 | 13528 | 3.953243717 | 0.017429728 | 0.351909036 | 26.60846204 |
| of binding | | | 201565_S_AT, 201566_X_AT, | | | | | | | |
| | | | 229376_AT, 208937_S_AT, | | | | | | | |
| | | | 201186_AT | | | | | | | |
| GO: 0030198~extracellular matrix | 8 | 1.649484536 | 215446_S_AT, 230319_AT, | 348 | 104 | 13528 | 2.990274094 | 0.017518877 | 0.35053264 | 26.72552854 |
| organization | | | 236028_AT, 37892_AT, 209462_AT, | | | | | | | |
| | | | 207370_AT, 204298_S_AT, | | | | | | | |
| | | | 204932_AT, 228121_AT, | | | | | | | |
| | | | 209909_S_AT, 204320_AT, | | | | | | | |
| | | | 204933_S_AT, 227747_AT | | | | | | | |
| GO: 0008361~regulation of cell size | 12 | 2.474226804 | 209875_S_AT, 203140_AT, | 348 | 206 | 13528 | 2.264479411 | 0.017568106 | 0.348541085 | 26.79009969 |
| | | | 228758_AT, 201539_S_AT, | | | | | | | |
| | | | 211126_S_AT, 218995_S_AT, | | | | | | | |
| | | | 223454_AT, 228121_AT, | | | | | | | |
| | | | 202410_X_AT, 210299_S_AT, | | | | | | | |
| | | | 202409_AT, 210757_X_AT, | | | | | | | |
| | | | 203638_S_AT, 209909_S_AT, | | | | | | | |
| | | | 201540_AT, 207030_S_AT, | | | | | | | |
| | | | 209230_S_AT, 236439_AT, | | | | | | | |
| | | | 214505_S_AT, 210511_S_AT, | | | | | | | |
| | | | 222802_AT | | | | | | | |
| GO: 0007179~transforming growth | 6 | 1.237113402 | 207145_AT, 228121_AT, | 348 | 60 | 13528 | 3.887356322 | 0.018627194 | 0.362475596 | 28.16630275 |
| factor beta receptor signaling | | | 207069_S_AT, 208944_AT, | | | | | | | |
| pathway | | | 209909_S_AT, 208937_S_AT, | | | | | | | |
| | | | 207334_S_AT, 202709_AT | | | | | | | |
| GO: 0030097~hemopoiesis | 13 | 2.680412371 | 266_S_AT, 209771_X_AT, | 348 | 236 | 13528 | 2.141340347 | 0.018997383 | 0.365385275 | 28.64155263 |
| | | | 208944_AT, 203140_AT, 228758_AT, | | | | | | | |
| | | | 212587_S_AT, 203868_S_AT, | | | | | | | |
| | | | 210347_S_AT, 203903_S_AT, | | | | | | | |
| | | | 216933_X_AT, 209619_AT, | | | | | | | |
| | | | 201565_S_AT, 201566_X_AT, | | | | | | | |
| | | | 219497_S_AT, 228121_AT, | | | | | | | |
| | | | 209909_S_AT, 212588_AT, | | | | | | | |
| | | | 207238_S_AT, 236439_AT, | | | | | | | |
| | | | 210511_S_AT, 200878_AT, | | | | | | | |
| | | | 207334_S_AT, 222891_S_AT | | | | | | | |
| GO: 0006955~immune response | 28 | 5.773195876 | 266_S_AT, 202869_AT, 205403_AT, | 348 | 690 | 13528 | 1.577477928 | 0.019191055 | 0.365552551 | 28.88900654 |
| | | | 205552_S_AT, 218400_AT, | | | | | | | |
| | | | 202948_AT, 202688_AT, 204972_AT, | | | | | | | |
| | | | 215388_S_AT, 211799_X_AT, | | | | | | | |
| | | | 211906_S_AT, 202687_S_AT, | | | | | | | |
| | | | 209032_S_AT, 212588_AT, | | | | | | | |
| | | | 209031_AT, 204415_AT, | | | | | | | |
| | | | 205285_S_AT, 209771_X_AT, | | | | | | | |
| | | | 207069_S_AT, 213800_AT, | | | | | | | |
| | | | 201743_AT, 212587_S_AT, | | | | | | | |
| | | | 227266_S_AT, 201998_AT, | | | | | | | |
| | | | 200904_AT, 209619_AT, 206336_AT, | | | | | | | |
| | | | 205905_S_AT, 211372_S_AT, | | | | | | | |
| | | | 205904_AT, 206157_AT, 206584_AT, | | | | | | | |
| | | | 223454_AT, 209030_S_AT, | | | | | | | |
| | | | 228607_AT, 219209_AT, 204439_AT, | | | | | | | |
| | | | 201641_AT, 207238_S_AT, | | | | | | | |
| | | | 203799_AT, 211795_S_AT | | | | | | | |
| GO: 0002683~negative regulation | 7 | 1.443298969 | 266_S_AT, 209771_X_AT, | 348 | 83 | 13528 | 3.278493283 | 0.01972049 | 0.370748926 | 29.56133912 |
| of immune system process | | | 203140_AT, 228758_AT, | | | | | | | |
| | | | 212587_S_AT, 209619_AT, | | | | | | | |
| | | | 228121_AT, 202410_X_AT, | | | | | | | |
| | | | 202409_AT, 209909_S_AT, | | | | | | | |
| | | | 212588_AT, 207238_S_AT, | | | | | | | |
| | | | 236439_AT, 210511_S_AT | | | | | | | |
| GO: 0040007~growth | 11 | 2.268041237 | 227376_AT, 205201_AT, | 348 | 183 | 13528 | 2.33666227 | 0.019727842 | 0.368102237 | 29.57063272 |
| | | | 201539_S_AT, 211126_S_AT, | | | | | | | |
| | | | 207145_AT, 228121_AT, | | | | | | | |
| | | | 210299_S_AT, 205258_AT, | | | | | | | |
| | | | 203638_S_AT, 209909_S_AT, | | | | | | | |
| | | | 201540_AT, 207030_S_AT, | | | | | | | |
| | | | 212124_AT, 209230_S_AT, | | | | | | | |
| | | | 214505_S_AT, 210511_S_AT, | | | | | | | |
| | | | 228101_AT | | | | | | | |
| GO: 0000122~negative regulation | 14 | 2.886597938 | 204689_AT, 203140_AT, 235668_AT, | 348 | 266 | 13528 | 2.045977011 | 0.02015059 | 0.371602353 | 30.10310702 |
| of transcription from RNA | | | 217707_X_AT, 228758_AT, | | | | | | | |
| polymerase II promoter | | | 201566_X_AT, 201565_S_AT, | | | | | | | |
| | | | 228964_AT, 203395_S_AT, | | | | | | | |
| | | | 213721_AT, 218839_AT, 203221_AT, | | | | | | | |
| | | | 207826_S_AT, 230258_AT, | | | | | | | |
| | | | 229376_AT, 44783_S_AT, | | | | | | | |
| | | | 208937_S_AT, 215933_S_AT, | | | | | | | |
| | | | 208436_S_AT, 236439_AT, | | | | | | | |
| | | | 229435_AT, 203222_S_AT, | | | | | | | |
| | | | 228284_AT | | | | | | | |
| GO: 0002684~positive regulation of | 13 | 2.680412371 | 266_S_AT, 209771_X_AT, | 348 | 238 | 13528 | 2.12334589 | 0.020178556 | 0.369300516 | 30.13819734 |
| immune system process | | | 208944_AT, 203140_AT, 213800_AT, | | | | | | | |
| | | | 228758_AT, 212587_S_AT, | | | | | | | |
| | | | 203868_S_AT, 215388_S_AT, | | | | | | | |
| | | | 209619_AT, 205905_S_AT, | | | | | | | |
| | | | 205904_AT, 213506_AT, 227314_AT, | | | | | | | |
| | | | 228121_AT, 209030_S_AT, | | | | | | | |
| | | | 209909_S_AT, 209032_S_AT, | | | | | | | |
| | | | 212588_AT, 209031_AT, | | | | | | | |
| | | | 207238_S_AT, 236439_AT, | | | | | | | |
| | | | 207334_S_AT, 206653_AT | | | | | | | |
| GO: 0009101~glycoprotein | 10 | 2.06185567 | 201722_S_AT, 214985_AT, | 348 | 158 | 13528 | 2.460352102 | 0.020700703 | 0.374146652 | 30.79032025 |
| biosynthetic process | | | 201998_AT, 203824_AT, 206756_AT, | | | | | | | |
| | | | 218927_S_AT, 218313_S_AT, | | | | | | | |
| | | | 219797_AT, 203188_AT, | | | | | | | |
| | | | 201724_S_AT, 201723_S_AT, | | | | | | | |
| | | | 202013_S_AT, 226039_AT, | | | | | | | |
| | | | 1568618_A_AT | | | | | | | |
| GO: 0050910~detection of | 3 | 0.618556701 | 204320_AT, 225835_AT, 37892_AT, | 348 | 9 | 13528 | 12.95785441 | 0.020963744 | 0.375211821 | 31.11666057 |
| mechanical stimulus involved in | | | 204404_AT, 213721_AT | | | | | | | |
| sensory perception of sound | | | | | | | | | | |
| GO: 0009125~nucleoside | 3 | 0.618556701 | 1553995_A_AT, 206757_AT, | 348 | 9 | 13528 | 12.95785441 | 0.020963744 | 0.375211821 | 31.11666057 |
| monophosphate catabolic process | | | 1553994_AT, 228962_AT, | | | | | | | |
| | | | 227088_AT, 240088_AT | | | | | | | |
| GO: 0060541~respiratory system | 8 | 1.649484536 | 227376_AT, 208944_AT, 205201_AT, | 348 | 108 | 13528 | 2.879523201 | 0.021106679 | 0.374570968 | 31.29338254 |
| development | | | 215446_S_AT, 213902_AT, | | | | | | | |
| | | | 213702_X_AT, 203395_S_AT, | | | | | | | |
| | | | 213721_AT, 204298_S_AT, | | | | | | | |
| | | | 210980_S_AT, 229376_AT, | | | | | | | |
| | | | 207334_S_AT, 200878_AT | | | | | | | |
| GO: 0045664~regulation of neuron | 9 | 1.855670103 | 209875_S_AT, 266_S_AT, | 348 | 133 | 13528 | 2.630541872 | 0.021263865 | 0.37413994 | 31.48723104 |
| differentiation | | | 224722_AT, 200953_S_AT, | | | | | | | |
| | | | 209771_X_AT, 204105_S_AT, | | | | | | | |
| | | | 231798_AT, 233401_AT, | | | | | | | |
| | | | 203382_S_AT, 203395_S_AT, | | | | | | | |
| | | | 213721_AT | | | | | | | |
| GO: 0032870~cellular response to | 9 | 1.855670103 | 213221_S_AT, 200953_S_AT, | 348 | 133 | 13528 | 2.630541872 | 0.021263865 | 0.37413994 | 31.48723104 |
| hormone stimulus | | | 202410_X_AT, 235392_AT, | | | | | | | |
| | | | 227314_AT, 202409_AT, 204115_AT, | | | | | | | |
| | | | 203680_AT, 223430_AT, 226213_AT, | | | | | | | |
| | | | 224964_S_AT, 202454_S_AT | | | | | | | |
| GO: 0051047~positive regulation of | 8 | 1.649484536 | 244802_AT, 206825_AT, | 348 | 109 | 13528 | 2.853105557 | 0.022077819 | 0.382737567 | 32.48280787 |
| secretion | | | 218995_S_AT, 228121_AT, | | | | | | | |
| | | | 202410_X_AT, 202409_AT, | | | | | | | |
| | | | 209909_S_AT, 205258_AT, | | | | | | | |
| | | | 209030_S_AT, 209032_S_AT, | | | | | | | |
| | | | 209031_AT, 210511_S_AT, | | | | | | | |
| | | | 222802_AT | | | | | | | |
| GO: 0045619~regulation of | 6 | 1.237113402 | 266_S_AT, 209771_X_AT, | 348 | 63 | 13528 | 3.702244116 | 0.022540521 | 0.386372143 | 33.04265674 |
| lymphocyte differentiation | | | 203140_AT, 208944_AT, 228758_AT, | | | | | | | |
| | | | 212587_S_AT, 209619_AT, | | | | | | | |
| | | | 212588_AT, 236439_AT, | | | | | | | |
| | | | 207238_S_AT, 210511_S_AT, | | | | | | | |
| | | | 207334_S_AT | | | | | | | |
| GO: 0030879~mammary gland | 6 | 1.237113402 | 227376_AT, 229576_S_AT, | 348 | 63 | 13528 | 3.702244116 | 0.022540521 | 0.386372143 | 33.04265674 |
| development | | | 235392_AT, 227314_AT, 205201_AT, | | | | | | | |
| | | | 226213_AT, 225544_AT, | | | | | | | |
| | | | 202454_S_AT, 219682_S_AT, | | | | | | | |
| | | | 206825_AT | | | | | | | |
| GO: 0010647~positive regulation of | 16 | 3.298969072 | 266_S_AT, 235392_AT, | 348 | 329 | 13528 | 1.890507634 | 0.022966149 | 0.389433769 | 33.5537779 |
| cell communication | | | 211355_X_AT, 202688_AT, | | | | | | | |
| | | | 226213_AT, 213721_AT, | | | | | | | |
| | | | 202687_S_AT, 227314_AT, | | | | | | | |
| | | | 202409_AT, 209909_S_AT, | | | | | | | |
| | | | 212588_AT, 221958_S_AT, | | | | | | | |
| | | | 209771_X_AT, 204689_AT, | | | | | | | |
| | | | 211356_X_AT, 209894_AT, | | | | | | | |
| | | | 212587_S_AT, 202454_S_AT, | | | | | | | |
| | | | 206825_AT, 226498_AT, 228121_AT, | | | | | | | |
| | | | 227095_AT, 202410_X_AT, | | | | | | | |
| | | | 228949_AT, 215933_S_AT, | | | | | | | |
| | | | 201641_AT, 207238_S_AT, | | | | | | | |
| | | | 204584_AT | | | | | | | |
| GO: 0030218~erythrocyte | 5 | 1.030927835 | 203140_AT, 228758_AT, | 348 | 43 | 13528 | 4.52018177 | 0.023926671 | 0.399397204 | 34.69374566 |
| differentiation | | | 203903_S_AT, 201565_S_AT, | | | | | | | |
| | | | 201566_X_AT, 236439_AT, | | | | | | | |
| | | | 210511_S_AT, 200878_AT | | | | | | | |
| GO: 0051726~regulation of cell | 16 | 3.298969072 | 218723_S_AT, 228758_AT, | 348 | 331 | 13528 | 1.879084627 | 0.02417482 | 0.399967207 | 34.9852413 |
| cycle | | | 201566_X_AT, 218995_S_AT, | | | | | | | |
| | | | 202409_AT, 209909_S_AT, | | | | | | | |
| | | | 207826_S_AT, 215506_S_AT, | | | | | | | |
| | | | 212588_AT, 229376_AT, 225544_AT, | | | | | | | |
| | | | 225532_AT, 229576_S_AT, | | | | | | | |
| | | | 203140_AT, 207069_S_AT, | | | | | | | |
| | | | 212587_S_AT, 216933_X_AT, | | | | | | | |
| | | | 201565_S_AT, 228121_AT, | | | | | | | |
| | | | 202410_X_AT, 200953_S_AT, | | | | | | | |
| | | | 236439_AT, 207238_S_AT, | | | | | | | |
| | | | 210511_S_AT, 222802_AT, | | | | | | | |
| | | | 219682_S_AT | | | | | | | |
| GO: 0042733~embryonic digit | 4 | 0.824742268 | 227376_AT, 229576_S_AT, | 348 | 25 | 13528 | 6.219770115 | 0.025340745 | 0.412091323 | 36.33847755 |
| morphogenesis | | | 231798_AT, 205201_AT, 225544_AT, | | | | | | | |
| | | | 34697_AT, 219682_S_AT, | | | | | | | |
| | | | 205606_AT | | | | | | | |
| GO: 0050777~negative regulation | 4 | 0.824742268 | 202410_X_AT, 228121_AT, | 348 | 25 | 13528 | 6.219770115 | 0.025340745 | 0.412091323 | 36.33847755 |
| of immune response | | | 203140_AT, 202409_AT, 228758_AT, | | | | | | | |
| | | | 209909_S_AT, 212587_S_AT, | | | | | | | |
| | | | 212588_AT, 236439_AT, | | | | | | | |
| | | | 207238_S_AT | | | | | | | |
| GO: 0060341~regulation of cellular | 13 | 2.680412371 | 212183_AT, 227376_AT, 244802_AT, | 348 | 248 | 13528 | 2.037727104 | 0.026802649 | 0.427374156 | 37.99772039 |
| localization | | | 205201_AT, 216933_X_AT, | | | | | | | |
| | | | 206303_S_AT, 206825_AT, | | | | | | | |
| | | | 218995_S_AT, 202410_X_AT, | | | | | | | |
| | | | 228121_AT, 206857_S_AT, | | | | | | | |
| | | | 203998_S_AT, 202409_AT, | | | | | | | |
| | | | 209030_S_AT, 205258_AT, | | | | | | | |
| | | | 209909_S_AT, 212181_S_AT, | | | | | | | |
| | | | 209032_S_AT, 209031_AT, | | | | | | | |
| | | | 210511_S_AT, 206302_S_AT, | | | | | | | |
| | | | 222802_AT | | | | | | | |
| GO: 0009991~response to | 12 | 2.474226804 | 209875_S_AT, 219181_AT, | 348 | 220 | 13528 | 2.120376176 | 0.026880057 | 0.425638013 | 38.08443095 |
| extracellular stimulus | | | 229339_AT, 208944_AT, | | | | | | | |
| | | | 211355_X_AT, 211356_X_AT, | | | | | | | |
| | | | 209894_AT, 206001_AT, 237206_AT, | | | | | | | |
| | | | 202016_AT, 235591_AT, 213721_AT, | | | | | | | |
| | | | 204932_AT, 227314_AT, 227095_AT, | | | | | | | |
| | | | 203439_S_AT, 204933_S_AT, | | | | | | | |
| | | | 207334_S_AT | | | | | | | |
| GO: 0009123~nucleoside | 6 | 1.237113402 | 212183_AT, 1553995_A_AT, | 348 | 66 | 13528 | 3.533960293 | 0.026950997 | 0.423848207 | 38.16379474 |
| monophosphate metabolic process | | | 206757_AT, 212181_S_AT, | | | | | | | |
| | | | 1553994_AT, 228962_AT, | | | | | | | |
| | | | 206303_S_AT, 227088_AT, | | | | | | | |
| | | | 203817_AT, 206302_S_AT, | | | | | | | |
| | | | 240088_AT, 208447_S_AT | | | | | | | |
| GO: 0000271~polysaccharide | 5 | 1.030927835 | 203188_AT, 214985_AT, 206756_AT, | 348 | 45 | 13528 | 4.319284802 | 0.027766793 | 0.430873871 | 39.06959956 |
| biosynthetic process | | | 202013_S_AT, 218927_S_AT | | | | | | | |
| GO: 0007584~response to nutrient | 9 | 1.855670103 | 209875_S_AT, 204932_AT, | 348 | 140 | 13528 | 2.499014778 | 0.027846808 | 0.429193313 | 39.15776546 |
| | | | 219181_AT, 227314_AT, 208944_AT, | | | | | | | |
| | | | 203439_S_AT, 204933_S_AT, | | | | | | | |
| | | | 202016_AT, 235591_AT, | | | | | | | |
| | | | 207334_S_AT, 213721_AT | | | | | | | |
| GO: 0001558~regulation of cell | 11 | 2.268041237 | 209875_S_AT, 205111_S_AT, | 348 | 194 | 13528 | 2.20417111 | 0.02806467 | 0.429131692 | 39.39721125 |
| growth | | | 229339_AT, 203140_AT, 228758_AT, | | | | | | | |
| | | | 237206_AT, 233401_AT, 223454_AT, | | | | | | | |
| | | | 228121_AT, 202410_X_AT, | | | | | | | |
| | | | 204105_S_AT, 202409_AT, | | | | | | | |
| | | | 210757_X_AT, 209909_S_AT, | | | | | | | |
| | | | 236439_AT, 203382_S_AT, | | | | | | | |
| | | | 210511_S_AT | | | | | | | |
| GO: 0030166~proteoglycan | 4 | 0.824742268 | 214985_AT, 206756_AT, | 348 | 26 | 13528 | 5.980548187 | 0.028115241 | 0.427148342 | 39.4526661 |
| biosynthetic process | | | 202013_S_AT, 218927_S_AT | | | | | | | |
| GO: 0040008~regulation of growth | 16 | 3.298969072 | 209875_S_AT, 205111_S_AT | 348 | 341 | 13528 | 1.823979506 | 0.030406687 | 0.450340296 | 41.9157452 |
| | | | 229339_AT, 203140_AT, 208944_AT, | | | | | | | |
| | | | 228758_AT, 237206_AT, 233401_AT, | | | | | | | |
| | | | 207145_AT, 223454_AT, | | | | | | | |
| | | | 202410_X_AT, 228121_AT, | | | | | | | |
| | | | 204105_S_AT, 231798_AT, | | | | | | | |
| | | | 202409_AT, 210757_X_AT, | | | | | | | |
| | | | 209909_S_AT, 202150_S_AT, | | | | | | | |
| | | | 229376_AT, 236439_AT, | | | | | | | |
| | | | 210511_S_AT, 203382_S_AT, | | | | | | | |
| | | | 207334_S_AT | | | | | | | |
| GO: 0022409~positive regulation of | 3 | 0.618556701 | 266_S_AT, 203333_AT, | 348 | 11 | 13528 | 10.60188088 | 0.030965108 | 0.453827866 | 42.50150477 |
| cell-cell adhesion | | | 209771_X_AT, 204584_AT | | | | | | | |
| GO: 0003002~regionalization | 11 | 2.268041237 | 227376_AT, 224722_AT, | 348 | 197 | 13528 | 2.170605053 | 0.030994258 | 0.451555848 | 42.53192797 |
| | | | 229576_S_AT, 207069_S_AT, | | | | | | | |
| | | | 204689_AT, 205201_AT, | | | | | | | |
| | | | 216933_X_AT, 34697_AT, | | | | | | | |
| | | | 203395_S_AT, 218995_S_AT, | | | | | | | |
| | | | 222696_AT, 231798_AT, 225544_AT, | | | | | | | |
| | | | 215933_S_AT, 219682_S_AT, | | | | | | | |
| | | | 222802_AT, 205606_AT | | | | | | | |
| GO: 0031667~response to nutrient | 11 | 2.268041237 | 209875_S_AT, 219181_AT, | 348 | 197 | 13528 | 2.170605053 | 0.030994258 | 0.451555848 | 42.53192797 |
| levels | | | 208944_AT, 211355_X_AT, | | | | | | | |
| | | | 211356_X_AT, 209894_AT, | | | | | | | |
| | | | 206001_AT, 202016_AT, 235591_AT, | | | | | | | |
| | | | 213721_AT, 204932_AT, 227314_AT, | | | | | | | |
| | | | 227095_AT, 203439_S_AT, | | | | | | | |
| | | | 204933_S_AT, 207334_S_AT | | | | | | | |
| GO: 0051606~detection of stimulus | 8 | 1.649484536 | 37892_AT, 204404_AT, 213721_AT, | 348 | 118 | 13528 | 2.635495811 | 0.032239116 | 0.462242909 | 43.8170981 |
| | | | 206584_AT, 227314_AT, | | | | | | | |
| | | | 203998_S_AT, 204320_AT, | | | | | | | |
| | | | 209030_S_AT, 225835_AT, | | | | | | | |
| | | | 209032_S_AT, 206062_AT, | | | | | | | |
| | | | 1255_G_AT, 209031_AT | | | | | | | |
| GO: 0007218~neuropeptide | 7 | 1.443298969 | 212950_AT, 232267_AT, 244680_AT, | 348 | 93 | 13528 | 2.925967124 | 0.032289162 | 0.460195291 | 43.86819372 |
| signaling pathway | | | 212070_AT, 206001_AT, 205280_AT, | | | | | | | |
| | | | 235591_AT, 212951_AT, | | | | | | | |
| | | | 210473_S_AT | | | | | | | |
| GO: 0032582~negative regulation | 5 | 1.030927835 | 204689_AT, 217707_X_AT, | 348 | 48 | 13528 | 4.049329502 | 0.034178344 | 0.477049606 | 45.76525028 |
| of gene-specific transcription | | | 229376_AT, 44783_S_AT, | | | | | | | |
| | | | 215933_S_AT, 203395_S_AT, | | | | | | | |
| | | | 218839_AT | | | | | | | |
| GO: 0048534~hemopoietic or | 13 | 2.680412371 | 266_S_AT, 209771_X_AT, | 348 | 260 | 13528 | 1.943678161 | 0.036490888 | 0.497272336 | 48.00514581 |
| lymphoid organ development | | | 208944_AT, 203140_AT, 228758_AT, | | | | | | | |
| | | | 212587_S_AT, 203868_S_AT, | | | | | | | |
| | | | 210347_S_AT, 203903_S_AT, | | | | | | | |
| | | | 216933_X_AT, 209619_AT, | | | | | | | |
| | | | 201565_S_AT, 201566_X_AT, | | | | | | | |
| | | | 219497_S_AT, 228121_AT, | | | | | | | |
| | | | 209909_S_AT, 212588_AT, | | | | | | | |
| | | | 207238_S_AT, 236439_AT, | | | | | | | |
| | | | 210511_S_AT, 200878_AT, | | | | | | | |
| | | | 207334_S_AT, 222891_S_AT | | | | | | | |
| GO: 0034101~erythrocyte | 5 | 1.030927835 | 203140_AT, 228758_AT, | 348 | 49 | 13528 | 3.966690124 | 0.036490951 | 0.494666746 | 48.00520587 |
| homeostasis | | | 203903_S_AT, 201565_S_AT, | | | | | | | |
| | | | 201566_X_AT, 236439_AT, | | | | | | | |
| | | | 210511_S_AT, 200878_AT | | | | | | | |
| GO: 0060420~regulation of heart | 3 | 0.618556701 | 231798_AT, 208944_AT, 229376_AT, | 348 | 12 | 13528 | 9.718390805 | 0.036538442 | 0.492542274 | 48.05027734 |
| growth | | | 207334_S_AT | | | | | | | |
| GO: 0014068~positive regulation of | 3 | 0.618556701 | 202410_X_AT, 228121_AT, | 348 | 12 | 13528 | 9.718390805 | 0.036538442 | 0.492542274 | 48.05027734 |
| phosphoinositide 3-kinase cascade | | | 202409_AT, 209909_S_AT, | | | | | | | |
| | | | 226213_AT, 202454_S_AT | | | | | | | |
| GO: 0016202~regulation of striated | 5 | 1.030927835 | 207145_AT, 229576_S_AT, | 348 | 50 | 13528 | 3.887356322 | 0.038891694 | 0.512055301 | 50.23804777 |
| muscle tissue development | | | 229339_AT, 231798_AT, 208944_AT, | | | | | | | |
| | | | 225544_AT, 237206_AT, | | | | | | | |
| | | | 207334_S_AT, 219682_S_AT | | | | | | | |
| GO: 0031349~positive regulation of | 6 | 1.237113402 | 266_S_AT, 227314_AT, | 348 | 73 | 13528 | 3.195087388 | 0.039266786 | 0.51290013 | 50.5786294 |
| defense response | | | 209771_X_AT, 209030_S_AT, | | | | | | | |
| | | | 209032_S_AT, 209031_AT, | | | | | | | |
| | | | 205905_S_AT, 208436_S_AT, | | | | | | | |
| | | | 205904_AT, 206653_AT | | | | | | | |
| GO: 0003007~heart morphogenesis | 6 | 1.237113402 | 224722_AT, 229576_S_AT, | 348 | 73 | 13528 | 3.195087388 | 0.039266786 | 0.51290013 | 50.5786294 |
| | | | 228121_AT, 204320_AT, | | | | | | | |
| | | | 209909_S_AT, 212124_AT, | | | | | | | |
| | | | 37892_AT, 229376_AT, 225544_AT, | | | | | | | |
| | | | 219682_S_AT | | | | | | | |
| GO: 0048634~regulation of muscle | 5 | 1.030927835 | 207145_AT, 229576_S_AT, | 348 | 51 | 13528 | 3.811133649 | 0.041380679 | 0.529189138 | 52.45728838 |
| development | | | 229339_AT, 231798_AT, 208944_AT, | | | | | | | |
| | | | 225544_AT, 237206_AT, | | | | | | | |
| | | | 207334_S_AT, 219682_S_AT | | | | | | | |
| GO: 0009967~positive regulation of | 14 | 2.886597938 | 266_S_AT, 235392_AT, | 348 | 295 | 13528 | 1.844847068 | 0.041485928 | 0.527530983 | 52.5490425 |
| signal transduction | | | 211355_X_AT, 202688_AT, | | | | | | | |
| | | | 226213_AT, 213721_AT, | | | | | | | |
| | | | 202687_S_AT, 202409_AT, | | | | | | | |
| | | | 209909_S_AT, 212588_AT, | | | | | | | |
| | | | 221958_S_AT, 209771_X_AT, | | | | | | | |
| | | | 204689_AT, 211356_X_AT, | | | | | | | |
| | | | 209894_AT, 212587_S_AT, | | | | | | | |
| | | | 202454_S_AT, 226498_AT, | | | | | | | |
| | | | 228121_AT, 227095_AT, | | | | | | | |
| | | | 202410_X_AT, 228949_AT, | | | | | | | |
| | | | 215933_S_AT, 201641_AT, | | | | | | | |
| | | | 207238_S_AT, 204584_AT | | | | | | | |
| GO: 0030324~lung development | 7 | 1.443298969 | 204298_S_AT, 227376_AT, | 348 | 99 | 13528 | 2.748635783 | 0.04187941 | 0.528389801 | 52.89059347 |
| | | | 208944_AT, 215446_S_AT, | | | | | | | |
| | | | 205201_AT, 210980_S_AT, | | | | | | | |
| | | | 213902_AT, 213702_X_AT, | | | | | | | |
| | | | 229376_AT, 200878_AT, | | | | | | | |
| | | | 203395_S_AT, 207334_S_AT | | | | | | | |
| GO: 0014066~regulation of | 3 | 0.618556701 | 202410_X_AT, 228121_AT, | 348 | 13 | 13528 | 8.970822281 | 0.04246325 | 0.530865657 | 53.39310963 |
| phosphoinositide 3-kinase cascade | | | 202409_AT, 209909_S_AT, | | | | | | | |
| | | | 226213_AT, 202454_S_AT | | | | | | | |
| GO: 0007389~pattern specification | 13 | 2.680412371 | 227376_AT, 229576_S_AT, | 348 | 267 | 13528 | 1.892720307 | 0.043241603 | 0.534936908 | 54.05518551 |
| process | | | 224722_AT, 207069_S_AT, | | | | | | | |
| | | | 208944_AT, 204689_AT, 205201_AT, | | | | | | | |
| | | | 216933_X_AT, 34697_AT, | | | | | | | |
| | | | 203395_S_AT, 218995_S_AT, | | | | | | | |
| | | | 226498_AT, 222696_AT, 231798_AT, | | | | | | | |
| | | | 225544_AT, 215933_S_AT, | | | | | | | |
| | | | 207334_S_AT, 219682_S_AT, | | | | | | | |
| | | | 222802_AT, 205606_AT | | | | | | | |
| GO: 0042110~T cell activation | 8 | 1.649484536 | 212587_S_AT, 210347_S_AT, | 348 | 126 | 13528 | 2.468162744 | 0.043560184 | 0.53509307 | 54.32360745 |
| | | | 216933_X_AT, 209619_AT, | | | | | | | |
| | | | 205905_S_AT, 219497_S_AT, | | | | | | | |
| | | | 205904_AT, 202410_X_AT, | | | | | | | |
| | | | 206857_S_AT, 202962_AT, | | | | | | | |
| | | | 202409_AT, 212588_AT, | | | | | | | |
| | | | 207238_S_AT, 222891_S_AT | | | | | | | |
| GO: 0051130~positive regulation of | 10 | 2.06185567 | 266_S_AT, 224722_AT, | 348 | 181 | 13528 | 2.147710675 | 0.043867748 | 0.53515642 | 54.5813433 |
| cellular component organization | | | 209771_X_AT, 216933_X_AT, | | | | | | | |
| | | | 206157_AT, 206825_AT, | | | | | | | |
| | | | 218995_S_AT, 227314_AT, | | | | | | | |
| | | | 228121_AT, 202410_X_AT, | | | | | | | |
| | | | 202409_AT, 209909_S_AT, | | | | | | | |
| | | | 229376_AT, 222802_AT | | | | | | | |
| GO: 0048661~positive regulation of | 4 | 0.824742268 | 226498_AT, 227314_AT, 208944_AT, | 348 | 31 | 13528 | 5.015943641 | 0.044285038 | 0.536123689 | 54.92883605 |
| smooth muscle cell proliferation | | | 207334_S_AT, 222802_AT, | | | | | | | |
| | | | 218995_S_AT | | | | | | | |
| GO: 0043434~response to peptide | 9 | 1.855670103 | 203560_AT, 235392_AT, | 348 | 154 | 13528 | 2.271831617 | 0.044983321 | 0.539364997 | 55.50471878 |
| hormone stimulus | | | 213221_S_AT, 206938_AT, | | | | | | | |
| | | | 235945_AT, 226213_AT, | | | | | | | |
| | | | 202454_S_AT, 206825_AT, | | | | | | | |
| | | | 202410_X_AT, 200953_S_AT, | | | | | | | |
| | | | 202409_AT, 223430_AT, 226636_AT | | | | | | | |
| GO: 0007517~muscle organ | 11 | 2.268041237 | 1569512_AT, 202566_S_AT, | 348 | 211 | 13528 | 2.026583864 | 0.045364087 | 0.539991389 | 55.8158103 |
| development | | | 201539_S_AT, 37892_AT, | | | | | | | |
| | | | 226213_AT, 202454_S_AT, | | | | | | | |
| | | | 219829_AT, 204688_AT, | | | | | | | |
| | | | 211126_S_AT, 207145_AT, | | | | | | | |
| | | | 228121_AT, 209829_AT, | | | | | | | |
| | | | 210299_S_AT, 202565_S_AT, | | | | | | | |
| | | | 204320_AT, 209909_S_AT, | | | | | | | |
| | | | 201540_AT, 207030_S_AT, | | | | | | | |
| | | | 229376_AT, 206707_X_AT, | | | | | | | |
| | | | 214505_S_AT | | | | | | | |
| GO: 0050727~regulation of | 6 | 1.237113402 | 1553995_A_AT, 266_S_AT, | 348 | 76 | 13528 | 3.068965517 | 0.045440595 | 0.538170315 | 55.87807031 |
| inflammatory response | | | 202410_X_AT, 227314_AT, | | | | | | | |
| | | | 209771_X_AT, 203140_AT, | | | | | | | |
| | | | 202409_AT, 1553994_AT, | | | | | | | |
| | | | 228758_AT, 236439_AT, | | | | | | | |
| | | | 203382_S_AT | | | | | | | |
| GO: 0016053~organic acid | 9 | 1.855670103 | 244802_AT, 219429_AT, | 348 | 155 | 13528 | 2.257174638 | 0.046423488 | 0.543578074 | 56.67059831 |
| biosynthetic process | | | 219975_X_AT, 209619_AT, | | | | | | | |
| | | | 207076_S_AT, 223062_S_AT, | | | | | | | |
| | | | 224901_AT, 222802_AT, 226636_AT, | | | | | | | |
| | | | 218995_S_AT | | | | | | | |
| GO: 0046394~carboxylic acid | 9 | 1.855670103 | 244802_AT, 219429_AT, | 348 | 155 | 13528 | 2.257174638 | 0.046423488 | 0.543578074 | 56.67059831 |
| biosynthetic process | | | 219975_X_AT, 209619_AT, | | | | | | | |
| | | | 207076_S_AT, 223062_S_AT, | | | | | | | |
| | | | 224901_AT, 222802_AT, 226636_AT, | | | | | | | |
| | | | 218995_S_AT | | | | | | | |
| GO: 0030323~respiratory tube | 7 | 1.443298969 | 204298_S_AT, 227376_AT, | 348 | 102 | 13528 | 2.667793554 | 0.047281014 | 0.547884988 | 57.35105517 |
| development | | | 208944_AT, 215446_S_AT, | | | | | | | |
| | | | 205201_AT, 210980_S_AT, | | | | | | | |
| | | | 213902_AT, 213702_X_AT, | | | | | | | |
| | | | 229376_AT, 200878_AT, | | | | | | | |
| | | | 203395_S_AT, 207334_S_AT | | | | | | | |
| GO: 0000165~MAPKKK cascade | 10 | 2.06185567 | 205111_S_AT, 212587_S_AT, | 348 | 184 | 13528 | 2.112693653 | 0.047813939 | 0.549607425 | 57.76884519 |
| | | | 209619_AT, 206825_AT, 226498_AT, | | | | | | | |
| | | | 204035_AT, 202410_X_AT, | | | | | | | |
| | | | 213107_AT, 202409_AT, | | | | | | | |
| | | | 211828_S_AT, 209840_S_AT, | | | | | | | |
| | | | 212588_AT, 209539_AT, | | | | | | | |
| | | | 207238_S_AT, 239288_AT, | | | | | | | |
| | | | 209841_S_AT | | | | | | | |
| GO: 0051153~regulation of striated | 4 | 0.824742268 | 229576_S_AT, 229339_AT, | 348 | 32 | 13528 | 4.859195402 | 0.047968481 | 0.548408979 | 57.88927558 |
| muscle cell differentiation | | | 229376_AT, 225544_AT, 237206_AT, | | | | | | | |
| | | | 219682_S_AT, 222802_AT, | | | | | | | |
| | | | 218995_S_AT | | | | | | | |
| GO: 0030204~chondroitin sulfate | 3 | 0.618556701 | 230319_AT, 206756_AT, | 348 | 14 | 13528 | 8.330049261 | 0.048717944 | 0.55174839 | 58.46873127 |
| metabolic process | | | 218927_S_AT | | | | | | | |
| GO: 0006027~glycosaminoglycan | 3 | 0.618556701 | 210619_S_AT, 202838_AT, | 348 | 14 | 13528 | 8.330049261 | 0.048717944 | 0.55174839 | 58.46873127 |
| catabolic process | | | 212334_AT, 212335_AT | | | | | | | |
| GO: 0048844~artery morphogenesis | 3 | 0.618556701 | 212124_AT, 229376_AT, | 348 | 14 | 13528 | 8.330049261 | 0.048717944 | 0.55174839 | 58.46873127 |
| | | | 203382_S_AT | | | | | | | |
| GO: 0030098~lymphocyte | 7 | 1.443298969 | 266_S_AT, 209771_X_AT, | 348 | 103 | 13528 | 2.641892646 | 0.049173001 | 0.552819511 | 58.81688599 |
| differentiation | | | 203140_AT, 228758_AT, | | | | | | | |
| | | | 212587_S_AT, 203868_S_AT, | | | | | | | |
| | | | 210347_S_AT, 216933_X_AT, | | | | | | | |
| | | | 209619_AT, 219497_S_AT, | | | | | | | |
| | | | 212588_AT, 207238_S_AT, | | | | | | | |
| | | | 236439_AT, 222891_S_AT | | | | | | | |
| GO: 0042483~negative regulation | 2 | 0.412371134 | 204932_AT, 204933_S_AT, | 348 | 2 | 13528 | 38.87356322 | 0.050644905 | 0.561385036 | 59.92425202 |
| of odontogenesis | | | 216933_X_AT | | | | | | | |
| GO: 0060298~positive regulation of | 2 | 0.412371134 | 229376_AT, 222802_AT, | 348 | 2 | 13528 | 38.87356322 | 0.050644905 | 0.561385036 | 59.92425202 |
| sarcomere organization | | | 218995_S_AT | | | | | | | |
| GO: 0002521~leukocyte | 8 | 1.649484536 | 266_S_AT, 209771_X_AT, | 348 | 131 | 13528 | 2.373958059 | 0.051795371 | 0.567390694 | 60.77017117 |
| differentiation | | | 208944_AT, 203140_AT, 228758_AT, | | | | | | | |
| | | | 212587_S_AT, 203868_S_AT, | | | | | | | |
| | | | 210347_S_AT, 216933_X_AT, | | | | | | | |
| | | | 209619_AT, 219497_S_AT, | | | | | | | |
| | | | 212588_AT, 207238_S_AT, | | | | | | | |
| | | | 236439_AT, 207334_S_AT, | | | | | | | |
| | | | 222891_S_AT | | | | | | | |
| GO: 0045665~negative regulation | 4 | 0.824742268 | 266_S_AT, 224722_AT, | 348 | 33 | 13528 | 4.711947057 | 0.05179734 | 0.565074915 | 60.77160462 |
| of neuron differentiation | | | 209771_X_AT, 203395_S_AT, | | | | | | | |
| | | | 213721_AT | | | | | | | |
| GO: 0051048~negative regulation | 5 | 1.030927835 | 202410_X_AT, 202409_AT, | 348 | 55 | 13528 | 3.533960293 | 0.052217937 | 0.565769308 | 61.07662644 |
| of secretion | | | 205258_AT, 226213_AT, | | | | | | | |
| | | | 202454_S_AT, 210511_S_AT, | | | | | | | |
| | | | 222802_AT, 218995_S_AT | | | | | | | |
| GO: 0016477~cell migration | 13 | 2.680412371 | 266_S_AT, 209771_X_AT, | 348 | 276 | 13528 | 1.831001166 | 0.053153505 | 0.570083284 | 61.74710855 |
| | | | 203868_S_AT, 216933_X_AT, | | | | | | | |
| | | | 34697_AT, 233401_AT, 226498_AT, | | | | | | | |
| | | | 204035_AT, 223454_AT, 228121_AT, | | | | | | | |
| | | | 204105_S_AT, 209909_S_AT, | | | | | | | |
| | | | 208134_X_AT, 208937_S_AT, | | | | | | | |
| | | | 228396_AT, 201860_S_AT, | | | | | | | |
| | | | 205606_AT | | | | | | | |
| GO: 0002520~immune system | 13 | 2.680412371 | 266_S_AT, 209771_X_AT, | 348 | 276 | 13528 | 1.831001166 | 0.053153505 | 0.570083284 | 61.74710855 |
| development | | | 208944_AT, 203140_AT, 228758_AT, | | | | | | | |
| | | | 212587_S_AT, 203868_S_AT, | | | | | | | |
| | | | 210347_S_AT, 203903_S_AT, | | | | | | | |
| | | | 216933_X_AT, 209619_AT, | | | | | | | |
| | | | 201565_S_AT, 201566_X_AT, | | | | | | | |
| | | | 219497_S_AT, 228121_AT, | | | | | | | |
| | | | 209909_S_AT, 212588_AT, | | | | | | | |
| | | | 207238_S_AT, 236439_AT, | | | | | | | |
| | | | 210511_S_AT, 200878_AT, | | | | | | | |
| | | | 207334_S_AT, 222891_S_AT | | | | | | | |
| GO: 0048598~embryonic | 14 | 2.886597938 | 227376_AT, 229576_S_AT, | 348 | 307 | 13528 | 1.772735782 | 0.053877007 | 0.572839195 | 62.25812676 |
| morphogenesis | | | 214985_AT, 224722_AT, 208944_AT, | | | | | | | |
| | | | 205201_AT, 37892_AT, 34697_AT, | | | | | | | |
| | | | 203395_S_AT, 213721_AT, | | | | | | | |
| | | | 218995_S_AT, 226701_AT, | | | | | | | |
| | | | 231798_AT, 204320_AT, 229376_AT, | | | | | | | |
| | | | 225544_AT, 202013_S_AT, | | | | | | | |
| | | | 207334_S_AT, 219682_S_AT, | | | | | | | |
| | | | 205606_AT, 222802_AT | | | | | | | |
| GO: 0007243~protein kinase | 16 | 3.298969072 | 213221_S_AT, 235392_AT, | 348 | 370 | 13528 | 1.68101895 | 0.054547705 | 0.575185132 | 62.72609188 |
| cascade | | | 226213_AT, 218995_S_AT, | | | | | | | |
| | | | 202409_AT, 212588_AT, 209539_AT, | | | | | | | |
| | | | 239288_AT, 209841_S_AT, | | | | | | | |
| | | | 205285_S_AT, 205111_S_AT, | | | | | | | |
| | | | 212587_S_AT, 227266_S_AT, | | | | | | | |
| | | | 209619_AT, 202454_S_AT, | | | | | | | |
| | | | 206825_AT, 206584_AT, 226498_AT, | | | | | | | |
| | | | 204035_AT, 213107_AT, | | | | | | | |
| | | | 202410_X_AT, 211828_S_AT, | | | | | | | |
| | | | 209840_S_AT, 223430_AT, | | | | | | | |
| | | | 207238_S_AT, 222802_AT, | | | | | | | |
| | | | 211795_S_AT | | | | | | | |
| GO: 0009612~response to | 5 | 1.030927835 | 227314_AT, 208944_AT, 204320_AT, | 348 | 56 | 13528 | 3.470853859 | 0.055146522 | 0.577004675 | 63.13927432 |
| mechanical stimulus | | | 225835_AT, 37892_AT, 204404_AT, | | | | | | | |
| | | | 207334_S_AT, 213721_AT | | | | | | | |
| GO: 0016055~Wnt receptor | 8 | 1.649484536 | 266_S_AT, 209771_X_AT, | 348 | 133 | 13528 | 2.338259442 | 0.05534602 | 0.576103596 | 63.27596562 |
| signaling pathway | | | 204689_AT, 216933_X_AT, | | | | | | | |
| | | | 34697_AT, 224022_X_AT, | | | | | | | |
| | | | 222696_AT, 203221_AT, | | | | | | | |
| | | | 215933_S_AT, 228186_S_AT, | | | | | | | |
| | | | 205606_AT, 203222_S_AT, | | | | | | | |
| | | | 228284_AT | | | | | | | |
| GO: 0051094~positive regulation of | 13 | 2.680412371 | 227376_AT, 208944_AT, 205201_AT, | 348 | 278 | 13528 | 1.817828496 | 0.055498786 | 0.574896971 | 63.38031403 |
| developmental process | | | 212587_S_AT, 216933_X_AT, | | | | | | | |
| | | | 209619_AT, 201566_X_AT, | | | | | | | |
| | | | 201565_S_AT, 233401_AT, | | | | | | | |
| | | | 206825_AT, 213721_AT, | | | | | | | |
| | | | 202410_X_AT, 228121_AT, | | | | | | | |
| | | | 204105_S_AT, 202409_AT, | | | | | | | |
| | | | 209909_S_AT, 212588_AT, | | | | | | | |
| | | | 229376_AT, 207238_S_AT, | | | | | | | |
| | | | 210511_S_AT, 207334_S_AT | | | | | | | |
| GO: 0050900~leukocyte migration | 5 | 1.030927835 | 204035_AT, 266_S_AT, 223454_AT, | 348 | 57 | 13528 | 3.409961686 | 0.058162093 | 0.590269055 | 65.15518105 |
| | | | 228121_AT, 209771_X_AT, | | | | | | | |
| | | | 209909_S_AT, 203868_S_AT | | | | | | | |
| GO: 0051960~regulation of nervous | 10 | 2.06185567 | 209875_S_AT, 266_S_AT, | 348 | 192 | 13528 | 2.024664751 | 0.059536498 | 0.596855446 | 66.03905407 |
| system development | | | 224722_AT, 200953_S_AT, | | | | | | | |
| | | | 209771_X_AT, 204105_S_AT, | | | | | | | |
| | | | 231798_AT, 233401_AT, | | | | | | | |
| | | | 203382_S_AT, 203395_S_AT, | | | | | | | |
| | | | 206825_AT, 213721_AT | | | | | | | |
| GO: 0043408~regulation of | 7 | 1.443298969 | 266_S_AT, 209771_X_AT, | 348 | 109 | 13528 | 2.496467363 | 0.061495526 | 0.606888796 | 67.26240804 |
| MAPKKK cascade | | | 211355_X_AT, 211356_X_AT, | | | | | | | |
| | | | 209894_AT, 212587_S_AT, | | | | | | | |
| | | | 216933_X_AT, 218995_S_AT, | | | | | | | |
| | | | 202410_X_AT, 227095_AT, | | | | | | | |
| | | | 228121_AT, 202409_AT, | | | | | | | |
| | | | 209909_S_AT, 212588_AT, | | | | | | | |
| | | | 207238_S_AT, 222802_AT | | | | | | | |
| GO: 0030539~male genitalia | 3 | 0.618556701 | 229576_S_AT, 206938_AT, | 348 | 16 | 13528 | 7.288793103 | 0.062134969 | 0.608605176 | 67.6526392 |
| development | | | 235945_AT, 225544_AT, | | | | | | | |
| | | | 219682_S_AT, 213721_AT | | | | | | | |
| GO: 0045620~negative regulation | 3 | 0.618556701 | 203140_AT, 228758_AT, 209619_AT, | 348 | 16 | 13528 | 7.288793103 | 0.062134969 | 0.608605176 | 67.6526392 |
| of lymphocyte differentiation | | | 236439_AT, 210511_S_AT | | | | | | | |
| GO: 0045577~regulation of B cell | 3 | 0.618556701 | 266_S_AT, 209771_X_AT, | 348 | 16 | 13528 | 7.288793103 | 0.062134969 | 0.608605176 | 67.6526392 |
| differentiation | | | 212587_S_AT, 212588_AT, | | | | | | | |
| | | | 207238_S_AT, 210511_S_AT | | | | | | | |
| GO: 0002698~negative regulation | 3 | 0.618556701 | 202410_X_AT, 203140_AT, | 348 | 16 | 13528 | 7.288793103 | 0.062134969 | 0.608605176 | 67.6526392 |
| of immune effector process | | | 202409_AT, 228758_AT, | | | | | | | |
| | | | 212587_S_AT, 212588_AT, | | | | | | | |
| | | | 236439_AT, 207238_S_AT | | | | | | | |
| GO: 0007090~regulation of S phase | 3 | 0.618556701 | 203140_AT, 207069_S_AT, | 348 | 16 | 13528 | 7.288793103 | 0.062134969 | 0.608605176 | 67.6526392 |
| of mitotic cell cycle | | | 228758_AT, 229376_AT, 236439_AT | | | | | | | |
| GO: 0007204~elevation of cytosolic | 7 | 1.443298969 | 266_S_AT, 206857_S_AT, | 348 | 110 | 13528 | 2.473772205 | 0.063711836 | 0.615924021 | 68.59628484 |
| calcium ion concentration | | | 205111_S_AT, 209771_X_AT, | | | | | | | |
| | | | 212587_S_AT, 212588_AT, | | | | | | | |
| | | | 207238_S_AT, 222802_AT, | | | | | | | |
| | | | 206825_AT, 218995_S_AT, | | | | | | | |
| | | | 213506_AT | | | | | | | |
| GO: 0030193~regulation of blood | 4 | 0.824742268 | 203824_AT, 203382_S_AT, | 348 | 36 | 13528 | 4.319284802 | 0.064132666 | 0.616244168 | 68.84369787 |
| coagulation | | | 222802_AT, 218995_S_AT, | | | | | | | |
| | | | 213506_AT | | | | | | | |
| GO: 0046634~regulation of alpha- | 4 | 0.824742268 | 266_S_AT, 209771_X_AT, | 348 | 36 | 13528 | 4.319284802 | 0.064132666 | 0.616244168 | 68.84369787 |
| beta T cell activation | | | 203140_AT, 208944_AT, 228758_AT, | | | | | | | |
| | | | 212587_S_AT, 212588_AT, | | | | | | | |
| | | | 236439_AT, 207238_S_AT, | | | | | | | |
| | | | 207334_S_AT | | | | | | | |
| GO: 0050906~detection of stimulus | 4 | 0.824742268 | 227314_AT, 204320_AT, 225835_AT, | 348 | 36 | 13528 | 4.319284802 | 0.064132666 | 0.616244168 | 68.84369787 |
| involved in sensory perception | | | 37892_AT, 204404_AT, 213721_AT | | | | | | | |
| GO: 0046887~positive regulation of | 4 | 0.824742268 | 244802_AT, 205258_AT, | 348 | 36 | 13528 | 4.319284802 | 0.064132666 | 0.616244168 | 68.84369787 |
| hormone secretion | | | 210511_S_AT, 222802_AT, | | | | | | | |
| | | | 218995_S_AT | | | | | | | |
| GO: 0009890~negative regulation | 22 | 4.536082474 | 227376_AT, 235668_AT, 205201_AT, | 348 | 573 | 13528 | 1.492527733 | 0.064217107 | 0.614575945 | 68.89311996 |
| of biosynthetic process | | | 228758_AT, 217707_X_AT, | | | | | | | |
| | | | 201566_X_AT, 228964_AT, | | | | | | | |
| | | | 203395_S_AT, 213721_AT, | | | | | | | |
| | | | 218995_S_AT, 202409_AT, | | | | | | | |
| | | | 205258_AT, 207826_S_AT, | | | | | | | |
| | | | 230258_AT, 44783_S_AT, | | | | | | | |
| | | | 229376_AT, 225544_AT, | | | | | | | |
| | | | 208436_S_AT, 203382_S_AT, | | | | | | | |
| | | | 203222_S_AT, 228284_AT, | | | | | | | |
| | | | 229576_S_AT, 203140_AT, | | | | | | | |
| | | | 204689_AT, 204867_AT, | | | | | | | |
| | | | 201565_S_AT, 218839_AT, | | | | | | | |
| | | | 202410_X_AT, 203221_AT, | | | | | | | |
| | | | 208937_S_AT, 215933_S_AT, | | | | | | | |
| | | | 236439_AT, 229435_AT, | | | | | | | |
| | | | 210511_S_AT, 219682_S_AT, | | | | | | | |
| | | | 222802_AT | | | | | | | |
| GO: 0050767~regulation of | 9 | 1.855670103 | 209875_S_AT, 266_S_AT, | 348 | 166 | 13528 | 2.107602825 | 0.06426862 | 0.612725588 | 68.92323397 |
| neurogenesis | | | 224722_AT, 200953_S_AT, | | | | | | | |
| | | | 209771_X_AT, 204105_S_AT, | | | | | | | |
| | | | 231798_AT, 233401_AT, | | | | | | | |
| | | | 203382_S_AT, 203395_S_AT, | | | | | | | |
| | | | 213721_AT | | | | | | | |
| GO: 0003001~generation of a signal | 6 | 1.237113402 | 206857_S_AT, 229576_S_AT, | 348 | 85 | 13528 | 2.744016227 | 0.067251856 | 0.627844276 | 70.62103207 |
| involved in cell-cell signaling | | | 203998_S_AT, 225544_AT, | | | | | | | |
| | | | 241652_X_AT, 219682_S_AT, | | | | | | | |
| | | | 222802_AT, 218995_S_AT, | | | | | | | |
| | | | 228101_AT | | | | | | | |
| GO: 0009953~dorsal/ventral pattern | 5 | 1.030927835 | 227376_AT, 231798_AT, | 348 | 60 | 13528 | 3.239463602 | 0.067725312 | 0.62838472 | 70.88230047 |
| formation | | | 207069_S_AT, 205201_AT, | | | | | | | |
| | | | 216933_X_AT, 222802_AT, | | | | | | | |
| | | | 218995_S_AT | | | | | | | |
| GO: 0009952~anterior/posterior | 8 | 1.649484536 | 227376_AT, 229576_S_AT, | 348 | 140 | 13528 | 2.22134647 | 0.068945193 | 0.63306965 | 71.54540987 |
| pattern formation | | | 224722_AT, 204689_AT, 205201_AT, | | | | | | | |
| | | | 216933_X_AT, 34697_AT, | | | | | | | |
| | | | 203395_S_AT, 222696_AT, | | | | | | | |
| | | | 215933_S_AT, 225544_AT, | | | | | | | |
| | | | 205606_AT, 219682_S_AT | | | | | | | |
| GO: 0002474~antigen processing | 3 | 0.618556701 | 200904_AT, 205905_S_AT, | 348 | 17 | 13528 | 6.860040568 | 0.069258361 | 0.632689819 | 71.7133311 |
| and presentation of peptide antigen | | | 211799_X_AT, 205904_AT | | | | | | | |
| via MHC class I | | | | | | | | | | |
| GO: 0043405~regulation of MAP | 8 | 1.649484536 | 266_S_AT, 209771_X_AT, | 348 | 141 | 13528 | 2.205592239 | 0.0710376 | 0.640272088 | 72.64976221 |
| kinase activity | | | 205111_S_AT, 212587_S_AT, | | | | | | | |
| | | | 209619_AT, 212558_AT, | | | | | | | |
| | | | 218995_S_AT, 209840_S_AT, | | | | | | | |
| | | | 212588_AT, 207238_S_AT, | | | | | | | |
| | | | 203382_S_AT, 222802_AT, | | | | | | | |
| | | | 209841_S_AT | | | | | | | |
| GO: 0030100~regulation of | 5 | 1.030927835 | 224722_AT, 227314_AT, 239472_AT, | 348 | 61 | 13528 | 3.186357641 | 0.071083045 | 0.638432297 | 72.67329302 |
| endocytosis | | | 213413_AT, 206157_AT | | | | | | | |
| GO: 0050866~negative regulation | 5 | 1.030927835 | 266_S_AT, 209771_X_AT, | 348 | 61 | 13528 | 3.186357641 | 0.071083045 | 0.638432297 | 72.67329302 |
| of cell activation | | | 203140_AT, 228758_AT, 209619_AT, | | | | | | | |
| | | | 236439_AT, 210511_S_AT, | | | | | | | |
| | | | 203382_S_AT | | | | | | | |
| GO: 0016049~cell growth | 5 | 1.030927835 | 210299_S_AT, 228121_AT, | 348 | 61 | 13528 | 3.186357641 | 0.071083045 | 0.638432297 | 72.67329302 |
| | | | 201540_AT, 209909_S_AT, | | | | | | | |
| | | | 203638_S_AT, 201539_S_AT, | | | | | | | |
| | | | 207030_S_AT, 209230_S_AT, | | | | | | | |
| | | | 214505_S_AT, 211126_S_AT | | | | | | | |
| GO: 0045597~positive regulation of | 11 | 2.268041237 | 227376_AT, 208944_AT, 205201_AT, | 348 | 229 | 13528 | 1.867289063 | 0.071852053 | 0.640468285 | 73.06859029 |
| cell differentiation | | | 212587_S_AT, 209619_AT, | | | | | | | |
| | | | 216933_X_AT, 201565_S_AT, | | | | | | | |
| | | | 201566_X_AT, 233401_AT, | | | | | | | |
| | | | 213721_AT, 228121_AT, | | | | | | | |
| | | | 202410_X_AT, 204105_S_AT, | | | | | | | |
| | | | 202409_AT, 209909_S_AT, | | | | | | | |
| | | | 212588_AT, 207238_S_AT, | | | | | | | |
| | | | 210511_S_AT, 207334_S_AT | | | | | | | |
| GO: 0009187~cyclic nucleotide | 4 | 0.824742268 | 212183_AT, 206757_AT, | 348 | 38 | 13528 | 4.091954023 | 0.073037029 | 0.644655189 | 73.66716774 |
| metabolic process | | | 212181_S_AT, 228962_AT, | | | | | | | |
| | | | 206303_S_AT, 227088_AT, | | | | | | | |
| | | | 203817_AT, 206302_S_AT, | | | | | | | |
| | | | 240088_AT | | | | | | | |
| GO: 0032844~regulation of | 7 | 1.443298969 | 209875_S_AT, 204932_AT, | 348 | 114 | 13528 | 2.38697318 | 0.0730421 | 0.642633255 | 73.66970205 |
| homeostatic process | | | 266_S_AT, 206857_S_AT, | | | | | | | |
| | | | 209771_X_AT, 212587_S_AT, | | | | | | | |
| | | | 204933_S_AT, 212588_AT, | | | | | | | |
| | | | 204415_AT, 207238_S_AT, | | | | | | | |
| | | | 210511_S_AT | | | | | | | |
| GO: 0010564~regulation of cell | 7 | 1.443298969 | 207069_S_AT, 203140_AT, | 348 | 114 | 13528 | 2.38697318 | 0.0730421 | 0.642633255 | 73.66970205 |
| cycle process | | | 228758_AT, 212587_S_AT, | | | | | | | |
| | | | 216933_X_AT, 218995_S_AT, | | | | | | | |
| | | | 202410_X_AT, 202409_AT, | | | | | | | |
| | | | 212588_AT, 229376_AT, | | | | | | | |
| | | | 207238_S_AT, 236439_AT, | | | | | | | |
| | | | 222802_AT | | | | | | | |
| GO: 0032147~activation of protein | 7 | 1.443298969 | 226498_AT, 202410_X_AT, | 348 | 114 | 13528 | 2.38697318 | 0.0730421 | 0.642633255 | 73.66970205 |
| kinase activity | | | 228121_AT, 205111_S_AT, | | | | | | | |
| | | | 208944_AT, 202409_AT, | | | | | | | |
| | | | 209909_S_AT, 203680_AT, | | | | | | | |
| | | | 207334_S_AT, 222802_AT, | | | | | | | |
| | | | 218995_S_AT | | | | | | | |
| GO: 0070365~hepatocyte | 2 | 0.412371134 | 227314_AT, 229376_AT | 348 | 3 | 13528 | 25.91570881 | 0.074999941 | 0.650703398 | 74.631187 |
| differentiation | | | | | | | | | | |
| GO: 0030910~olfactory placode | 2 | 0.412371134 | 229376_AT, 213721_AT | 348 | 3 | 13528 | 25.91570881 | 0.074999941 | 0.650703398 | 74.631187 |
| formation | | | | | | | | | | |
| GO: 0033630~positive regulation of | 2 | 0.412371134 | 266_S_AT, 228121_AT, | 348 | 3 | 13528 | 25.91570881 | 0.074999941 | 0.650703398 | 74.631187 |
| cell adhesion mediated by integrin | | | 209771_X_AT, 209909_S_AT | | | | | | | |
| GO: 0010692~regulation of alkaline | 2 | 0.412371134 | 228121_AT, 227314_AT, | 348 | 3 | 13528 | 25.91570881 | 0.074999941 | 0.650703398 | 74.631187 |
| phosphatase activity | | | 209909_S_AT | | | | | | | |
| GO: 0032278~positive regulation of | 2 | 0.412371134 | 205258_AT, 210511_S_AT | 348 | 3 | 13528 | 25.91570881 | 0.074999941 | 0.650703398 | 74.631187 |
| gonadotropin secretion | | | | | | | | | | |
| GO: 0060297~regulation of | 2 | 0.412371134 | 229376_AT, 222802_AT, | 348 | 3 | 13528 | 25.91570881 | 0.074999941 | 0.650703398 | 74.631187 |
| sarcomere organization | | | 218995_S_AT | | | | | | | |
| GO: 0046628~positive regulation of | 2 | 0.412371134 | 202410_X_AT, 235392_AT, | 348 | 3 | 13528 | 25.91570881 | 0.074999941 | 0.650703398 | 74.631187 |
| insulin receptor signaling pathway | | | 202409_AT | | | | | | | |
| GO: 0045750~positive regulation of | 2 | 0.412371134 | 207069_S_AT, 229376_AT | 348 | 3 | 13528 | 25.91570881 | 0.074999941 | 0.650703398 | 74.631187 |
| S phase of mitotic cell cycle | | | | | | | | | | |
| GO: 0046881~positive regulation of | 2 | 0.412371134 | 205258_AT, 210511_S_AT | 348 | 3 | 13528 | 25.91570881 | 0.074999941 | 0.650703398 | 74.631187 |
| follicle-stimulating hormone | | | | | | | | | | |
| secretion | | | | | | | | | | |
| GO: 0014850~response to muscle | 2 | 0.412371134 | 207145_AT, 227314_AT | 348 | 3 | 13528 | 25.91570881 | 0.074999941 | 0.650703398 | 74.631187 |
| activity | | | | | | | | | | |
| GO: 0045892~negative regulation | 15 | 3.092783505 | 235668_AT, 228758_AT, | 348 | 356 | 13528 | 1.637931034 | 0.075429904 | 0.65086368 | 74.83785772 |
| of transcription, DNA-dependent | | | 217707_X_AT, 201566_X_AT, | | | | | | | |
| | | | 228964_AT, 203395_S_AT, | | | | | | | |
| | | | 213721_AT, 207826_S_AT, | | | | | | | |
| | | | 230258_AT, 44783_S_AT, | | | | | | | |
| | | | 229376_AT, 225544_AT, | | | | | | | |
| | | | 208436_S_AT, 203222_S_AT, | | | | | | | |
| | | | 228284_AT, 229576_S_AT, | | | | | | | |
| | | | 203140_AT, 204689_AT, | | | | | | | |
| | | | 201565_S_AT, 218839_AT, | | | | | | | |
| | | | 203221_AT, 215933_S_AT, | | | | | | | |
| | | | 208937_S_AT, 236439_AT, | | | | | | | |
| | | | 229435_AT, 219682_S_AT | | | | | | | |
| GO: 0006790~sulfur metabolic | 7 | 1.443298969 | 204140_AT, 214985_AT, 230319_AT, | 348 | 115 | 13528 | 2.366216892 | 0.075490664 | 0.649158095 | 74.86693486 |
| process | | | 206756_AT, 202013_S_AT, | | | | | | | |
| | | | 218927_S_AT, 203921_AT | | | | | | | |
| GO: 0048568~embryonic organ | 9 | 1.855670103 | 227376_AT, 208944_AT, 235668_AT, | 348 | 172 | 13528 | 2.034081796 | 0.075582025 | 0.647617715 | 74.9105964 |
| development | | | 205201_AT, 37892_AT, 228964_AT, | | | | | | | |
| | | | 213721_AT, 218995_S_AT, | | | | | | | |
| | | | 231798_AT, 204320_AT, 229376_AT, | | | | | | | |
| | | | 207334_S_AT, 200878_AT, | | | | | | | |
| | | | 222802_AT | | | | | | | |
| GO: 0050869~negative regulation | 3 | 0.618556701 | 266_S_AT, 209771_X_AT, | 348 | 18 | 13528 | 6.478927203 | 0.076633702 | 0.650910285 | 75.40807351 |
| of B cell activation | | | 203140_AT, 228758_AT, 236439_AT, | | | | | | | |
| | | | 210511_S_AT | | | | | | | |
| GO: 0050654~chondroitin sulfate | 3 | 0.618556701 | 230319_AT, 206756_AT, | 348 | 18 | 13528 | 6.478927203 | 0.076633702 | 0.650910285 | 75.40807351 |
| proteoglycan metabolic process | | | 218927_S_AT | | | | | | | |
| GO: 0010921~regulation of | 3 | 0.618556701 | 206857_S_AT, 228121_AT, | 348 | 18 | 13528 | 6.478927203 | 0.076633702 | 0.650910285 | 75.40807351 |
| phosphatase activity | | | 227314_AT, 209909_S_AT | | | | | | | |
| GO: 0051046~regulation of | 10 | 2.06185567 | 244802_AT, 226213_AT, | 348 | 202 | 13528 | 1.924433823 | 0.076924956 | 0.650380554 | 75.54419204 |
| secretion | | | 202454_S_AT, 206825_AT, | | | | | | | |
| | | | 218995_S_AT, 228121_AT, | | | | | | | |
| | | | 202410_X_AT, 203998_S_AT, | | | | | | | |
| | | | 202409_AT, 209030_S_AT, | | | | | | | |
| | | | 209909_S_AT, 205258_AT, | | | | | | | |
| | | | 209032_S_AT, 209031_AT, | | | | | | | |
| | | | 210511_S_AT, 222802_AT | | | | | | | |
| GO: 0016042~lipid catabolic | 9 | 1.855670103 | 209420_S_AT, 219181_AT, | 348 | 173 | 13528 | 2.022324098 | 0.077577219 | 0.651640769 | 75.84645621 |
| process | | | 205111_S_AT, 206938_AT, | | | | | | | |
| | | | 200789_AT, 235945_AT, | | | | | | | |
| | | | 203896_S_AT, 213222_AT, | | | | | | | |
| | | | 203382_S_AT, 226636_AT, | | | | | | | |
| | | | 216230_X_AT | | | | | | | |
| GO: 0051147~regulation of muscle | 4 | 0.824742268 | 229576_S_AT, 229339_AT, | 348 | 39 | 13528 | 3.987032125 | 0.077684811 | 0.650211672 | 75.89597552 |
| cell differentiation | | | 229376_AT, 225544_AT, 237206_AT, | | | | | | | |
| | | | 219682_S_AT, 222802_AT, | | | | | | | |
| | | | 218995_S_AT | | | | | | | |
| GO: 0032103~positive regulation of | 5 | 1.030927835 | 204035_AT, 266_S_AT, 227314_AT, | 348 | 64 | 13528 | 3.036997126 | 0.081655138 | 0.667331673 | 77.65778374 |
| response to external stimulus | | | 209771_X_AT, 206001_AT, | | | | | | | |
| | | | 213506_AT | | | | | | | |
| GO: 0051480~cytosolic calcium ion | 7 | 1.443298969 | 266_S_AT, 206857_S_AT, | 348 | 118 | 13528 | 2.306058835 | 0.083113135 | 0.672162798 | 78.27371024 |
| homeostasis | | | 205111_S_AT, 209771_X_AT, | | | | | | | |
| | | | 212587_S_AT, 212588_AT, | | | | | | | |
| | | | 207238_S_AT, 222802_AT, | | | | | | | |
| | | | 206825_AT, 218995_S_AT, | | | | | | | |
| | | | 213506_AT | | | | | | | |
| GO: 0015674~di-, tri-valent | 9 | 1.855670103 | 206857_S_AT, 203710_AT, | 348 | 176 | 13528 | 1.987852665 | 0.083750873 | 0.673163454 | 78.53805527 |
| inorganic cation transport | | | 212587_S_AT, 225627_S_AT, | | | | | | | |
| | | | 203903_S_AT, 212588_AT, | | | | | | | |
| | | | 206001_AT, 224220_X_AT, | | | | | | | |
| | | | 207238_S_AT, 1554830_A_AT, | | | | | | | |
| | | | 227623_AT | | | | | | | |
| GO: 0051253~negative regulation | 15 | 3.092783505 | 235668_AT, 228758_AT, | 348 | 362 | 13528 | 1.610783006 | 0.083764204 | 0.671315081 | 78.54354817 |
| of RNA metabolic process | | | 217707_X_AT, 201566_X_AT, | | | | | | | |
| | | | 228964_AT, 203395_S_AT, | | | | | | | |
| | | | 213721_AT, 207826_S_AT, | | | | | | | |
| | | | 230258_AT, 44783_S_AT, | | | | | | | |
| | | | 229376_AT, 225544_AT, | | | | | | | |
| | | | 208436_S_AT, 203222_S_AT, | | | | | | | |
| | | | 228284_AT, 229576_S_AT, | | | | | | | |
| | | | 203140_AT, 204689_AT, | | | | | | | |
| | | | 201565_S_AT, 218839_AT, | | | | | | | |
| | | | 203221_AT, 215933_S_AT, | | | | | | | |
| | | | 208937_S_AT, 236439_AT, | | | | | | | |
| | | | 229435_AT, 219682_S_AT | | | | | | | |
| GO: 0006692~prostanoid metabolic | 3 | 0.618556701 | 209160_AT, 209619_AT, 222802_AT, | 348 | 19 | 13528 | 6.137931034 | 0.084243466 | 0.67159602 | 78.74015727 |
| process | | | 218995_S_AT | | | | | | | |
| GO: 0050926~regulation of positive | 3 | 0.618556701 | 204035_AT, 227314_AT, 213506_AT | 348 | 19 | 13528 | 6.137931034 | 0.084243466 | 0.67159602 | 78.74015727 |
| chemotaxis | | | | | | | | | | |
| GO: 0050927~positive regulation of | 3 | 0.618556701 | 204035_AT, 227314_AT, 213506_AT | 348 | 19 | 13528 | 6.137931034 | 0.084243466 | 0.67159602 | 78.74015727 |
| positive chemotaxis | | | | | | | | | | |
| GO: 0021545~cranial nerve | 3 | 0.618556701 | 227376_AT, 205201_AT, 226213_AT, | 348 | 19 | 13528 | 6.137931034 | 0.084243466 | 0.67159602 | 78.74015727 |
| development | | | 202454_S_AT, 203395_S_AT | | | | | | | |
| GO: 0034103~regulation of tissue | 3 | 0.618556701 | 209875_S_AT, 204932_AT, | 348 | 19 | 13528 | 6.137931034 | 0.084243466 | 0.67159602 | 78.74015727 |
| remodeling | | | 266_S_AT, 209771_X_AT, | | | | | | | |
| | | | 204933_S_AT | | | | | | | |
| GO: 0006693~prostaglandin | 3 | 0.618556701 | 209160_AT, 209619_AT, 222802_AT, | 348 | 19 | 13528 | 6.137931034 | 0.084243466 | 0.67159602 | 78.74015727 |
| metabolic process | | | 218995_S_AT | | | | | | | |
| GO: 0009582~detection of abiotic | 5 | 1.030927835 | 227314_AT, 204320_AT, 225835_AT, | 348 | 65 | 13528 | 2.990274094 | 0.085342396 | 0.67466035 | 79.18457821 |
| stimulus | | | 37892_AT, 1255_G_AT, 206062_AT, | | | | | | | |
| | | | 204404_AT, 213721_AT | | | | | | | |
| GO: 0048754~branching | 5 | 1.030927835 | 226498_AT, 227376_AT, | 348 | 65 | 13528 | 2.990274094 | 0.085342396 | 0.67466035 | 79.18457821 |
| morphogenesis of a tube | | | 229576_S_AT, 208944_AT, | | | | | | | |
| | | | 205201_AT, 225544_AT, | | | | | | | |
| | | | 207334_S_AT, 219682_S_AT, | | | | | | | |
| | | | 222802_AT, 218995_S_AT | | | | | | | |
| GO: 0009891~positive regulation of | 25 | 5.154639175 | 227376_AT, 235392_AT, 229339_AT, | 348 | 695 | 13528 | 1.398329612 | 0.085749754 | 0.674601855 | 79.34708154 |
| biosynthetic process | | | 205201_AT, 217707_X_AT, | | | | | | | |
| | | | 209595_AT, 242794_AT, | | | | | | | |
| | | | 203395_S_AT, 213721_AT, | | | | | | | |
| | | | 218995_S_AT, 227314_AT, | | | | | | | |
| | | | 202409_AT, 209909_S_AT, | | | | | | | |
| | | | 202986_AT, 230258_AT, 229376_AT, | | | | | | | |
| | | | 225544_AT, 209211_AT, 200878_AT, | | | | | | | |
| | | | 203382_S_AT, 229576_S_AT, | | | | | | | |
| | | | 204689_AT, 1553394_A_AT, | | | | | | | |
| | | | 237206_AT, 206157_AT, 207145_AT, | | | | | | | |
| | | | 202410_X_AT, 228121_AT, | | | | | | | |
| | | | 212124_AT, 215933_S_AT, | | | | | | | |
| | | | 229435_AT, 209212_S_AT, | | | | | | | |
| | | | 210511_S_AT, 222802_AT, | | | | | | | |
| | | | 219682_S_AT | | | | | | | |
| GO: 0001570~vasculogenesis | 4 | 0.824742268 | 229339_AT, 208944_AT, 212124_AT, | 348 | 41 | 13528 | 3.792542753 | 0.087356066 | 0.679824942 | 79.97628341 |
| | | | 44783_S_AT, 237206_AT, | | | | | | | |
| | | | 207334_S_AT, 218839_AT | | | | | | | |
| GO: 0050818~regulation of | 4 | 0.824742268 | 203824_AT, 203382_S_AT, | 348 | 41 | 13528 | 3.792542753 | 0.087356066 | 0.679824942 | 79.97628341 |
| coagulation | | | 222802_AT, 218995_S_AT, | | | | | | | |
| | | | 213506_AT | | | | | | | |
| GO: 0010553~negative regulation | 4 | 0.824742268 | 204689_AT, 217707_X_AT, | 348 | 41 | 13528 | 3.792542753 | 0.087356066 | 0.679824942 | 79.97628341 |
| of specific transcription from RNA | | | 44783_S_AT, 215933_S_AT, | | | | | | | |
| polymerase II promoter | | | 203395_S_AT, 218839_AT | | | | | | | |
| GO: 0042471~ear morphogenesis | 5 | 1.030927835 | 231798_AT, 204320_AT, 37892_AT, | 348 | 66 | 13528 | 2.94496691 | 0.089109583 | 0.685555029 | 80.64248732 |
| | | | 229376_AT, 222802_AT, 213721_AT, | | | | | | | |
| | | | 218995_S_AT | | | | | | | |
| GO: 0030099~myeloid cell | 6 | 1.237113402 | 203140_AT, 208944_AT, 228758_AT, | 348 | 93 | 13528 | 2.507971821 | 0.090743593 | 0.690639237 | 81.24442218 |
| differentiation | | | 203903_S_AT, 201565_S_AT, | | | | | | | |
| | | | 201566_X_AT, 236439_AT, | | | | | | | |
| | | | 210511_S_AT, 200878_AT, | | | | | | | |
| | | | 207334_S_AT | | | | | | | |
| GO: 0032535~regulation of cellular | 12 | 2.474226804 | 209875_S_AT, 203140_AT, | 348 | 271 | 13528 | 1.721338593 | 0.090931529 | 0.689598327 | 81.31251042 |
| component size | | | 228758_AT, 201539_S_AT, | | | | | | | |
| | | | 211126_S_AT, 218995_S_AT, | | | | | | | |
| | | | 223454_AT, 228121_AT, | | | | | | | |
| | | | 202410_X_AT, 210299_S_AT, | | | | | | | |
| | | | 202409_AT, 210757_X_AT, | | | | | | | |
| | | | 203638_S_AT, 209909_S_AT, | | | | | | | |
| | | | 201540_AT, 207030_S_AT, | | | | | | | |
| | | | 209230_S_AT, 236439_AT, | | | | | | | |
| | | | 214505_S_AT, 210511_S_AT, | | | | | | | |
| | | | 222802_AT | | | | | | | |
| GO: 0048663~neuron fate | 4 | 0.824742268 | 227376_AT, 228121_AT, 205201_AT, | 348 | 42 | 13528 | 3.702244116 | 0.092373068 | 0.693769079 | 81.82707452 |
| commitment | | | 209909_S_AT, 203395_S_AT, | | | | | | | |
| | | | 213721_AT | | | | | | | |
| GO: 0001817~regulation of | 9 | 1.855670103 | 266_S_AT, 209771_X_AT, | 348 | 181 | 13528 | 1.932939608 | 0.094665443 | 0.70128105 | 82.61791628 |
| cytokine production | | | 203140_AT, 228758_AT, 201743_AT, | | | | | | | |
| | | | 228121_AT, 202410_X_AT, | | | | | | | |
| | | | 202409_AT, 209030_S_AT, | | | | | | | |
| | | | 209909_S_AT, 205258_AT, | | | | | | | |
| | | | 209032_S_AT, 209031_AT, | | | | | | | |
| | | | 236439_AT, 210511_S_AT, | | | | | | | |
| | | | 206653_AT | | | | | | | |
| GO: 0032869~cellular response to | 5 | 1.030927835 | 213221_S_AT, 200953_S_AT, | 348 | 68 | 13528 | 2.858350237 | 0.096879314 | 0.708253246 | 83.35077378 |
| insulin stimulus | | | 202410_X_AT, 235392_AT, | | | | | | | |
| | | | 202409_AT, 223430_AT, 226213_AT, | | | | | | | |
| | | | 202454_S_AT | | | | | | | |
| GO: 0043583~ear development | 6 | 1.237113402 | 231798_AT, 204320_AT, 37892_AT, | 348 | 95 | 13528 | 2.455172414 | 0.097201457 | 0.707734464 | 83.4549516 |
| | | | 229376_AT, 203395_S_AT, | | | | | | | |
| | | | 222802_AT, 213721_AT, | | | | | | | |
| | | | 218995_S_AT | | | | | | | |
| GO: 0008283~cell proliferation | 17 | 3.505154639 | 219181_AT, 205111_S_AT, | 348 | 436 | 13528 | 1.515712327 | 0.097320427 | 0.706430312 | 83.49326953 |
| | | | 204689_AT, 221530_S_AT, | | | | | | | |
| | | | 212587_S_AT, 226731_AT, | | | | | | | |
| | | | 209619_AT, 224964_S_AT, | | | | | | | |
| | | | 206001_AT, 211126_S_AT, | | | | | | | |
| | | | 202289_S_AT, 227314_AT, | | | | | | | |
| | | | 228121_AT, 206857_S_AT, | | | | | | | |
| | | | 210757_X_AT, 209909_S_AT, | | | | | | | |
| | | | 207030_S_AT, 214660_AT, | | | | | | | |
| | | | 212588_AT, 215933_S_AT, | | | | | | | |
| | | | 201641_AT, 207238_S_AT, | | | | | | | |
| | | | 201186_AT, 1560359_AT | | | | | | | |
| GO: 0048870~cell motility | 13 | 2.680412371 | 266_S_AT, 209771_X_AT, | 348 | 307 | 13528 | 1.646111798 | 0.098654082 | 0.709825864 | 83.91712584 |
| | | | 203868_S_AT, 216933_X_AT, | | | | | | | |
| | | | 34697_AT, 233401_AT, 226498_AT, | | | | | | | |
| | | | 204035_AT, 223454_AT, 228121_AT, | | | | | | | |
| | | | 204105_S_AT, 209909_S_AT, | | | | | | | |
| | | | 208134_X_AT, 208937_S_AT, | | | | | | | |
| | | | 228396_AT, 201860_S_AT, | | | | | | | |
| | | | 205606_AT | | | | | | | |
| GO: 0051674~localization of cell | 13 | 2.680412371 | 266_S_AT, 209771_X_AT, | 348 | 307 | 13528 | 1.646111798 | 0.098654082 | 0.709825864 | 83.91712584 |
| | | | 203868_S_AT, 216933_X_AT, | | | | | | | |
| | | | 34697_AT, 233401_AT, 226498_AT, | | | | | | | |
| | | | 204035_AT, 223454_AT, 228121_AT, | | | | | | | |
| | | | 204105_S_AT, 209909_S_AT, | | | | | | | |
| | | | 208134_X_AT, 208937_S_AT, | | | | | | | |
| | | | 228396_AT, 201860_S_AT, | | | | | | | |
| | | | 205606_AT | | | | | | | |
| GO: 0010923~negative regulation | 2 | 0.412371134 | 206857_S_AT, 228121_AT, | 348 | 4 | 13528 | 19.43678161 | 0.09873192 | 0.708376484 | 83.94154413 |
| of phosphatase activity | | | 209909_S_AT | | | | | | | |
| GO: 0032277~negative regulation | 2 | 0.412371134 | 205258_AT, 210511_S_AT | 348 | 4 | 13528 | 19.43678161 | 0.09873192 | 0.708376484 | 83.94154413 |
| of gonadotropin secretion | | | | | | | | | | |
| GO: 0032276~regulation of | 2 | 0.412371134 | 205258_AT, 210511_S_AT | 348 | 4 | 13528 | 19.43678161 | 0.09873192 | 0.708376484 | 83.94154413 |
| gonadotropin secretion | | | | | | | | | | |
| GO: 0046880~regulation of follicle- | 2 | 0.412371134 | 205258_AT, 210511_S_AT | 348 | 4 | 13528 | 19.43678161 | 0.09873192 | 0.708376484 | 83.94154413 |
| stimulating hormone secretion | | | | | | | | | | |
| GO: 0015014~heparan sulfate | 2 | 0.412371134 | 214985_AT, 202013_S_AT | 348 | 4 | 13528 | 19.43678161 | 0.09873192 | 0.708376484 | 83.94154413 |
| proteoglycan biosynthetic process, | | | | | | | | | | |
| polysaccharide chain biosynthetic | | | | | | | | | | |
| process | | | | | | | | | | |
| GO: 0006907~pinocytosis | 2 | 0.412371134 | 239472_AT, 210757_X_AT | 348 | 4 | 13528 | 19.43678161 | 0.09873192 | 0.708376484 | 83.94154413 |
| GO: 0046882~negative regulation | 2 | 0.412371134 | 205258_AT, 210511_S_AT | 348 | 4 | 13528 | 19.43678161 | 0.09873192 | 0.708376484 | 83.94154413 |
| of follicle-stimulating hormone | | | | | | | | | | |
| secretion | | | | | | | | | | |
| GO: 0006874~cellular calcium ion | 9 | 1.855670103 | 266_S_AT, 209771_X_AT, | 348 | 183 | 13528 | 1.911814585 | 0.099248776 | 0.708613825 | 84.10280048 |
| homeostasis | | | 205111_S_AT, 203710_AT, | | | | | | | |
| | | | 212587_S_AT, 206825_AT, | | | | | | | |
| | | | 213506_AT, 218995_S_AT, | | | | | | | |
| | | | 206857_S_AT, 212588_AT, | | | | | | | |
| | | | 207238_S_AT, 203382_S_AT, | | | | | | | |
| | | | 222802_AT | | | | | | | |
| GO: 0000904~cell morphogenesis | 11 | 2.268041237 | 216933_X_AT, 226213_AT, | 348 | 244 | 13528 | 1.752496702 | 0.099863985 | 0.709223052 | 84.29275185 |
| involved in differentiation | | | 202454_S_AT, 203395_S_AT, | | | | | | | |
| | | | 233401_AT, 203188_AT, 228121_AT, | | | | | | | |
| | | | 231798_AT, 204105_S_AT, | | | | | | | |
| | | | 209909_S_AT, 210757_X_AT, | | | | | | | |
| | | | 213438_AT, 229376_AT, 204584_AT | | | | | | | |

TABLE 3
miRNAs list for direct hematopoietic lineage conversion that are used in
the transdetermination procedure #2.
miRNAs list for direct hematopoietic conversion

Has-miR-let7-b
Hsa-miR-let7-c
Hsa-miR-10a
Hsa-miR-22
Hsa-miR-23a, 27a, 24-2 (cluster)
Hsa-miR-23b
Hsa-miR-24-1, 3074 (cluster)
Hsa-miR-25, 93 (cluster)
Hsa-miR-26a
Has-mir-26b
Hsa-miR-29a
Hsa-miR-29b-1
Hsa-miR-29c
Hsa-miR-99a
Hsa-miR-99b
Hsa-miR-125a-5p
Hsa-miR-125b-1
Hsa-miR-126
Hsa-miR-144, 451 (cluster)
Hsa-miR-146a
Hsa-miR-146b-5p
Hsa-miR-155
Hsa-miR-181a-1
Hsa-miR-181c, 181d (cluster)
Hsa-miR-191
Hsa-miR-221
Hsa-miR-222
Hsa-miR-223
Hsa-miR-517c
Has-miR-518a2
Hsa-miR-519d
Hsa-miR-520h
Hsa-miR-551b

Statistical Evaluation:

Statistical analyses of all endpoints were performed by using the standard unpaired Student t test. All data are presented as mean±standard deviation of the mean and represent a minimum of two independent experiments with at least two technical duplicates.

### Example 2

### Generation of Hematopoietic Stem Cells

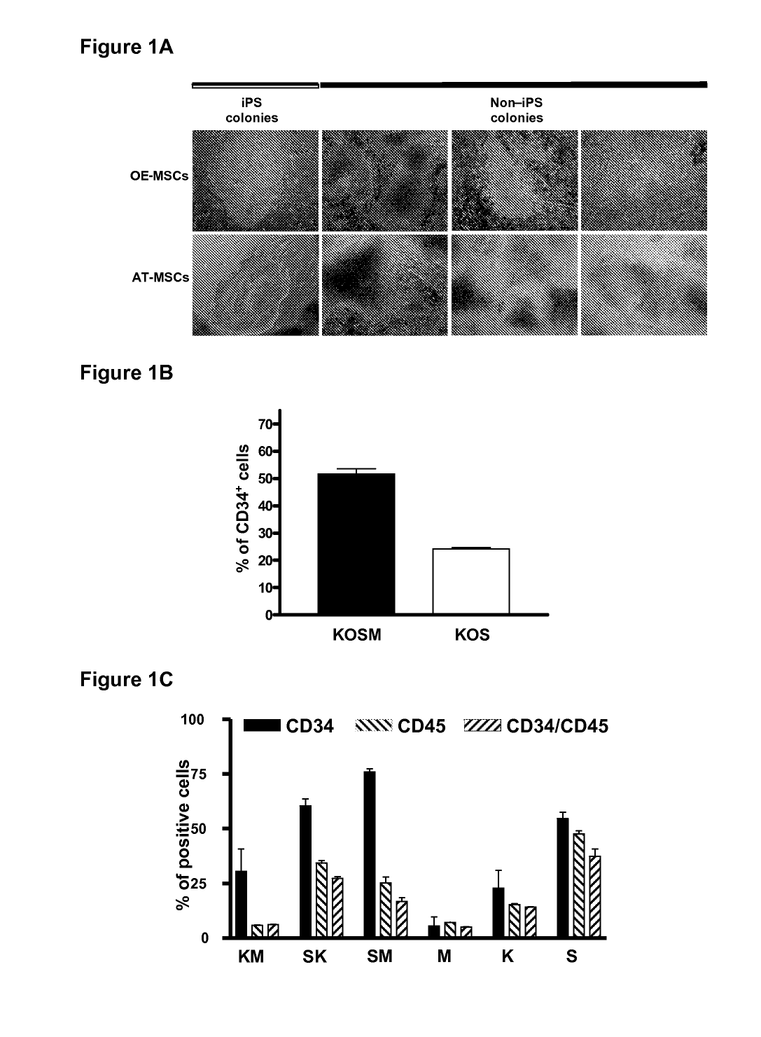

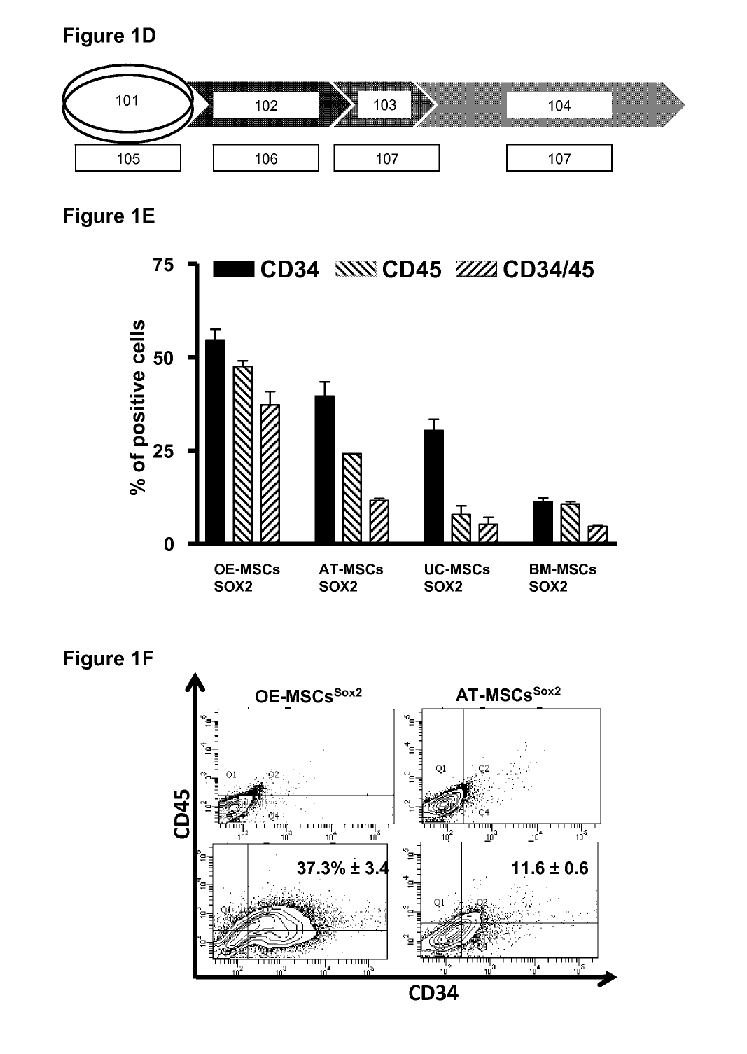

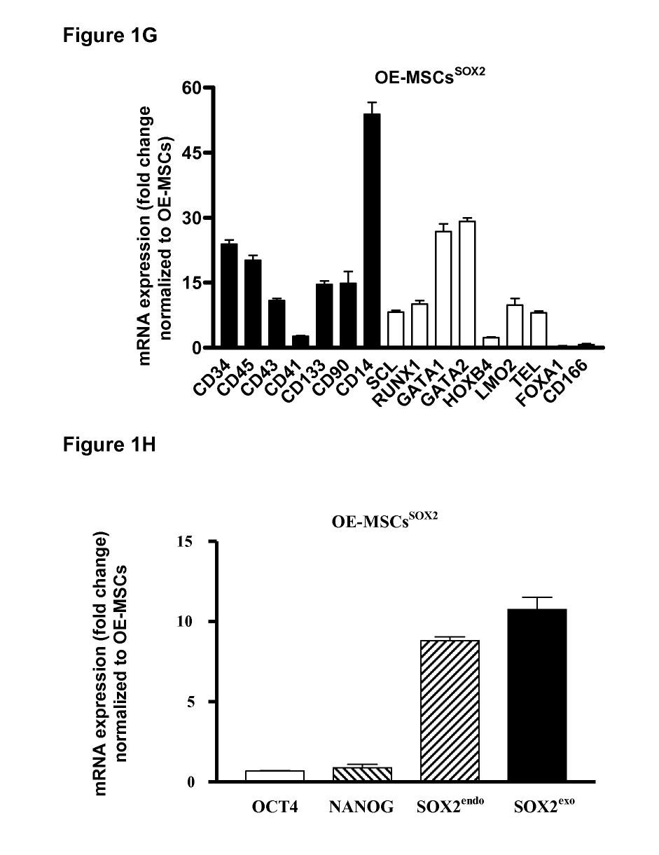

A schematic representation for the transgeneration of Hematopoietic Stem Cells is shown in FIG. 1A. For comparison, Olfactory-Epithelia and Adipose-Tissue derived Mesenchymal Stem Cells (OEMSCs and ATMSCs, respectively) were transduced with a retroviral vector containing one or more of the Yamanaka factors KLF4, Oct4, Sox2 and c-Myc (KOSM) under conditions suitable for reprogramming. After one month, fully reprogrammed induced pluripotent stem (iPS) colonies were observed, as well as colonies with hematopoietic-like morphology representing populations “partially-reprogrammed” cells (FIG. 1A). Flow cytometry analysis confirmed the identity of the partially reprogrammed cells as hematopoietic-like progenitor cells (HPCs) as measured by expression of the hematopoietic progenitor markers CD34 (FIG. 1B).

Applicants first sought to exclude the oncogene, cMYC from the KOSM cocktail. As shown in FIG. 1B, the absence of c-Myc did not impair the capacity of MSCs to generate HPCs.

In order to exclude the possibility that HPCs derived from iPS cells underwent spontaneous differentiation towards the hematopoietic lineage, fully reprogrammed MSC-derived-iPS single colonies were picked and clonally expanded to obtain pure cultures. Expression of CD34 and CD45 on these iPS clones was not significantly upregulated. Thus, under the culture conditions described herein, HPCs are generated from partially reprogrammed cells and not from iPS colonies.

Applicants next sought to investigate whether HPCs could be generated in the complete absence of factors and conditions permissive for full reprogramming and iPS generation. Accordingly, Applicants applied different combinations of factors in the complete absence of Oct4, considered the master regulator and only factor required for iPS generation.

In a further attempt to make the HPC generation “clinic-friendly,” Applicants established a culture system lacking any feeder layer and/or matrigel matrix, which had been considered necessary for iPS generation. Upon transduction of different factors, Applicants observed HPC generation with varying efficiencies, depending on the exogenous gene/s employed (FIG. 1C). Every combination including Sox2 yielded higher number of progenitors as compared to any other single factor or dual combinations (FIG. 1C). Indeed, Sox2 alone led to higher amounts of CD45+ cells as well as CD34+CD45+ double positive cells.

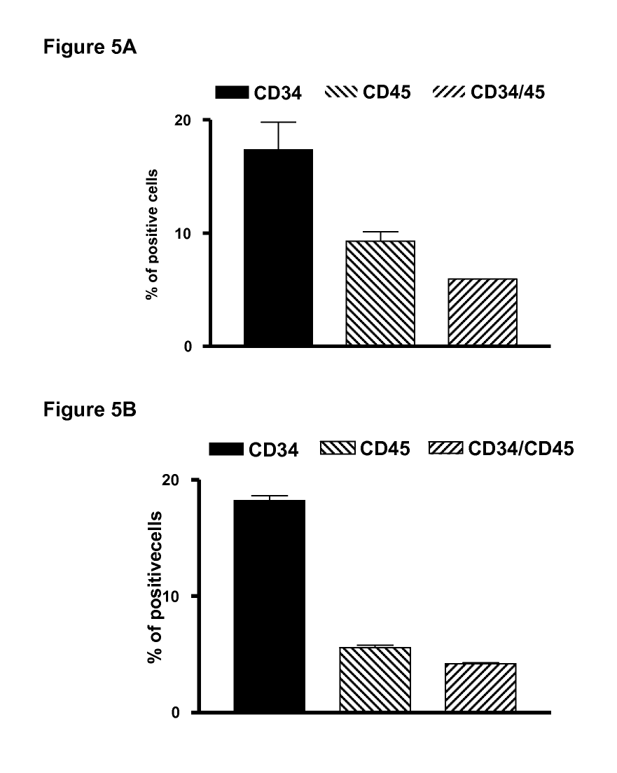

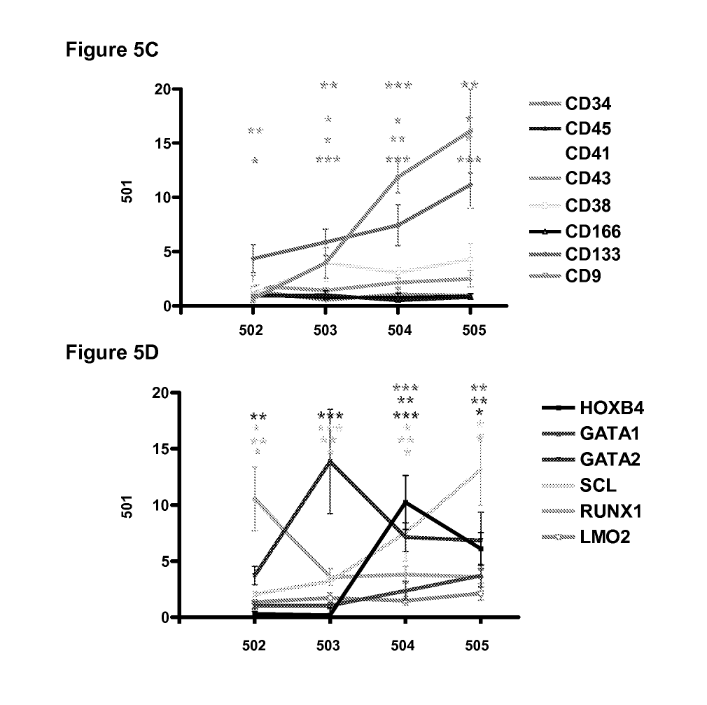

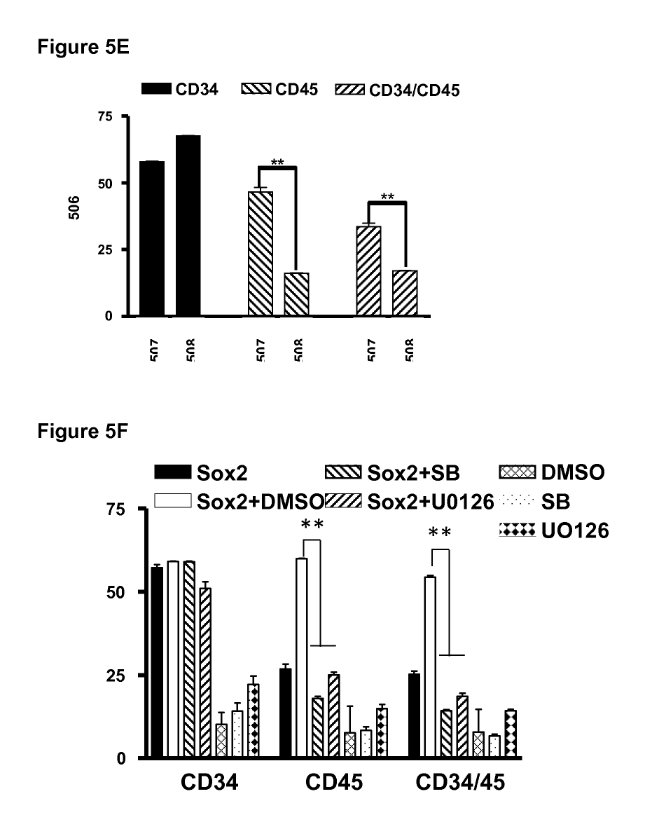

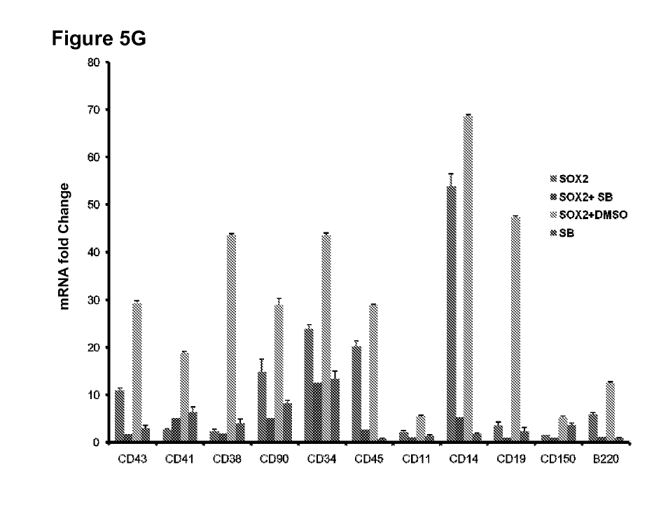

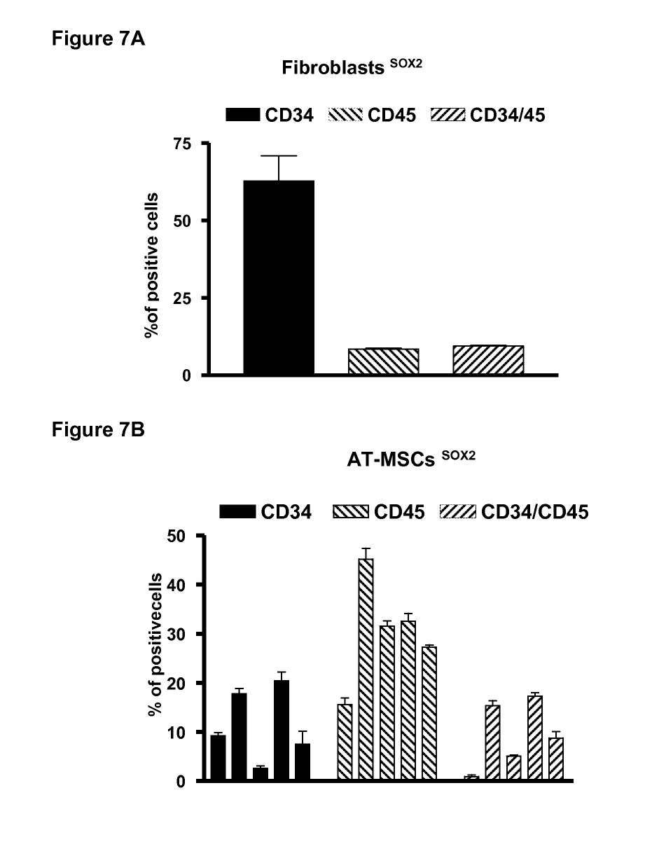

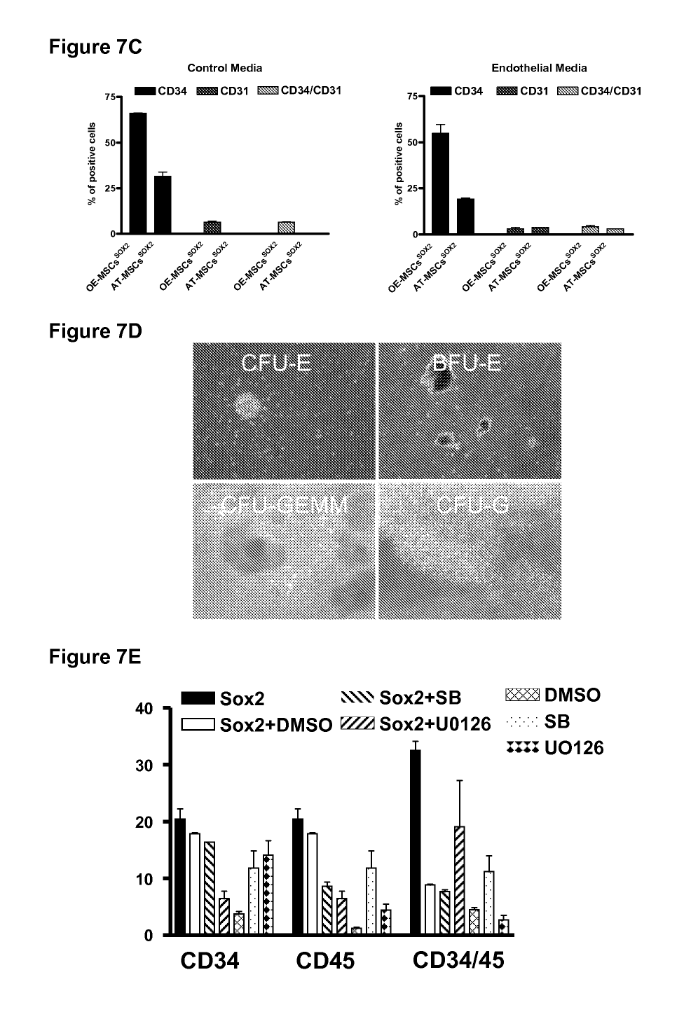

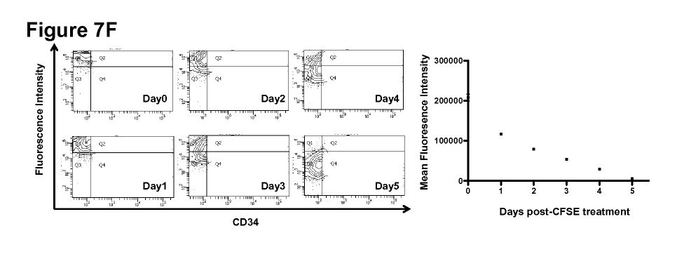

Many of the conditions tested yielded relatively high amounts of HPCs. In order to avoid use of oncogenes Klf4 and c-Myc, Applicants focused on single factor transduction using only Sox2. Applicants found that culture of OEMSCs and ATMSCs in “non-permissive” iPS conditions (single Sox2 transduction and plastic surfaces) led to the efficient transdetermination of MSCs towards HPCs, as summarized in FIG. 1D. While Applicants observed slightly different transdetermination kinetics and efficiency with the different viral cocktails used, infection with Sox2 alone robustly yielded a total of 40-70% CD34+ cells in a time period not exceeding 8 days in vitro (FIG. 1B-E, FIG. 5a and FIG. 7).

Applicants next wondered whether the observed commitment towards the hematopoietic lineage was restricted to OEMSCs or particular OEMSC lines. To address this issue, Applicants compared the expression of CD34 and CD45 in three different OEMSC lines derived from different patients and observed no differences in their ability to generate HPCs. Furthermore, Applicants tested MSCs derived from different tissues, including Bone Marrow MSCs, Adipose Tissue MSCs, and Umbilical Cord MSCs in their capacity to differentiate towards the hematopoietic lineage. Applicants found that OEMSCs were the most efficient source for the generation of HPCs (FIG. 1E).

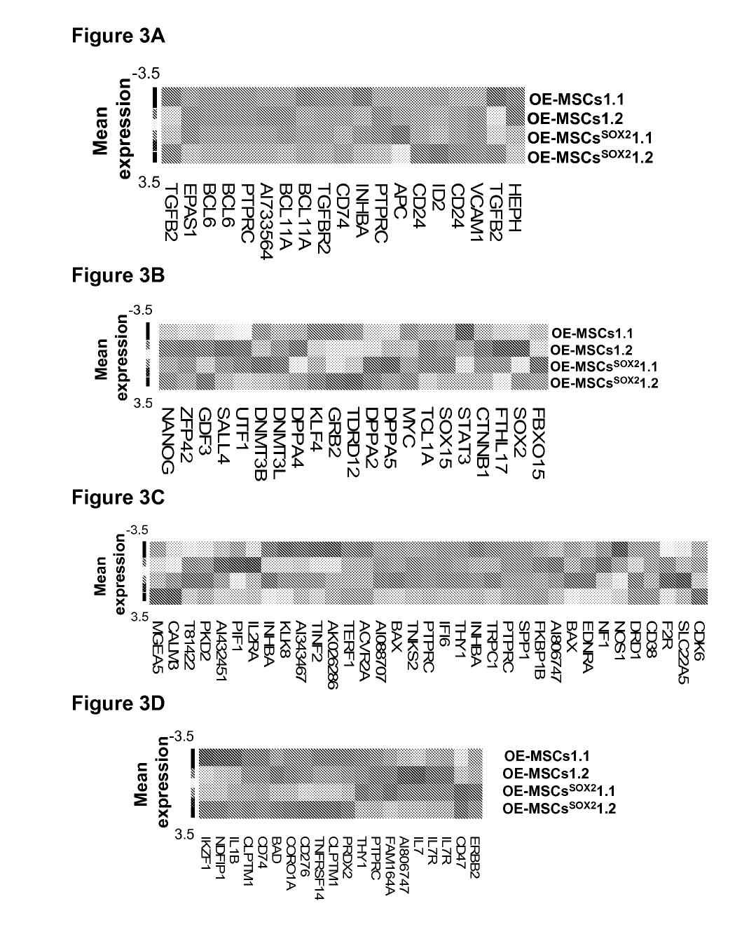

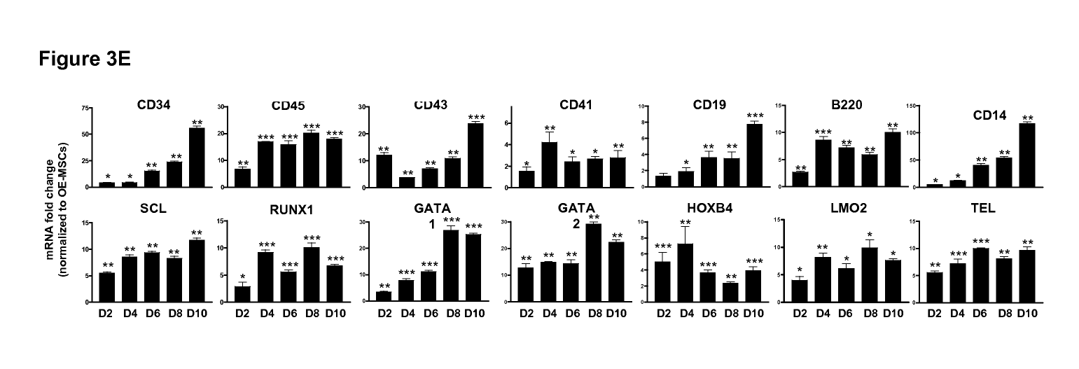

To further demonstrate that the transdetermined cells were following the HPC lineage, Applicants investigate the expression levels of a series of well-established hematopoietic markers. Applicants found that the early hematopoietic markers SCL, Runx1, CD41 and CD43 were strongly upregulated at the RNA and protein levels, thus demonstrating the hematopoietic nature of the transdetermined cells (FIG. 1G, FIG. 3B).

### Example 3

### Exclusion of iPS Generation

Applicants modified the original protocol of transgenerating MSCs to HPCs by excluding the possibility of parallel iPS generation. To this end, Applicants performed a series of experiments comparing transgeneration efficiency in different substrates, including the original mouse embryonic fibroblasts (MEF) feeder layer, matrigel coating, and basic plastic. Applicants' results showed no differences between the growing conditions tested. Thus, Applicants decided to use plastic, previously considered a non-permissive condition for iPS generation, as Applicants' transgeneration culture system.

### Example 4

### Time Frame for In Vitro Transdetermination

In order to effect an efficient transdetermination protocol suitable for clinical translation and therapy, Applicants investigated the temporal aspect of the transdetermination process. To this end, Applicants observed the rate of HPC generation every other day over a total period of ten days (FIG. 3). Applicants' results showed rapid generation of HPCs, with the first appearance as soon as two days after transdetermination induction (FIG. 3A). At early time-points, the majority of progenitor cells included a population of single CD34+ cells whereas CD34+CD45+ cells started to appear between days 2-6. At days 8 and 10, Applicants did not observe any additional changes in the relative distribution of each population, and Applicants consistently observed between 40-60% CD34+, from which up to 30-35% were comprised of a double positive CD34+CD45+ population. Moreover, at day 8 Applicants observed the appearance of single CD45+ populations comprising up to 25% of the total number of cells (FIG. 3A and FIG. 7).

Applicants' transdetermination procedure thus promotes normal human hematopoietic development in which CD34+ cells mature towards a more definitive CD34+CD45+ progenitor state, which leads to the generation of progenitors of the myeloid and lymphoid lineage, i.e., CD45+ cells (FIG. 3A and FIG. 7). Furthermore, the protocol results in generation of CD34+ early progenitor cells in two days of in vitro transdetermination, thus defining a time-window that could be applied to the treatment of acute malignancies.

### Example 5

### Gene Expression Levels Associated with Transdetermination Follow Normal Hematopoietic Cell Development

In order to further validate the identity of the transdetermined cells, Applicants performed qPCR (i.e., quantitative PCR or real-time PCR) analysis every other day of transdetermination (FIG. 3C and FIG. 7). Applicants observed rapid upregulation of the early hematopoietic markers SCL, CD41, CD43, Runx1 over the first 2-4 days, thus demonstrating the hematopoietic nature of the newly generated cells. The transition from early progenitors towards late progenitors is also observed at protein level.

Applicants also observed a small subpopulation of CD133+ cells among the CD34+ population, representing up to 3% of the total number of cells and about one fifth of the total CD34+ population by day 2. In addition, expression of CD43, an intermediate hematopoietic progenitor marker, peaked after four days of in vitro transdetermination. This represents an additional subpopulation of intermediate progenitors comprising up to 13% CD34+CD43+ cells (FIG. 3B).

Reduced CD43 protein expression is then observed, indicative of progression towards more mature progenitor states (FIG. 3B). In this regard, Applicants' method results in normal development representing the transition of CD34− to CD34+ cells and then to a more committed progenitor state characterized by CD45 expression, a lymphoid and myeloid marker.

### Example 6

### Enrichment of HPC Populations

CD34 has been routinely used as a marker for the isolation of a population of cells containing human HPCs. Accordingly, Applicants performed enrichment of CD34+ cells by Magnetic Activated Cell Sorting (MACS). The purity of the sorted cell population routinely lies between 85-95% (FIG. 7). As expected, sorting of CD34+ cells gave rise to all major blood lineages in colony-forming assays, including rapid formation of BFU-E and CFU-E colonies, thus demonstrating the multilineage potential of the transdetermined cells (FIG. 3D).

### Example 7

### Proliferative Capacity of Transdetermined HPCs

One of the major hurdles for clinical application of HPCs has been the capacity to efficiently expand isolated populations of CD34+ HPCs. Cell number is typically a limiting factor for both developmental and studies and clinical application. Furthermore, iPS-derived CD34+ cells have been reported to undergo rapid senescence and apoptosis.

Considering that MSCs have an inherently high proliferative potential, Applicants sought to determine whether the transdetermined cells would retain proliferation capacity and allow efficient expansion of the transdetermined HPCs. To this end, Applicants performed cell proliferation studies using CFSE staining. If the initially generated CD34+ cells do not proliferate further, Applicants would expect a different population of cells to retain high levels of CFSE, while the rest of the CD34− cells belonging to the MSC lineage show a significant reduction in CFSE fluorescence intensity as transdetermination progressed.

Applicants did not observe two distinct, high-fluorescence versus low-fluorescence, populations but a homogenous reduction of fluorescence intensity (FIG. 3E and FIG. 7), confirming that Applicants' in vitro system allows for expansion of transdetermined CD34+ cells.

Applicants next sought to identify the fraction of MSCs that apparently did not progress, despite the nearly 100% transduction efficiency. Applicants hypothesized that non-transdetermined MSCs might be competing with the newly generated CD34+ in terms of nutrients and/or that the CD34+ population might be actively repressing further transdetermination of MSCs. Accordingly, Applicants allowed transdetermination to progress for 4 days prior to depletion of the CD34+ population. Applicants observed that depletion of CD34+ cells by day 4, and re-plating of the negative fraction for 6 more days allowed for further transdetermination of MSCs to levels comparable to the non-depleted control. Thus, transdetermination potential is not totally inherent to stochasticity of transduction of the initial MSC population.

### Example 8

### Inhibition of TGF Beta Signaling can Effect Cell Reprogramming

While the SOX2 transduction method results in a very efficient, feeder-free, rapid and non-pluripotent method for the generation of HPCs, it still involves induction of HPCs by virus transduction. Accordingly, Applicants next focused Applicants' efforts on the development of virus-free, non-integrative approach (i.e., no integration of exogenous genetic material).

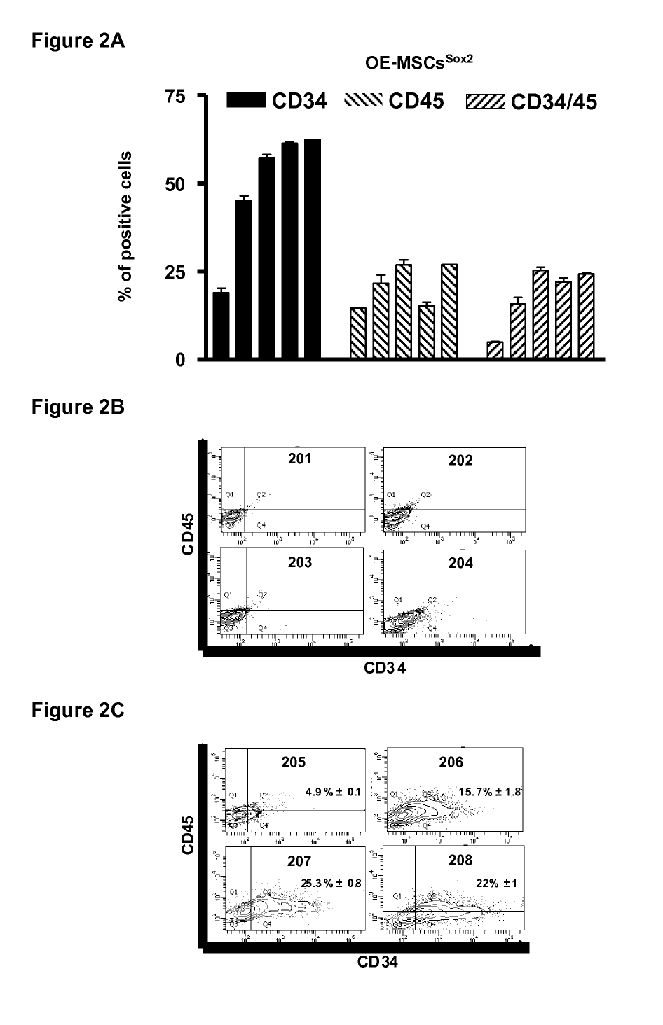

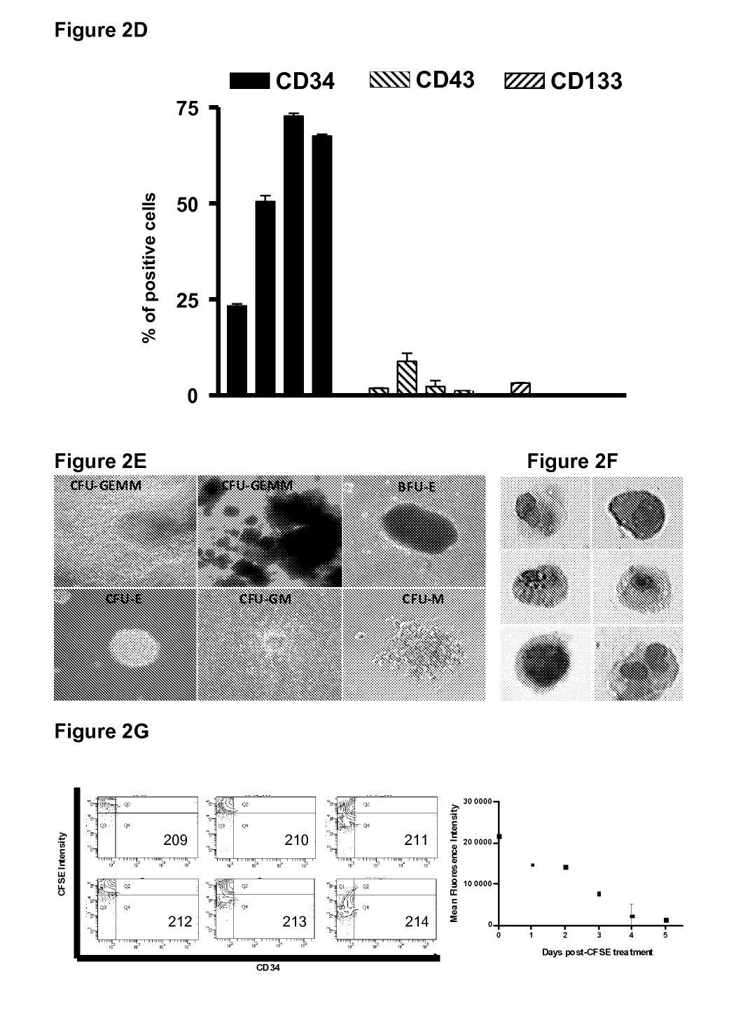

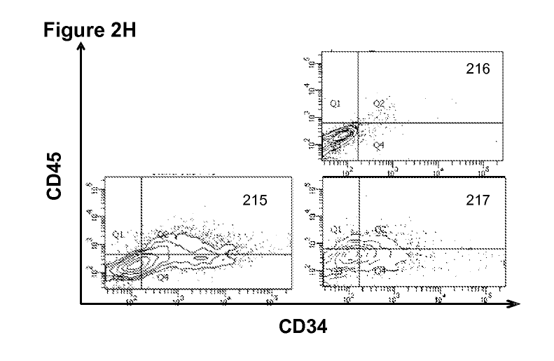

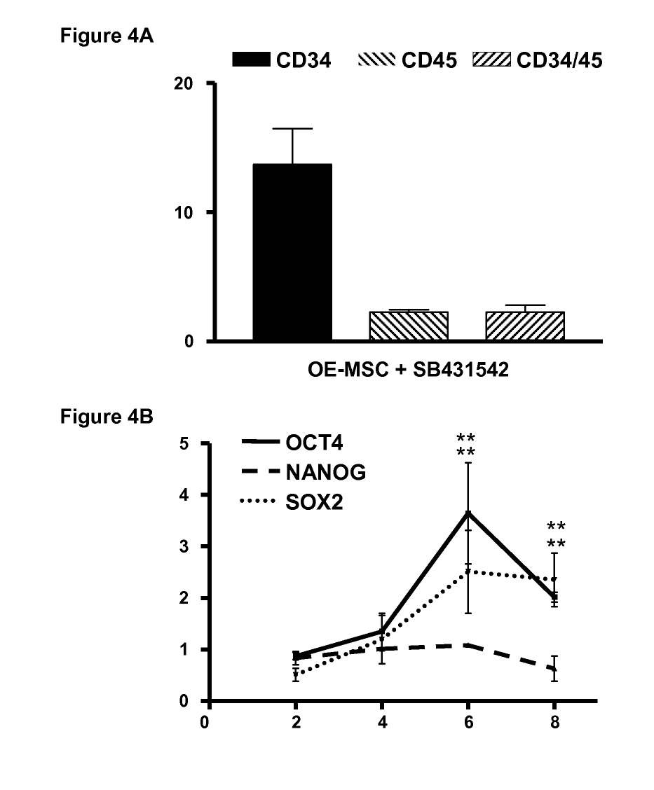

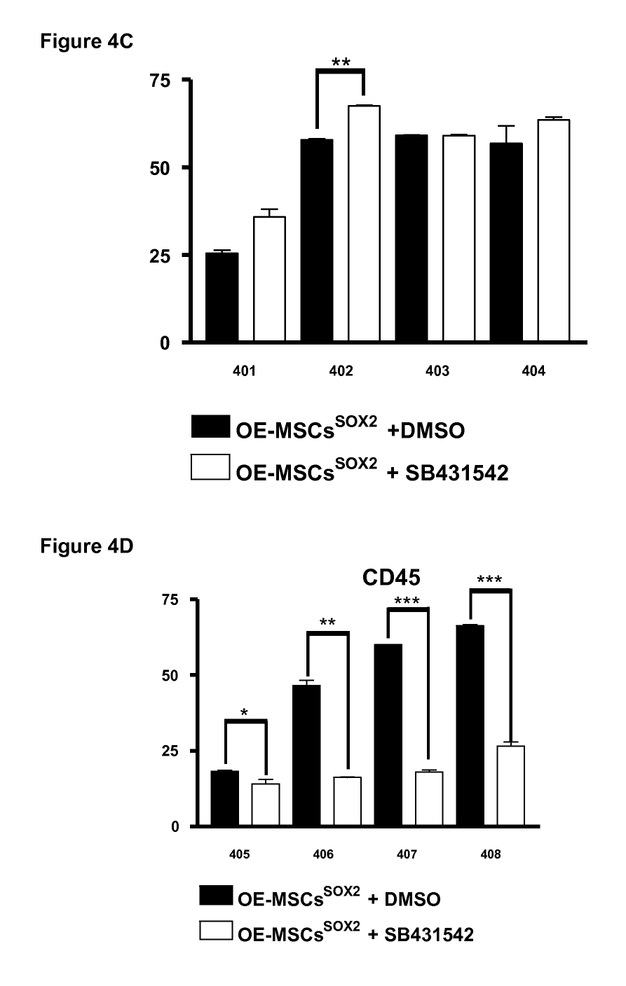

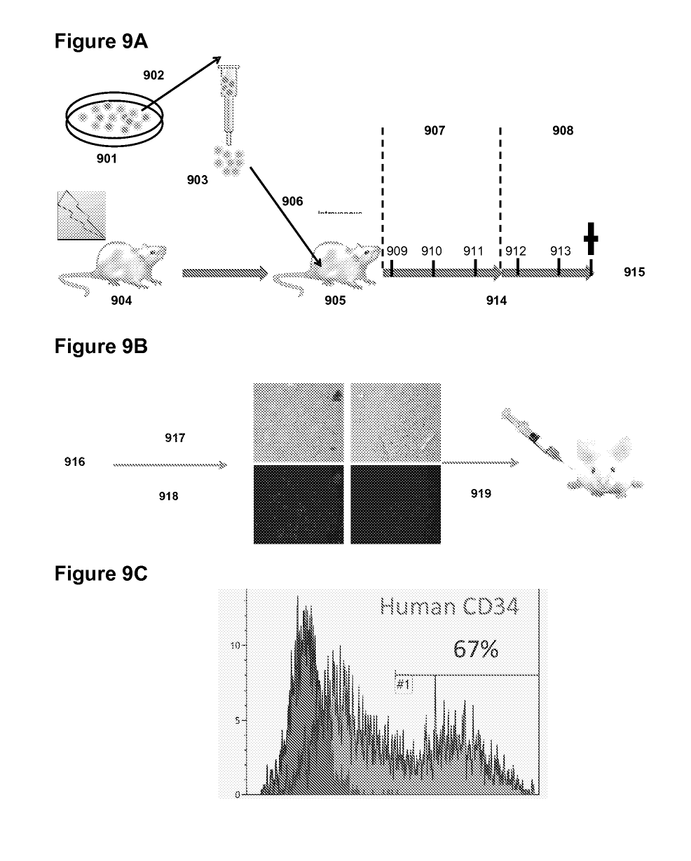

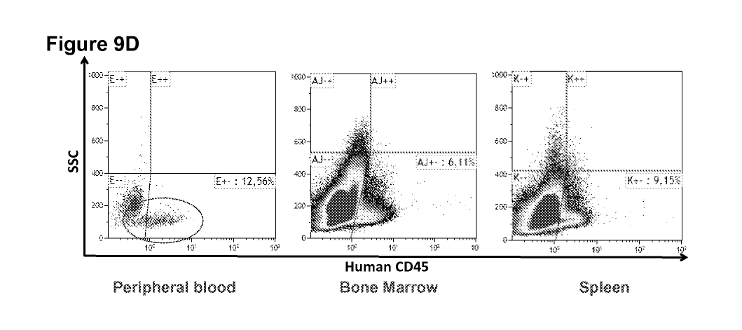

TGF beta is reported to be an upstream modulator of Sox2 (Li (2010) Cell Reprogram 12:3; Lee et al. (2009) Stem Cells 27: 1858). Thus, Applicants decided to evaluate the role of TGF beta signaling pathway during the transgeneration process and whether it might functionally replace exogenous Sox2 expression. Even though inhibition of the TGF beta pathway during ten days consistently produces around 10% CD34+CD45+ cells as compared to its respective DMSO control, it did not fully replace Sox2. Interestingly, when combined with Applicants' “standard” transgeneration protocol (FIG. 1D), TGF beta inhibition strongly attenuates the generation of CD45+ cells. Moreover, RNA expression analysis showed efficient upregulation of several hematopoietic markers including CD34, CD45 and the early markers CD41 and CD43 and downregulation of markers associated with sternness (FIG. 4 and FIG. 9). The observed changes on gene expression were significantly abrogated by TGF beta inhibition. See FIG. 4. Thus, Applicants' results show that TGF beta signaling can mediate the transition of CD34+ cells towards CD34+CD45+ double positive cells. See FIG. 2 and FIG. 4. Similarly, long-term inhibition of TGF beta pathway during a one month period in culture leads to increased accumulation of up to 75% CD34+ cells as compared to the standard protocol. See FIGS. 1G, 2A, and 4.

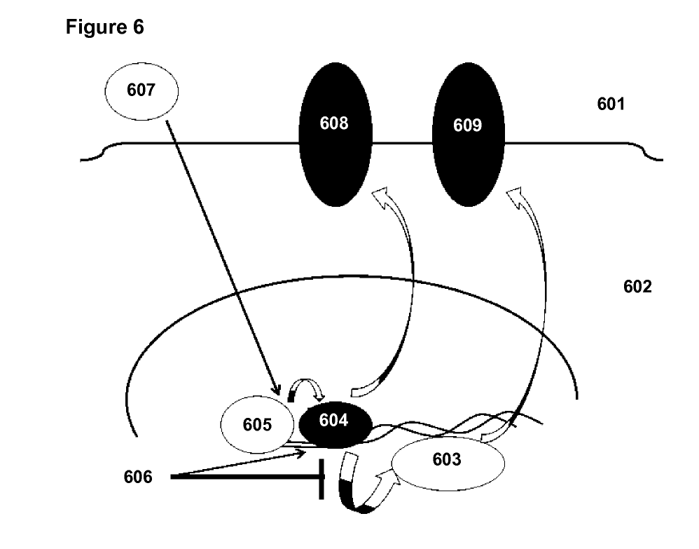

TGF signaling involves a series of serine/threonine phosphorylation events, and its regulation is related to the activity of the MAPK-MEK-ERK pathway, a receptor tyrosine kinase (RTK) activated pathway. Thus, in order to address potential crosstalk involving tyrosine phosphorylation-dependent pathways Applicants investigated the effect of Applicants' different protocols on tyrosine phosphorylation of multiple cellular proteins by western blotting. As shown in FIG. 2B, standard transgeneration conditions result in strong tyrosine phosphorylation of two major cellular proteins. Furthermore, TGF beta inhibition further enhances tyrosine phosphorylation, likely resulting from a negative feedback loop involving ERK and SMADs (see also FIG. 6).

Applicants also analyzed RNA expression by microarray. Sorted, transdetermined CD34+ cells were compared to the initial population of MSCs and CD34+ progenitors isolated from bone marrow (FIGS. 5A and B). Gene Ontology studies showed strong a correlation of MSC-derived-CD34+ cells into hematopoietic related categories comparable to bone marrow-derived-CD34+ cells. Pathway analysis of the microarray data pointed out a major role for TGFβ signaling (FIG. 9). Moreover, Applicants identified 372 genes commonly regulated between MSC-derived-CD34+ and BM-derived-CD34+ cells (FIG. 5B).

Applicants next compared TGFβ inhibition to Sox2 protein transduction, and found that Sox2 protein transduction consistently gave rise to around 20-25% CD34+ cells in a five-day period (FIG. 5C). Thus, protein transduction can also be used as a safe way to obtain MSC-derived-HPCs (FIG. 6).

Applicants found that inhibition of the TGFβ pathway consistently produces between 10-25% CD34+ cells. Strikingly, Applicants also observed strong attenuation of the progression towards a CD34+CD45+ intermediate progenitor state (FIG. 5D and FIG. 7) when combined with Applicants' “standard” viral Sox2 transduction protocol (FIGS. 1C, 1E, 5D and 7). Addition of DMSO (solvent for the TGF beta inhibitor) resulted in variable CD34+ levels.

RNA expression analysis of the cells showed efficient upregulation of different hematopoietic markers including CD34, CD45 and the early hematopoietic markers SCL, Runx1, CD41 and CD43 among others (FIG. 1F and FIG. 7). The observed changes in gene expression were significantly abrogated by TGFβ inhibition (FIG. 5D and FIG. 7). The results show a predominant role for TGFβ signaling for mediating the transition of early CD34+ progenitor cells towards a more mature phenotype including CD34+CD45+ double positive cells (FIGS. 1E, 5D, 6, and 7).

### Example 9

### Role for ERK Signaling in Cell Reprogramming

Upregulation and activation of TGF leads to SMAD-mediated transcription of downstream target genes required for hematopoietic development (Larsson (2005) Oncogene 24:5676). The present results show that inhibition of TGF signaling leads to downregulation of early hematopoietic markers such as GATA2, Runx1, CD43 and CD41, demonstrating that TGF signaling has a role in the progression towards more mature hematopoietic progenitors. Moreover, inhibition of TGF signaling led to downregulation of endogenous Sox2 expression, while exogenous Sox2 led to upregulation of endogenous Sox2. Sox2 target genes include a variety of components of both TGF and MAPK signaling pathways (Lee et al. 2009 Stem Cells 27:1858; Zhong et al. (2010) Zhongguo Ying Yong Sheng Li Xue Za Zhi 26:15), and ERK is a negative regulator of TGF signaling. See FIG. 6.

Applicants next examined whether inhibition of ERK alone is sufficient to bypass the need for ectopic Sox2. Indeed, chronic inhibition of ERK functionally replaced Sox2 in the generation of up to 35% early CD34+ progenitors (FIG. 5D and FIG. 7). Thus, ERK inhibition contributes to the generation of early CD34+ HPCs, further allowing TGFβ signaling to drive the progression towards more mature progenitor phenotypes. The present results show that modulation of signaling pathways can safely generate HPCs with no exogenous DNA integration, thus allowing for successful transition into the clinic.

### Example 10

### Differentiation Capacity of HPCs Toward the Erythroid Lineage

One of the main challenges to obtaining functional blood lineages in vitro involves the differentiation of HPCs towards the erythroid lineage. TGFβ might actually impair the capacity of the CD34+ cells to give rise to hematopoietic lineages in favor of more endothelial lineages.

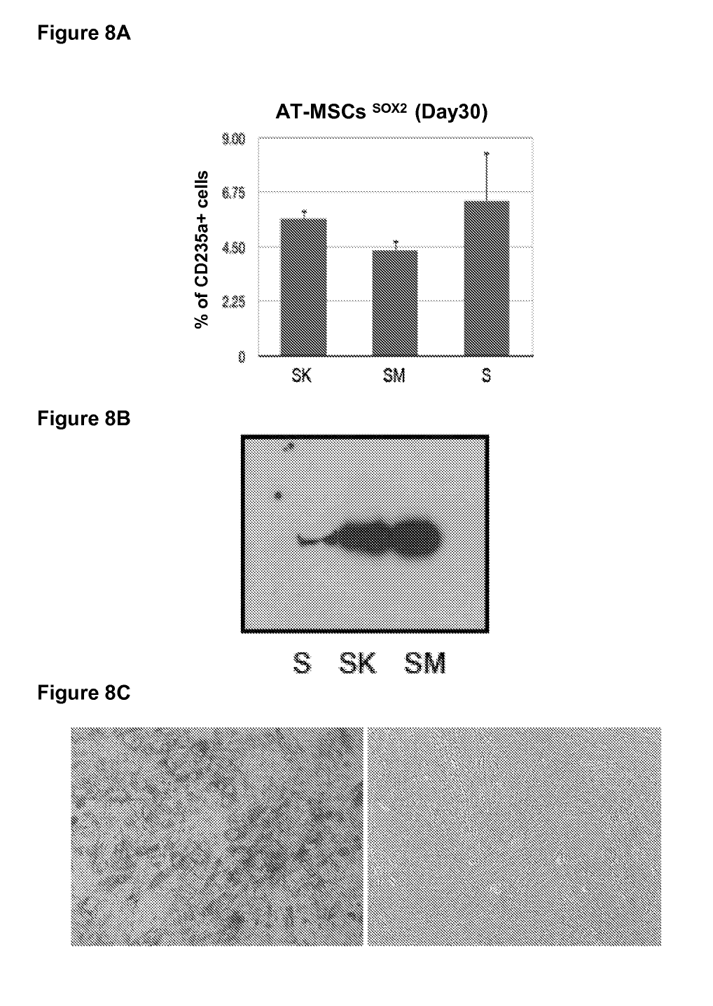

Applicants analyzed the differentiation potential towards the hematopoietic lineage of the CD34+ cells accumulated during chronic inhibition of TGFβ, which showed that sorted CD34+ cells retain multilineage potential and give rise to every major hematopoietic lineage (FIG. 8). Thus, TGFβ inhibition can be used for further accumulation of primitive CD34+ progenitor cells when a high number of cells are required. Applicants also observed unbiased differentiation towards the erythroid lineage that might be explained by the primitive nature of CD34+CD45− cells (FIGS. 3D and 8). Interestingly, not only did Applicants observe erythroid lineage differentiation in Colony-Forming-Assays but Applicants also observed low but consistent percentages of cells expressing the erythroid marker CD235a in long-term cultures without the addition of exogenous hematopoietic cytokines such as EPO (FIG. 8).

In view of the MSC-derived HPCs potential to generate cells belonging to the erythroid lineage, Applicants next evaluated the differentiation capacities of isolated HPCs. CD34 has been long speculated to represent a bona fide marker for the isolation of a population of cells containing human HPCs and has been routinely used for isolation of cord blood derived HPCs. Taking advantage of this surface marker, Applicants performed enrichments of CD34+ cells by Magnetic Activated Cell Sorting (MACS). The purity of the sorted cell population routinely lies between 85-95% (FIG. 8A). After enrichment, standard Hematopoietic Colony-Forming-Assays were performed. Applicants observed that transgenerated CD34+ cells are able to generate all major hematopoietic lineages, thus demonstrating their multipotent nature.

### Example 11

### Protocol Description for Hematopoietic Lineage Conversion

On day −1 somatic cells (fibroblasts, mesenchymal stem cells or any other cell type) are seeded the day before cell transduction. The cell number is estimate to 7.500-10.000 cells per cm². Plates are either plastic or coated with Matrigel®. Cells are maintained in an incubator (5% CO2, 37° C.). At this time, the culture media is a media described by the literature as supporting the growth of the starting cell type (referred to as Medium #1). On day 0 cells are transduced with either a retrovirus or lentivirus containing the human transcription factor Sox2 under the control of a promoter driving its expression in human cells. Plates are centrifuged at 1850 rpm for 1 hour in presence of polybrene (4 μg/ml) and then return back to an incubator (5% CO2, 37° C.). At this time, cells are maintained in Medium #1 in which virus and polybrene have been added. On day 1 culture medium is removed and replaced with Medium #2:DMEM-F12, Knockout Serum (20%), non essential amino acids (1%), L-glutamin (1%), 2-β Mercaptoethanol (0.1 mM), basic FGF-2 (10 ng/ml). Medium is changed every day and plates are maintained in an incubator (5% CO2, 37° C.). On day 3 culture medium is removed and replaced with Medium #3:IMDM, human serum (2%), Insulin-transferrin-selenium (1×), Human albumin (0.4%), basic FGF-2 (1 ng/ml). Media is changed every day and plates are maintained in an incubator (5% CO2, 37° C.). On day 7 the whole population is enzymatically collected and sorted for the marker CD34 by using either FACS or magnetic sorting procedures. Immediately after sorting the positive fraction is re-plated at 5.000 cells per cm². Plates are either plastic or coated with Matrigel®. Cells are maintained in an incubator (5% CO2, 37° C.) in medium#3. Half of the medium#3 is added every other day until collection between day 12 to 16.

### Example 12

### Alternative Protocol Including miRNA(s) Transduction for Hematopoietic Lineage Conversion (Procedure#2)

This protocol is identical to the one in Example 11 until day 9. On day 9 cells are transduced with retrovirus or lentivirus containing the precursor sequence of one or a combination of the hsa-miRNA(s) mentioned (see the list of miRNAs in Table 5) under the control of a promoter driving its expression in human cells. Cells are maintained in an incubator (5% CO2, 37° C.) in the medium#3. Half of the medium#3 is added every other day until collection between day 12 to 16.

miRNA(s) overexpression can be achieved by using different strategies. There are three main approaches to delivering miRNA(s) to cells: viruses, transfection reagents, and electroporation. (1) A virus-based approach with a 3^{rd} generation lentiviral system is used. This technology requires the engineering of constructs that will express the small RNA as a precursor miRNA, which is then expressed in the cell and processed to form a functional microRNA mimic. Briefly, we firstly generate lentiviral particles and then transduce somatic cells by adding concentrated viral particles and polybrene (4 μg/ml) to the cell media. (2) Libraries of miRNAmimics (mimic oligos) and inhibitors (antagomiroligos) are available and can be delivered with a lipid-transfection reagent, as with siRNA. This possibility was tested by using a GFP siRNA and use a lipofectamine-based transfection in human fibroblasts and MSCs by following the manufacturer's instructions (Invitrogen®). Achieved efficiencies ranged from 80-100% as measured by flow cytometry. 93) Electroporation approach use electrical pulses that induce pore formation in cellular membranes to allow plasmids entering the cells. Electroporations are performed accordingly to manufacturer's instructions (Amaxanucleofector®).

1. A method of forming a CD34+ hematopoietic progenitor cell, comprising:
(i) contacting a mesenchymal stem cell with a SOX2 signaling agonist in vitro in an absence of an exogenous nucleic acid encoding a KLF4 protein, an absence of an exogenous nucleic acid encoding an OCT4 protein, and an absence of an exogenous nucleic acid encoding a cMYC protein;
(ii) introducing a miR-125b-1 nucleic acid into the mesenchymal stem cell; and
(iii) culturing the mesenchymal stem cell in vitro;
thereby forming the CD34+ hematopoietic progenitor cell.

2. (canceled)

3. The method of claim 2, wherein said method is conducted in the absence of feeder cells.

4. The method of claim 1, wherein said SOX2 signaling agonist is a TGF beta signaling antagonist or an ERK signaling antagonist.

5-7. (canceled)

8. A method of producing a population of CD34+ hematopoietic progenitor cells, comprising:
(i) contacting a plurality of mesenchymal stem cells in vitro with a SOX2 signaling agonist;
(ii) introducing miR-125b-1 nucleic acid into the plurality of mesenchymal stem cells; and
(iii) culturing said plurality of mesenchymal stem cells to form a population of CD34+ hematopoietic stem cells.

9. The method of claim 8, further comprising
separating the population of CD34+ hematopoietic progenitor cells, thereby forming a plurality of separated hematopoietic progenitor cells.

10. The method of claim 9, wherein said separating is carried out 4 or more days after said contacting.

11. (canceled)

12. The method of claim 9, further comprising introducing the plurality of separated hematopoietic progenitor cells into a mammal.

13. The method of claim 12, wherein said mammal is selected from a mouse, rat, rabbit, non-human primate, and human.

14. The method of claim 12, wherein said plurality of separated hematopoietic progenitor cells is autologous to said mammal.

15. The method of claim 12, wherein said plurality of separated hematopoietic progenitor cells is allogeneic to said mammal.

16. (canceled)

17. The method of claim 8, wherein said plurality of mesenchymal stem cells are obtained from olfactory tissue or adipose tissue.

18. The method of claim 1, wherein contacting said mesenchymal stem cell with a SOX2 signaling agonist in vitro comprises transducing said mesenchymal stem cell with a SOX2 protein or a SOX2 nucleic acid.

19. A method of forming a hematopoietic progenitor cell (HPC), said method comprising:
(i) transducing a mesenchymal stem cell (MSC) with a SOX2 protein or a SOX2 nucleic acid; and
(ii) allowing said MSC to form a HPC.

20-46. (canceled)

47. The method of claim 1, wherein the SOX-2 signaling agonist comprises SB431542.

48. A method of producing a human CD34+ hematopoietic progenitor cell, comprising:
(i) contacting a human mesenchymal stem cell in vitro with a SOX2 agonist in the absence of: (a) an exogenous nucleic acid encoding a KLF4 protein, (b) an exogenous nucleic acid encoding an OCT4 protein, (c) an exogenous nucleic acid encoding a cMYC protein, and (d) a feeder layer; and
(ii) introducing miRNA-125b-1 nucleic acid into said human mesenchymal stem cell, thereby producing said human CD34+ hematopoietic progenitor cell.

49. The method of claim 48, wherein contacting said human mesenchymal stem cell in vitro with a SOX2 agonist comprises:
(a) transducing said human mesenchymal stem cell with a nucleic acid encoding SOX2;
(b) contacting said human mesenchymal stem cell with a SOX2 protein;
(c) contracting said human mesenchymal stem cell with SB431542, or
(d) a combination of (a)-(c).

50. The method of claim 48, wherein the human mesenchymal stem cell is a human olfactory epithelial mesenchymal stem cell.

51. The method of claim 48, wherein the human CD34+ hematopoietic progenitor cell expresses CD45.

52. A method of treating a subject with a hematopoietic deficiency, comprising:
forming a population of separated CD34+ hematopoietic progenitor cells by the method of claim 9; and
introducing a therapeutically effective amount of the separated population of CD34+ cells to the subject, thereby treating the hematopoietic deficiency subject.

53. The method of claim 52, wherein the CD34+ hematopoietic progenitor cells are autologous to the subject.

54. The method of claim 52, wherein the CD34+ hematopoietic progenitor cells are allogeneic to the subject.
